(19) United States
(12) Patent Application Publication
(10) Pub. No.: US 2016/0215268 A1
(43) Pub. Date: Jul. 28, 2016

(54) Suspension Culturing of Pluripotent Stem Cells

(71) Applicant: Janssen Biotech, Inc., Horsham, PA (US)

(72) Inventors: Benjamin Fryer, Horsham, PA (US); Daina Laniauskas, Raritan, NJ (US)

(73) Assignee: Janssen Biotech, Inc., Horsham, PA (US)

(21) Appl. No.: 14/963,730

(22) Filed: Dec. 9, 2015

Related U.S. Application Data
(60) Provisional application No. 62/094,509, filed on Dec. 19, 2014.

(51) Int. Cl. C12N 5/071

(52) U.S. Cl. CPC C12N 5/0676; C12N 2506/02

(57) ABSTRACT

The present invention provides methods of differentiating pluripotent cells into beta cell using suspension clustering. The methods of the invention use control of one or more of pH, cell concentration, and retinoid concentration to generate a nearly homogenous population of PDX1/NKX6.1 co-expressing cells by suppressing precocious NGN3 expression and promoting NKX6.1 expression. Also, the nearly homogenous population of PDX1/NKX6.1 co-expressing cells may be further differentiated in vitro to form a population of pancreatic endocrine cells that co-express PDX1, NKX6.1, insulin and MAFA.

### CROSS REFERENCE TO RELATED APPLICATION

The present application claims the benefit of U.S. Provisional Patent Application Ser. No. 62/094,509, filed Dec. 19, 2014, which is incorporated herein by reference in its entirety for all purpose.

### FIELD OF THE INVENTION

The present invention relates to the differentiation of pluripotent cells to pancreatic endocrine progenitor cells and pancreatic endocrine cells. In particular, the invention relates to methods that utilize control of pH, cell concentration and retinoid concentration in the differentiation process to facilitate production of a homogeneous population of NKX6.1 and PDX1 co-expressing pancreatic endocrine progenitor cells that, when differentiated further in vitro, yield a more mature population, when compared to conventional differentiation methods, of pancreatic endocrine cells that co-express PDX1, NKX6.1, insulin and MAFA.

### BACKGROUND

Advances in cell-replacement therapy for Type I diabetes mellitus and a shortage of transplantable islets of Langerhans have focused interest on developing sources of insulin-producing cells, or β cells, appropriate for engraftment. One approach is the generation of functional β cells from pluripotent stem cells, such as, embryonic stem cells.

In vertebrate embryonic development, a pluripotent cell gives rise to a group of cells comprising three germ layers (ectoderm, mesoderm, and endoderm) in a process known as gastrulation. Tissues such as, thyroid, thymus, pancreas, gut, and liver, will develop from the endoderm, via an intermediate stage. The intermediate stage in this process is the formation of definitive endoderm.

By the end of gastrulation, the endoderm is partitioned into anterior-posterior domains that can be recognized by the expression of a panel of factors that uniquely mark anterior, mid, and posterior regions of the endoderm. For example, HHEX, and SOX2 identify the anterior region while CDX1, 2, and 4 identify the posterior region of the endoderm.

Migration of endoderm tissue brings the endoderm into close proximity with different mesodermal tissues that help in regionalization of the gut tube. This is accomplished by a plethora of secreted factors, such as fibroblast growth factors (“FGFs”), wingless type MMTV integration site (“WNTS”), transforming growth factor betas (“TGF-Bs”), retinoic acid (“RA”), and bone morphogenic protein (“BMP”) ligands and their antagonists. For example, FGF4 and BMP are reported to promote CDX2 expression in the presumptive hindgut endoderm and repress expression of the anterior genes HHEX and SOX2 (2000 Development, 127:1563-1567). WNT signaling has also been shown to work in parallel to FGF signaling to promote hindgut development and inhibit foregut fate (2007 Development, 134:2207-2217). Lastly, secreted retinoic acid by mesenchyme regulates the foregut-hindgut boundary (2002 Curr Biol, 12:1215-1220).

The level of expression of specific transcription factors may be used to designate the identity of a tissue. During transformation of the definitive endoderm into a primitive gut tube, the gut tube becomes regionalized into broad domains that can be observed at the molecular level by restricted gene expression patterns. For example, the regionalized pancreas domain in the gut tube shows a very high expression of PDX1 and very low expression of CDX2 and SOX2. PDX1, NKX6.1, pancreas transcription factor 1 subunit alpha (“PTF1A”), and NKX2.2 are highly expressed in pancreatic tissue; and expression of CDX2 is high in intestine tissue.

Formation of the pancreas arises from the differentiation of definitive endoderm into pancreatic endoderm. Dorsal and ventral pancreatic domains arise from the foregut epithelium. Foregut also gives rise to the esophagus, trachea, lungs, thyroid, stomach, liver, pancreas, and bile duct system.

Cells of the pancreatic endoderm express the pancreatic-duodenal homeobox gene PDX1. In the absence of PDX1, the pancreas fails to develop beyond the formation of ventral and dorsal buds. Thus, PDX1 expression marks a critical step in pancreatic organogenesis. The mature pancreas contains both, exocrine tissue and endocrine tissue arising from the differentiation of pancreatic endoderm.

D'Amour et al. describes the production of enriched cultures of human embryonic stem cell-derived definitive endoderm in the presence of a high concentration of activin and low serum (Nature Biotechnol 2005, 23:1534-1541; U.S. Pat. No. 7,704,738). Transplanting these cells under the kidney capsule of mice reportedly resulted in differentiation into more mature cells with characteristics of endodermal tissue (U.S. Pat. No. 7,704,738). Human embryonic stem cell derived definitive endoderm cells can be further differentiated into PDX1 positive cells after addition of FGF10 and retinoic acid (U.S. Patent App. Pub. No. 2005/0266554A1). Subsequent transplantation of these pancreatic precursor cells in the fat pad of immune deficient mice resulted in the formation of functional pancreatic endocrine cells following a 3-4 month maturation phase (U.S. Pat. No. 7,993,920 and U.S. Pat. No. 7,534,608).

Fisk et al. report a system for producing pancreatic islet cells from human embryonic stem cells (U.S. Pat. No. 7,033,831). Small molecule inhibitors have also been used for induction of pancreatic endocrine precursor cells. For example, small molecule inhibitors of TGF-β receptor and BMP receptors (Development 2011, 138:861-871; Diabetes 2011, 60:239-247) have been used to significantly enhance the number of pancreatic endocrine cells. In addition, small molecule activators have also been used to generate definitive endoderm cells or pancreatic precursor cells (Curr Opin Cell Biol 2009, 21:727-732; Nature Chem Biol 2009, 5:258-265).

Great strides have been made in improving protocols for culturing progenitor cells such as pluripotent stem cells. PCT Publication No. WO2007/026353 (Amit et al.) discloses maintaining human embryonic stem cells in an undifferentiated state in a two-dimensional culture system. Ludwig et al., 2006 (Nature Biotechnology, 24: 185-7) discloses a TeSR1 defined medium for culturing human embryonic stem cells on a matrix. U.S. Patent App. Pub. No. 2007/0155013 (Akaike et al.) discloses a method of growing pluripotent stem cells in suspension using a carrier that adheres to the pluripotent stem cells, and U.S. Patent App. Pub. No. 2009/0029462 (Beardsley et al.) discloses methods of expanding pluripotent stem cells in suspension using microcarriers or cell encapsulation. PCT Publication No. WO 2008/015682 (Amit et al.) discloses a method of expanding and maintaining human embryonic stem cells in a suspension culture under culturing conditions devoid of substrate adherence. U.S. Patent App. Pub. No. 2008/0159994 (Mantalaris et al.) discloses a method of culturing human embryonic stem cells encapsulated within alginate beads in a three-dimensional culture system.

The art, including Rezania et. al. (Nature Biotechnology, 32:1121-1133 (2014)), Pagliuca et al (Cell, 159: 428-439 (2014)) and U.S Pat. No. 8,859,286 (Agulnick) teaches the need for the addition of components to modulate TGF-β or BMP signaling through either the direct blocking of BMP by using components such as BMP binders, for example Noggin, or a BMP receptor inhibitor, such as (6-(4-(2-(piperidin-1-yl)ethoxy)phenyl)-3-(pyridin-4-yl)pyrazolo[1,5-a]pyrimidine, hydrochloride or, alternatively, adding a TGF-β family member to occupy the receptors and indirectly block BMP signaling. Finally, it is taught that the use of a sonic hedgehog inhibitor in Stage 3, such as SANT-1 or cyclopamine, is advantageous because repression of sonic hedgehog signaling can permit PDX1 and insulin expression (Hebrok et al, Genes & Development, 12:1705-1713 (1998)).

Despite these advances, a need still remains for improved methods to culture pluripotent stem cells in a three-dimensional culture system that may differentiate to functional endocrine cells.

### BRIEF DESCRIPTION OF THE DRAWINGS

FIG. 1A is a graph of the partial oxygen pressure from daily culture medium samples plotted as a function of time (days of differentiation) over the course of the differentiation protocols of Example 1.

FIG. 1B is a graph of the glucose levels from daily culture medium samples plotted as a function of time (days of differentiation) over the course of the differentiation protocols of Example 1.

FIG. 1C is a graph of the lactate levels from daily culture medium samples plotted as a function of time (days of differentiation) over the course of the differentiation protocols of Example 1.

FIG. 1D is a graph of the pH levels from daily culture medium samples plotted as a function of time (days of differentiation) over the course of the differentiation protocols of Example 1.

FIG. 2A is a graph of real time polymerase chain reaction (qRT-PCR) results for expression of PDX1 over the course of the differentiation protocols of Example 1 from Stage 1 through day 1 of Stage 5.1

FIG. 2B is a graph of real time polymerase chain reaction (qRT-PCR) results for expression of NKX6.1 over the course of the differentiation protocols of Example 1 from Stage 1 through day 1 of Stage 5.

FIG. 2C is a graph of real time polymerase chain reaction (qRT-PCR) results for expression of PAX4 over the course of the differentiation protocols of Example 1 from Stage 1 through day 1 of Stage 5.

FIG. 2D is a graph of real time polymerase chain reaction (qRT-PCR) results for expression of PAX6 over the course of the differentiation protocols of Example 1 from Stage 1 through day 1 of Stage 5.

FIG. 2E is a graph of real time polymerase chain reaction (qRT-PCR) results for expression of NEUROG3 (NGN3) over the course of the differentiation protocols of Example 1 from Stage 1 through day 1 of Stage 5.

FIG. 2F is a graph of real time polymerase chain reaction (qRT-PCR) results for expression of ABCC8 over the course of the differentiation protocols of Example 1 from Stage 1 through day 1 of Stage 5.

FIG. 2G is a graph of real time polymerase chain reaction (qRT-PCR) results for expression of chromogranin A (CHGA) over the course of the differentiation protocols of Example 1 from Stage 1 through day 1 of Stage 5.

FIG. 2H is a graph of real time polymerase chain reaction (qRT-PCR) results for expression of G6PC2 over the course of the differentiation protocols of Example 1 from Stage 1 through day 1 of Stage 5.

FIG. 2I is a graph of real time polymerase chain reaction (qRT-PCR) results for expression of IAPP over the course of the differentiation protocols of Example 1 from Stage 1 through day 1 of Stage 5.

FIG. 2J is a graph of real time polymerase chain reaction (qRT-PCR) results for expression of insulin over the course of the differentiation protocols of Example 1 from Stage 1 through day 1 of Stage 5.

FIG. 2K is a graph of real time polymerase chain reaction (qRT-PCR) results for expression of GC6 over the course of the differentiation protocols of Example 1 from Stage 1 through day 1 of Stage 5.

FIG. 2L is a graph of real time polymerase chain reaction (qRT-PCR) results for expression of PTF1A over the course of the differentiation protocols of Example 1 from Stage 1 through day 1 of Stage 5.

FIG. 2M is a graph of real time polymerase chain reaction (qRT-PCR) results for expression of NEUROD1 over the course of the differentiation protocols of Example 1 from Stage 1 through day 1 of Stage 5.

FIG. 3A is a graph of real time polymerase chain reaction (qRT-PCR) results for expression of PDX1 over the course of the differentiation protocols of Example 1 from Stage 5, day 3 through day 7 of Stage 6.

FIG. 3B is a graph of real time polymerase chain reaction (qRT-PCR) results for expression of NKX6.1 over the course of the differentiation protocols of Example 1 from Stage 5, day 3 through day 7 of Stage 6.

FIG. 3C is a graph of real time polymerase chain reaction (qRT-PCR) results for expression of PAX6 over the course of the differentiation protocols of Example 1 from Stage 5, day 3 through day 7 of Stage 6.

FIG. 3D is a graph of real time polymerase chain reaction (qRT-PCR) results for expression of NEUROD1 over the course of the differentiation protocols of Example 1 from Stage 5, day 3 through day 7 of Stage 6.

FIG. 3E is a graph of real time polymerase chain reaction (qRT-PCR) results for expression of NEUROG3 (NGN3) over the course of the differentiation protocols of Example 1 from Stage 5, day 3 through day 7 of Stage 6.

FIG. 3F is a graph of real time polymerase chain reaction (qRT-PCR) results for expression of SLC2A1 over the course of the differentiation protocols of Example 1 from Stage 5, day 3 through day 7 of Stage 6.

FIG. 3G is a graph of real time polymerase chain reaction (qRT-PCR) results for expression of PAX4 over the course of the differentiation protocols of Example 1 from Stage 5, day 3 through day 7 of Stage 6.

FIG. 3H is a graph of real time polymerase chain reaction (qRT-PCR) results for expression of PCSK2 over the course of the differentiation protocols of Example 1 from Stage 5, day 3 through day 7 of Stage 6.

FIG. 3I is a graph of real time polymerase chain reaction (qRT-PCR) results for expression of chromogranin A (CHGA) over the course of the differentiation protocols of Example 1 from Stage 5, day 3 through day 7 of Stage 6.

FIG. 3J is a graph of real time polymerase chain reaction (qRT-PCR) results for expression of chromogranin B (CHGB) over the course of the differentiation protocols of Example 1 from Stage 5, day 3 through day 7 of Stage 6.

FIG. 3K is a graph of real time polymerase chain reaction (qRT-PCR) results for expression of pancreatic polypeptide over the course of the differentiation protocols of Example 1 from Stage 5, day 3 through day 7 of Stage 6.

FIG. 3L is a graph of real time polymerase chain reaction (qRT-PCR) results for expression of PCSK1 over the course of the differentiation protocols of Example 1 from Stage 5, day 3 through day 7 of Stage 6.

FIG. 3M is a graph of real time polymerase chain reaction (qRT-PCR) results for expression of G6PC2 over the course of the differentiation protocols of Example 1 from Stage 5, day 3 through day 7 of Stage 6.

FIG. 3N is a graph of real time polymerase chain reaction (qRT-PCR) results for expression of glucagon over the course of the differentiation protocols of Example 1 from Stage 5, day 3 through day 7 of Stage 6.

FIG. 3O is a graph of real time polymerase chain reaction (qRT-PCR) results for expression of insulin over the course of the differentiation protocols of Example 1 from Stage 5, day 3 through day 7 of Stage 6.

FIG. 4 is a graph of FACS profiles of Stage 1 cells, differentiated according to the protocols of Example 1, and stained for: CD184/CXCR4 (Y-axis) co-stained with CD9 (X-axis); and CD184/CXCR4 (Y-axis) co-stained with CD99 (X-axis).

FIG. 5A is a graph of FACS profiles of Stage 4 cells, differentiated according to the protocols of Example 1, and stained for: chromogranin A (X-axis) co-stained with NKX6.1 (Y-axis); and PDX1 (X-axis) co-stained with Ki67 (Y-axis).

FIG. 5B is a graph of FACS profiles of Stage 4 cells, differentiated according to the protocols of Example 1, and stained for: chromogranin A (X-axis) co-stained with NKX2.2 (Y-axis); and NEUROD1 (X-axis) co-stained with APC-A (Y-axis).

FIG. 6A is a graph of FACS profiles of Stage 5 cells, differentiated according to the protocol of Example 1, condition A, and stained for:chromogranin A (X-axis) co-stained with NKX6.1 (Y-axis); chromogranin A (X-axis) co-stained with NKX.2 (Y-axis); C-peptide (X-axis) co-stained with NKX6.1 (Y-axis); and insulin (X-axis) co-stained with glucagon (Y-axis).

FIG. 6B is a graph of FACS profiles of Stage 5 cells, differentiated according to the protocol of Example 1, condition A and stained for: PDX1 (X-axis) co-stained with Ki67 (Y-axis); PAX6 (X-axis) co-stained with OCT4 (Y-axis); NEUROD1 (X-axis) co-stained with NKX6.1 (Y-axis); insulin (X-axis) co-stained with NKX6.1 (Y-axis); and PDX1 (X-axis) co-stained with NKX6.1 (Y-axis).

FIG. 7A is a graph of FACS profiles of Stage 5 cells, differentiated according to the protocol of Example 1, condition B, and stained for: chromogranin A (X-axis) co-stained with NKX6.1 (Y-axis); chromogranin A (X-axis) co-stained with NKX2.2 (Y-axis); C-peptide (X-axis) co-stained with NKX6.1 (Y-axis); and insulin (X-axis) co-stained with glucagon (Y-axis).

FIG. 7B is a graph of FACS profiles of Stage 5 cells, differentiated according to the protocol of Example 1, condition B and stained for: PDX1 (X-axis) co-stained with Ki67 (Y-axis); PAX6 (X-axis) co-stained with OCT4 (Y-axis); NEUROD1 (X-axis) co-stained with NKX6.1 (Y-axis); insulin (X-axis) co-stained with NKX6.1 (Y-axis); and PDX1 (X-axis) co-stained with NKX6.1 (Y-axis) .

FIG. 8A is a graph of FACS profiles of Stage 5 cells, differentiated according to the protocol of Example 1, condition C, and stained for: chromogranin A (X-axis) co-stained with NKX6.1 (Y-axis); chromogranin A (X-axis) co-stained with NKX2.2 (Y-axis); C-peptide (X-axis) co-stained with NKX6.1 (Y-axis); and insulin (X-axis) co-stained with glucagon (Y-axis).

FIG. 8B is a graph of FACS profiles of Stage 5 cells, differentiated according to the protocol of Example 1, condition C and stained for: PDX1 (X-axis) co-stained with Ki67 (Y-axis); PAX6 (X-axis) co-stained with OCT4 (Y-axis); NEUROD1 (X-axis) co-stained with NKX6.1 (Y-axis); insulin (X-axis) co-stained with NKX6.1 (Y-axis); and PDX1 (X-axis) co-stained with NKX6.1 (Y-axis).

FIG. 9A is a graph of FACS profiles of Stage 6 cells, differentiated according to the protocol of Example 1, condition A, and stained for: chromogranin A (X-axis) co-stained with NKX6.1 (Y-axis); chromogranin A (X-axis) co-stained with NKX2.2 (Y-axis); insulin (X-axis) co-stained with glucagon (Y-axis); C-peptide (X-axis) co-stained with NKX6.1 (Y-axis); and C-peptide (X-axis) co-stained with insulin (Y-axis).

FIG. 9B is a graph of FACS profiles of Stage 6 cells, differentiated according to the protocol of Example 1, condition A and stained for: PDX1 (X-axis) co-stained with Ki67 (Y-axis); PAX6 (X-axis) co-stained with OCT4 (Y-axis); NEUROD1 (X-axis) co-stained with NKX6.1 (Y-axis); insulin (X-axis) co-stained with NKX6.1 (Y-axis); and PDX1 (X-axis) co-stained with NKX6.1 (Y-axis).

FIG. 10A is a graph of FACS profiles of Stage 6 cells, differentiated according to the protocol of Example 1, condition B, and stained for: chromogranin A (X-axis) co-stained with NKX6.1 (Y-axis); chromogranin A (X-axis) co-stained with NKX.2 (Y-axis); insulin (X-axis) co-stained with glucagon (Y-axis); C-peptide (X-axis) co-stained with NKX6.1 (Y-axis); and C-peptide (X-axis) co-stained with insulin (Y-axis).

FIG. 10B is a graph of FACS profiles of Stage 6 cells, differentiated according to the protocol of Example 1, condition B and stained for: PDX1 (X-axis) co-stained with Ki67 (Y-axis); PAX6 (X-axis) co-stained with OCT4 (Y-axis); NEUROD1 (X-axis) co-stained with NKX6.1 (Y-axis); insulin (X-axis) co-stained with NKX6.1 (Y-axis); and PDX1 (X-axis) co-stained with NKX6.1 (Y-axis).

FIG. 11A is a graph of FACS profiles of Stage 6 cells, differentiated according to the protocol of Example 1, condition C, and stained for: chromogranin A (X-axis) co-stained with NKX6.1 (Y-axis); chromogranin A (X-axis) co-stained with NKX.2 (Y-axis); insulin (X-axis) co-stained with glucagon (Y-axis); C-peptide (X-axis) co-stained with NKX6.1 (Y-axis); and C-peptide (X-axis) co-stained with insulin (Y-axis).

FIG. 11B is a graph of FACS profiles of Stage 6 cells, differentiated according to the protocol of Example 1, condition C and stained for: PDX1 (X-axis) co-stained with Ki67 (Y-axis); PAX6 (X-axis) co-stained with OCT4 (Y-axis); NEUROD1 (X-axis) co-stained with NKX6.1 (Y-axis); insulin (X-axis) co-stained with NKX6.1 (Y-axis); and PDX1 (X-axis) co-stained with NKX6.1 (Y-axis).

FIG. 12 is a graph of quantitative reverse transcription polymerase chain reaction (qRT-PCR) results for expression of MAFA of Stage 4 cells (day 15), Stage 5 cells (days 19 and 22), and Stage 6 cells (days 25 and 29), differentiated according to the protocols of Example 1.

FIG. 13 is a micrograph of the expression of MAFA at day 7 of Stage 6 cells.

FIG. 14 is a flow diagram of the set points for pH, dissolved oxygen, and cell concentration for Stages 3 through 4 of Example 2.

FIG. 15A depicts two graphs showing the pH levels during continuous monitoring of pH from the initiation of Stage 3 through Stage 4, day 3 for the differentiation carried out in accordance with Example 2.

FIG. 15B depicts two graphs showing the dissolved oxygen levels during continuous monitoring of DO from the initiation of Stage 3 through Stage 4, day 3 for the differentiation carried out in accordance with Example 2.

FIG. 16A is a graph of the glucose levels from a daily culture medium sample plotted as a function of time from the initiation of Stage 3 through Stage 4, day 3 for the differentiation carried out in accordance with Example 2.

FIG. 16B is a graph of lactate levels from a daily culture medium sample plotted as a function of time from the initiation of Stage 3 through Stage 4, day 3 for the differentiation carried out in accordance with Example 2.

FIG. 17 is a graph of the cell counts from a daily culture medium sample plotted as a function of time from the initiation of Stage 3 through Stage 4, day 3 for the differentiation carried out in accordance with Example 2.

FIG. 18A is a graph of real time qRT-PCR results for expression of PDX1 over the course of the differentiation protocols of Example 2 from Stage 3, day 1 through day 2 of Stage 4.

FIG. 18B is a graph of real time qRT-PCR results for expression of NKX6.1 over the course of the differentiation protocols of Example 2 from Stage 3, day 1 through day 2 of Stage 4.

FIG. 18C is a graph of real time qRT-PCR results for expression of PAX4 over the course of the differentiation protocols of Example 2 from Stage 3, day 1 through day 2 of Stage 4.

FIG. 18D is a graph of real time qRT-PCR results for expression of PAX6 over the course of the differentiation protocols of Example 2 from Stage 3, day 1 through day 2 of Stage 4.

FIG. 18E is a graph of real time qRT-PCR results for expression of NEUROG3 (NGN3) over the course of the differentiation protocols of Example 2 from Stage 3, day 1 through day 2 of Stage 4.

FIG. 18F is a graph of real time qRT-PCR results for expression of ABCC8 over the course of the differentiation protocols of Example 2 from Stage 3, day 1 through day 2 of Stage 4.

FIG. 18G is a graph of real time qRT-PCR results for expression of chromogranin A over the course of the differentiation protocols of Example 2 from Stage 3, day 1 through day 2 of Stage 4.

FIG. 18H is a graph of real time qRT-PCR results for expression of chromogranin B over the course of the differentiation protocols of Example 2 from Stage 3, day 1 through day 2 of Stage 4.

FIG. 18I is a graph of real time qRT-PCR results for expression of ARX over the course of the differentiation protocols of Example 2 from Stage 3, day 1 through day 2 of Stage 4.

FIG. 18J is a graph of real time qRT-PCR results for expression of ghrelin over the course of the differentiation protocols of Example 2 from Stage 3, day 1 through day 2 of Stage 4.

FIG. 18K is a graph of real time qRT-PCR results for expression of IAPP over the course of the differentiation protocols of Example 2 from Stage 3, day 1 through day 2 of Stage 4.

FIG. 18L is a graph of real time qRT-PCR results for expression of PTF1A over the course of the differentiation protocols of Example 2 from Stage 3, day 1 through day 2 of Stage 4.

FIG. 18M is a graph of real time qRT-PCR results for expression of NEUROD1 over the course of the differentiation protocols of Example 2 from Stage 3, day 1 through day 2 of Stage 4.

FIG. 18N is a graph of real time qRT-PCR results for expression of NKX2.2 over the course of the differentiation protocols of Example 2 from Stage 3, day 1 through day 2 of Stage 4.

FIG. 19 depicts graphs of FACS profiles of Stage 3 cells, differentiated according to the protocols of Example 2 with pH set points of 7.0 and 7.4 at Stage 3, and stained for: NKX6.1 (Y-axis) co-stained with NEUROD1 (X-axis).

FIG. 20 depicts graphs of FACS profiles of Stage 4 cells differentiated according to the protocols of Example 2 with pH set points of 7.0 and 7.4, at Stage 3 and stained for: NKX6.1 (Y-axis) co-stained with NEUROD1 (X-axis).

FIG. 21A is a graph of real time qRT-PCR results for expression of NEUROG3 over the course of the differentiation protocols of Example 2 from Stage 4, day 2 through day 7 of Stage 5.

FIG. 21B is a graph of real time qRT-PCR results for expression of NEUROD1 over the course of the differentiation protocols of Example 2 from Stage 4, day 2 through day 7 of Stage 5.

FIG. 21C is a graph of real time qRT-PCR results for expression of NKX2.2 over the course of the differentiation protocols of Example 2 from Stage 4, day 2 through day 7 of Stage 5.

FIG. 21D is a graph of real time qRT-PCR results for expression of ARX over the course of the differentiation protocols of Example 2 from Stage 4, day 2 through day 7 of Stage 5.

FIG. 21E is a graph of real time qRT-PCR results for expression of chromogranin A over the course of the differentiation protocols of Example 2 from Stage 4, day 2 through day 7 of Stage 5.

FIG. 21F is a graph of real time qRT-PCR results for expression of PCSK2 over the course of the differentiation protocols of Example 2 from Stage 4, day 2 through day 7 of Stage 5.

FIG. 21G is a graph of real time qRT-PCR results for expression of ABCC8 over the course of the differentiation protocols of Example 2 from Stage 4, day 2 through day 7 of Stage 5.

FIG. 21H is a graph of real time qRT-PCR results for expression of G6PC2 over the course of the differentiation protocols of Example 2 from Stage 4, day 2 through day 7 of Stage 5.

FIG. 21I is a graph of real time qRT-PCR results for expression of insulin over the course of the differentiation protocols of Example 2 from Stage 4, day 2 through day 7 of Stage 5.

FIG. 21J is a graph of real time qRT-PCR results for expression of ISL1 over the course of the differentiation protocols of Example 2 from Stage 4, day 2 through day 7 of Stage 5.

FIG. 21K is a graph of real time qRT-PCR results for expression of SLC2A1 over the course of the differentiation protocols of Example 2 from Stage 4, day 2 through day 7 of Stage 5.

FIG. 21L is a graph of real time qRT-PCR results for expression of SLC30A8 over the course of the differentiation protocols of Example 2 from Stage 4, day 2 through day 7 of Stage 5.

FIG. 21M is a graph of real time qRT-PCR results for expression of NKX6.1 over the course of the differentiation protocols of Example 2 from Stage 4, day 2 through day 7 of Stage 5.

FIG. 21N is a graph of real time qRT-PCR results for expression of UCN3 over the course of the differentiation protocols of Example 2 from Stage 4, day 2 through day 7 of Stage 5.

FIG. 21O is a graph of real time qRT-PCR results for expression of MAFA over the course of the differentiation protocols of Example 2 from Stage 4, day 2 through day 7 of Stage 5.

FIG. 21P is a graph of real time qRT-PCR results for expression of PPY over the course of the differentiation protocols of Example 2 from Stage 4, day 2 through day 7 of Stage 5.

FIG. 21Q is a graph of real time qRT-PCR results for expression of ghrelin over the course of the differentiation protocols of Example 2 from Stage 4, day 2 through day 7 of Stage 5.

FIG. 21R is a graph of real time qRT-PCR results for expression of GCG over the course of the differentiation protocols of Example 2 from Stage 4, day 2 through day 7 of Stage 5.

FIG. 21S is a graph of real time qRT-PCR results for expression of SST over the course of the differentiation protocols of Example 2 from Stage 4, day 2 through day 7 of Stage 5.

FIG. 22 depicts micrographs of the expression of insulin and MAFA in Stage 6, day 7 cells.

FIG. 23 depicts graphs of FACS profiles of Stage 5, day 6 cells, differentiated according to the protocols of Example 2 stained for: NKX6.1 (X-axis) co-stained with NEUROD1 (Y-axis), NKX6.1 (X-axis) as a function of cell count (Y-axis), and NEUROD1 (X-axis) as a function of cell count (Y-axis). The top graphs relate to condition A and bottom to condition C.

FIG. 24A depicts a graph of the pH levels during continuous monitoring of pH from the initiation of Stage 3 through Stage 5 for the differentiation carried out in reactors B, C, and D in accordance with Example 3.

FIG. 24B depicts a graph showing the dissolved oxygen levels during continuous monitoring of DO from the initiation of Stage 3 through Stage 5 for the differentiation carried out in reactors B, C, and D in accordance with Example 3.

FIG. 25 is a graph of cell counts from daily culture medium samples plotted as a function of time from the initiation of Stage 3 through Stage 5 for the differentiation carried out in reactors B C, and D in accordance with Example 3.

FIG. 26A is a graph of real time qRT-PCR results for expression of PDX1 over the course of the differentiation protocols of Example 3 in reactors B, C and D from Stage 3, day 1 through day 1 of Stage 5.

FIG. 26B is a graph of real time qRT-PCR results for expression of NKX6.1 over the course of the differentiation protocols of Example 3 in reactors B, C and D from Stage 3, day 1 through day 1 of Stage 5.

FIG. 26C is a graph of real time qRT-PCR results for expression of PAX4 over the course of the differentiation protocols of Example 3 in reactors B, C and D from Stage 3, day 1 through day 1 of Stage 5.

FIG. 26D is a graph of real time qRT-PCR results for expression of PAX6 over the course of the differentiation protocols of Example 3 in reactors B, C and D from Stage 3, day 1 through day 1 of Stage 5.

FIG. 26E is a graph of real time qRT-PCR results for expression of NEUROG3 over the course of the differentiation protocols of Example 3 in reactors B, C and D from Stage 3, day 1 through day 1 of Stage 5.

FIG. 26F is a graph of real time qRT-PCR results for expression of ABCC8 over the course of the differentiation protocols of Example 3 in reactors B, C and D from Stage 3, day 1 through day 1 of Stage 5.

FIG. 26G is a graph of real time qRT-PCR results for expression of chromogranin A over the course of the differentiation protocols of Example 3 in reactors B, C and D from Stage 3, day 1 through day 1 of Stage 5.

FIG. 26H is a graph of real time qRT-PCR results for expression of chromogranin B over the course of the differentiation protocols of Example 3 in reactors B, C and D from Stage 3, day 1 through day 1 of Stage 5.

FIG. 26I is a graph of real time qRT-PCR results for expression of ARX over the course of the differentiation protocols of Example 3 in reactors B, C and D from Stage 3, day 1 through day 1 of Stage 5.

FIG. 26J is a graph of real time qRT-PCR results for expression of ghrelin over the course of the differentiation protocols of Example 3 in reactors B, C and D from Stage 3, day 1 through day 1 of Stage 5.

FIG. 26K is a graph of real time qRT-PCR results for expression of IAPP over the course of the differentiation protocols of Example 3 in reactors B, C and D from Stage 3, day 1 through day 1 of Stage 5.

FIG. 26L is a graph of real time qRT-PCR results for expression of PFT1A over the course of the differentiation protocols of Example 3 in reactors B, C and D from Stage 3, day 1 through day 1 of Stage 5.

FIG. 26M is a graph of real time qRT-PCR results for expression of NEUROD1 over the course of the differentiation protocols of Example 3 in reactors B, C and D from Stage 3, day 1 through day 1 of Stage 5.

FIG. 26N is a graph of real time qRT-PCR results for expression of NKX2.2 over the course of the differentiation protocols of Example 3 in reactors B, C and D from Stage 3, day 1 through day 1 of Stage 5.

FIG. 27A is a graph of real time qRT-PCR results for expression of NEUROG3 over the course of the differentiation protocols of Example 4 from Stage 5, day 1 through day 7 of Stage 6.

FIG. 27B is a graph of real time qRT-PCR results for expression of NEUROD1 over the course of the differentiation protocols of Example 4 from Stage 5, day 1 through day 7 of Stage 6.

FIG. 27C is a graph of real time qRT-PCR results for expression of chromogranin A over the course of the differentiation protocols of Example 4 from Stage 5, day 1 through day 7 of Stage 6.

FIG. 27D is a graph of real time qRT-PCR results for expression of chromogranin B over the course of the differentiation protocols of Example 4 from Stage 5, day 1 through day 7 of Stage 6.

FIG. 27E is a graph of real time qRT-PCR results for expression of GCG over the course of the differentiation protocols of Example 4 from Stage 5, day 1 through day 7 of Stage 6.

FIG. 27F is a graph of real time qRT-PCR results for expression of IAPP over the course of the differentiation protocols of Example 4 from Stage 5, day 1 through day 7 of Stage 6.

FIG. 27G is a graph of real time qRT-PCR results for expression of ISL1 over the course of the differentiation protocols of Example 4 from Stage 5, day 1 through day 7 of Stage 6.

FIG. 27H is a graph of real time qRT-PCR results for expression of MAFB over the course of the differentiation protocols of Example 4 from Stage 5, day 1 through day 7 of Stage 6.

FIG. 27I is a graph of real time qRT-PCR results for expression of pancreatic polypeptide over the course of the differentiation protocols of Example 4 from Stage 5, day 1 through day 7 of Stage 6.

FIG. 27J is a graph of real time qRT-PCR results for expression of somatostatin over the course of the differentiation protocols of Example 4 from Stage 5, day 1 through day 7 of Stage 6.

FIG. 27K is a graph of real time qRT-PCR results for expression of insulin over the course of the differentiation protocols of Example 4 from Stage 5, day 1 through day 7 of Stage 6.

FIG. 27L is a graph of real time qRT-PCR results for expression of G6PC2 over the course of the differentiation protocols of Example 4 from Stage 5, day 1 through day 7 of Stage 6.

FIG. 27M is a graph of real time qRT-PCR results for expression of PCSK1 over the course of the differentiation protocols of Example 4 from Stage 5, day 1 through day 7 of Stage 6.

FIG. 27N is a graph of real time qRT-PCR results for expression of PCSK2 over the course of the differentiation protocols of Example 4 from Stage 5, day 1 through day 7 of Stage 6.

FIG. 27O is a graph of real time qRT-PCR results for expression of SLC30A8 over the course of the differentiation protocols of Example 4 from Stage 5, day 1 through day 7 of Stage 6.

FIG. 27P is a graph of real time qRT-PCR results for expression of NKX6.1 over the course of the differentiation protocols of Example 4 from Stage 5, day 1 through day 7 of Stage 6.

FIG. 27Q is a graph of real time qRT-PCR results for expression of NKX2.2 over the course of the differentiation protocols of Example 4 from Stage 5, day 1 through day 7 of Stage 6.

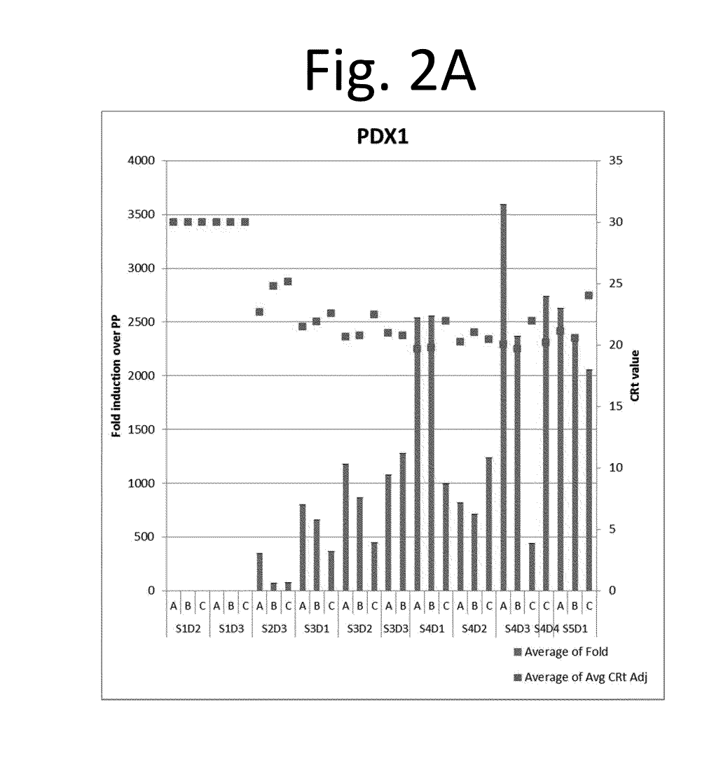

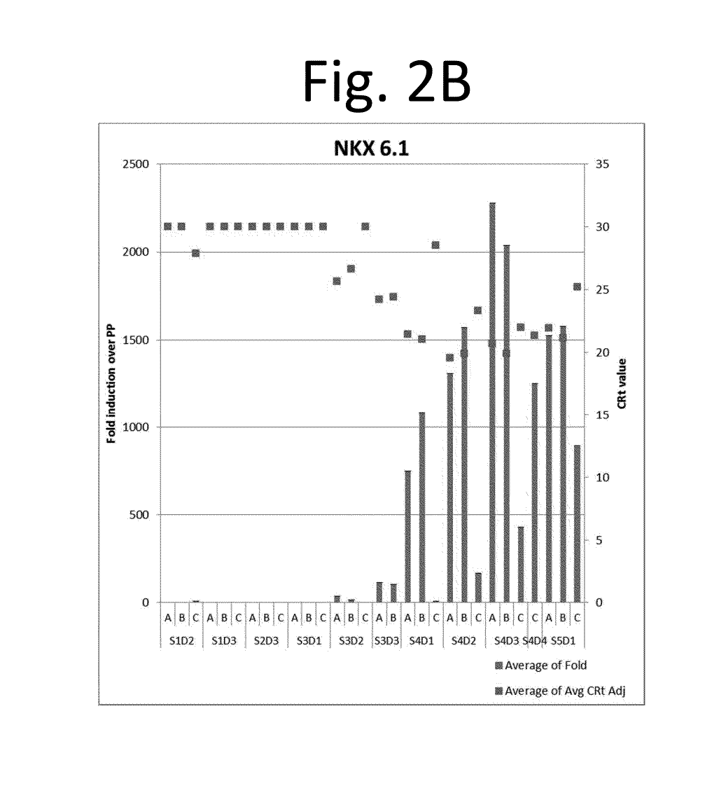

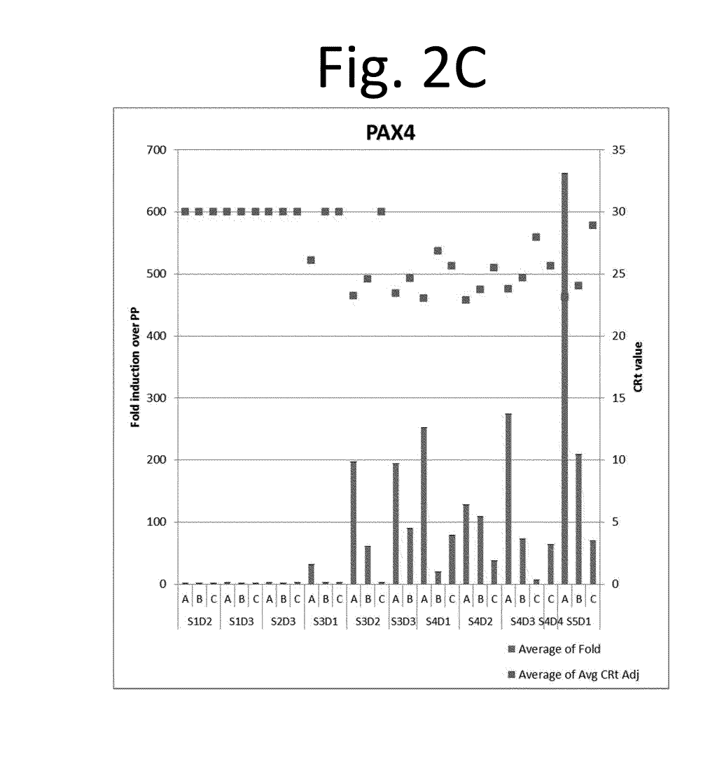

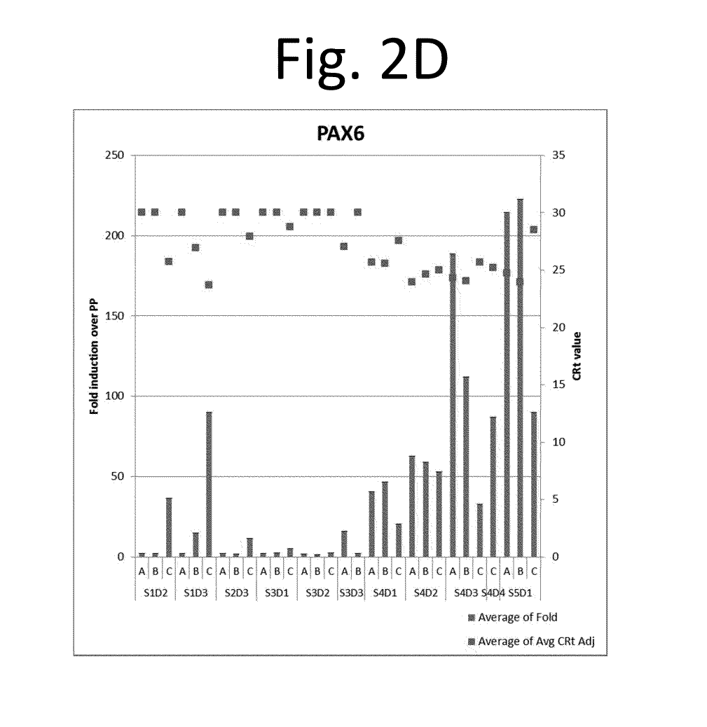

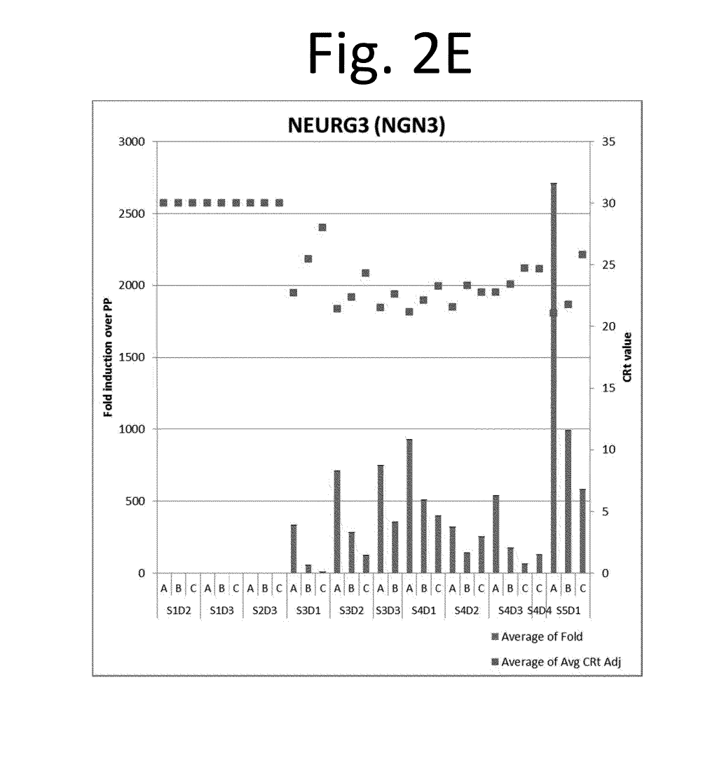

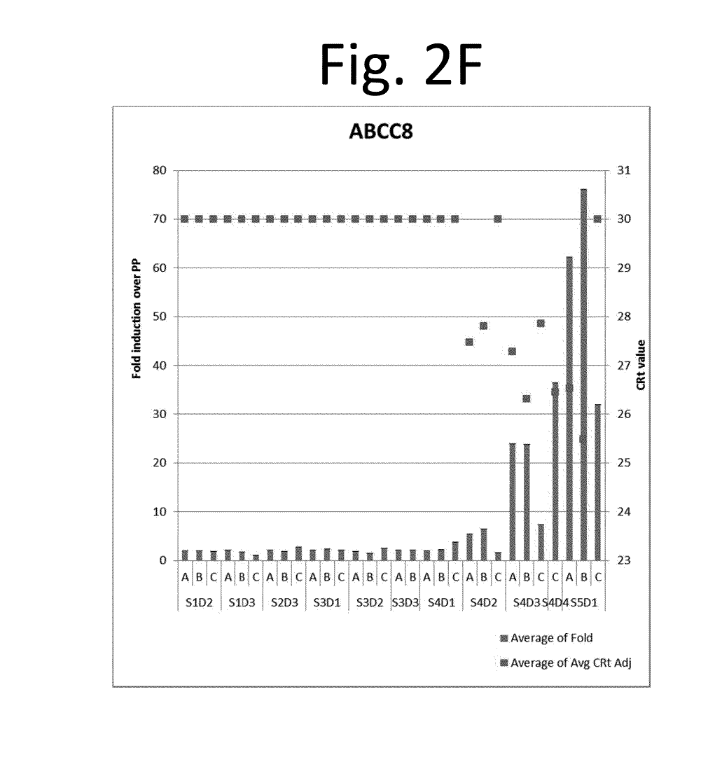

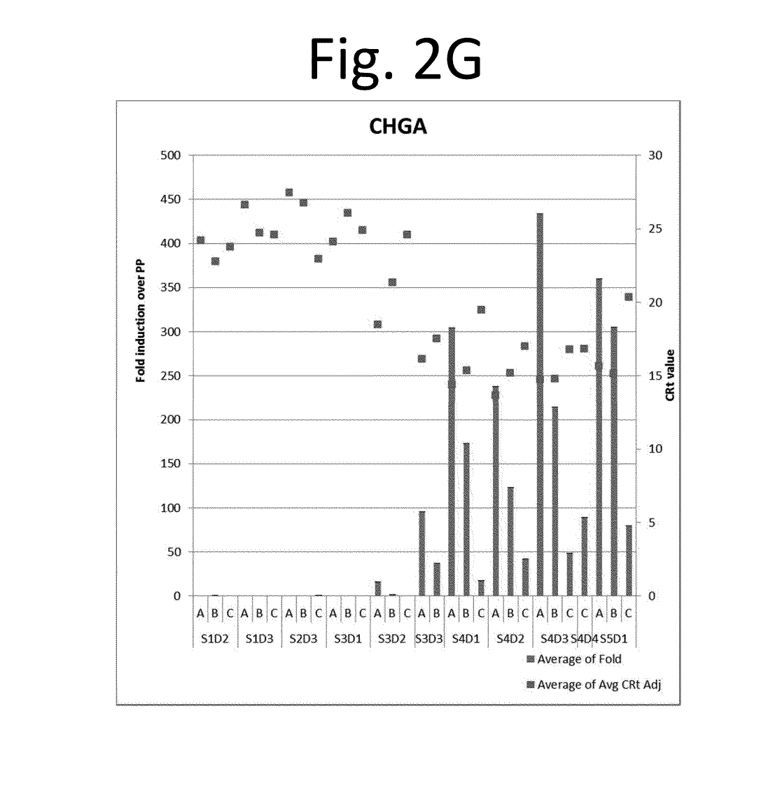

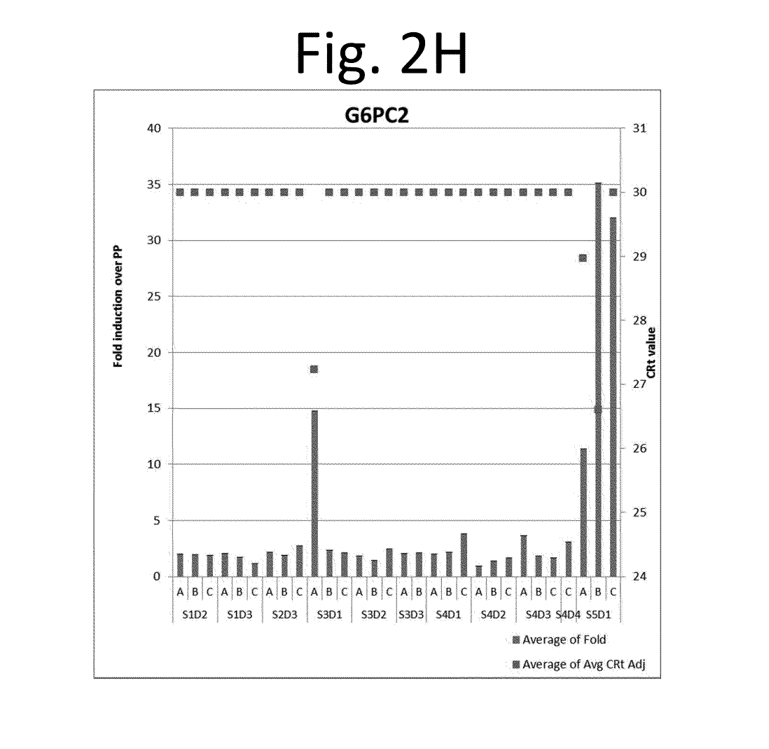

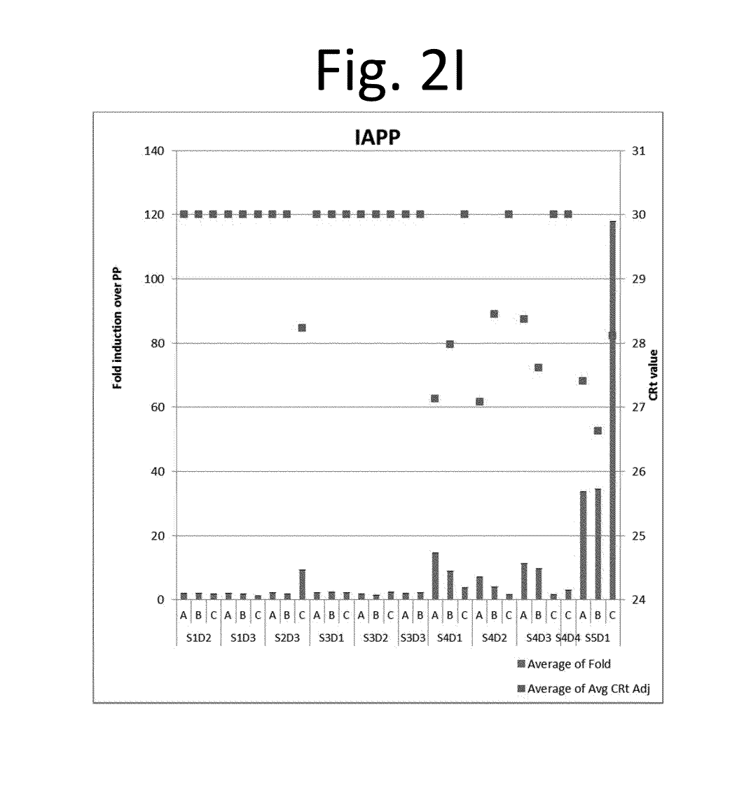

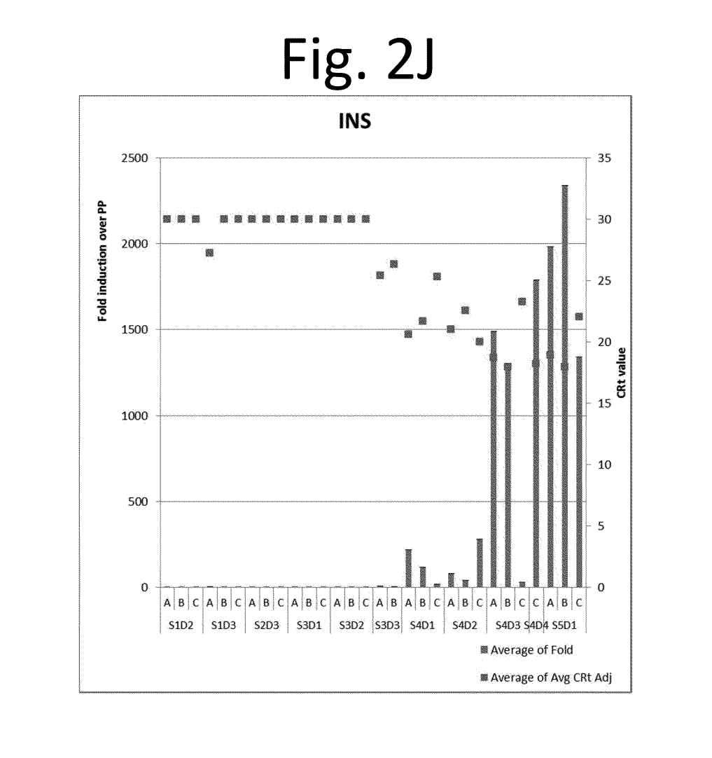

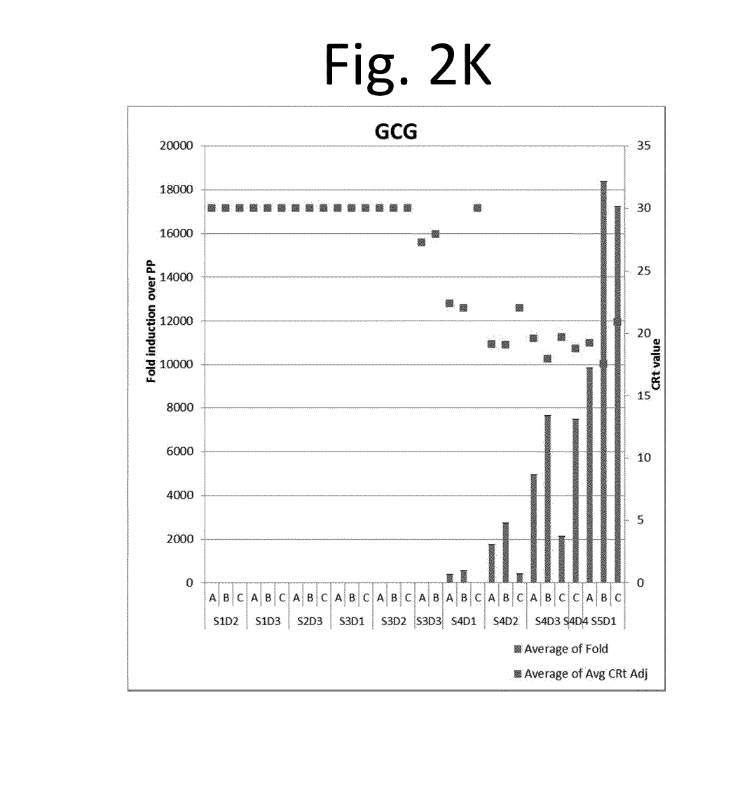

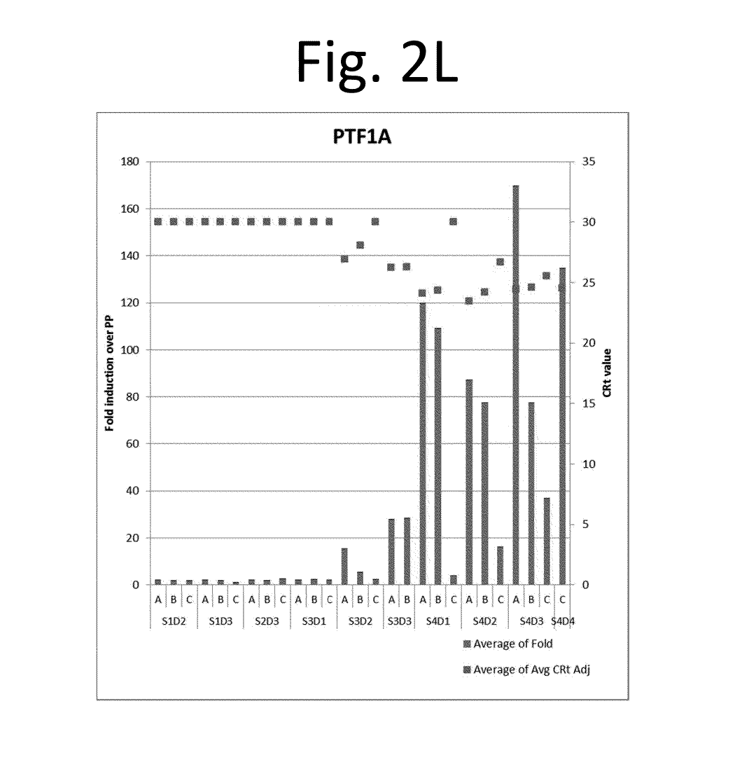

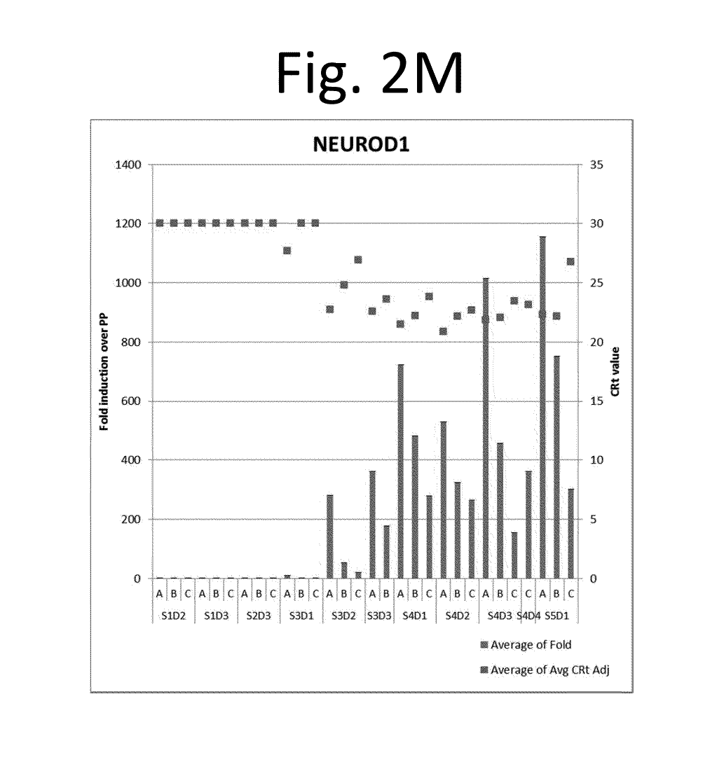

FIG. 2Y7R is a graph of real time qRT-PCR results for expression of MNX1 (HB9) over the course of the differentiation protocols of Example 4 from Stage 5, day 1 through day 7 of Stage 6.

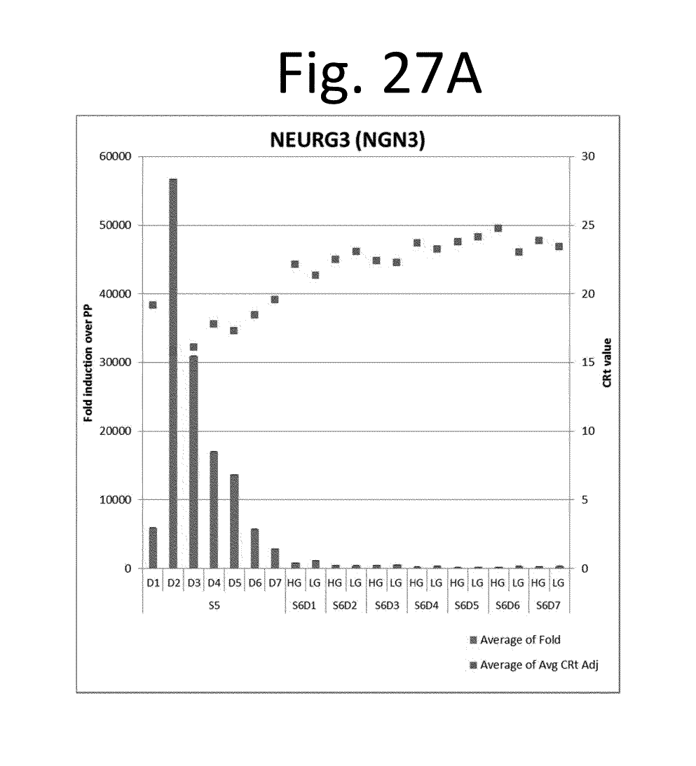

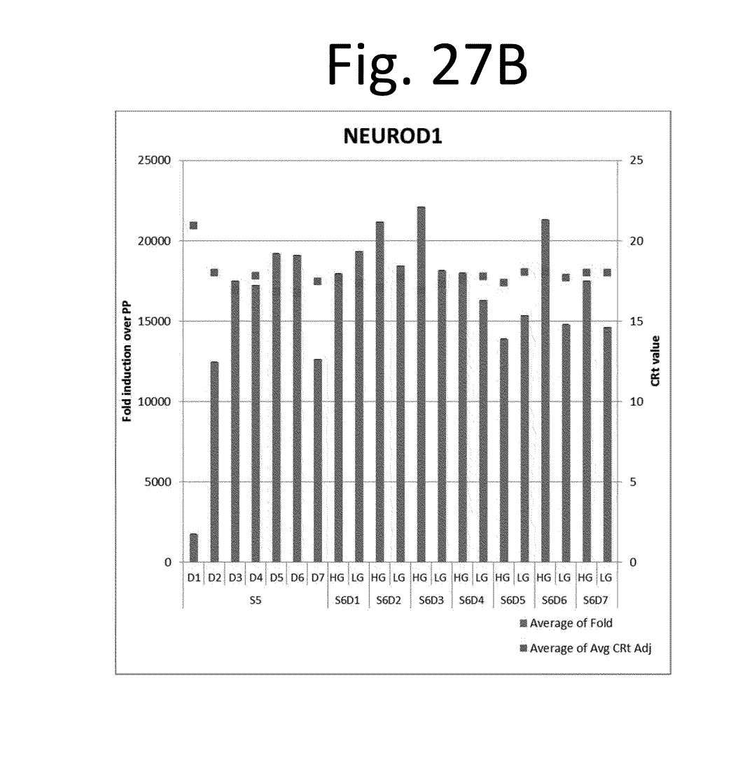

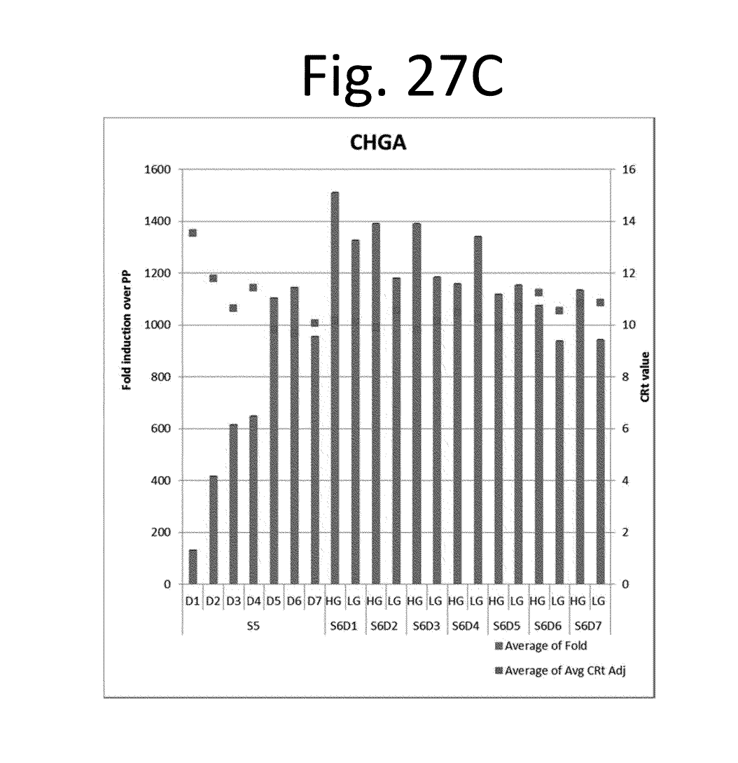

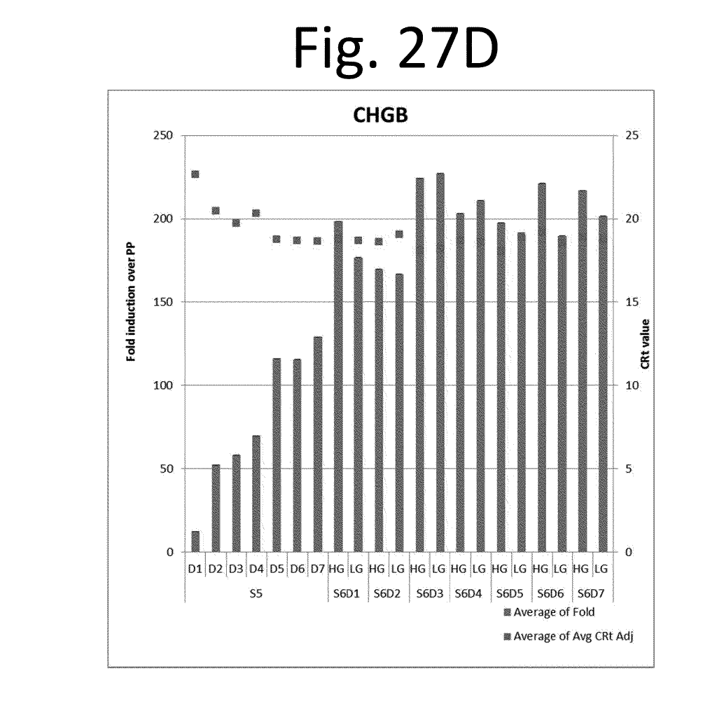

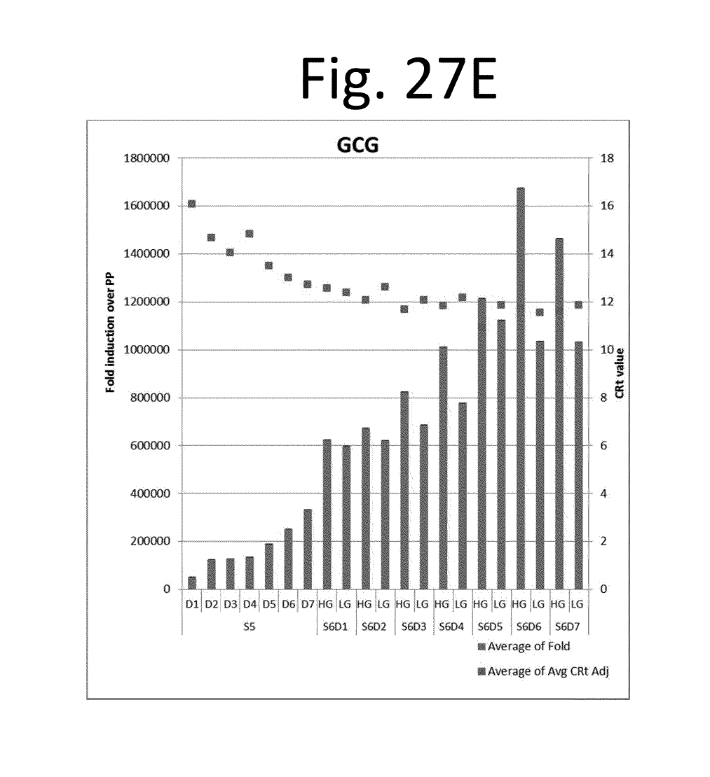

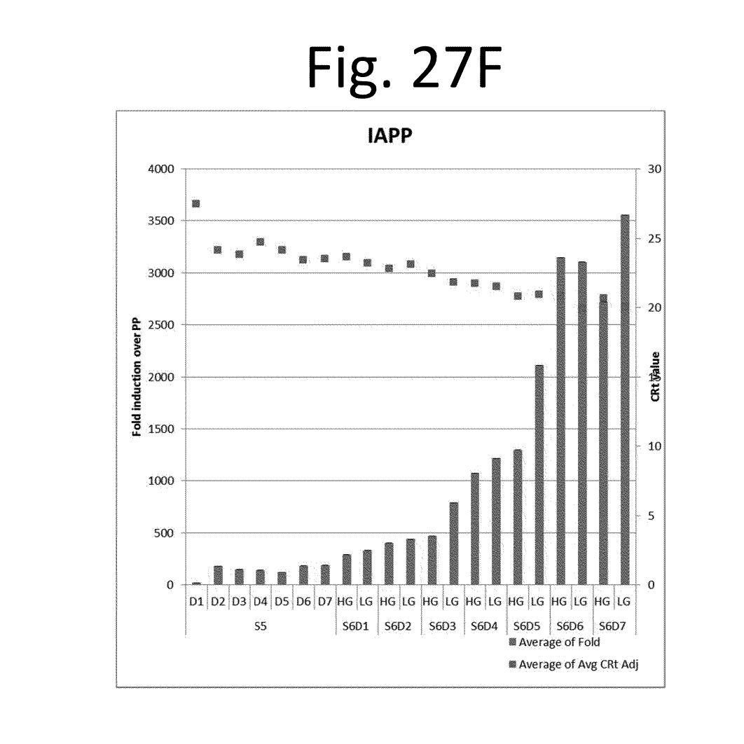

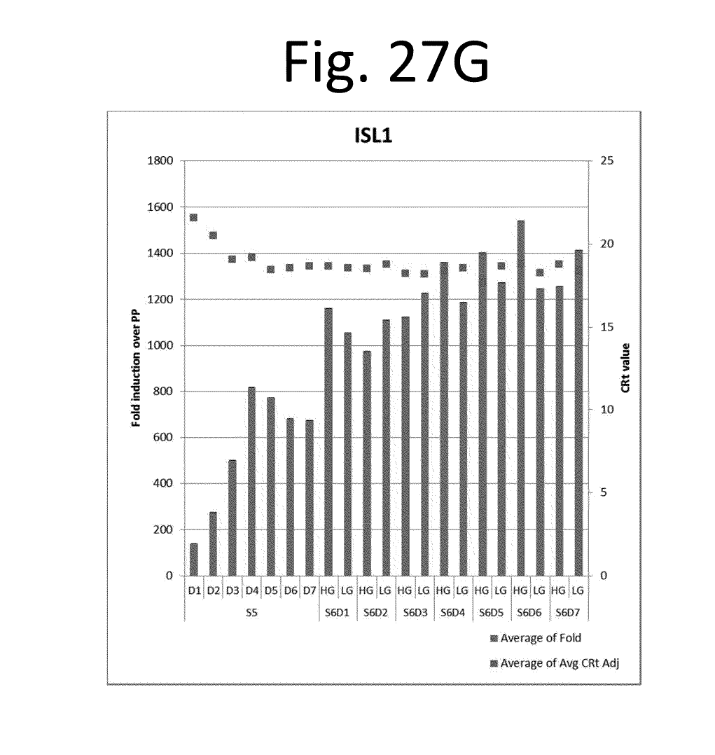

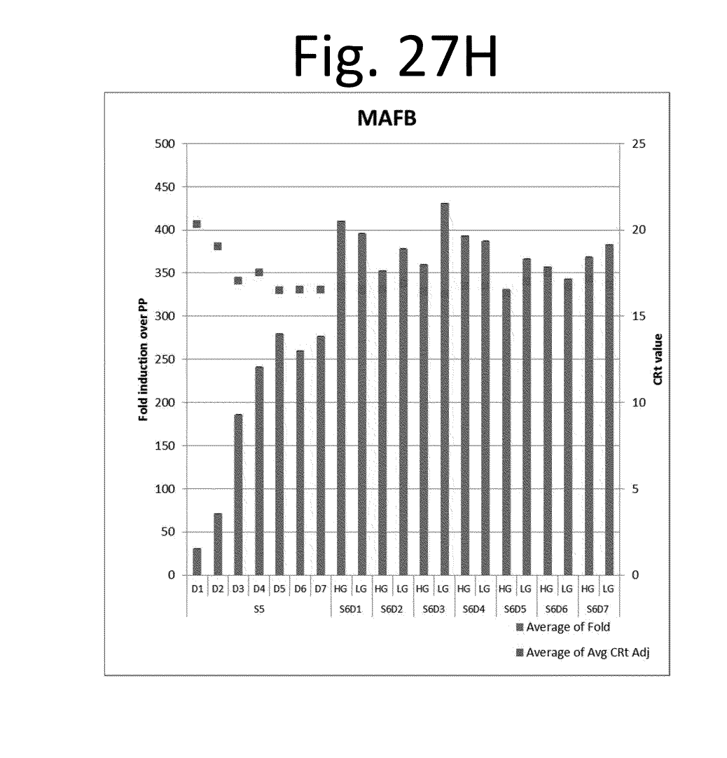

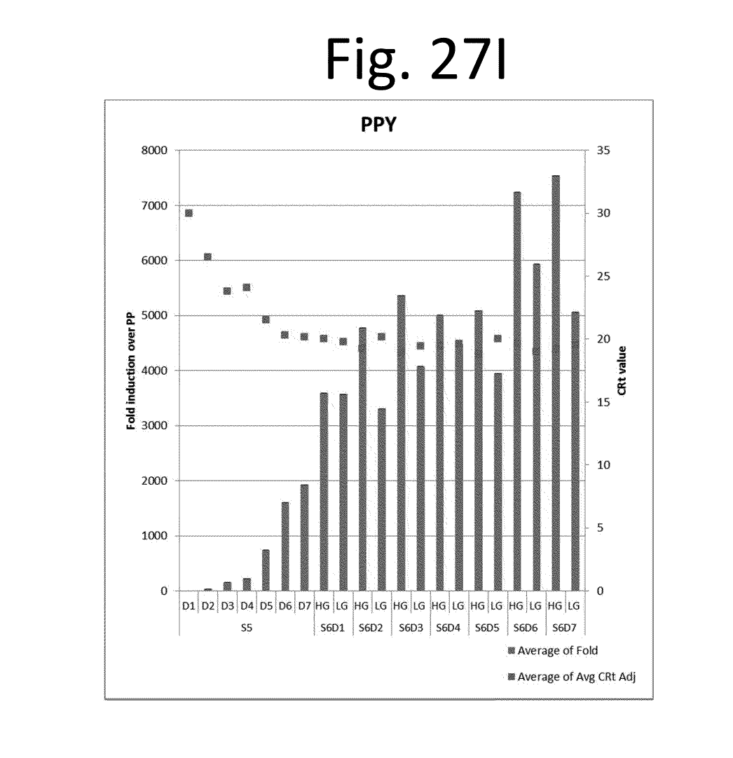

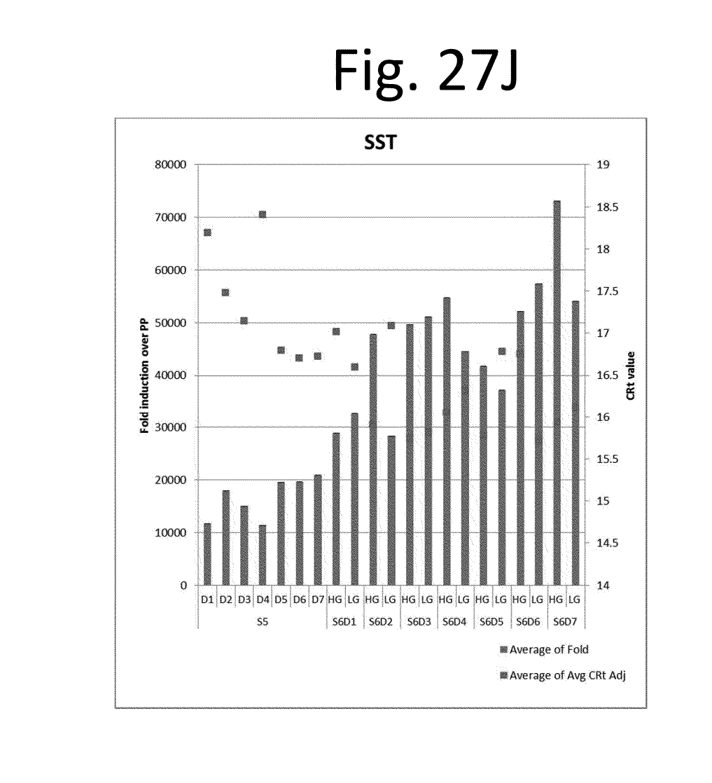

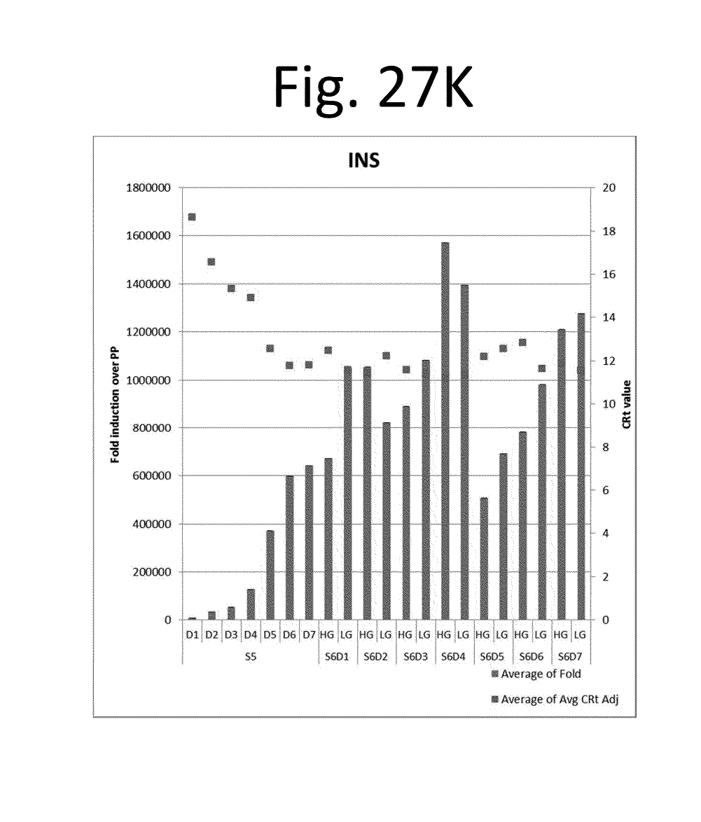

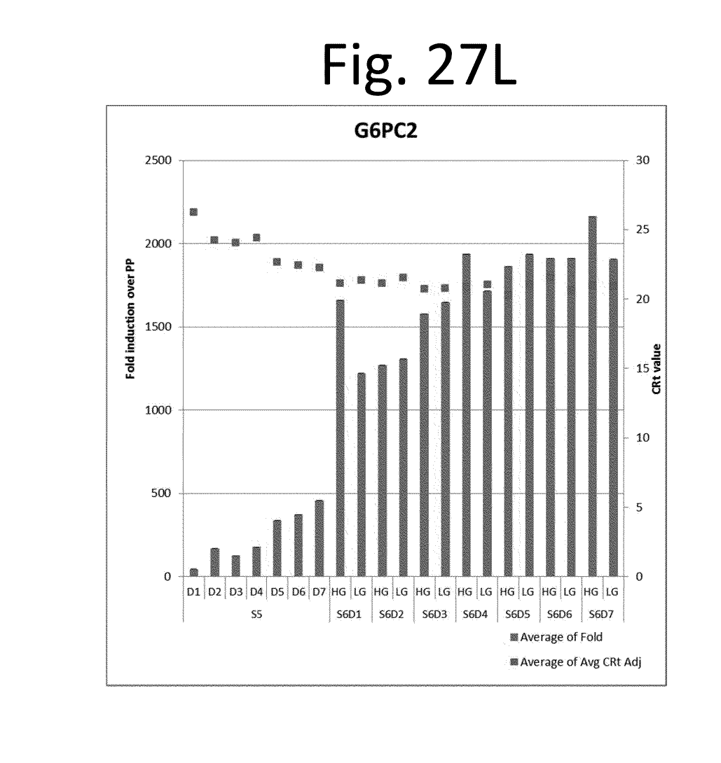

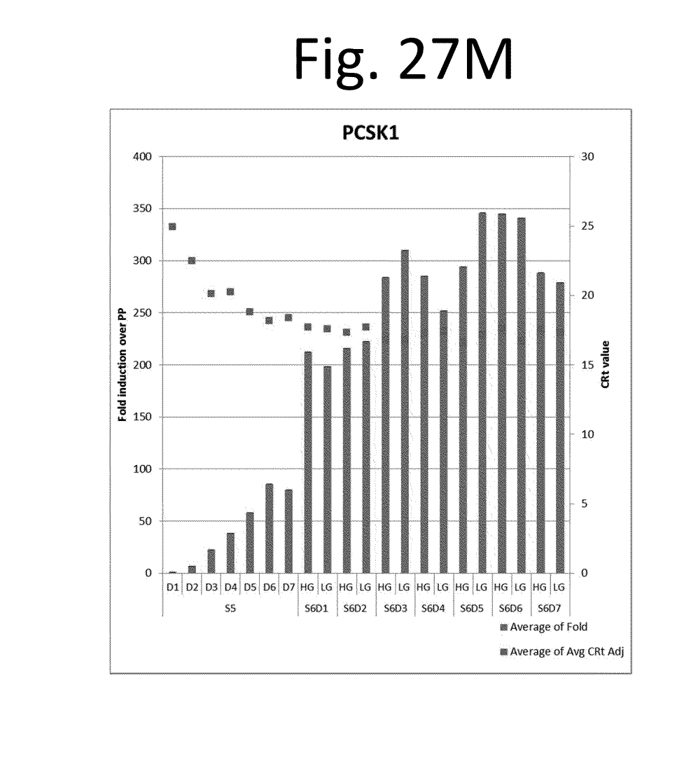

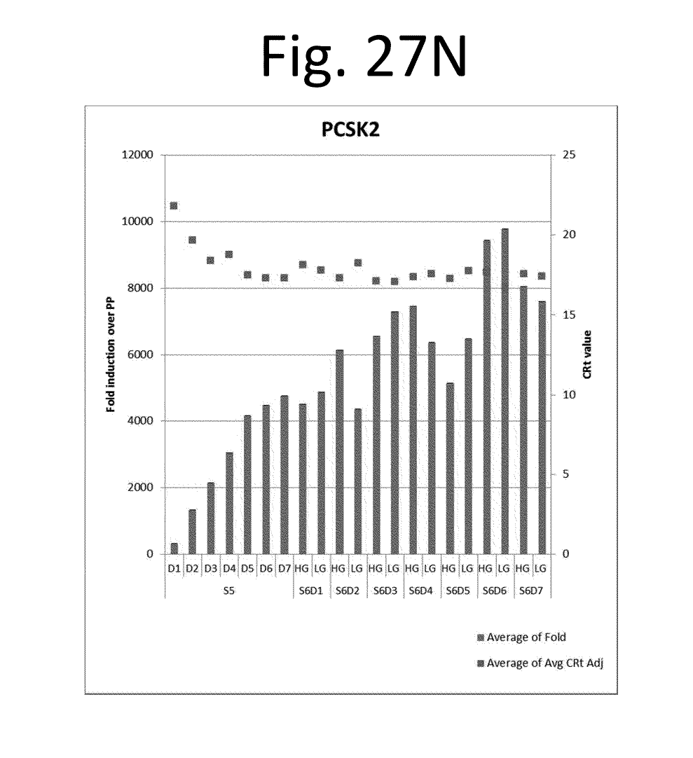

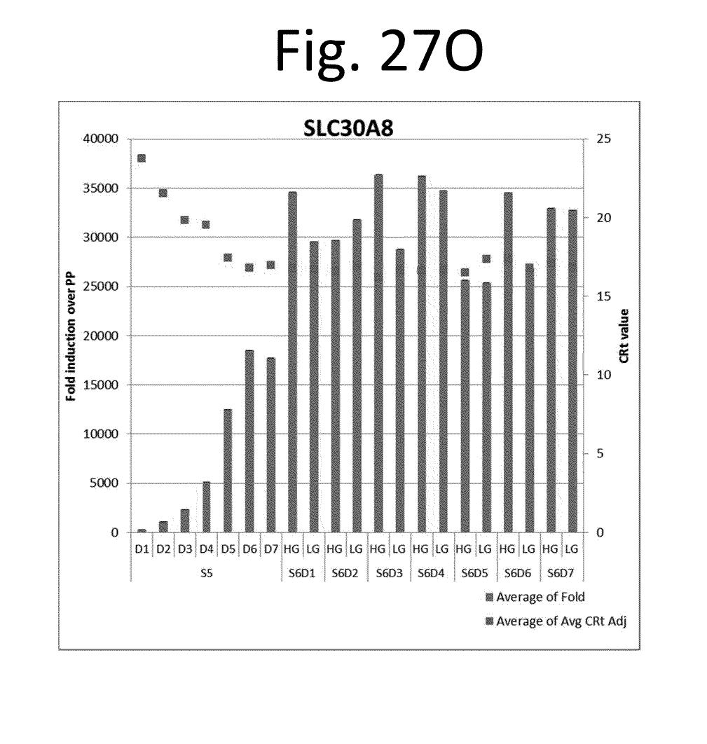

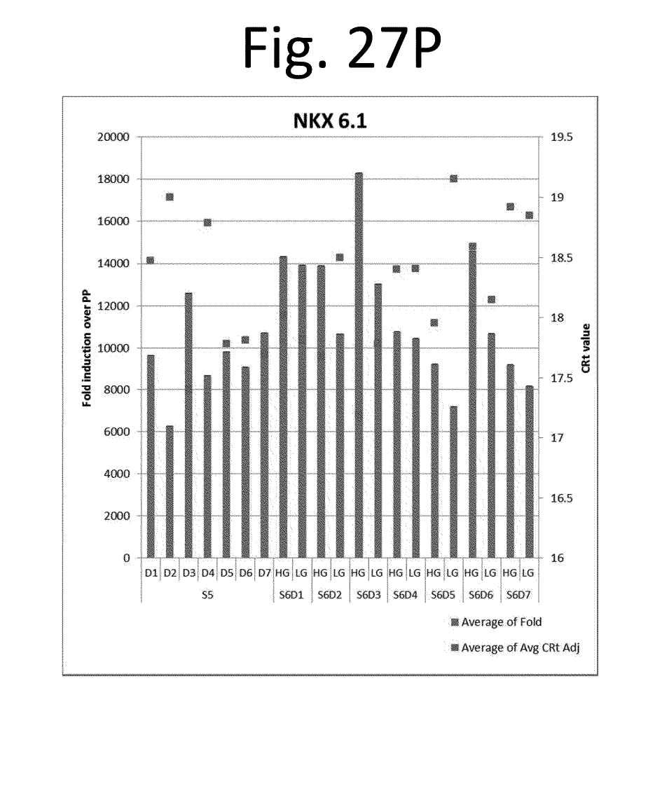

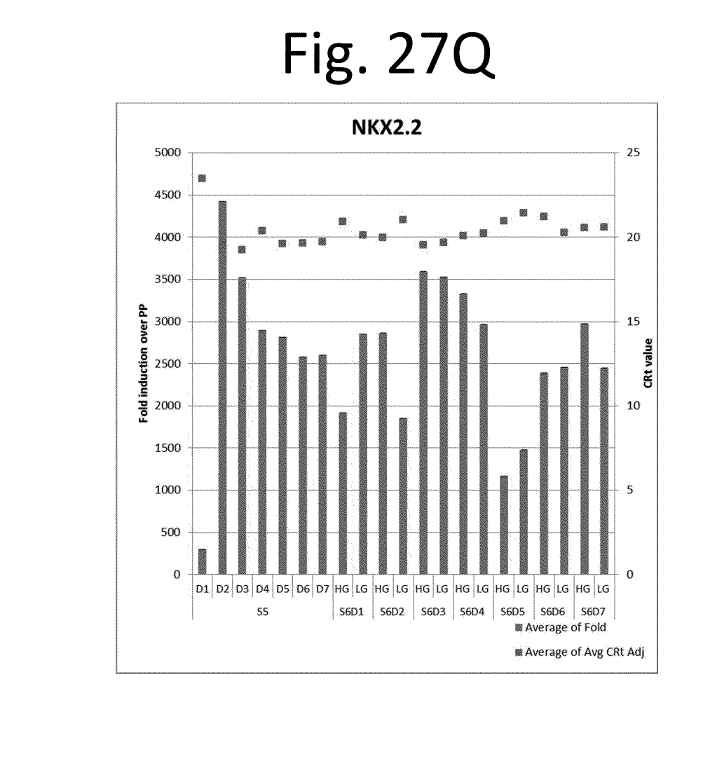

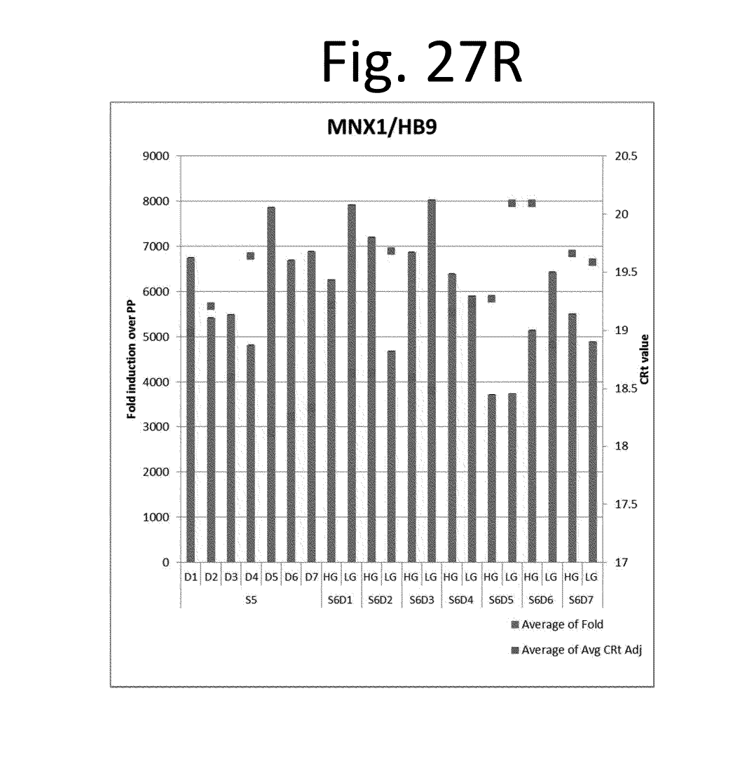

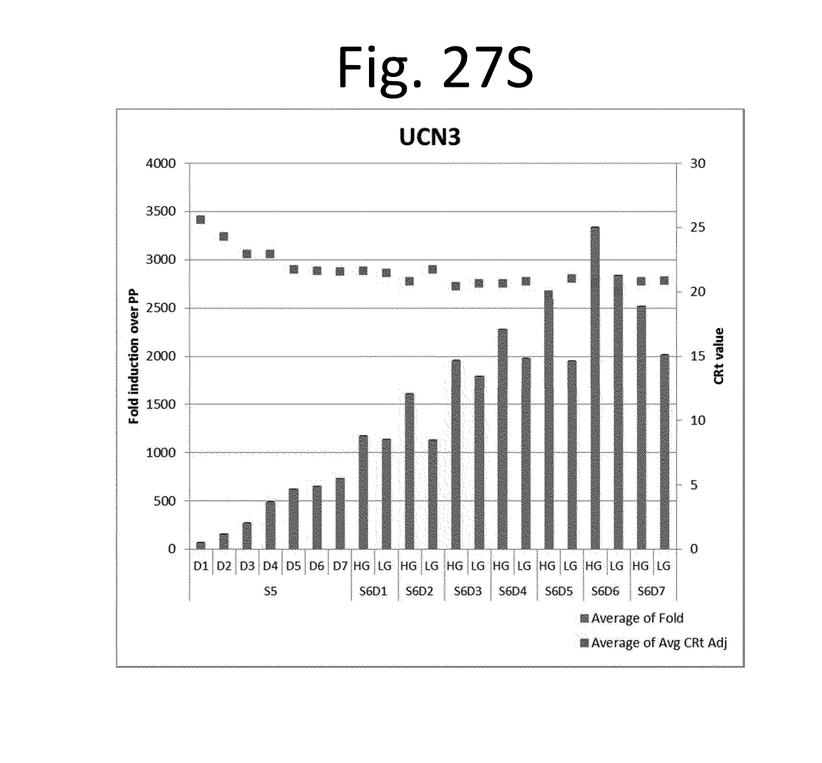

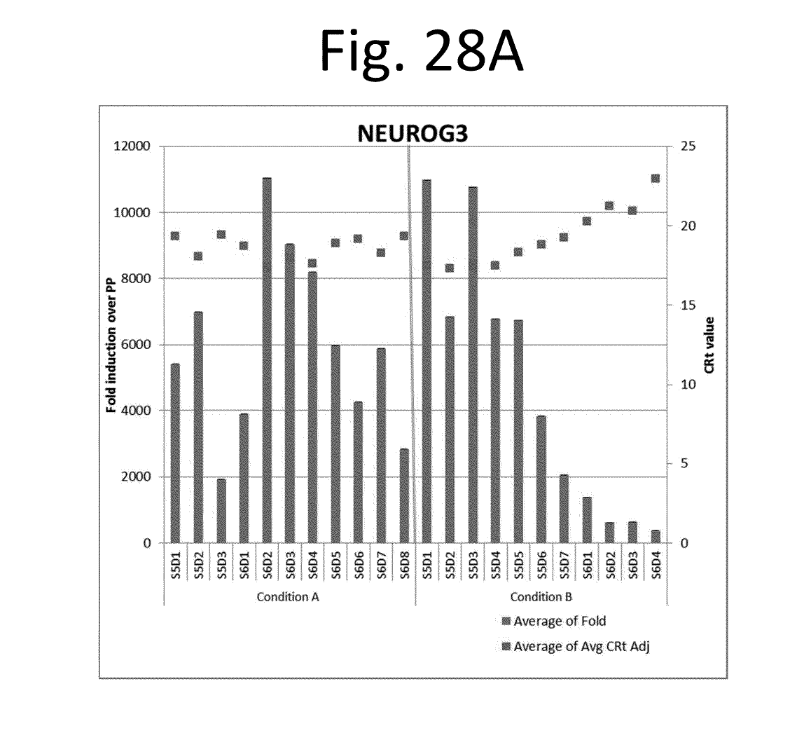

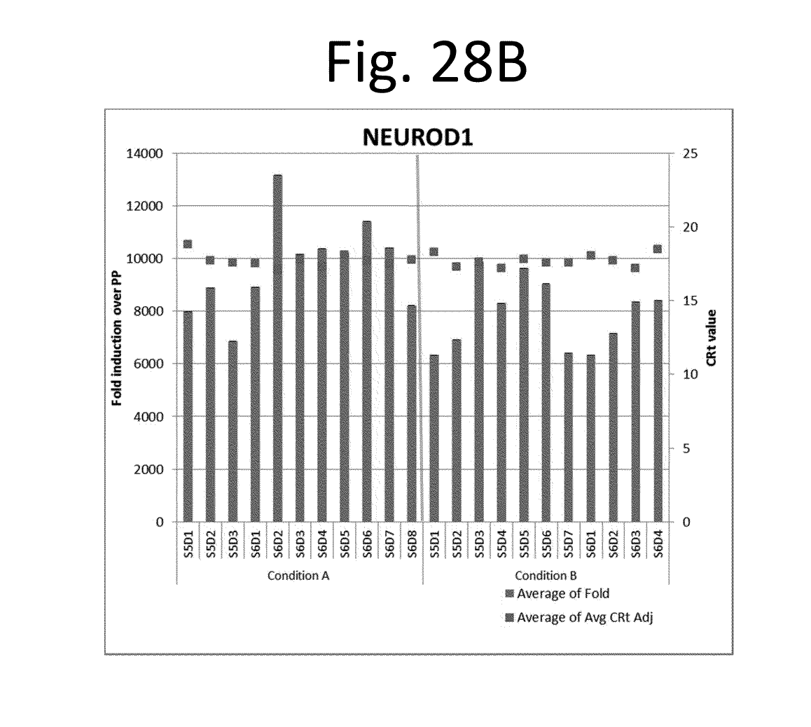

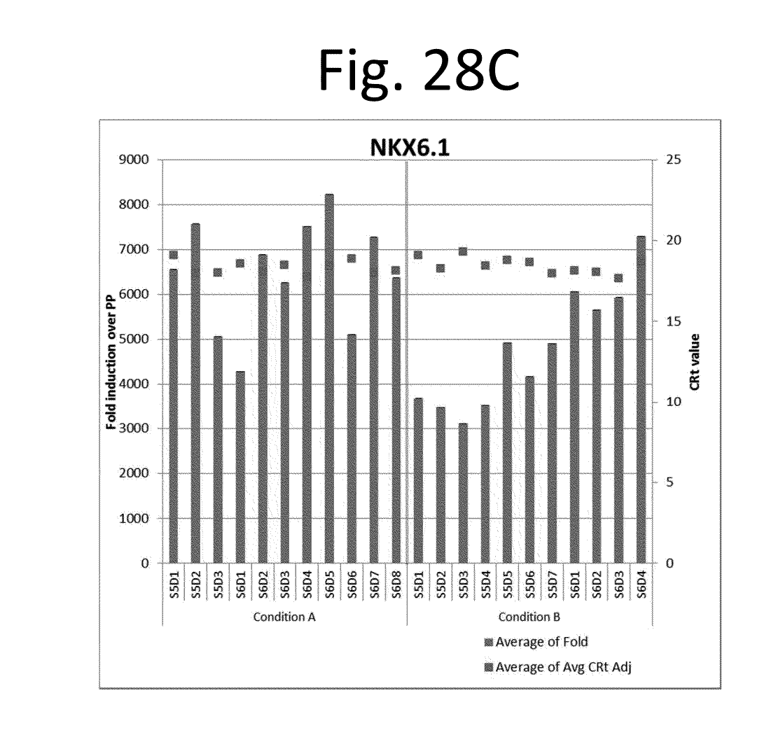

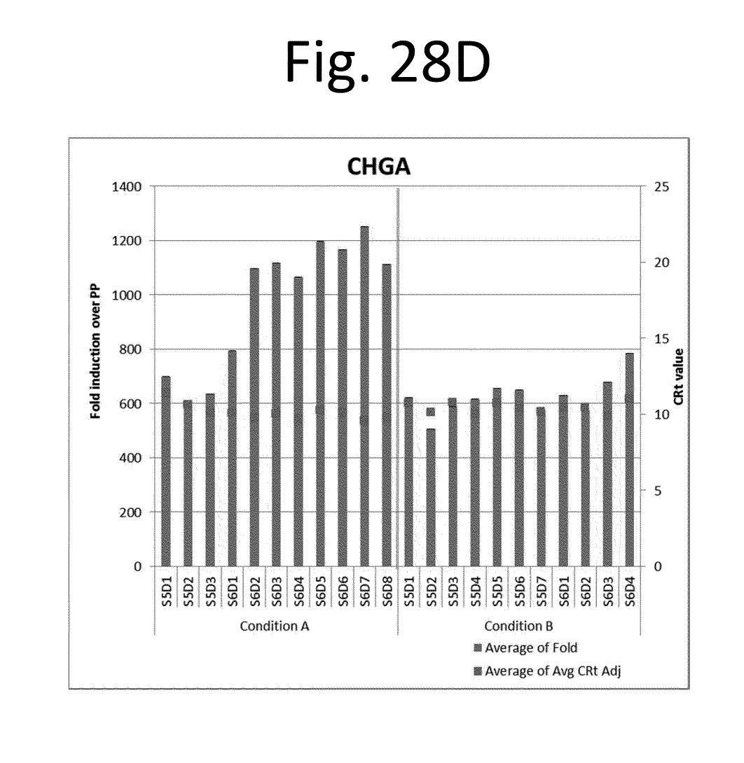

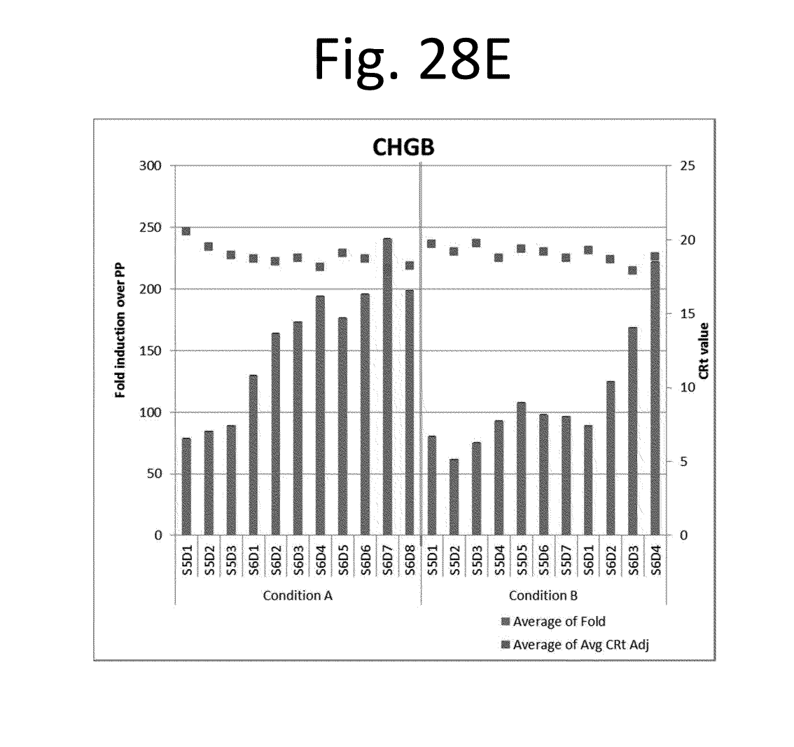

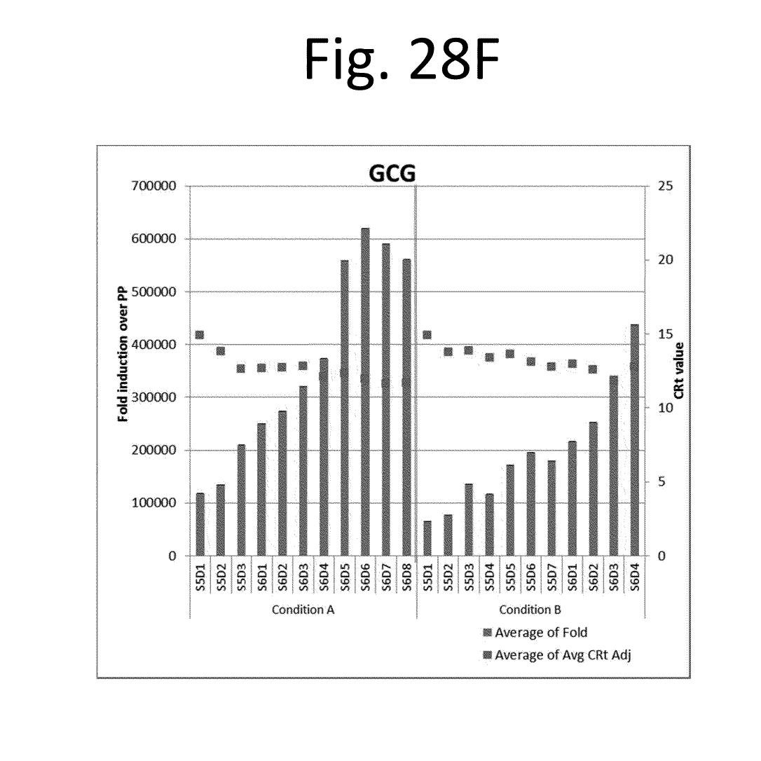

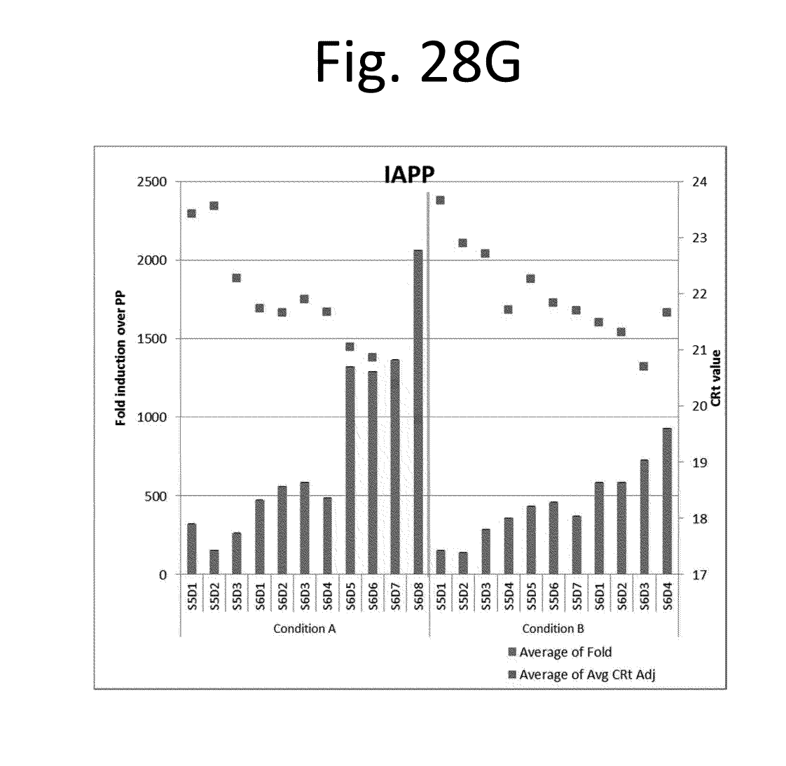

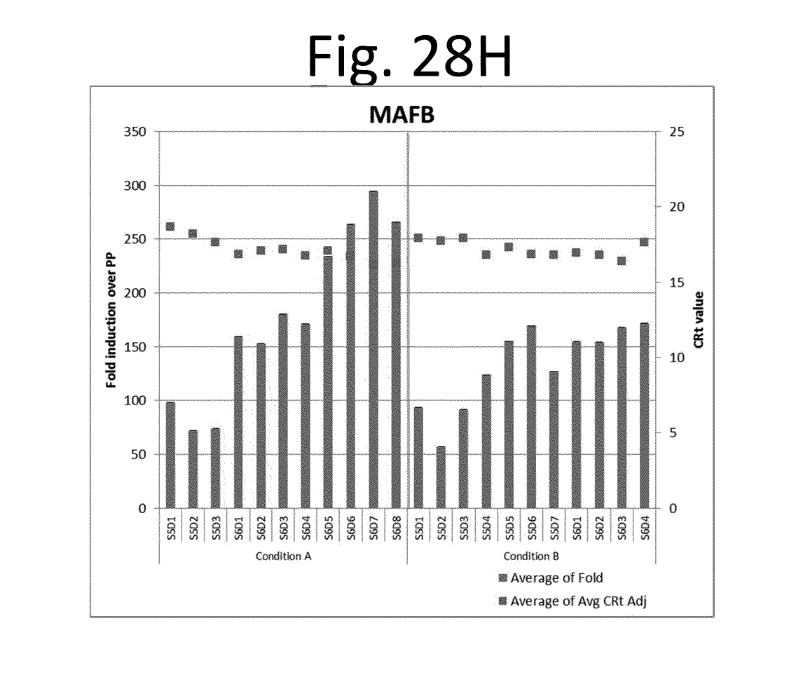

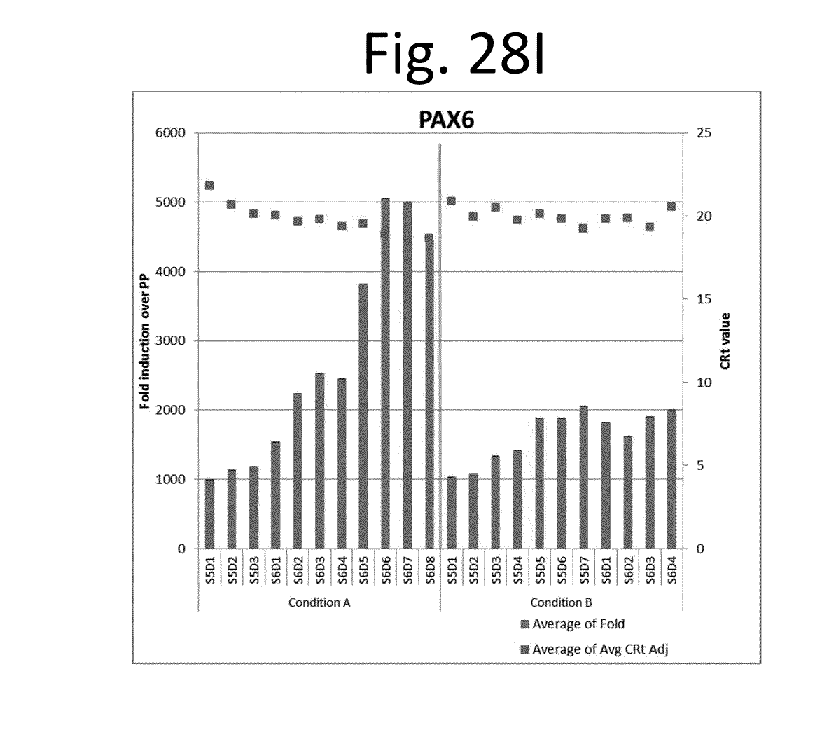

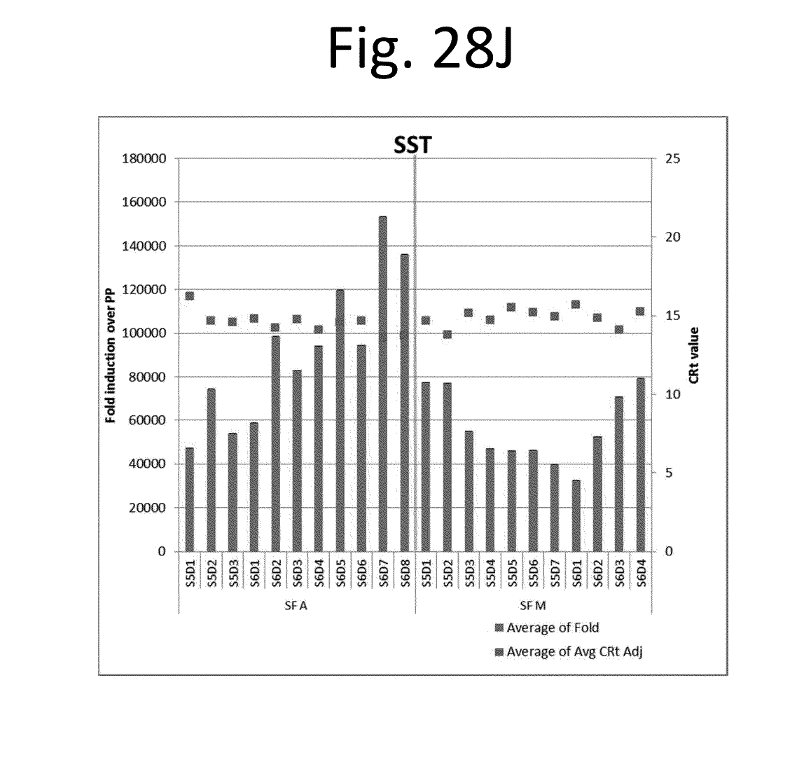

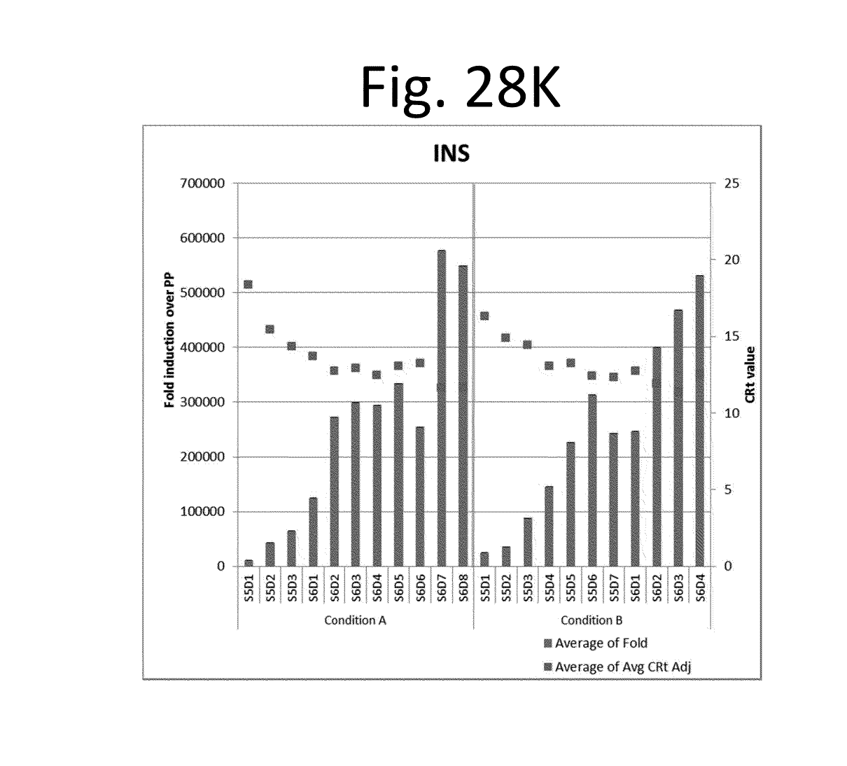

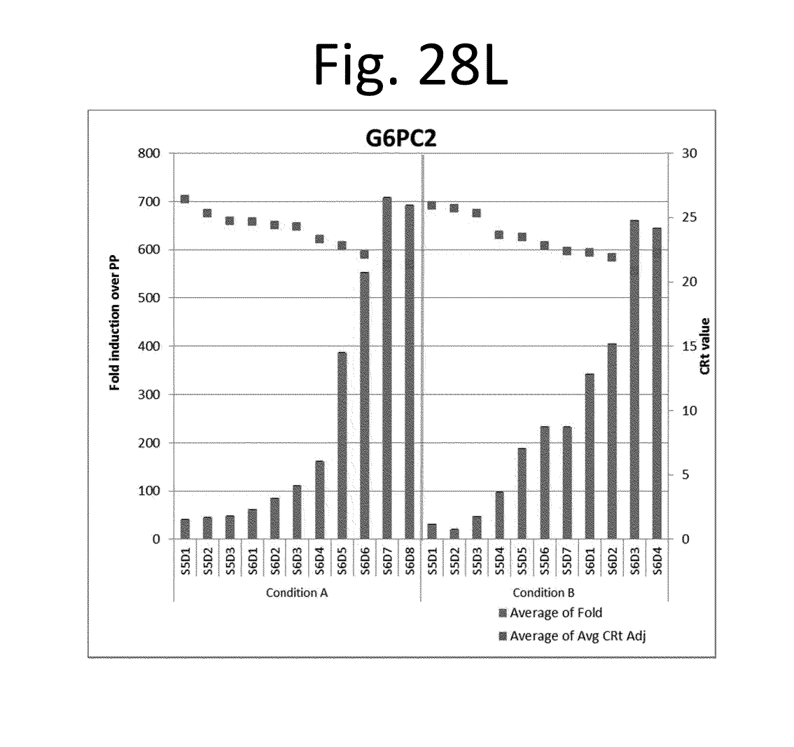

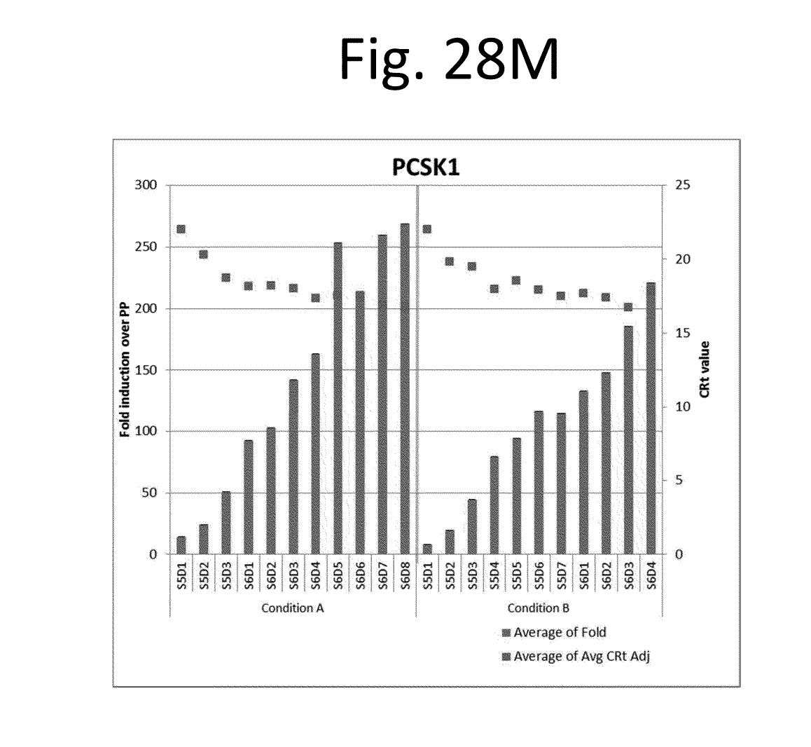

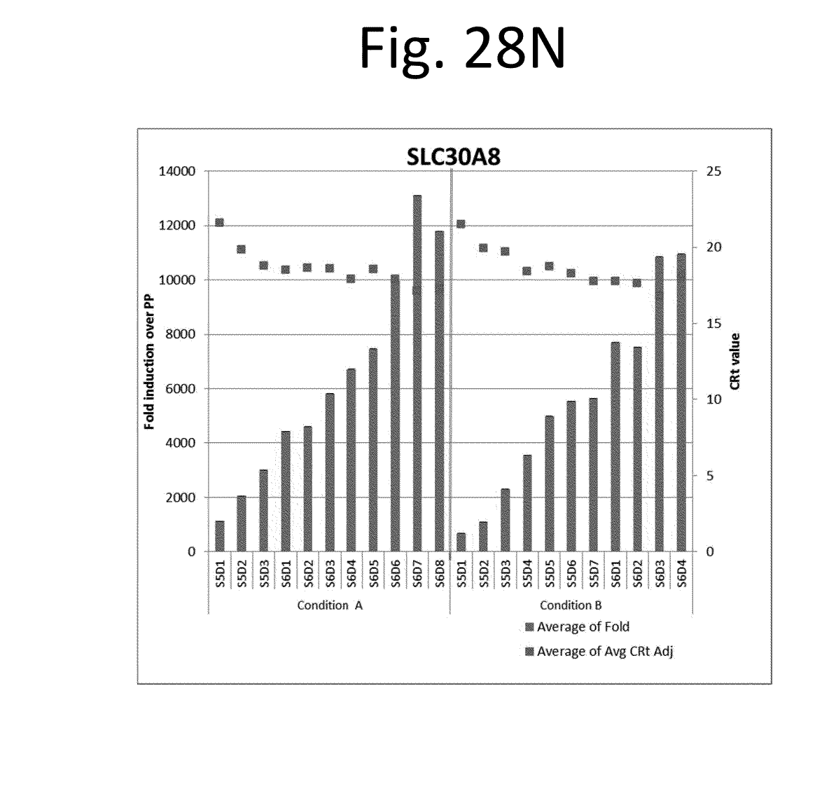

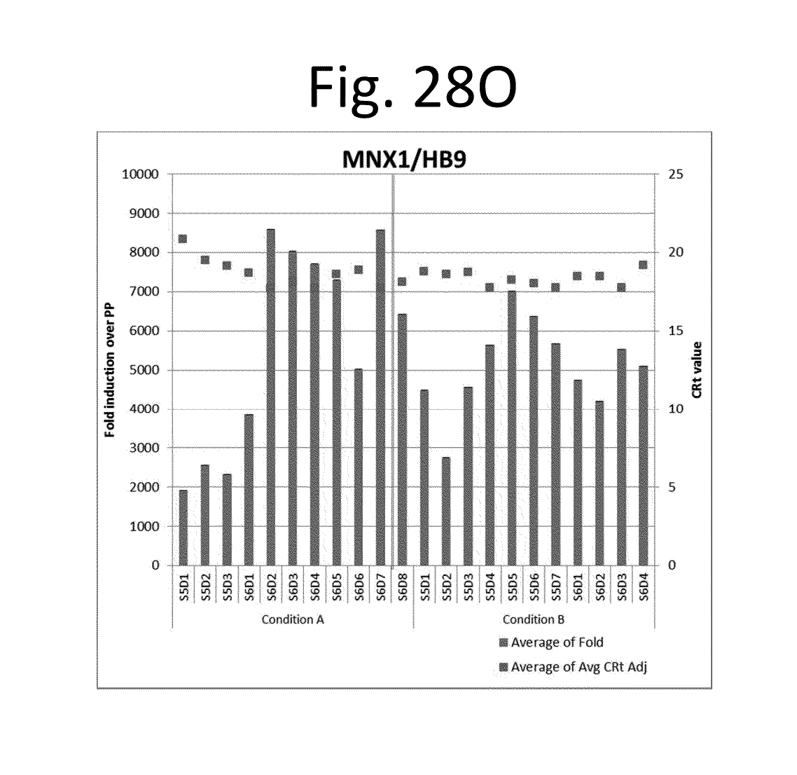

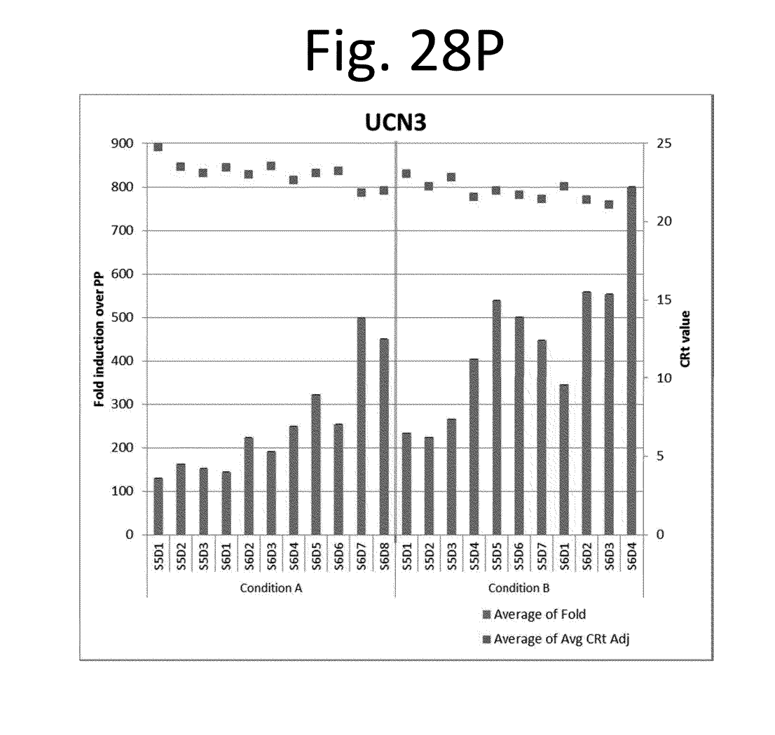

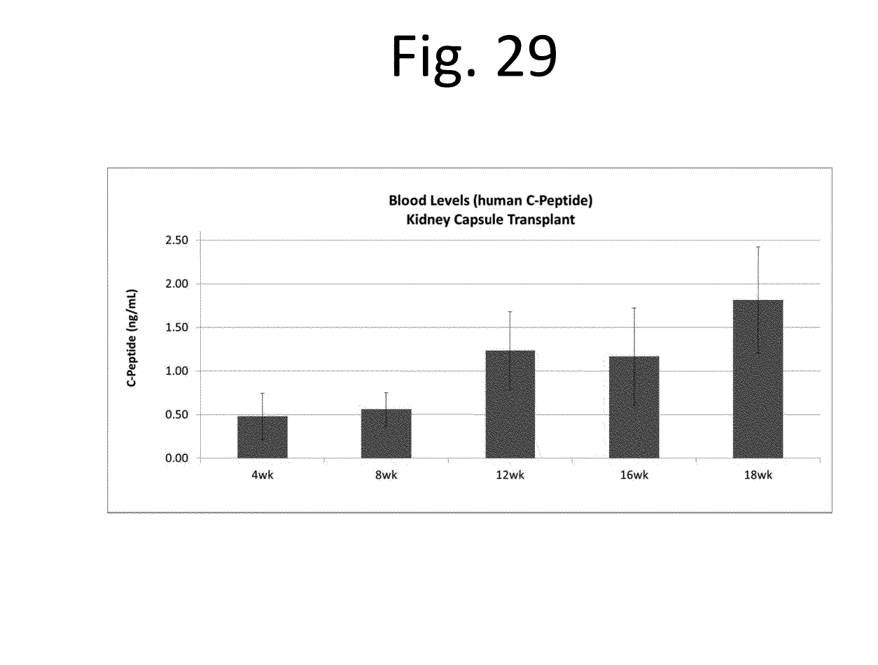

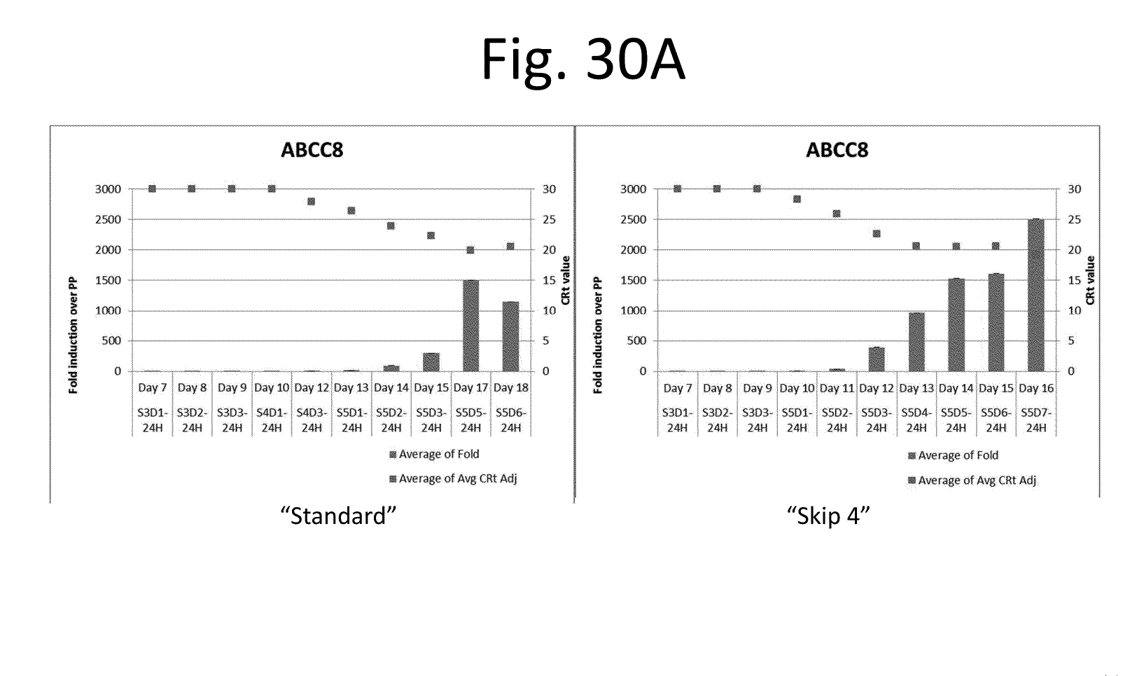

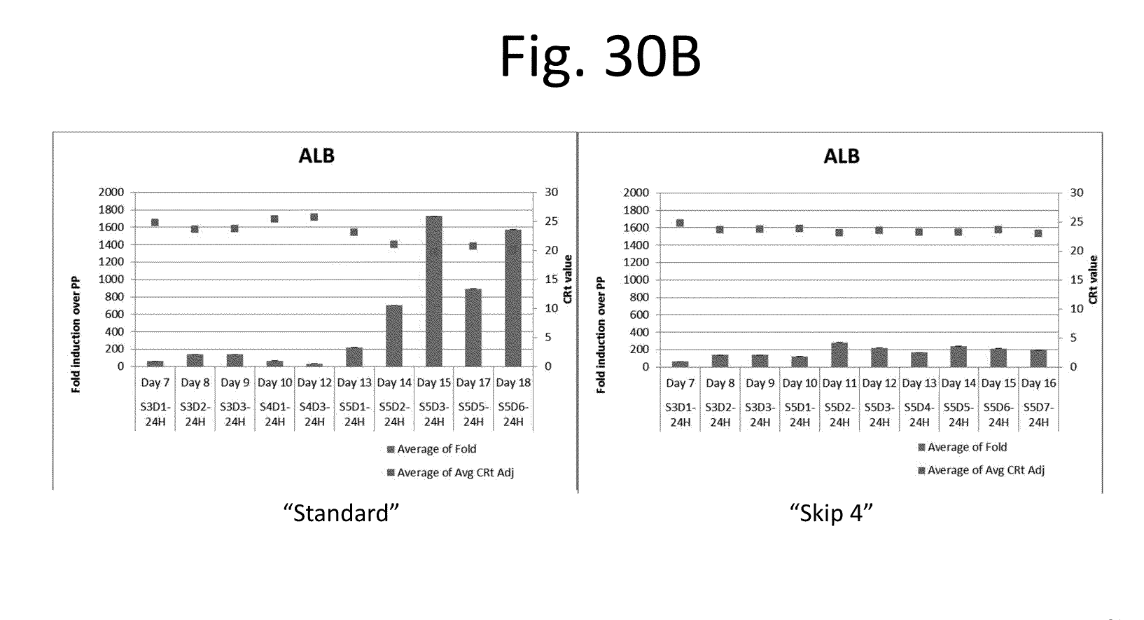

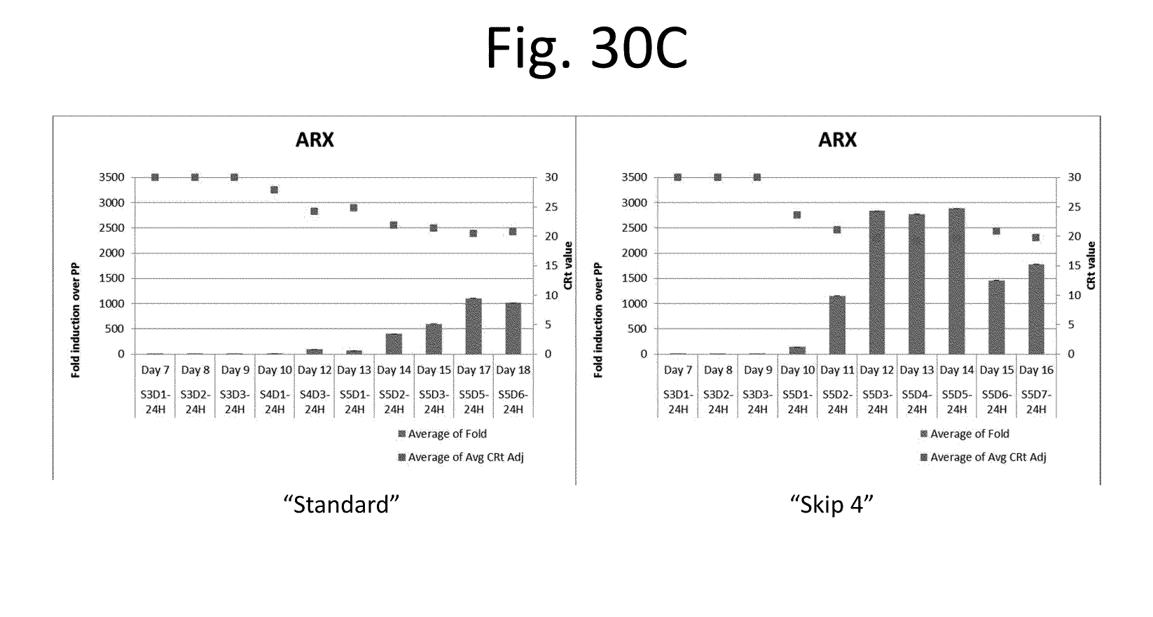

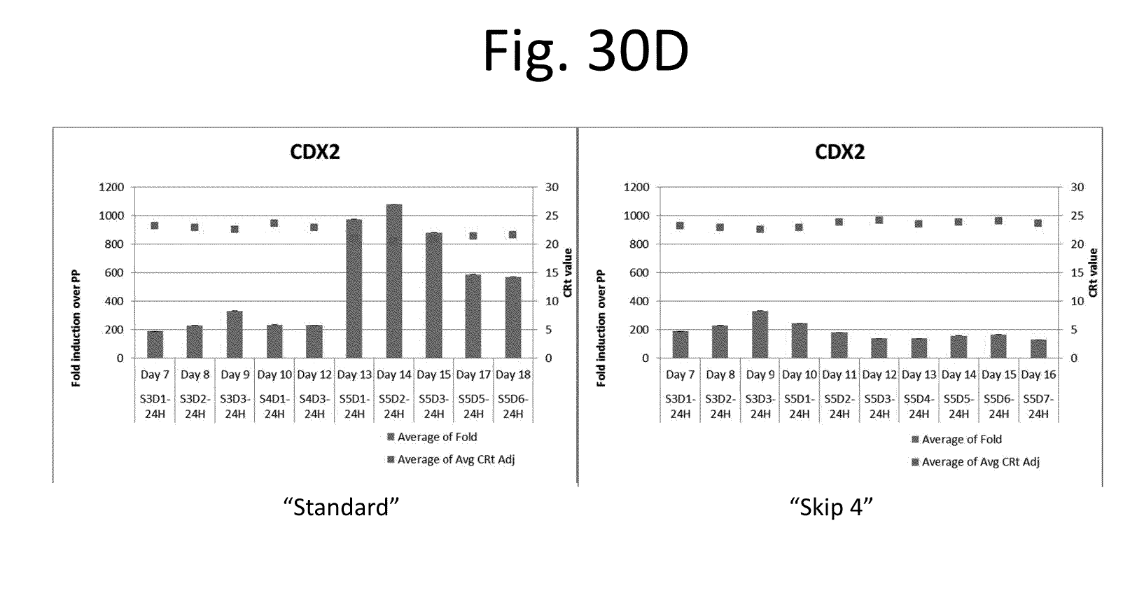

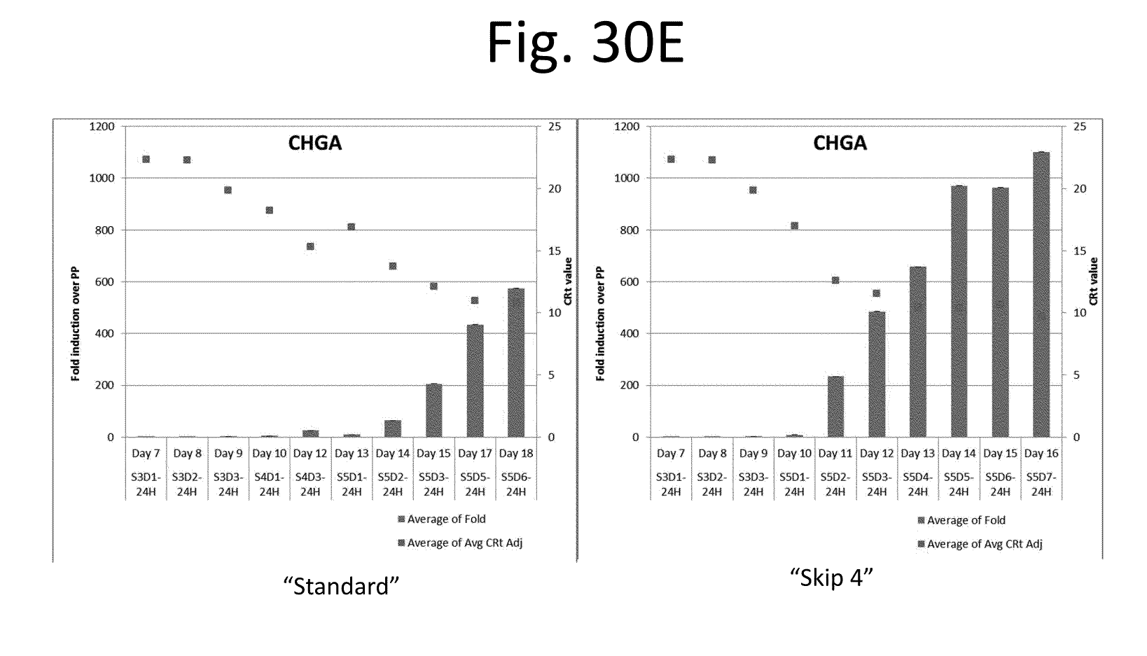

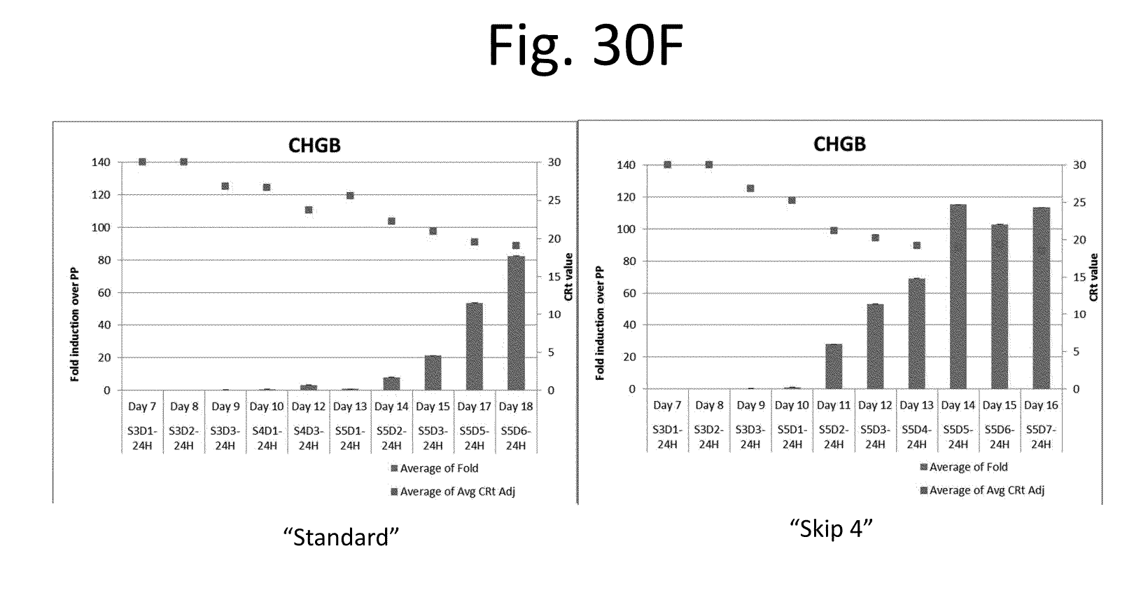

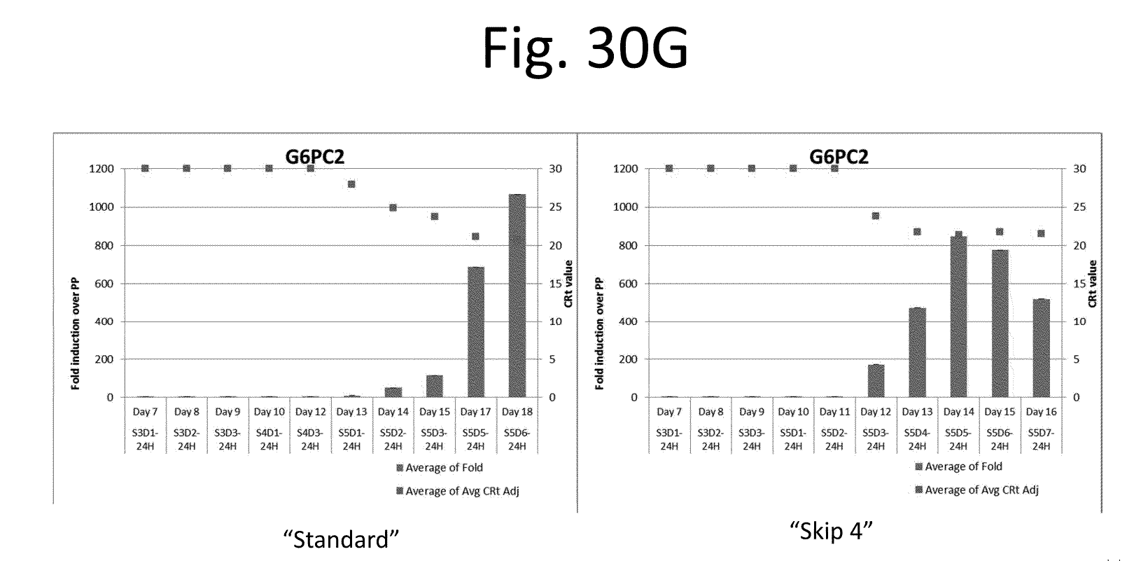

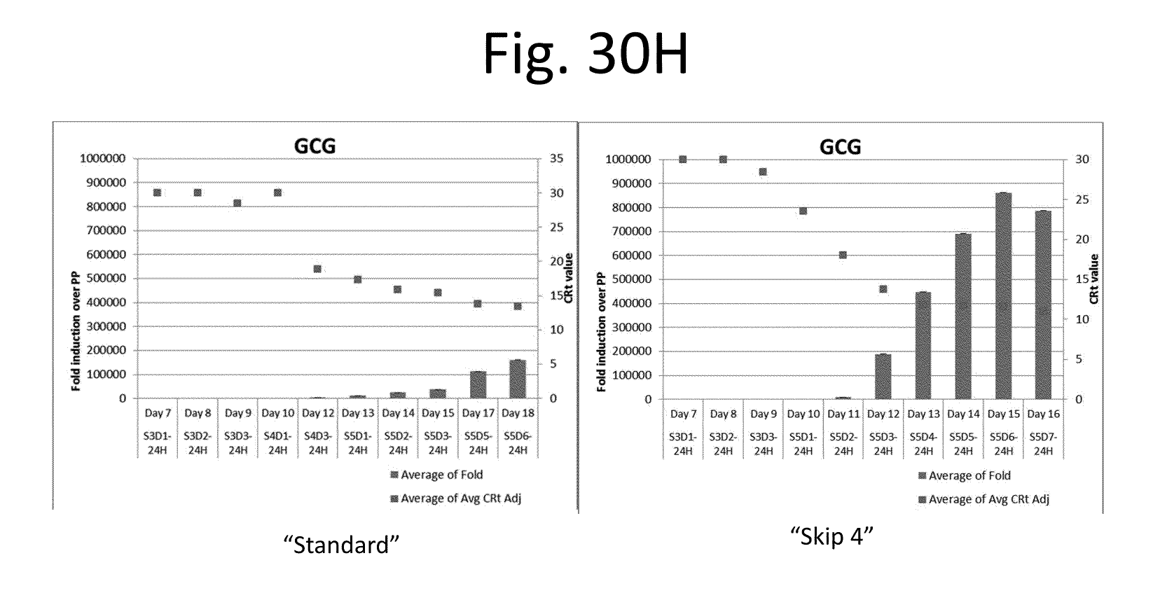

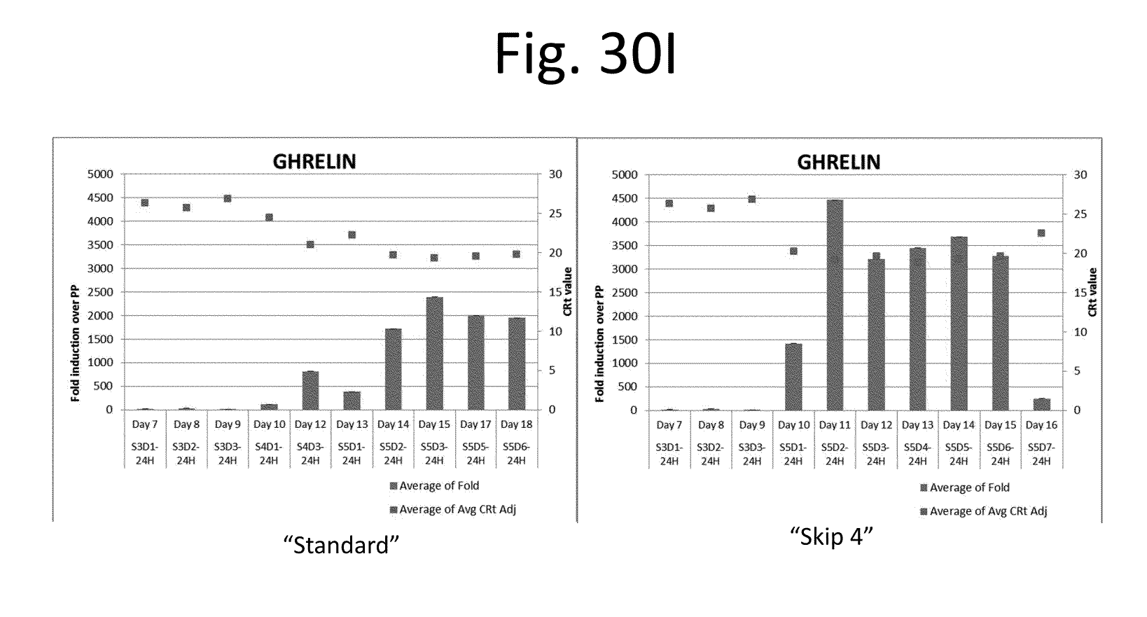

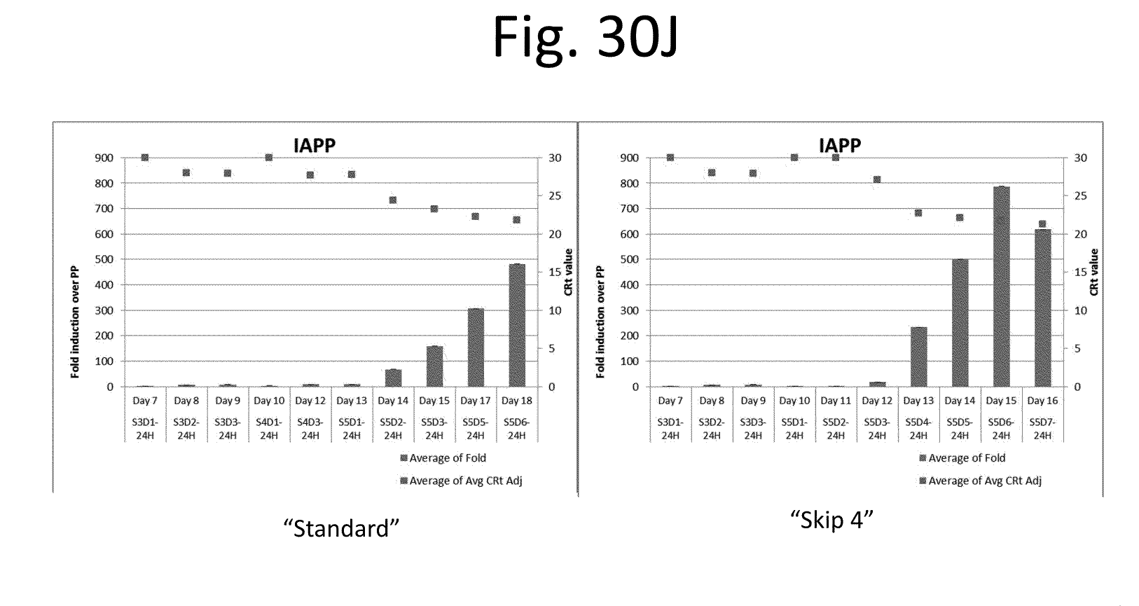

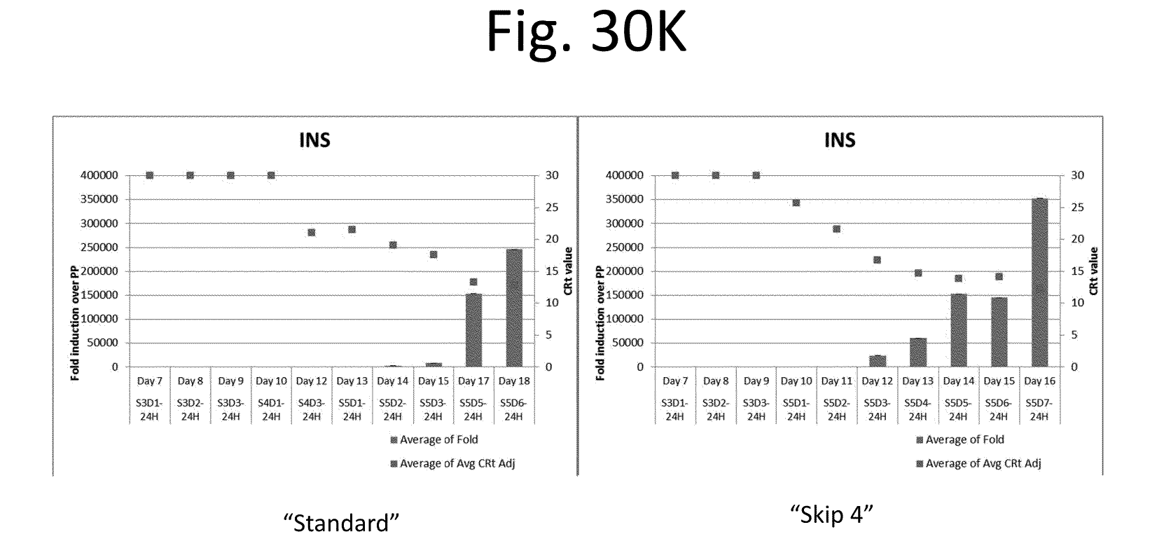

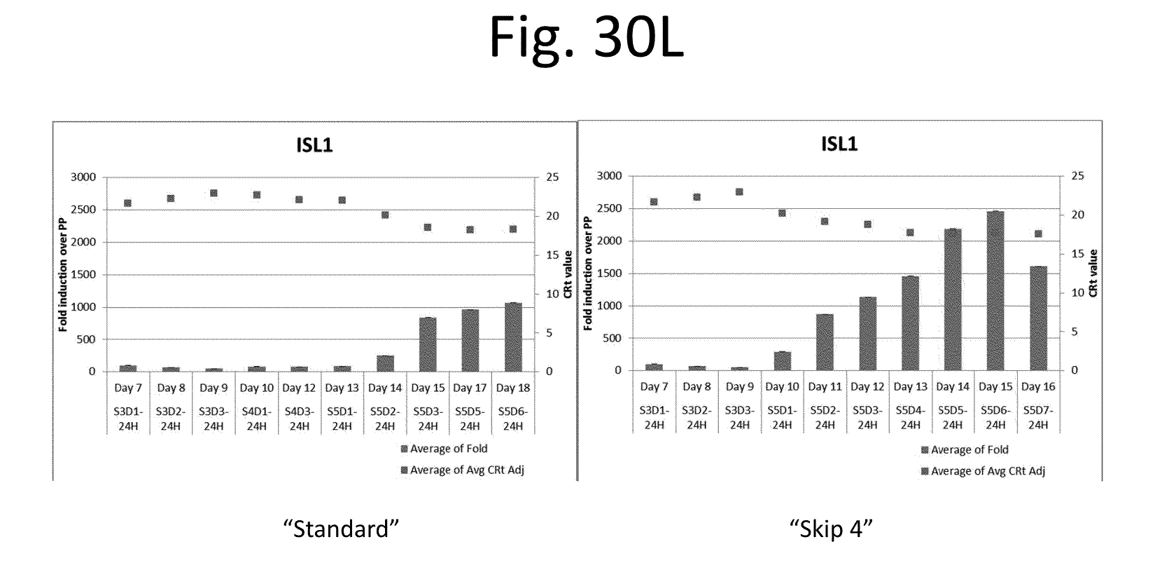

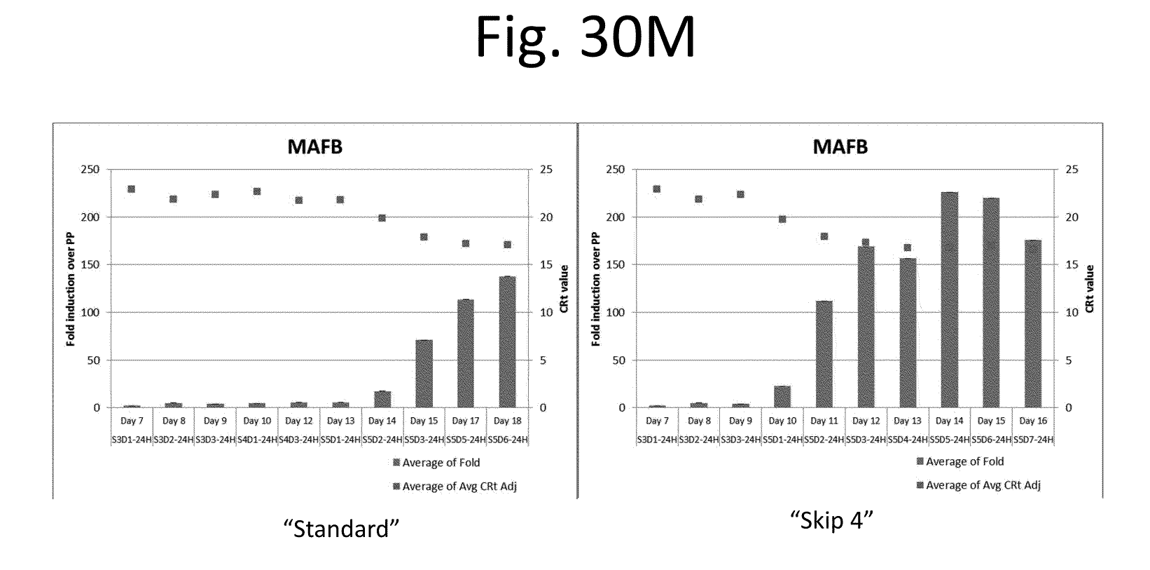

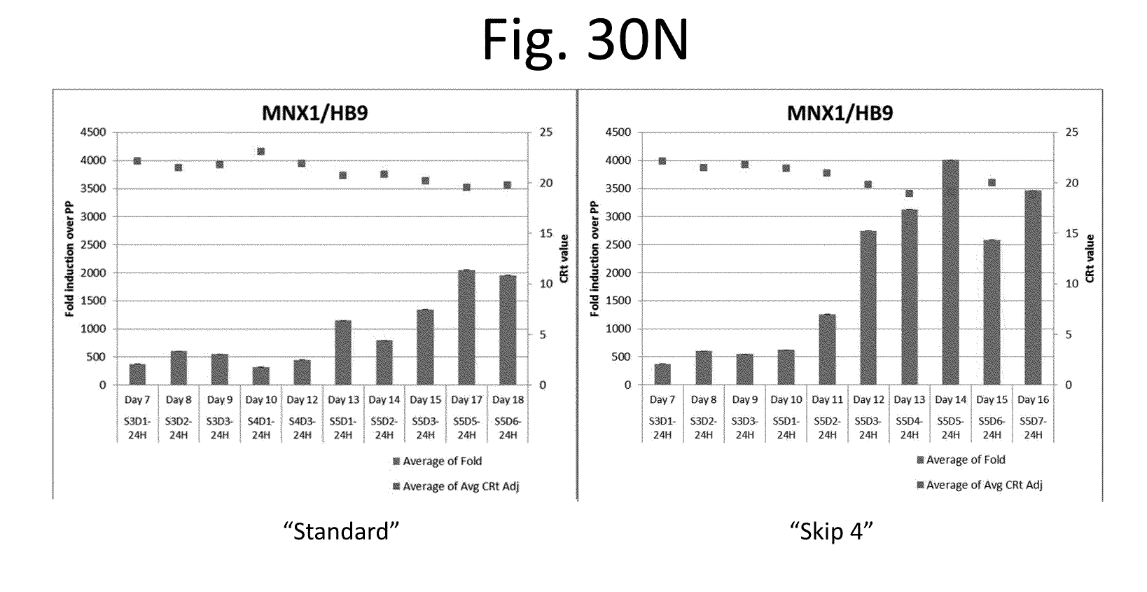

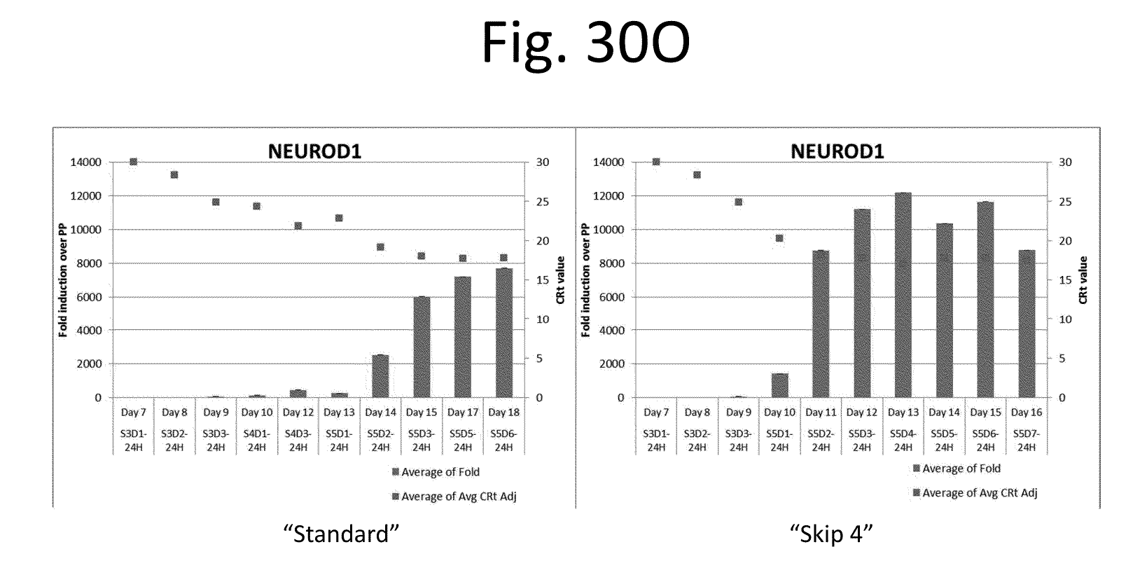

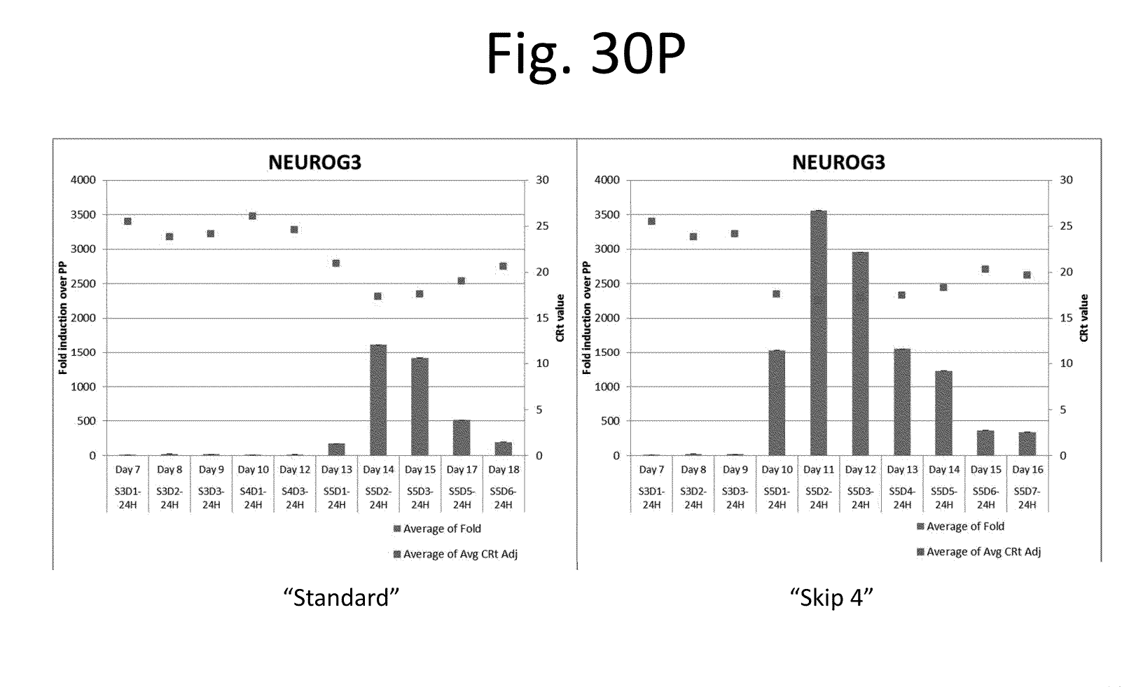

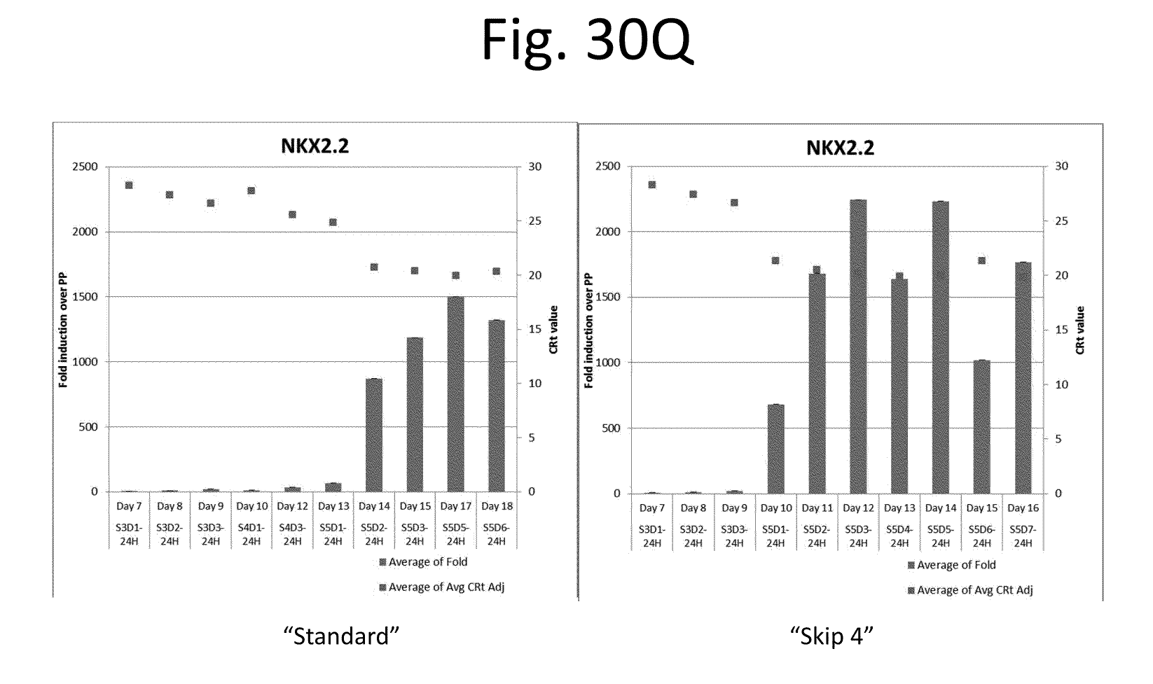

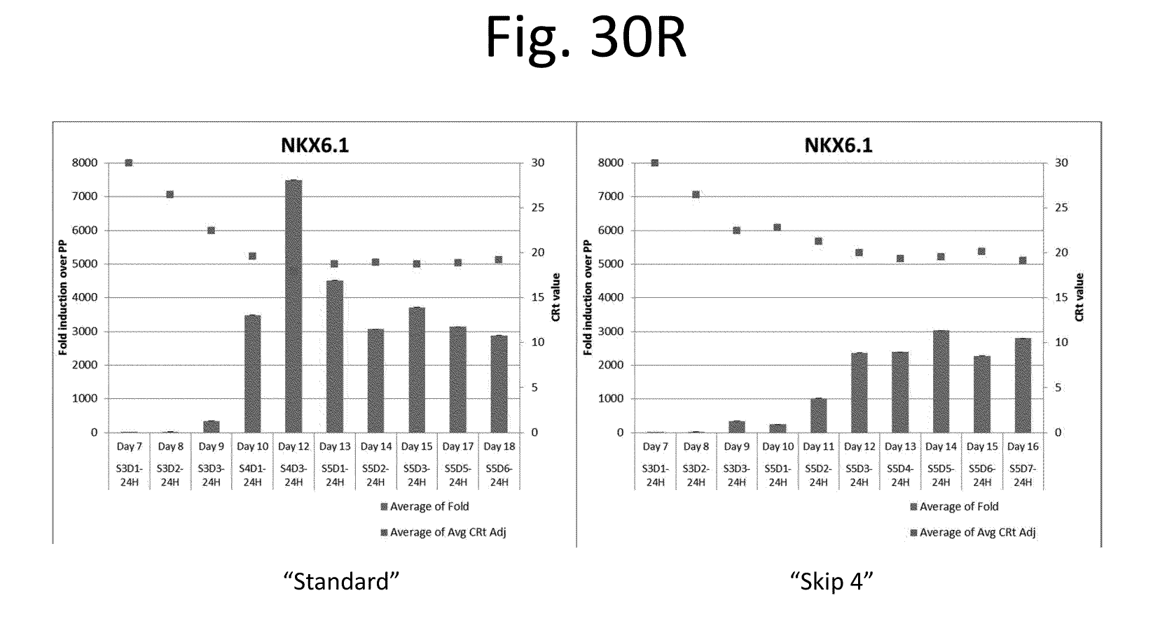

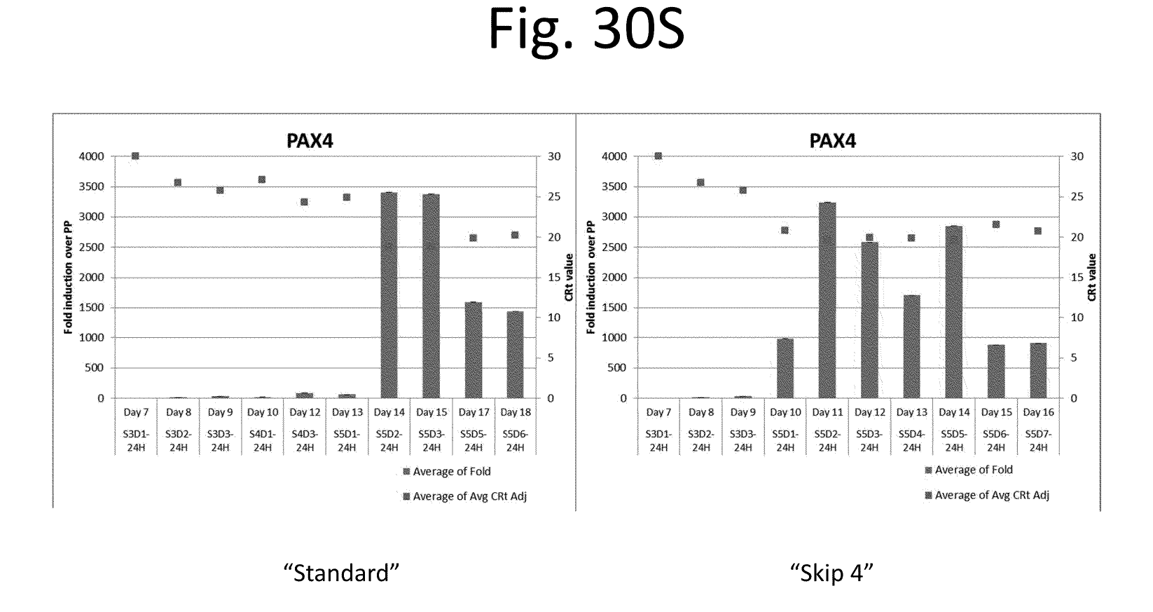

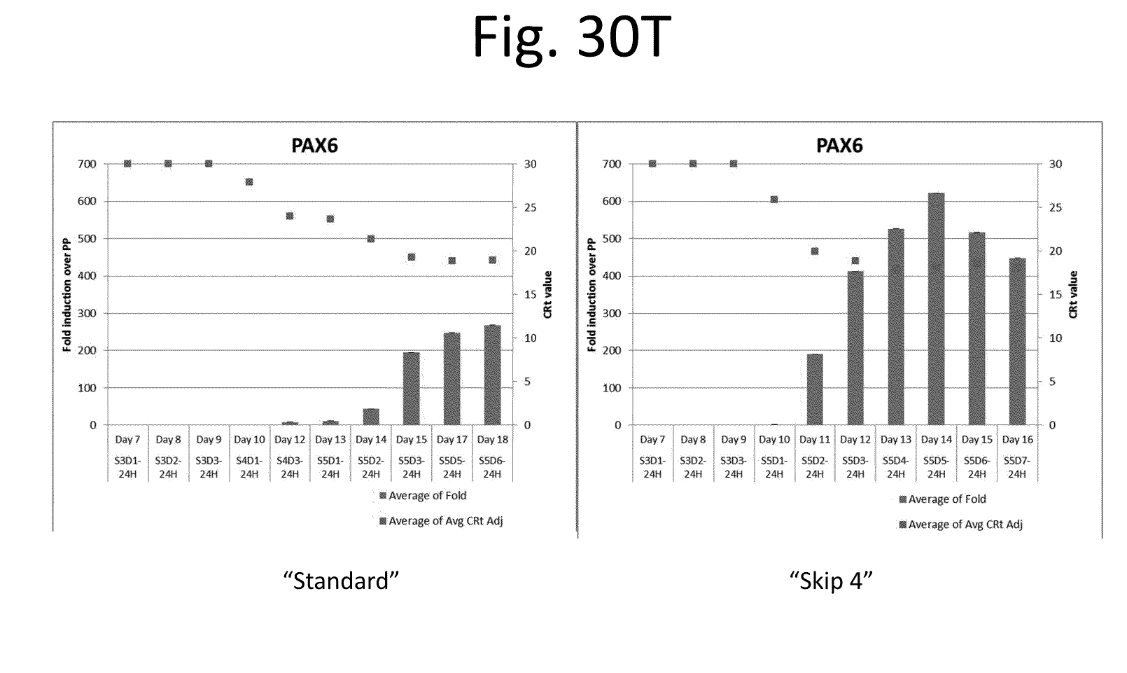

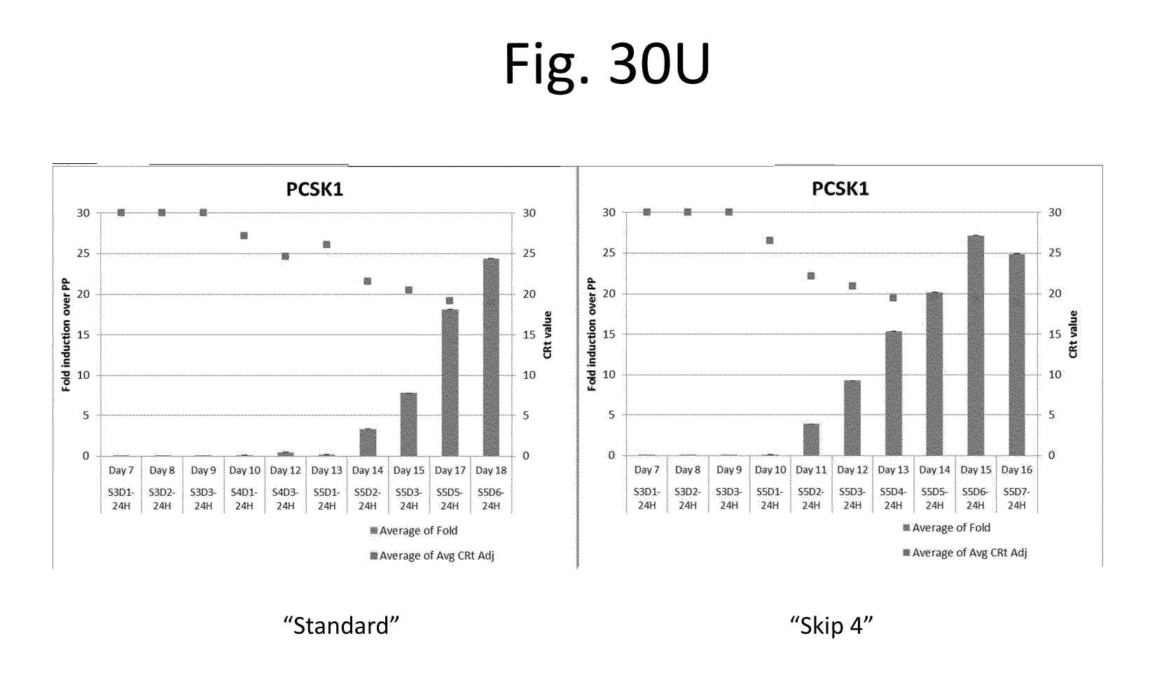

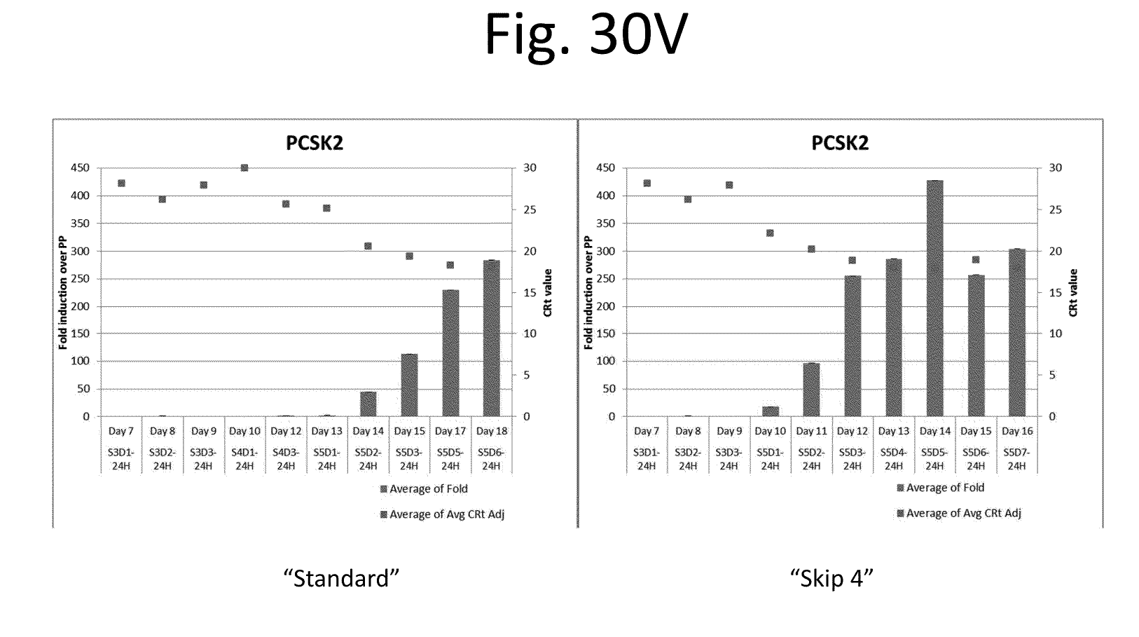

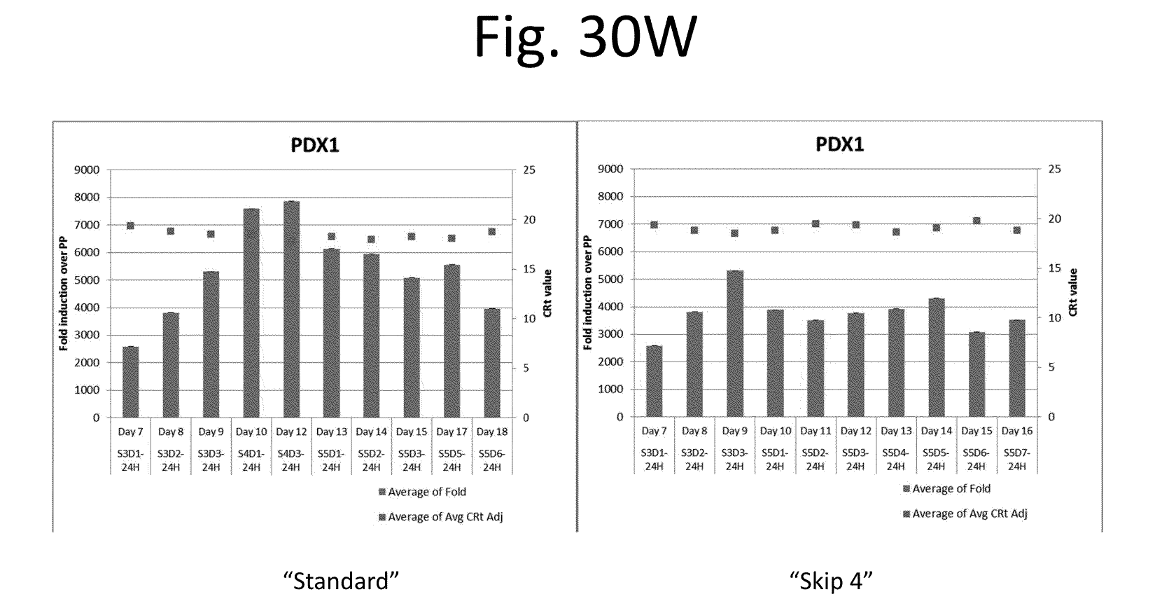

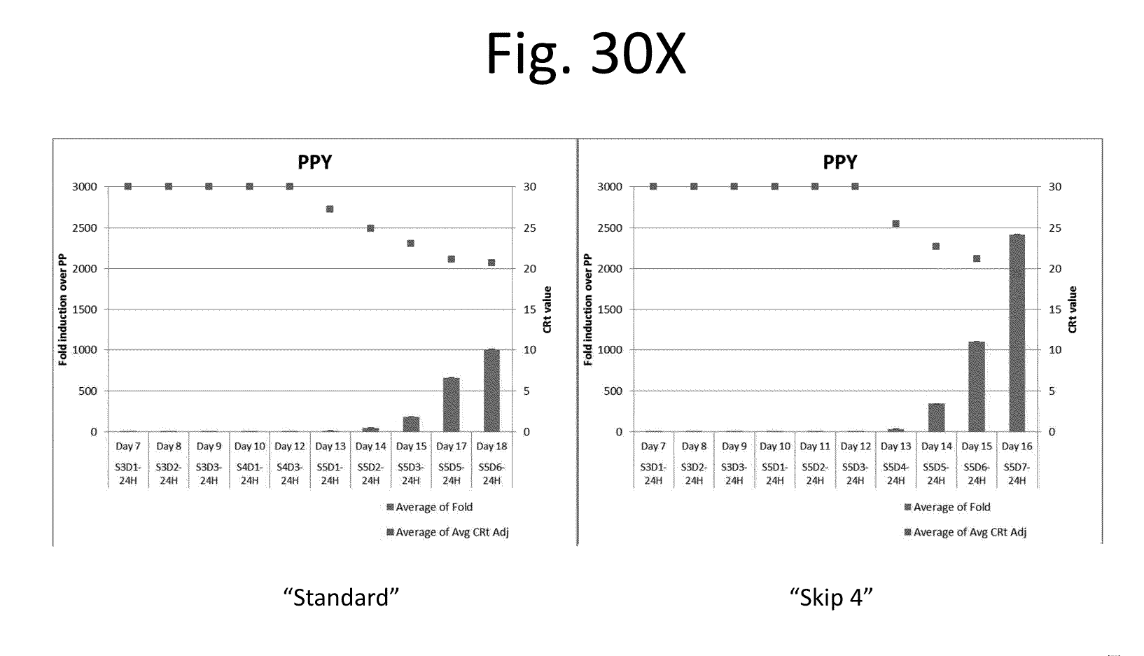

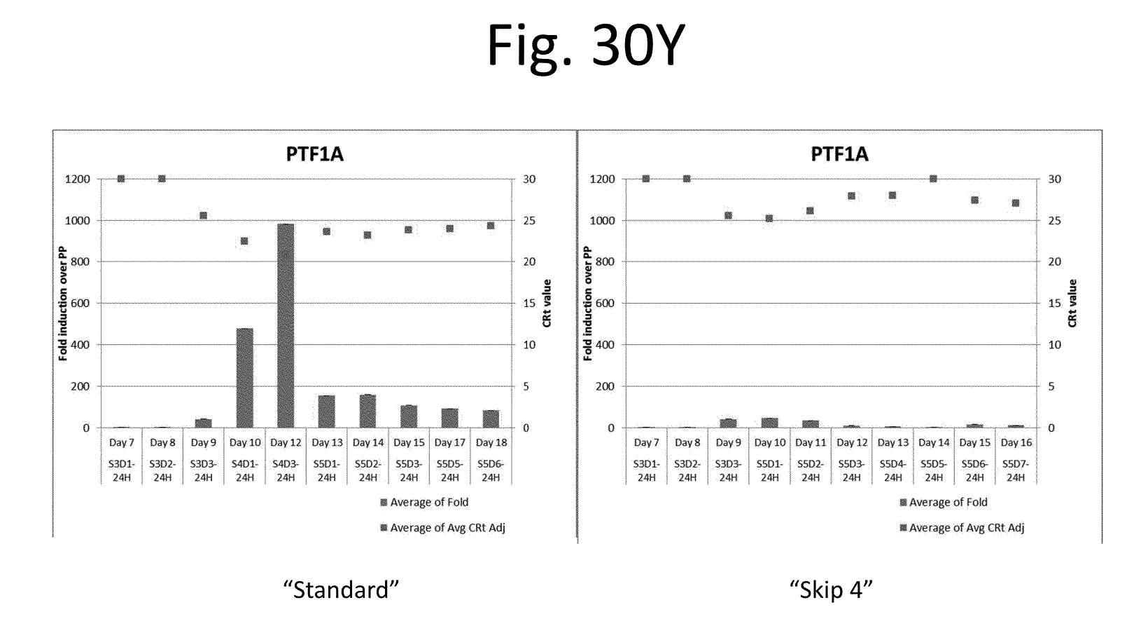

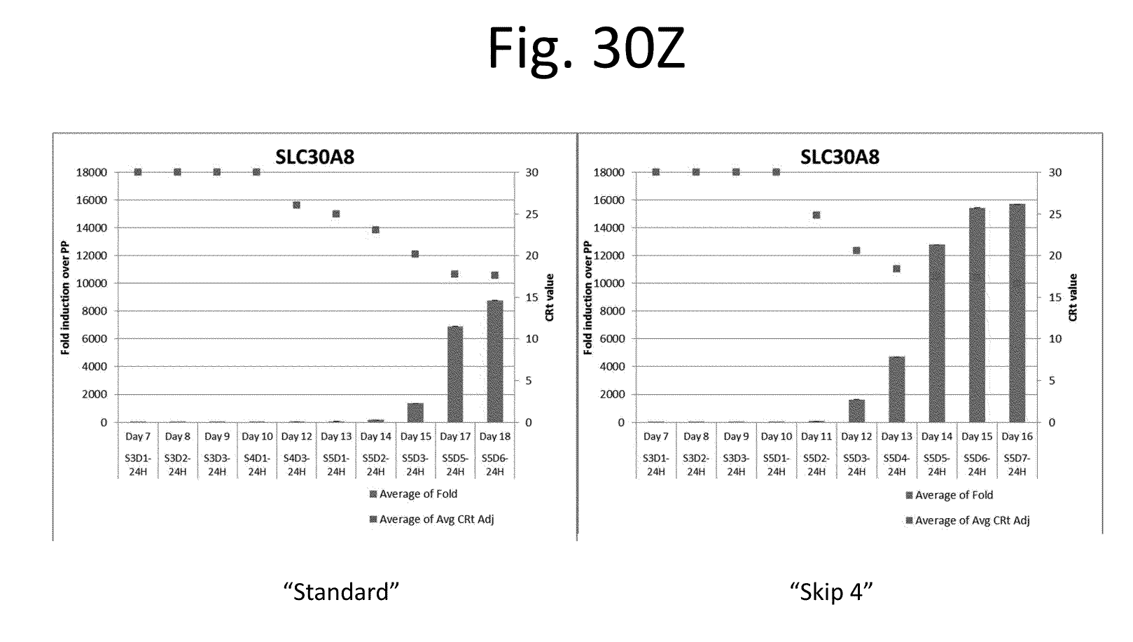

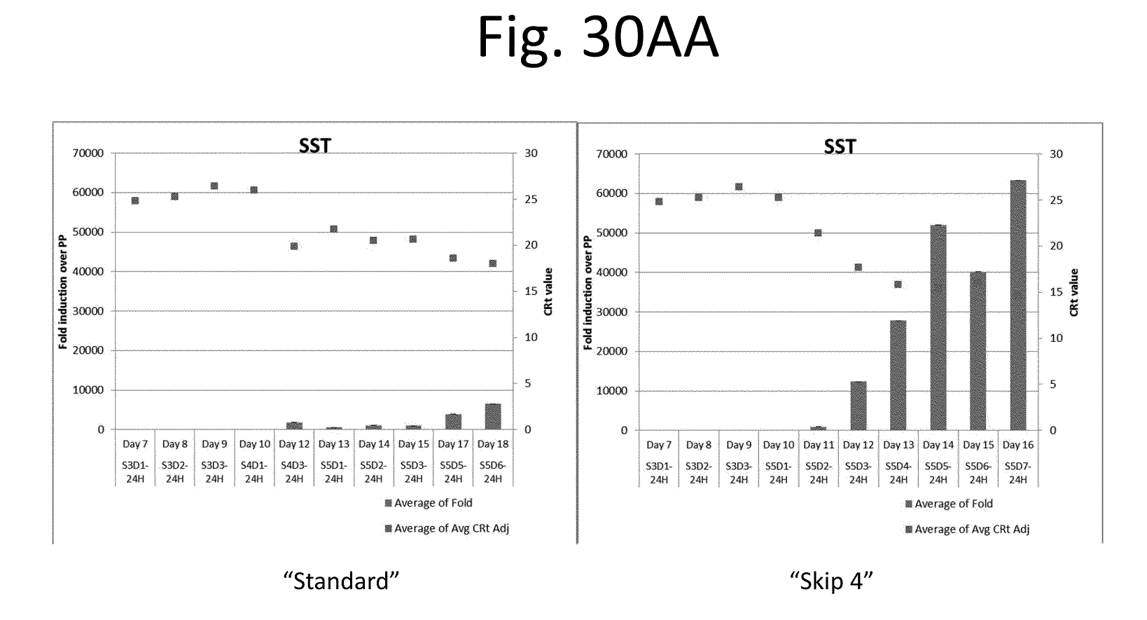

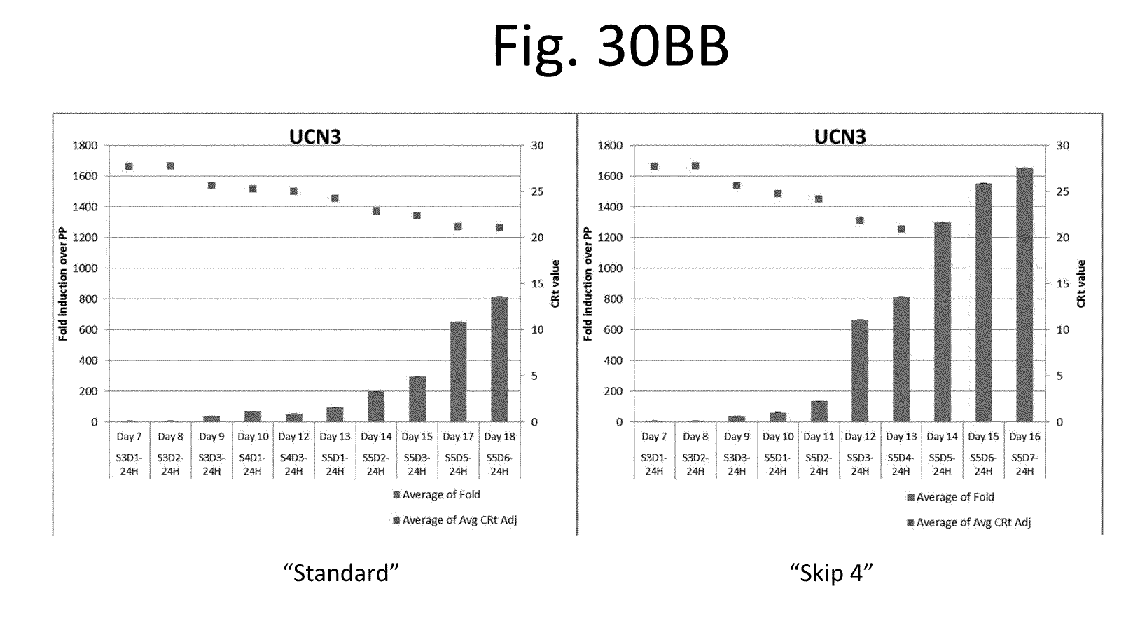

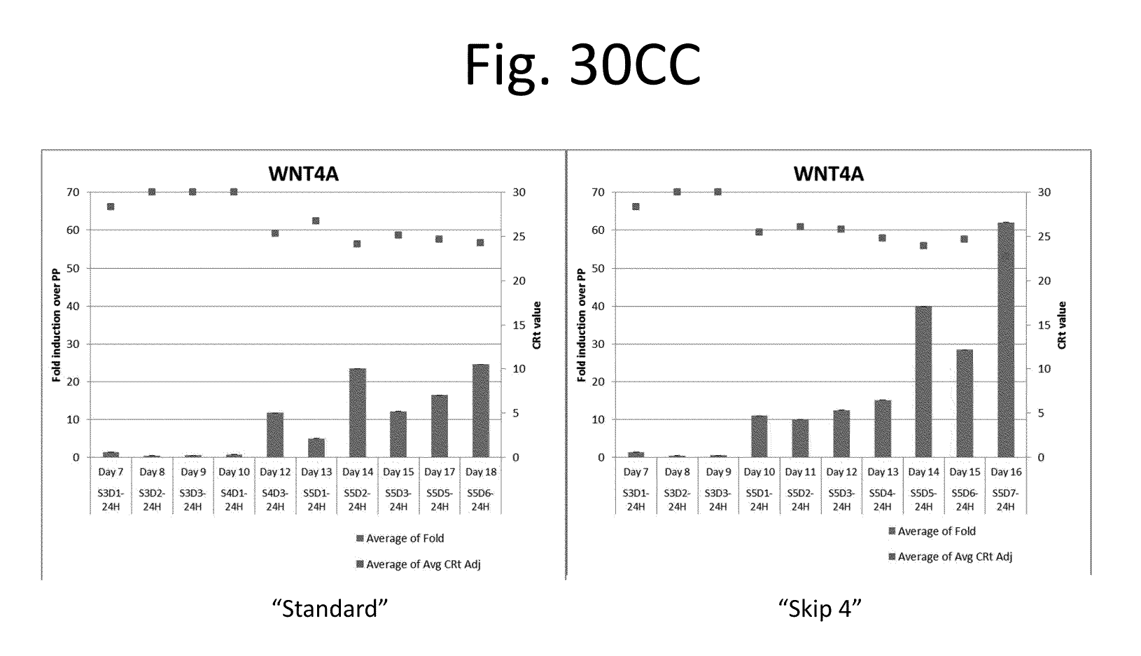

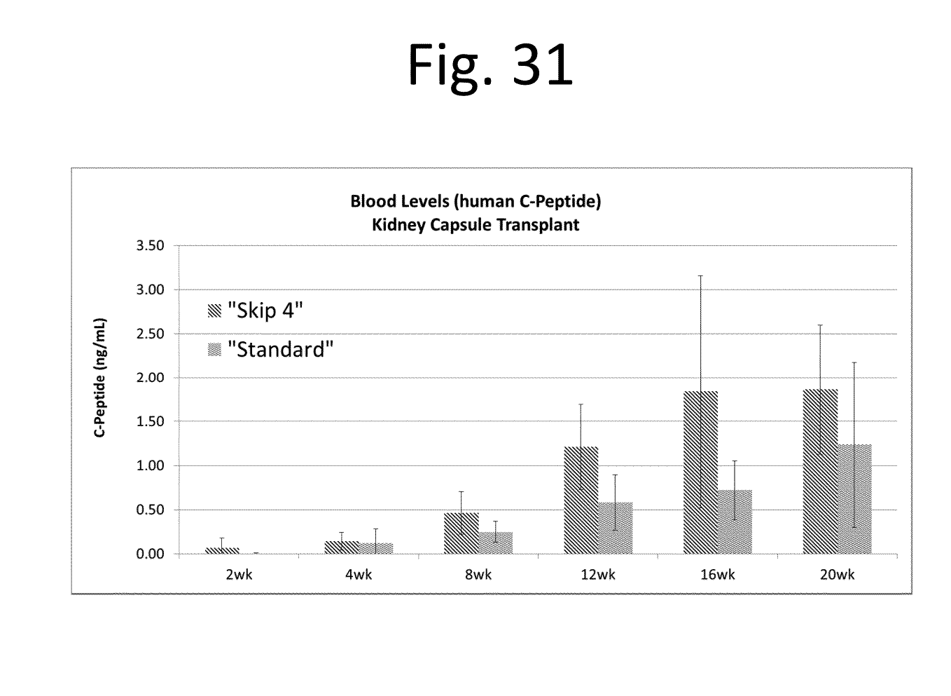

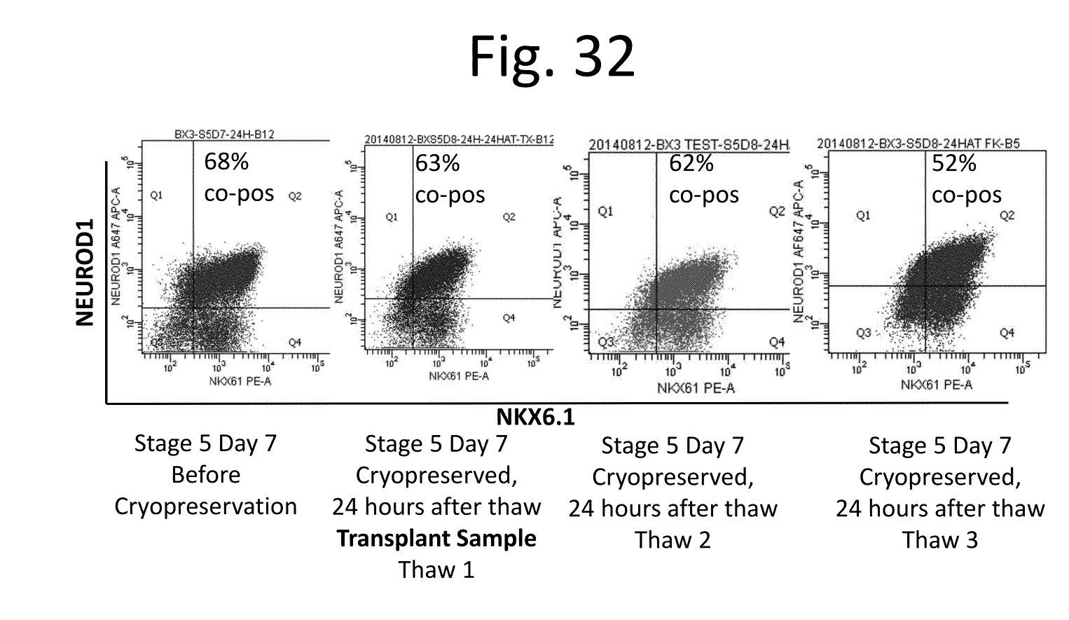

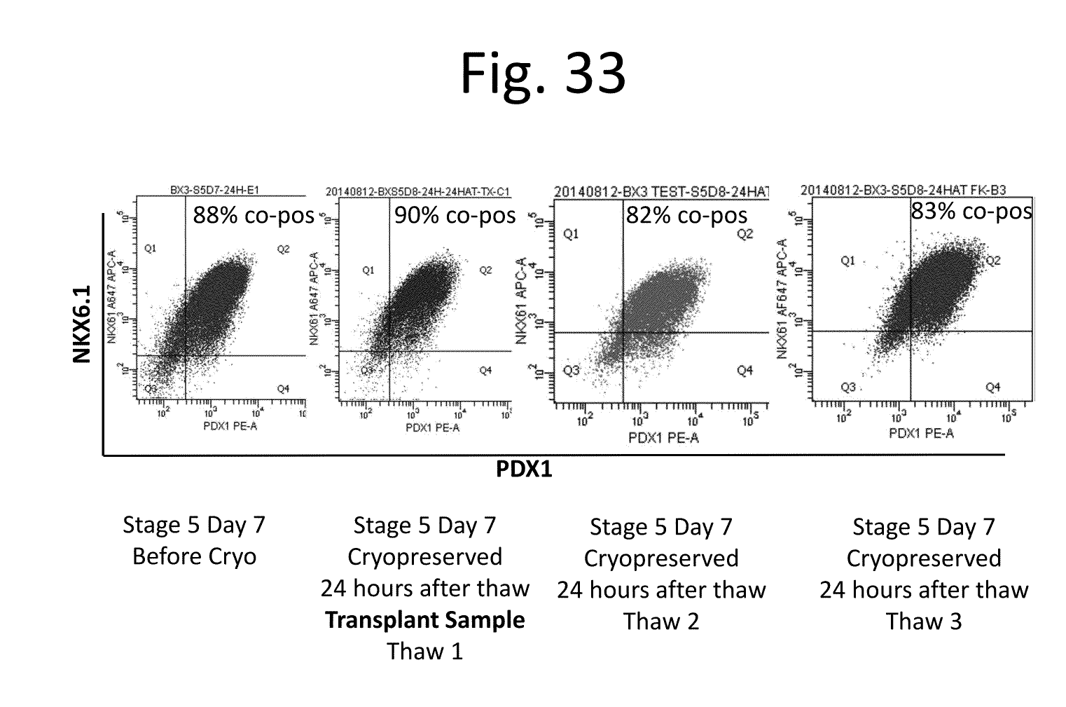

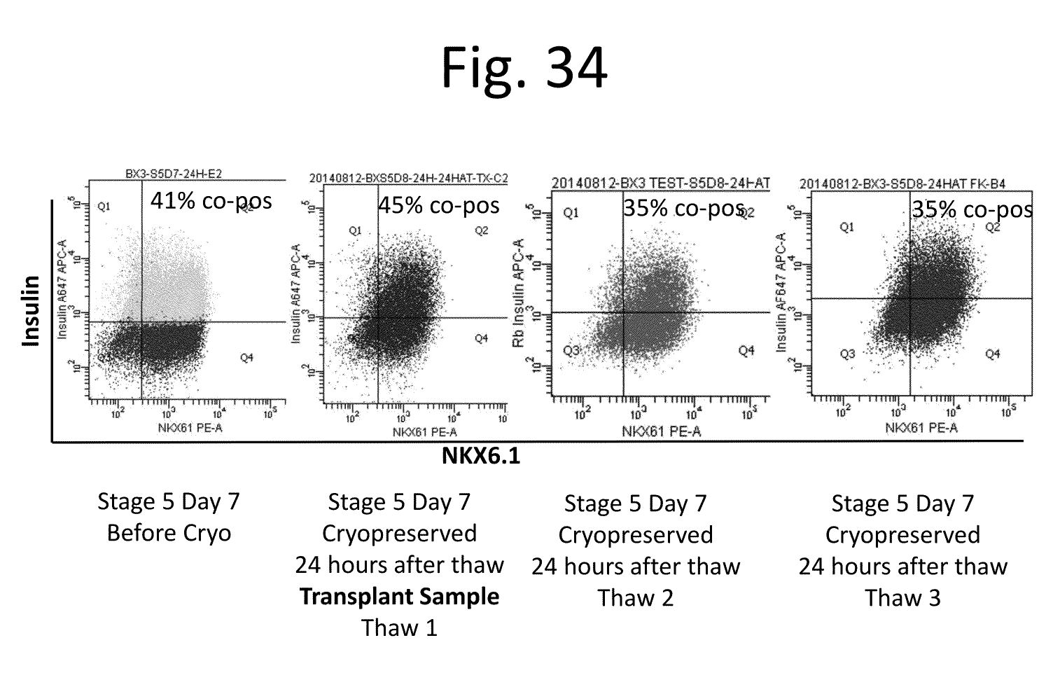

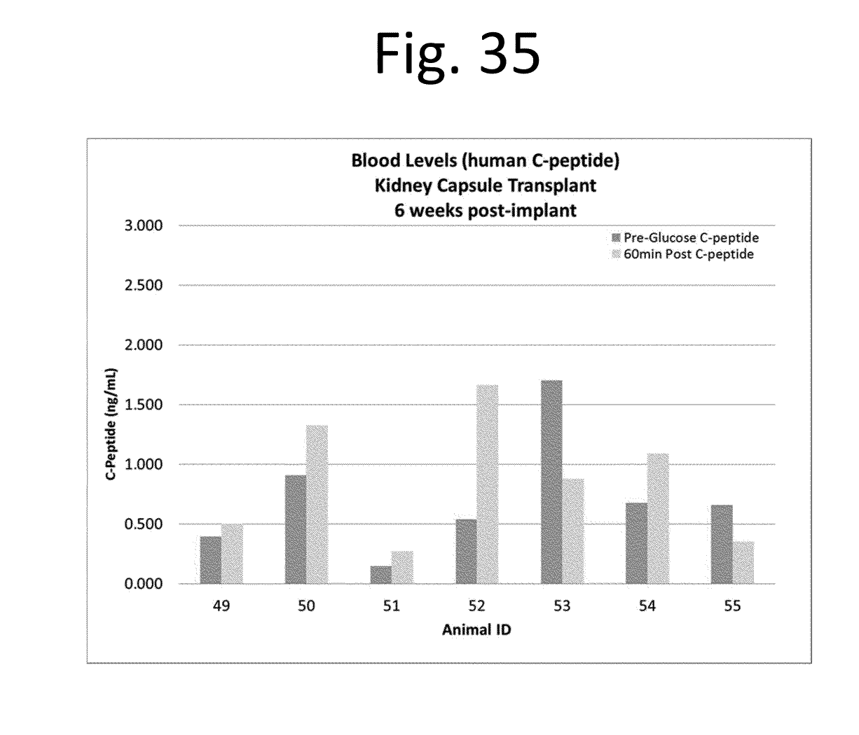

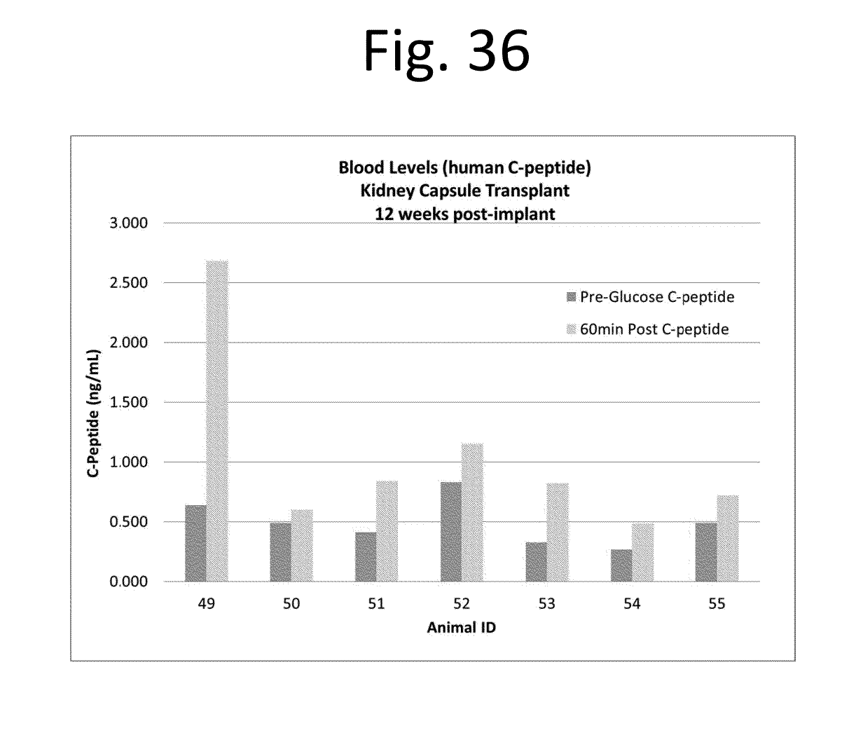

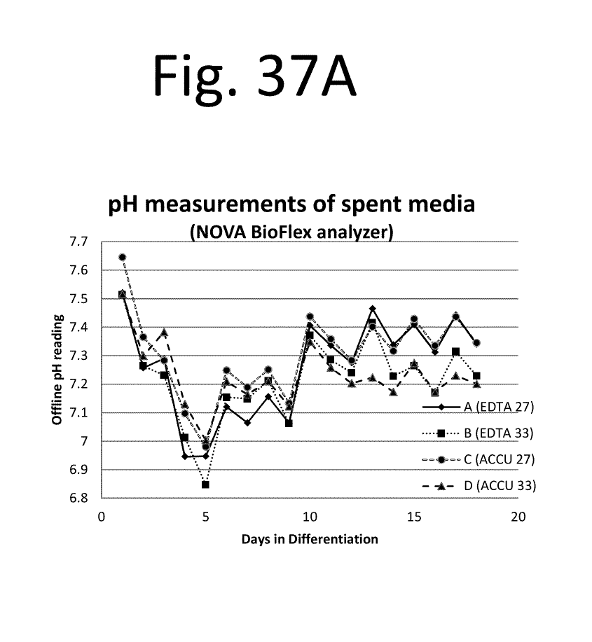

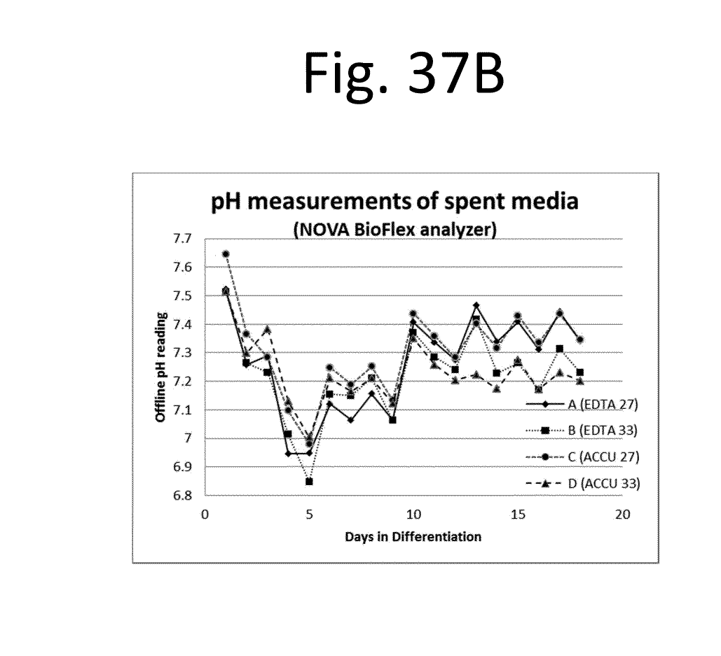

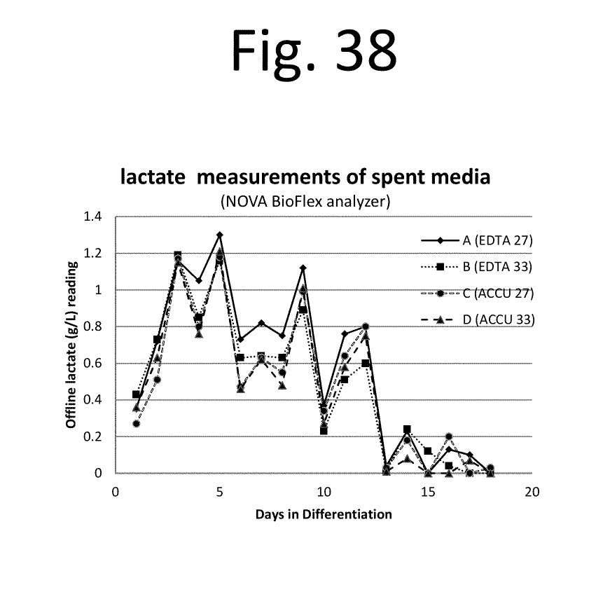

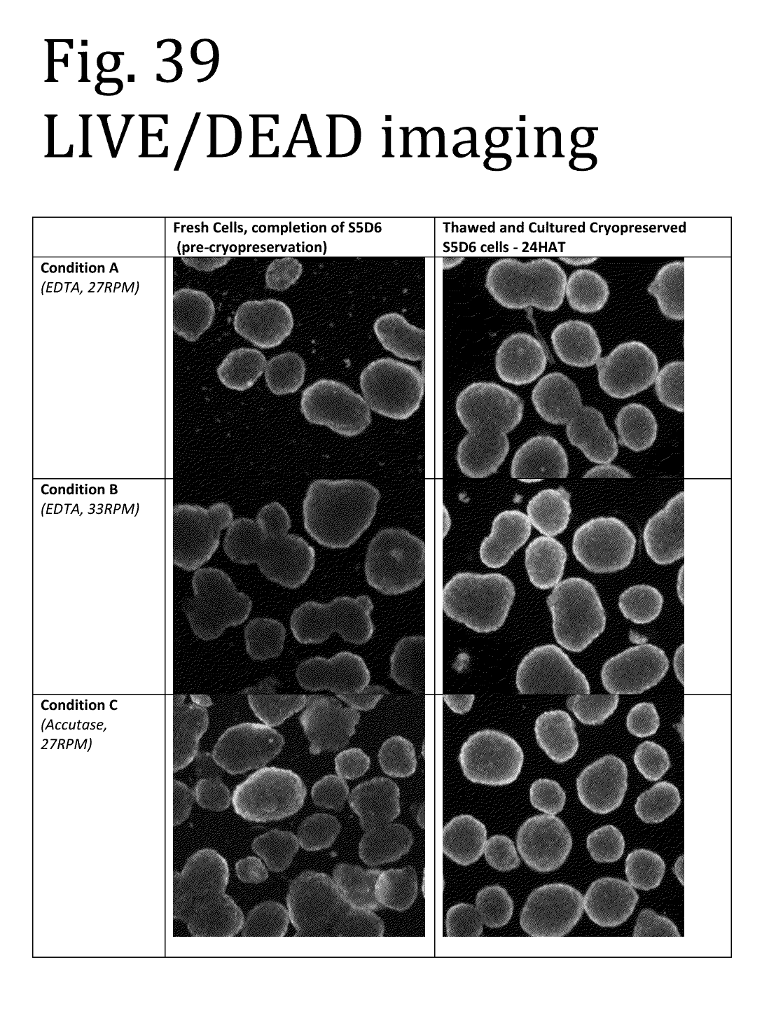

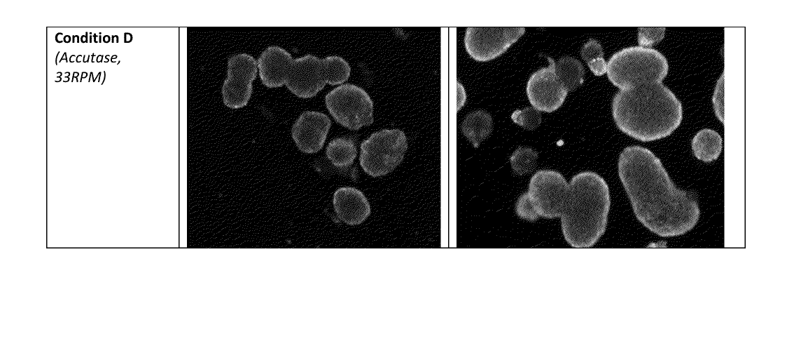

FIG. 27S is a graph of real time qRT-PCR results for expression of UCN3 over the course of the differentiation protocols of Example 4 from Stage 5, day 1 through day 7 of Stage 6.

FIG. 28A is a graph of real time qRT-PCR results for expression of NEUROG3 over the course of the differentiation protocols of Example 5 from Stage 5, day 1 through day 4 of Stage 6.

FIG. 28B is a graph of real time qRT-PCR results for expression of NEUROD1 over the course of the differentiation protocols of Example 5 from Stage 5, day 1 through day 4 of Stage 6.

FIG. 28C is a graph of real time qRT-PCR results for expression of NKX6.1 over the course of the differentiation protocols of Example 5 from Stage 5, day 1 through day 4 of Stage 6.

FIG. 28D is a graph of real time qRT-PCR results for expression of chromogranin A over the course of the differentiation protocols of Example 5 from Stage 5, day 1 through day 4 of Stage 6.

FIG. 28E is a graph of real time qRT-PCR results for expression of chromogranin B over the course of the differentiation protocols of Example 5 from Stage 5, day 1 through day 4 of Stage 6.

FIG. 28F is a graph of real time qRT-PCR results for expression of GCG over the course of the differentiation protocols of Example 5 from Stage 5, day 1 through day 4 of Stage 6.

FIG. 28G is a graph of real time qRT-PCR results for expression of IAPP over the course of the differentiation protocols of Example 5 from Stage 5, day 1 through day 4 of Stage 6.

FIG. 28H is a graph of real time qRT-PCR results for expression of MAFB over the course of the differentiation protocols of Example 5 from Stage 5, day 1 through day 4 of Stage 6.

FIG. 28I is a graph of real time qRT-PCR results for expression of PAX6 over the course of the differentiation protocols of Example 5 from Stage 5, day 1 through day 4 of Stage 6.

FIG. 28J is a graph of real time qRT-PCR results for expression of somatostatin over the course of the differentiation protocols of Example 5 from Stage 5, day 1 through day 4 of Stage 6.

FIG. 28K is a graph of real time qRT-PCR results for expression of insulin over the course of the differentiation protocols of Example 5 from Stage 5, day 1 through day 4 of Stage 6.

FIG. 28L is a graph of real time qRT-PCR results for expression of G6PC2 over the course of the differentiation protocols of Example 5 from Stage 5, day 1 through day 4 of Stage 6.

FIG. 28M is a graph of real time qRT-PCR results for expression of PCSK1 over the course of the differentiation protocols of Example 5 from Stage 5, day 1 through day 4 of Stage 6.

FIG. 28N is a graph of real time qRT-PCR results for expression of SLC30A8 over the course of the differentiation protocols of Example 5 from Stage 5, day 1 through day 4 of Stage 6.

FIG. 28O is a graph of real time qRT-PCR results for expression of MNX1 (HB9) over the course of the differentiation protocols of Example 5 from Stage 5, day 1 through day 4 of Stage 6.

FIG. 28P is a graph of real time qRT-PCR results for expression of UCN3 over the course of the differentiation protocols of Example 5 from Stage 5, day 1 through day 4 of Stage 6.

FIG. 29 is a graph of the c-peptide response to intra-peritoneal glucose injection of Example 5, Stage 6, day 1 cells transplanted under the kidney capsule of NSG mice.

FIG. 30A are graphs of real time qRT-PCR results for expression of ABCC8 over the course of the differentiation protocols of Example 6 from Stage 3, day 1 through the end of the differentiation protocols.

FIG. 30B are graphs of real time qRT-PCR results for expression of ALB over the course of the differentiation protocols of Example 6 from Stage 3, day 1 through the end of the differentiation protocols.

FIG. 30C are graphs of real time qRT-PCR results for expression of ARX over the course of the differentiation protocols of Example 6 from Stage 3, day 1 through the end of the differentiation protocols.

FIG. 307D are graphs of real time qRT-PCR results for expression of CDX2 over the course of the differentiation protocols of Example 6 from Stage 3, day 1 through the end of the differentiation protocols.

FIG. 30E are graphs of real time qRT-PCR results for expression of chromogranin A over the course of the differentiation protocols of Example 6 from Stage 3, day 1 through the end of the differentiation protocols.

FIG. 307F are graphs of real time qRT-PCR results for expression of chromogranin B over the course of the differentiation protocols of Example 6 from Stage 3, day 1 through the end of the differentiation protocols.

FIG. 30G are graphs of real time qRT-PCR results for expression of G6PC2 over the course of the differentiation protocols of Example 6 from Stage 3, day 1 through the end of the differentiation protocols.

FIG. 30H are graphs of real time qRT-PCR results for expression of GCG over the course of the differentiation protocols of Example 6 from Stage 3, day 1 through the end of the differentiation protocols.

FIG. 30I are graphs of real time qRT-PCR results for expression of ghrelin over the course of the differentiation protocols of Example 6 from Stage 3, day 1 through the end of the differentiation protocols.

FIG. 30J are graphs of real time qRT-PCR results for expression of IAPP over the course of the differentiation protocols of Example 6 from Stage 3, day 1 through the end of the differentiation protocols.

FIG. 30K are graphs of real time qRT-PCR results for expression of insulin over the course of the differentiation protocols of Example 6 from Stage 3, day 1 through the end of the differentiation protocols.

FIG. 30L are graphs of real time qRT-PCR results for expression of ISL1 over the course of the differentiation protocols of Example 6 from Stage 3, day 1 through the end of the differentiation protocols.

FIG. 30M are graphs of real time qRT-PCR results for expression of MAFB over the course of the differentiation protocols of Example 6 from Stage 3, day 1 through the end of the differentiation protocols.

FIG. 30N are graphs of real time qRT-PCR results for expression of MNX1 (HB9) over the course of the differentiation protocols of Example 6 from Stage 3, day 1 through the end of the differentiation protocols.

FIG. 30O are graphs of real time qRT-PCR results for expression of NEUROD1 over the course of the differentiation protocols of Example 6 from Stage 3, day 1 through the end of the differentiation protocols.

FIG. 30P are graphs of real time qRT-PCR results for expression of NEUROG3 over the course of the differentiation protocols of Example 6 from Stage 3, day 1 through the end of the differentiation protocols.

FIG. 30Q are graphs of real time qRT-PCR results for expression of NKX2.2 over the course of the differentiation protocols of Example 6 from Stage 3, day 1 through the end of the differentiation protocols.

FIG. 30R are graphs of real time qRT-PCR results for expression of NKX6.1 over the course of the differentiation protocols of Example 6 from Stage 3, day 1 through the end of the differentiation protocols.

FIG. 30S are graphs of real time qRT-PCR results for expression of PAX4 over the course of the differentiation protocols of Example 6 from Stage 3, day 1 through the end of the differentiation protocols.

FIG. 30T are graphs of real time qRT-PCR results for expression of PAX6 over the course of the differentiation protocols of Example 6 from Stage 3, day 1 through the end of the differentiation protocols.

FIG. 30U are graphs of real time qRT-PCR results for expression of PCSK1 over the course of the differentiation protocols of Example 6 from Stage 3, day 1 through the end of the differentiation protocols.

FIG. 30V are graphs of real time qRT-PCR results for expression of PCSK2 over the course of the differentiation protocols of Example 6 from Stage 3, day 1 through the end of the differentiation protocols.

FIG. 30W are graphs of real time qRT-PCR results for expression of PDX1 over the course of the differentiation protocols of Example 6 from Stage 3, day 1 through the end of the differentiation protocols.

FIG. 30X are graphs of real time qRT-PCR results for expression of pancreatic polypeptide over the course of the differentiation protocols of Example 6 from Stage 3, day 1 through the end of the differentiation protocols.

FIG. 30Y are graphs of real time qRT-PCR results for expression of PTF1A over the course of the differentiation protocols of Example 6 from Stage 3, day 1 through the end of the differentiation protocols.

FIG. 30Z are graphs of real time qRT-PCR results for expression of SLC30A8 over the course of the differentiation protocols of Example 6 from Stage 3, day 1 through the end of the differentiation protocols.

FIG. 30AA are graphs of real time qRT-PCR results for expression of SST over the course of the differentiation protocols of Example 6 from Stage 3, day 1 through the end of the differentiation protocols.

FIG. 30BB are graphs of real time qRT-PCR results for expression of UCN3 over the course of the differentiation protocols of Example 6 from Stage 3, day 1 through the end of the differentiation protocols.

FIG. 30CC are graphs of real time qRT-PCR results for expression of WNT4A over the course of the differentiation protocols of Example 6 from Stage 3, day 1 through the end of the differentiation protocols.

FIG. 31 is a graph (+/− standard deviation) of the average c-peptide response to intra-peritoneal glucose injection of Example 5 cells (Standard, N=7, and Skip 4, N=7) transplanted under the kidney capsule of NSG mice at Stage 5, day 7 of differentiation.

FIG. 32 are graphs of FACS profiles of Stage 5, day 7 cells differentiated according to the protocol of Example 7 and stained for NKX6.1 (X-axis) co-stained with NEUROD1 (Y-axis).

FIG. 33 are graphs of FACS profiles of Stage 5, day 7 cells differentiated according to the protocol of Example 7 and stained for PDX1 (X-axis) co-stained with NKX6.1 (Y-axis).

FIG. 34 are graphs of FACS profiles of Stage 5, day 7 cells differentiated according to the protocol of Example 7 and stained for NKX6.1 (X-axis) co-stained with insulin (Y-axis).

FIG. 35 is a graph of the c-peptide response, at 6 weeks post-implant, before and after intra-peritoneal glucose injection, for Stage 5, day 8 cells of Example 7 transplanted under the kidney capsule of NSG mice (N=7).

FIG. 36 is a graph of the c-peptide response, at 12 weeks post-implant, before and after intra-peritoneal glucose injection, for Stage 5, day 8 cells of Example 7 transplanted under the kidney capsule of NSG mice (N=7).

FIGS. 37A and 37B are graphs of pH profiles of the media within the spinner flasks of Example 8.

FIG. 38 is a graph of the lactate production of the cells of Example 8

FIG. 39 depicts LIVE/DEAD fluorescence imaging for cells of Example 8.

### DETAILED DESCRIPTION OF THE INVENTION

This invention is directed to preparing embryonic stem cells and other pluripotent cells that maintain pluripotency in aggregated cell clusters for differentiation to endocrine progenitor cells and pancreatic endocrine cells. It is a discovery of the invention that, by controlling one or more of pH, cell concentration and retinoid concentration, especially during the differentiation stages in which PDX1 and PDX1/NKX6.1 co-expressing cells are produced, one can generate a nearly homogenous population, meaning ≧80%, preferably ≧90% of the cell population, of PDX1/NKX6.1 co-expressing cells by suppressing precocious NGN3 expression and promoting NKX6.1 expression. When the nearly homogenous population of PDX1/NKX6.1 co-expressing cells is further differentiated in vitro, it matures to form a population of pancreatic endocrine cells that co-express PDX1, NKX6.1, insulin and MAFA.

It is an additional discovery of the invention that using a pH below the homeostatic level of pH 7.4 to a level of about 7.2 or less, preferably about 7.2 to about 7.0, more preferably about 7.0, during one or more stages of differentiation, while also using a cell density of equal to or greater than about 1.5 million cells/mL to about 3.0 million cells/mL, preferably about 1.8 million cells/mL to about 3.0 million cells/mL, more preferably about 2.0 million cells/mL to about 3.0 million cells/mL, the need for the addition of components to inhibit, block, activate or agonize TGF-β or BMP signaling and the use of sonic hedgehog inhibitors can be eliminated.

In the methods of the invention, foregut endoderm cells may be differentiated to pancreatic endoderm cells absent expression of PTF1A or NGN3. It is believed that the use of low pH, meaning equal to or less than about 7.2 to about 7.0, blocks the expression of NGN3. The PTF1A or NGN3 negative cells may be further enriched in a subsequent stage to a pancreatic endoderm cell population that has high levels of PDX1and NKX6.1 (equal to or greater than 96% positive) and that express some PTF1A, but still do not have NGN3 expression. Cells may be moved directly from the pancreatic endoderm absent expression of PTF1A or NGN3 stage directly into a stage in which pancreatic endocrine precursor cells, with high NGN3 expression, transition to pancreatic endocrine cells by the end of the stage. Furthermore, as soon as the pancreatic endoderm cells absent expression of PTF1A or NGN3n cells move into this stage, in which pancreatic endocrine cells are formed, the cells begin to show expression (by PCR) of MAFA, and this expression is detectable as protein by the end of the stage.

Stem cells useful in the invention are undifferentiated cells defined by their ability, at the single cell level, to both self-renew and differentiate. Stem cells may produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm, and ectoderm). Stem cells also give rise to tissues of multiple germ layers following transplantation and contribute substantially to most, if not all, tissues following injection into blastocysts.

Stem cells are classified by their developmental potential. “Cell culture” or “culturing” refer generally to cells taken from a living organism and grown under controlled conditions (“in culture” or “cultured”). A “primary cell culture” is a culture of cells, tissues, or organs taken directly from an organism before the first subculture. Cells are expanded in culture when they are placed in a growth medium under conditions that facilitate one or both of cell growth and division, resulting in a larger population of the cells. When cells are expanded in culture, the rate of cell proliferation is sometimes measured by the amount of time needed for the cells to double in number (referred to as “doubling time”).

“Expanding”, as used herein is the process of increasing the number of pluripotent stem cells by culturing, such as by at least about 5%, 10%, 15%, 20%, 25%, 30%, 35%, 40%, 45%, 50%, 60%, 75%, 90%, 100%, 200%, 500%, 1000% or more, and levels within these percentages. It is appreciated that the number of pluripotent stem cells which can be obtained from a single pluripotent stem cell depends on the proliferation capacity of the pluripotent stem cell. The proliferation capacity of the pluripotent stem cell can be calculated by the doubling time of the cell, i.e., the time needed for a cell to undergo a mitotic division in the culture, and the period that the pluripotent stem cell can be maintained in the undifferentiated state, which is equivalent to the number of passages multiplied by the days between each passage.

Differentiation is the process by which an unspecialized (“uncommitted”) or less specialized cell acquires the features of a specialized cell such as, a nerve cell or a muscle cell. A differentiated cell or a differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. “De-differentiation” refers to the process by which a cell reverts to a less specialized (or committed) position within the lineage of a cell. As used herein, the lineage of a cell defines the heredity of the cell, i.e., which cells it came from and to what cells it can give rise. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

“Markers”, as used herein, are nucleic acid or polypeptide molecules that are differentially expressed in a cell of interest. In this context, differential expression means an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art.

As used herein, a cell is “positive for” a specific marker or “positive” when the specific marker is sufficiently detected in the cell. Similarly, the cell is “negative for” a specific marker, or “negative” when the specific marker is not sufficiently detected in the cell. In particular, positive by FACS is usually greater than 2%, whereas the negative threshold by FACS is usually less than 1%. Positive by PCR, using the OpenArray® PCR system, is usually less than 30 cycles (Cts) and negative is usually 30 or more cycles. Positive by PCR, using the TaqMan® PCR assay, is usually less than 34 cycles (Cts) and negative by PCR is usually more than 34.5 cycles.

As used herein, “cell density” and “seeding density” are used interchangeably and refer to the number of cells seeded per unit area of a solid or semisolid planar or curved substrate.

“Cell concentration” is used to refer to the number of cells per given unit of volume.

As used herein, “suspension culture” refers to a culture of cells, single cells, clusters, or a mixture of single cells and clusters suspended in medium rather than adhering to a surface.

As used herein, “serum free” refers to being devoid of human or animal serum. Accordingly, a serum free culture medium does not comprise serum or portions of serum.

In attempts to replicate the differentiation of pluripotent stem cells into functional pancreatic endocrine cells in cell culture, the differentiation process is often viewed as progressing through a number of consecutive stages. As used herein, the various stages are defined by the culturing times, and reagents set forth in the examples included herein.

“Definitive endoderm”, as used herein, refers to cells which bear the characteristics of cells arising from the epiblast during gastrulation and which form the gastrointestinal tract and its derivatives. Definitive endoderm cells express at least one of the following markers: FOXA2 also known as hepatocyte nuclear factor 3-β (HNF3β)), GATA4, GATA6, MNX1, SOX17, CXCR4, Cerberus, OTX2, brachyury, goosecoid, C-Kit, CD99, and MIXL1. Markers characteristic of the definitive endoderm cells include CXCR4, FOXA2 and SOX17. Thus, definitive endoderm cells may be characterized by their expression of CXCR4, FOXA2, and SOX17. In addition, depending on the length of time cells are allowed to remain in the first stage of differentiation, an increase in HNF4a may be observed.

“Foregut endoderm cells,” as used herein, refers to endoderm cells that give rise to the esophagus, lungs, stomach, liver, pancreas, gall bladder, and a portion of the duodenum. Foregut endoderm cells express at least one of the following markers: PDX1, FOXA2, CDX2, SOX2, and HNF4α. Foregut endoderm cells may be characterized by an increase in expression of PDX1 compared to gut tube cells.

“Pancreatic foregut precursor cells,” as used herein, refers to cells that express at least one of the following markers: PDX1, NKX6.1, HNF6, NGN3, SOX9, PAX4, PAX6, ISL1, gastrin, FOXA2, PTF1A, PROX1 and HNF4α. Pancreatic foregut precursor cells may be characterized by being positive for the expression of PDX1, NKX6.1, and SOX9.

“Pancreatic endoderm cells,” as used herein, refers to cells that express at least one of the following markers: PDX1, NKX6.1, HNF1 β, PTF1A, HNF6, HNF4α, SOX9, NGN3; gastrin; HB9, or PROX1. Pancreatic endoderm cells may be characterized by their lack of substantial expression of CDX2 or SOX2.

“Pancreatic endocrine precursor cells,” as used herein, refers to pancreatic endoderm cells capable of becoming a pancreatic hormone expressing cell. Pancreatic endocrine precursor cells express at least one of the following markers: NGN3; NKX2.2; NeuroDl; ISL1; PAX4; PAX6; or ARX. Pancreatic endocrine precursor cells may be characterized by their expression of NKX2.2 and NEUROD 1.

“Pancreatic endocrine cells,” as used herein, refer to cells capable of expressing at least one of the following hormones: insulin, glucagon, somatostatin, ghrelin, and pancreatic polypeptide. In addition to these hormones, markers characteristic of pancreatic endocrine cells include one or more of NGN3, NeuroD1, ISL1, PDX1, NKX6.1, PAX4, ARX, NKX2.2, and PAX6. Pancreatic endocrine cells expressing markers characteristic of β cells can be characterized by their expression of insulin and at least one of the following transcription factors: PDX1, NKX2.2, NKX6.1, NEUROD1, ISL1, HNF3β, MAFA, PAX4, and PAX6.

By “retinoid” is meant retinoic acid or a compound that is a retinoic receptor agonist.

Used interchangeably herein are “d1”, “d 1”, and “day 1”; “d2”, “d 2”, and “day 2”; “d3”, “d 3”, and “day 3”, and so on. These number-letter combinations refer to a specific day of incubation in the different stages during the stepwise differentiation protocol of the instant application.

“Glucose” and “D-Glucose” are used interchangeably herein and refer to dextrose, a sugar commonly found in nature.

Pluripotent stem cells may express one or more of the designated TRA-1-60 and TRA-1-81 antibodies (Thomson et al. 1998, Science 282:1145-1147). Differentiation of pluripotent stem cells in vitro results in the loss of TRA-1-60, and TRA-1-81 expression. Undifferentiated pluripotent stem cells typically have alkaline phosphatase activity, which can be detected by fixing the cells with 4% paraformaldehyde, and then developing with Vector® Red as a substrate, as described by the manufacturer (Vector Laboratories, Inc., Burlingame, Calif.). Undifferentiated pluripotent stem cells also typically express OCT4 and TERT, as detected by RT-PCR.

Another desirable phenotype of propagated pluripotent stem cells is a potential to differentiate into cells of all three germinal layers: endoderm, mesoderm, and ectoderm tissues. Pluripotency of stem cells can be confirmed, for example, by injecting cells into severe combined immune-deficiency (“SCID”) mice, fixing the teratomas that form using 4% paraformaldehyde, and then examining histologically for evidence of cell types from these three germ layers. Alternatively, pluripotency may be determined by the creation of embryoid bodies and assessing the embryoid bodies for the presence of markers associated with the three germinal layers.

Propagated pluripotent stem cell lines may be karyotyped using a standard G-banding technique and compared to published karyotypes of the corresponding primate species. It is desirable to obtain cells that have a “normal karyotype,” which means that the cells are euploid, wherein all human chromosomes are present and not noticeably altered. Pluripotent cells may be readily expanded in culture using various feeder layers or by using matrix protein coated vessels. Alternatively, chemically defined surfaces in combination with defined media such as mTeSR®1 media (StemCell Technologies, Vancouver, BC, Canada) may be used for routine expansion of the cells.

Culturing in a suspension culture according to the method of some embodiments of the invention is effected by seeding the pluripotent stem cells in a culture vessel at a cell concentration that promotes cell survival and proliferation, but limits differentiation. Typically, a seeding density sufficient to maintains cells in a pluripotent, undifferentiated state is used. It will be appreciated that although single-cell suspensions of stem cells may be seeded, small clusters of cells may be advantageous.

To provide the pluripotent stem cells with a sufficient and constant supply of nutrients and growth factors while in the suspension culture, the culture medium can be replaced or replenished on a daily basis or at a pre-determined schedule such as every 1-5 days. Large clusters of pluripotent stem cells may cause cell differentiation, thus, measures may be taken to avoid large pluripotent stem cell aggregates. According to some embodiments of the invention, the formed pluripotent stem cell clusters are dissociated, for example, every 2-7 days and the single cells or small clumps of cells are either split into additional culture vessels (i.e., passaged) or retained in the same culture vessel and processed with replacement or additional culture medium.

Large pluripotent stem cell clumps, including a pellet of pluripotent stem cells resulting from centrifugation, can be subjected to one or both of enzymatic digestion and mechanical dissociation. Enzymatic digestion of pluripotent stem cell clumps can be performed by subjecting the clump to an enzyme, such as type IV Collagenase, Dispase® or Accutase®. Mechanical dissociation of large pluripotent stem cell clumps can be performed using a device designed to break the clumps to a predetermined size. Additionally, or alternatively, mechanical dissociation can be manually performed using a needle or pipette.

The culture vessel used for culturing the pluripotent stem cells in suspension according to the method of some embodiments of the invention can be any tissue culture vessel (e.g., with a purity grade suitable for culturing pluripotent stem cells) having an internal surface designed such that pluripotent stem cells cultured therein are unable to adhere or attach to such a surface (e.g., non-tissue culture treated vessel, to prevent attachment or adherence to the surface). Preferably to obtain a scalable culture, culturing according to some embodiments of the invention is effected using a controlled culturing system (preferably a computer-controlled culturing system) in which culture parameters such as temperature, agitation, pH, and oxygen are automatically monitored and controlled using a suitable device. Once the desired culture parameters are determined, the system may be set for automatic adjustment of culture parameters as needed to enhance pluripotent stem cell expansion and differentiation.

The pluripotent stem cells may be cultured under dynamic conditions (i.e., under conditions in which the pluripotent stem cells are subject to constant movement while in the suspension culture, e.g. a stirred suspension culture system) or under non-dynamic conditions (i.e., a static culture) while preserving their, proliferative, pluripotent capacity and karyotype stability over multiple passages.

For non-dynamic culturing of pluripotent stem cells, the pluripotent stem cells can be cultured in petri dishes, T-flasks, HyperFlasks® (Corning Incorporated, Corning, N.Y). Cell Stacks® (Corning Incorporated, Corning, N.Y.) or Cell Factories (NUNC™ Cell Factory™ Systems (Thermo Fisher Scientific, Inc., Pittsburgh, Pa.)) coated or uncoated. For dynamic culturing of pluripotent stem cells, the pluripotent stem cells can be cultured in a suitable vessel, such as spinner flasks or Erlenmeyer flasks, stainless steel, glass or single use plastic shaker or stirred tank vessels. The culture vessel can be connected to a control unit and thus present a controlled culturing system. The culture vessel (e.g., spinner flask or Erlenmeyer flask) may be agitated continuously or intermittently. Preferably the cultured vessel is agitated sufficiently to maintain the pluripotent stem cells in suspension.

The pluripotent stem cells may be cultured in any medium that provides sufficient nutrients and environmental stimuli to promote growth and expansion. Suitable media include E8™, IH3 and mTeSR®1 or mTeSR®2. The media may be changed periodically to refresh the nutrient supply and remove cellular by-products. According to some embodiments of the invention, the culture medium is changed daily.

Any pluripotent stem cell may be used in the methods of the invention. Exemplary types of pluripotent stem cells that may be used include established lines of pluripotent cells derived from tissue formed after gestation, including pre-embryonic tissue (such as, for example, a blastocyst), embryonic tissue, or fetal tissue taken any time during gestation, typically but not necessarily, before approximately 10 to 12 weeks gestation. Non-limiting examples are established lines of human embryonic stem cells (“hESCs”) or human embryonic germ cells, such as, for example the human embryonic stem cell lines H1, H7, and H9 (WiCell Research Institute, Madison, Wis., USA). Also suitable are cells taken from a pluripotent stem cell population already cultured in the absence of feeder cells.

Also suitable are inducible pluripotent cells (“IPS”) or reprogrammed pluripotent cells that can be derived from adult somatic cells using forced expression of a number of pluripotent related transcription factors, such as OCT4, NANOG, SOX2, KLF4, and ZFP42 (Annu Rev Genomics Hum Genet 2011, 12:165-185). The human embryonic stem cells used in the methods of the invention may also be prepared as described by Thomson et al. (U.S. Pat. No. 5,843,780; Science, 1998, 282:1145-1147; Curr Top Dev Biol 1998, 38:133-165; Proc Natl Acad Sci U.S.A. 1995, 92:7844-7848). Also suitable are mutant human embryonic stem cell lines, such as, for example, BG01v (BresaGen, Athens, Ga.), or cells derived from adult human somatic cells, such as, for example, cells disclosed in Takahashi et al., Cell 131: 1-12 (2007). Pluripotent stem cells suitable for use in the present invention may be derived according to the methods described in Li et al. (Cell Stem Cell 4: 16-19, 2009); Maherali et al. (Cell Stem Cell 1: 55-70, 2007); Stadtfeld et al. (Cell Stem Cell 2: 230-240); Nakagawa et al. (Nature Biotechnology 26: 101-106, 2008); Takahashi et al. (Cell 131: 861-872, 2007); and U.S. Patent App. Pub. No. 2011-0104805. Other sources of pluripotent stem cells include induced pluripotent cells (IPS, Cell, 126(4): 663-676). Other sources of cells suitable for use in the methods of invention include human umbilical cord tissue-derived cells, human amniotic fluid-derived cells, human placental-derived cells, and human parthenotes. In one embodiment, the umbilical cord tissue-derived cells may be obtained using the methods of U.S. Pat. No. 7,510,873, the disclosure of which is incorporated by reference in its entirety as it pertains to the isolation and characterization of the cells. In another embodiment, the placental tissue-derived cells may be obtained using the methods of U.S. App. Pub. No. 2005/0058631, the disclosure of which is incorporated by reference in its entirety as it pertains to the isolation and characterization of the cells. In another embodiment, the amniotic fluid-derived cells may be obtained using the methods of U.S. App. Pub. No. 2007/0122903, the disclosure of which is incorporated by reference in its entirety as it pertains to the isolation and characterization of the cells.

Characteristics of pluripotent stem cells are well known to those skilled in the art, and additional characteristics of pluripotent stem cells continue to be identified. Pluripotent stem cell markers include, for example, the expression of one or more (e.g. 1, 2, 3, 4, 5, 6, 7, 8, 9, 10, 11, 12, 13, 14 or all) of the following: ABCG2, cripto, FOXD3, CONNEXIN43, CONNEXIN45, OCT4, SOX2, NANOG, hTERT, UTF1, ZFP42, SSEA-3, SSEA-4, TRA-1-60, TRA-1-81. In one embodiment, the pluripotent stem cells suitable for use in the methods of the invention express one or more (e.g. 1, 2, 3 or all) of CD9, SSEA4, TRA-1-60, and TRA-1-81, and lack expression of a marker for differentiation CXCR4 (also known as CD184) as detected by flow cytometry. In another embodiment, the pluripotent stem cells suitable for use in the methods of the invention express one or more (e.g. 1, 2 or all) of CD9, NANOG and POU5F1/OCT4 as detected by RT-PCR.

Exemplary pluripotent stem cells include the human embryonic stem cell line H9 (NIH code: WA09), the human embryonic stem cell line H1 (NIH code: WA01), the human embryonic stem cell line H7 (NIH code: WA07), and the human embryonic stem cell line SA002 (Cellartis, Sweden). In one embodiment, the pluripotent stem cells are human embryonic stem cells, for example, H1 hES cells. In alternate embodiments, pluripotent stem cells of non-embryonic origin are used.

The present invention, in some of the embodiments as described below, relates to isolating and culturing stem cells, in particular culturing stem cell clusters, which retain pluripotency in a dynamic suspension culture system. Pluripotent cell clusters may be differentiated to produce functional β cells.

The pluripotent stem cells used in the methods of the present invention are preferably expanded in dynamic suspension culture prior to differentiation toward a desired end point. Advantageously, it has been found that the pluripotent stem cells can be cultured and expanded as clusters of cells in suspension in a suitable medium without loss of pluripotency. Such culturing may occur in a dynamic suspension culture system wherein the cells or cell clusters are kept moving sufficiently to prevent loss of pluripotency. Useful dynamic suspension culture systems include systems equipped with means to agitate the culture contents, such as via stirring, shaking, recirculation or the bubbling of gasses through the media. Such agitation may be intermittent or continuous, as long as sufficient motion of the cell clusters is maintained to facilitate expansion and prevent premature differentiation. Preferably, the agitation comprises continuous stirring such as via an impeller rotating at a particular rate. The impeller may have a rounded or flat bottom. The stir rate of the impeller should be such that the clusters are maintained in suspension and settling is minimized. Further, the angle of the impeller blade may be adjusted to aid in the upward movement of the cells and clusters to avoid settling. In addition, the impeller type, angle and rotation rate may all be coordinated such that the cells and clusters are in what appears as a uniform colloidal suspension.

Suspension culturing and expansion of pluripotent stem cell clusters may be accomplished by transfer of static cultured stem cells to an appropriate dynamic culture system such as a disposable plastic, reusable plastic, stainless steel or glass vessel, e.g. a spinner flask or an Erlenmeyer flask. For example, stem cells cultured in an adherent static environment, i.e., plate or dish surface, may first be removed from the surface by treatment with a chelating agent or enzyme. Suitable enzymes include, but are not limited to, type I Collagenase, Dispase® (Sigma Aldrich LLC, St. Louis, Mo.) or a commercially available formulation sold under the trade name Accutase® (Sigma Aldrich LLC, St. Louis, Mo.). Accutase® is a cell detachment solution comprising collagenolytic and proteolytic enzymes (isolated from crustaceans) and does not contain mammalian or bacterial derived products. Therefore, in one embodiment, the enzyme is a collagenolytic enzyme or a proteolytic enzyme or a cell detachment solution comprising collagenolytic and proteolytic enzymes. Suitable chelating agents include, but are not limited to, ethylenediaminetetraacetic acid (“EDTA”). In some embodiments, the pluripotent stem cell cultures are incubated with the enzyme or chelating agent, preferably until colony edges began to curl and lift, but prior to full detachment of colonies from the culture surface. In one embodiment, the cell cultures are incubated at room temperature. In one embodiment, the cells are incubated at a temperature of more than 20° C., more than 25° C., more than 30° C. or more than 35° C., for example, at a temperature of between about 20° C. and about 40° C., between about 25° C. and about 40° C., between about 30° C. and about 40° C., for example, about 37° C. In one embodiment, the cells are incubated for at least about 1, at least about 5, at least about 10, at least about 15, at least about 20 minutes, for example between about 1 and about 30 minutes, between about 5 and about 30 minutes, between about 10 and about 25 minutes, between about 15 and about 25 minutes, for example, about 20 minutes. In one embodiment, the method involves the step of removing the enzyme or chelating agent from the cell culture after treatment. In one embodiment, the cell culture is washed once or twice or more, after removal of the enzyme or chelating agent. In one embodiment the cell culture is washed with an appropriate culture medium, such as mTeSR®1 (Stem Cell Technologies, Vancouver, BC, Canada). In one embodiment, a Rho-kinase inhibitor (for example, Y-27632, Axxora Catalog#ALX-270-333, San Diego, Calif.). The Rho-kinase inhibitor may be at a concentration of about 1 to about 100 μM, about 1 to 90 μM, about 1 to about 80 μM, about 1 to about 70 μM, about 1 to about 60 μM, about 1 to about 50 μM, about 1 to about 40 μM, about 1 to about 30 μM, about 1 to about 20 μM, about 1 to about 15 μM, about 1 to about 10 μM, or about 10 μM. In one embodiment, the Rho-kinase inhibitor is added at least 1 μM, at least 5 μM or at least 10 μM. The cells may be lifted from the surface of the static culture system with a scraper or rubber policeman. Media and cells may be transferred to a dynamic culture system using a glass pipette or other suitable means. In a preferred embodiment, the media in the dynamic culture system is changed daily.

The invention provides, in one embodiment, methods of culturing and expanding pluripotent stem cells in a three-dimensional suspension culture. In particular, the methods provide for the culturing and expanding pluripotent stem cells by forming aggregated cell clusters of these pluripotent stem cells. The cell clusters may form as a result of treating pluripotent stem cell cultures with an enzyme (e.g. a neutral protease, for example Dispase®) or a chelating agent prior to culturing the cells. The cells may preferably be cultured in a stirred or shaken suspension culture system. In one embodiment, the invention further provides for formation of cells expressing markers characteristic of the pancreatic endoderm lineage from such clusters of pluripotent stem cells.

Preferably, the cell clusters are aggregated pluripotent stem cells. The aggregated stem cells express one or more markers of pluripotency, for example, one or more (e.g. 1, 2, 3 or all) of the markers CD9, SSEA4, TRA-1-60, and TRA-1-81, and lack expression of one or more markers for differentiation, for example, lack expression of CXCR4. In one embodiment, the aggregated stem cells express the markers for pluripotency CD9, SSEA4, TRA-1-60, and TRA-1-81, and lack expression of a marker for differentiation CXCR4.

One embodiment is a method of culturing pluripotent stem cells as cell clusters in suspension culture. The cell clusters are aggregated pluripotent stem cells, cultured in a dynamic stirred or shaken suspension culture system. The cell clusters may be transferred from a planar adherent culture using an enzyme, such as a neutral protease, for example Dispase, as a cell lifting agent to a stirred or shaken suspension culture system. Exemplary suitable enzymes include, but are not limited to, type IV Collagenase, Dispase® or Accutase®. The cells maintain pluripotency in a stirred or shaken suspension culture system, in particular a stirred suspension culture system.

Another embodiment of the invention is a method of culturing pluripotent stem cells as cell clusters in suspension culture, wherein the cell clusters are aggregated pluripotent stem cells transferred from a planar adherent culture using a chelating agent, for example EDTA, and cultured in a stirred or shaken suspension culture system. The cell clusters maintain pluripotency in a stirred or shaken suspension culture system, in particular a stirred (dynamically agitated) suspension culture system.

Another embodiment of the invention is a method of culturing pluripotent stem cells as cell clusters in suspension culture, wherein the cell clusters are aggregated pluripotent stem cells transferred from a planar adherent culture using the enzyme Accutase®, and cultured in a stirred or shaken suspension culture system. The cell clusters maintain pluripotency in the dynamically agitated suspension culture system.

The cell clusters of the invention may be differentiated into mesoderm cells, such as cardiac cells, ectoderm cells, such as neural cells, single hormone positive cells or pancreatic endoderm cells. The method may further include differentiation, for example differentiation of the pancreatic endoderm cells into pancreatic precursor cells and pancreatic hormone expressing cells. In another embodiment, pancreatic precursor cells are characterized by expression of β cell transcription factors PDX1 and NKX6.1.

In one embodiment, the step of differentiation is carried out after at least 12 hours, at least 24 hours, at least 36 hours, at least 48 hours, at least 72 hours, at least 96 hours, at least 120 hours, at least 144 hours, at least 168 hours, at least 196 hours or more, preferably about 48 hours to about 72 hours in the suspension culture system. Differentiation may be carried out using a stage-wise progression of media components, such as that described in the examples or Table A below.

In one embodiment, a three-dimensional cell cluster is produced by growing pluripotent stem cells in a planar adherent culture; expanding the pluripotent stem cells to aggregated cell clusters; and transferring the clusters of pluripotent stem cells from the planar adherent culture to a dynamic suspension culture using an enzyme or chelating agent. A further embodiment is a method of expanding and differentiating pluripotent stem cells in a dynamically agitated suspension culture system by growing pluripotent stem cells in a planar adherent culture; expanding the pluripotent stem cells to aggregated cell clusters; and transferring the clusters of pluripotent stem cells from the planar adherent culture to a dynamic suspension culture using an enzyme or chelating agent; and differentiating the pluripotent cell clusters in a dynamic agitated suspension culture system to generate a pancreatic precursor cell population.

Another embodiment is a transplantable stem cell derived cell product comprising differentiated stem cells prepared from suspension of expanded pluripotent stem cell clusters that are differentiated to pancreatic precursor cells. More particularly, a transplantable stem cell derived product is produced by growing pluripotent stem cells in a planar adherent culture; expanding the pluripotent stem cells to aggregated cell clusters; and transferring the clusters of pluripotent stem cells from the planar adherent culture to a dynamic suspension culture using an enzyme or chelating agent; and differentiating the pluripotent cell clusters in a dynamically agitated suspension culture system. The transplantable stem cell derived cell product is preferably used to treat diabetes.

In another embodiment, the method includes transplantation into a diabetic animal for further in vivo maturation to functional pancreatic endocrine cells.

Another embodiment is a method of expanding and differentiating pluripotent stem cells in a suspension culture system comprising growing pluripotent stem cells in a planar adherent culture; removing the pluripotent stem cells from the planar adherent culture using an enzyme; adhering the pluripotent stem cells to microcarriers in static culture; expanding the pluripotent cells in a dynamically agitated suspension culture system; and differentiating the pluripotent cells in a dynamically agitated suspension culture system to generate a pancreatic precursor cell population.

The microcarriers may be of any form known in the art for adhering cells, in particular the microcarriers may be beads. The microcarrier can be comprised of natural or synthetically-derived materials. Examples include collagen-based microcarriers, dextran-based microcarriers, or cellulose-based microcarriers. For example, microcarrier beads may be modified polystyrene beads with cationic trimethyl ammonium attached to the surface to provide a positively charged surface to the microcarrier. The bead diameter may range from about 90 to about 200 μm, alternately from about 100 to about 190 μm, alternatively from about 110 to about 180 μm, alternatively from about 125 to 175 μm in diameter. Microcarrier beads may also be a thin layer of denatured collagen chemically coupled to a matrix of cross-linked dextran. Microcarrier beads may be glass, ceramics, polymers (such as polystyrene), or metals. Further, microcarriers may be uncoated, or coated, such as with silicon or a protein such as collagen. In a further aspect the microcarrier can be comprised of, or coated with, compounds that enhance binding of the cell to the microcarrier and enhance release of the cell from the microcarrier including, but not limited to, sodium hyaluronate, poly(monostearoylglyceride co-succinic acid), poly-D,L-lactide-co-glycolide, fibronectin, laminin, elastin, lysine, n-isopropyl acrylamide, vitronectin, and collagen. Examples further include microcarriers that possess a microcurrent, such as microcarriers with a particulate galvanic couple of zinc and copper that produces low levels of biologically relevant electricity; or microcarriers that are paramagnetic, such as paramagnetic calcium-alginate microcarriers.

In some embodiments, the population of pancreatic endoderm cells is obtained by a stepwise differentiation of pluripotent cell clusters. In some embodiments, the pluripotent cells are human embryonic pluripotent stem cells. In one aspect of the present invention, a cell expressing markers characteristic of the definitive endoderm lineage is a primitive streak precursor cell. In an alternate aspect, a cell expressing markers characteristic of the definitive endoderm lineage is a mesendoderm cell.

In some embodiments, the present invention relates to a stepwise method of differentiating pluripotent cells comprising culturing stage 3-5 cells in a dynamic suspension culture. In some embodiments, the pancreatic endoderm population generated is transplanted into diabetic animals for further in vivo maturation to functional pancreatic endocrine cells. The invention also provides for systems or kits for use in the methods of the invention.

The invention also provides a cell or population of cells obtainable by a method of the invention. The invention also provides a cell or population of cells obtained by a method of the invention.

The invention provides methods of treatment. In particular, the invention provides methods for treating a patient suffering from, or at risk of developing, diabetes.

The invention also provides a cell or population of cells obtainable or obtained by a method of the invention for use in a method of treatment. In particular, the invention provides a cell or population of cells obtainable or obtained by a method of the invention for use in a method of treating a patient suffering from, or at risk of developing, diabetes. The diabetes may be Type 1 or Type 2 diabetes.

In one embodiment, the method of treatment comprises implanting cells obtained or obtainable by a method of the invention into a patient.

In one embodiment, the method of treatment comprises differentiating pluripotent stem cells in vitro into Stage 1, Stage 2, Stage 3, Stage 4, Stage 5, or Stage 6 cells, for example as described herein, and implanting the differentiated cells into a patient.

In one embodiment, the method further comprises the step of culturing pluripotent stem cells, for example as described herein, prior to the step of differentiating the pluripotent stem cells.

In one embodiment, the method further comprises the step of differentiating the cells in vivo, after the step of implantation.

In one embodiment, the patient is a mammal, preferably a human.

In one embodiment, the cells may be implanted as dispersed cells or formed into clusters that may be implanted or alternatively infused into the hepatic portal vein. Alternatively, cells may be provided in biocompatible degradable polymeric supports, porous non-degradable devices or encapsulated to protect from host immune response. The cells may be implanted into any appropriate site in a recipient. The implantation sites include, for example, the liver, natural pancreas, renal subcapsular space, omentum, peritoneum, subserosal space, intestine, stomach, or a subcutaneous pocket.

To enhance further differentiation, survival or activity of the implanted cells in vivo, additional factors, such as growth factors, antioxidants or anti-inflammatory agents, can be administered before, simultaneously with, or after the administration of the cells. These factors can be secreted by endogenous cells and exposed to the administered cells in situ. Implanted cells can be induced to differentiate by any combination of endogenous growth factors known in the art and exogenously administered growth factors known in the art.

The amount of cells used in implantation depends on a number of various factors including the patient's condition and response to the therapy, and can be determined by one skilled in the art.

In one embodiment, the method of treatment further comprises incorporating the cells into a three-dimensional support prior to implantation. The cells can be maintained in vitro on this support prior to implantation into the patient. Alternatively, the support containing the cells can be directly implanted in the patient without additional in vitro culturing. The support can optionally be incorporated with at least one pharmaceutical agent that facilitates the survival and function of the transplanted cells.

In certain embodiments of the invention, one or more of the components listed on Table A may be used in the methods of the invention:

TABLE A

| Component/Condition | Suitable Amounts/Concentrations |
|---|---|
| ALK5 inhibitor II | About 500 to about 30,000 nM (30 μM), about |
| | 600 to about 20,000 nM (20 μM), about 700 to |
| | about 10,000 nM (10 μM), about 800 to about |
| | 1000 nM (10 μM), about 10 μM, about 100 nM, |
| | about 500 nM or about 1 μM, from about 0.6 to |
| | about 10 μM, from about 0.6 to about 1 μM |
| Ascorbic acid | About 0 to about 250 μM |
| Betacellulin | About 0 to about 20 ng/mL |
| CHIR99021 | About 3 to about 30 μM |
| FAF-BSA | About 2%, 0.1% to about 2% |
| FGF7 | About 50 ng/mL, from about 30 ng/ml to about |
| | 60 ng/ml, from about 25 ng/ml to about 55 ng/ml |
| Gamma secretase | About 0 to about 1,000 nM, about 30 to about |
| inhibitor XX | 300 nM, about 100 nM to about 1 μM; about 100 |
| | nM; about 1 μM |
| Gamma secretase | About 0 to about 3,000 nM, about 100 nM to |
| inhibitor XXI | about 3000 nM, about 100 nM to about 1 μM; |
| | about 100 nM; about 1 μM |
| GDF8 | About 100 ng/mL, from about 80 ng/ml to about |
| | 150 ng/ml, from about 75 ng/ml to about 125 |
| | ng/ml, from about 75 ng/ml to about 150 ng/ml |
| Glucose | About 1 mM to about 50 mM; about 1 mM to |
| | about 25.5 mM, about 1 mM to about 20 mM, |
| | about 1 nM to about 10 nM, about 1 nM to about |
| | 10 nM, about 1 nM to about 8 nM, about 1 nM to |
| | about 5 nM |
| | About 2.5 mM to about 50 mM; about 2.5 mM to |
| | about 25.5 mM, about 2.5 mM to about 20 mM, |
| | about 2.5 nM to about 10 nM, about 2.5 nM to |
| | about 10 nM, about 2.5 nM to about 8 nM, about |
| | 2.5 nM to about 5 nM About 8 mM to about 50 mM; |
| | about 8 mM to about 25.5 mM, about 8 mM to about |
| | 20 mM, about 8 nM to about 10 nM, about 8 nM to about |
| | 10 nMAbout 10 mM to about 50 mM; about 10 mM to |
| | about 25.5 mM, about 10 mM to about 20 mM |
| | About 20 mM to about 50 mM; about 20 mM to |
| | about 25.5 mM,About 25.5 mM to about 50 mM |
| | About 2.5 mM, about 5.5 mM, about 8 mM, about |
| | 10 mM, about 20 mM, about 25 mM |
| ITS-X | About 1:50,000, about 1:200, about 1:1000, |
| | about 1:10,000 |
| LDN-1913189 | About 0 nM to about 150 nM, from about 50 nM |
| | to about 150 nM |
| MCX Compound | About 3 μM, about 2 μM, about 2 μM, about 0.5 |
| | μM, about 0.5 μM to about 5 μM, about 1 μM to |
| | about 4 μM, about 1 μM to about 3 μM, about 2 |
| | μM to about 3 μM |
| Retinoic Acid | About 2 μM, about 1 μM, about 0.5 μM, about |
| | 0.1 μM, from about 0.11 μM to about 3 μM, |
| | from about 0.5 μM to about 2.5 μM |
| SANT-1 | About 0, about 0.25 μM, from about 0 μM to |
| | about 0.3 μM, from about 0.1 to about 0.3 μM. |
| | from about 0.1 μM to about 0.25 μM |
| TppB or TPB | About 500 nM, about 100 nM, from about 50 nM |
| | to about 550 nM, from about 50 nM to about 150 |
| | nM, from about 200 nM to about 500 nM, from |
| | about 300 nM to about 550 nM, about 50 nM, |
| | from about 25 nM to about 75 nM |
| Y-27632 | About 10 μM, from about 5 μM to about 15 μM, |
| | from about 5 μM to about 10 μM |

As used herein, “MCX compound” is 14-Prop-2-en-1-yl-3,5,7,14,17,23,27-heptaazatetracyclo[19.3.1.1-2,6-˜.1-8,12.˜]heptacosa-1(25),2(27),3,5,8(26),9,11,21,23-non-aen-16-one, which has the following formula (Formula 1):

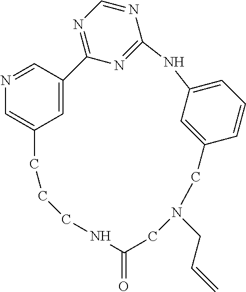 [SMILES: C=CCN1CC(=O)NCCCc2cncc(c2)-c2ncnc(n2)Nc2cccc(c2)C1]

Other cyclic aniline-pyridinotriazines may also be used instead of the above-described MCX compound. Such compounds include but are not limited to 14-Methyl-3,5,7,14,18,24,28-heptaazatetracyclo[20.3.1.1-2,6˜.-1˜8,12˜]octacosa-1(26),2(28),3,5, 8(27),9,11,22,24-nonaen-17-on-e and 5-Chloro-1,8,10,12,16,22,26,32-octaazapentacyclo[24.2.2.1˜3,7˜-1˜9,13˜.1˜14,18˜]tritriaconta-3(33),4,6,9(32),10-,12,14(31),15,17-nonaen-23 -one. These compounds are shown below (Formula 2 and Formula 3):

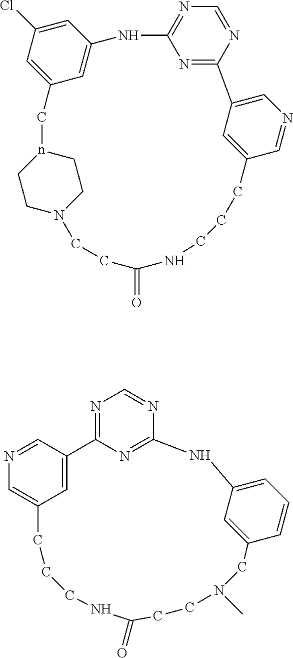

Exemplary suitable compounds are disclosed in U.S. Patent App. Pub. No. 2010/0015711, the disclosure of which is incorporated in its entirety as it pertains to the MCX compounds, related cyclic aniline-pyridinotriazines, and their synthesis.

Publications cited throughout this document are hereby incorporated by reference in their entirety.

### EXAMPLES

The present invention is further illustrated by the following non-limiting examples.

### Example 1

This example demonstrates formation of insulin expressing cells in a stirred suspension culture system using 0.5 liter spinner flasks. Media and gas were exchanged through removable side-arm caps. The insulin positive cells were formed in a step-wise process in which cells first expressed PDX1 and then also co-expressed NKX6.1, a protein transcription factor required for pancreatic beta cell formation and function. These co-expressing cells then gained expression of insulin and later MAFA, in combination with PDX1 and NKX6.1 while in suspension culture. When this population of cells was transplanted into the kidney capsule of immune-compromised mice, the graft produced detectable blood levels of human C-peptide within four weeks of engraftment.

Cells of the human embryonic stem cell line H1 (WA01 cells, WiCell Research Institute, Madison, Wis.) were grown in Essential 8™ (“E8™”) medium (Life Technologies Incorporated, Carlsbad, California; Catalog No. A15169-01) supplemented with 0.5% weight to volume (“w/v”) of a fatty acid free bovine serum albumin (“FAF-BSA”) (Proliant, Inc., Boone, Idaho; Catalog No. 68700) in dynamic suspension for >4 passages as round aggregated clusters. The clusters were then frozen as single cells and clusters of 2 to 10 cells per the following method. Approximately 600-1000 million cells in aggregated clusters were transferred to a centrifuge tube and washed using 100 mL of 1× Dulbecco's Phosphate Buffered Saline, without Calcium or Magnesium (“DPS −/−”) (Life Technologies; Catalog No. 14190-144). After the wash, the cell aggregates were then enzymatically disaggregated by adding a 30 mL solution of 50% StemPro®Accutase® enzyme (Life Technologies, Catalog No. A11105-01) and 50% DPBS −/− by volume to the loosened cell aggregate pellet. The cell clusters were pipetted up and down 1 to 3 times and then intermittently swirled for approximately 4 minutes at room temperature, then centrifuged for 5 min, at 80-200 ref. The Accutase® supernatant was then aspirated as completely as possible without disturbing the cell pellet. The centrifuge tube was then tapped against a hard surface for approximately 4 minutes, to disaggregate the clusters into single cells and clusters comprised of 2 -10 cells. After 4 minutes, the cells were re-suspended in 100 mL of E8TM media supplemented with 10 μM Y-27632 (Enzo Life Sciences, Inc., Farmingdale, N.Y.; Catalog No. ALX-270-333) and 0.5% w/v FAF-BSA, and centrifuged for 5 to 12 minutes at 80-200 ref. The supernatant was then aspirated and cold (<4° C.) Cryostor® Cell Preservation Media CS10 (Sigma-Aldrich; St. Louis, Mo.; Catalog No. C2874-100mL) was added drop-wise to achieve a final concentration of 100 to 150 million cells per mL. This cell solution was held in an ice bath while being aliquoted to 2 mL cryogenic vials (Corning Incorporated, Corning, N.Y.; Catalog No. 430488) after which the cells were frozen using a controlled rate freezer (CryoMed™ 34L Controlled-Rate Freezer, Thermo Fischer Scientific, Inc., Buffalo, N.Y.; Catalog No. 7452) as follows. The chamber was cooled to 4° C. and the temperature was held until a sample vial temperature reached 6° C. and then the chamber temperature was lowered 2° C. per minute until the sample reached −7° C. at which point the chamber was cooled 20° C./min. until the chamber reached −45° C. The chamber temperature was then allowed to briefly rise at 10° C./min. until the temperature reached −25° C., and then the chamber was cooled further at 0.8° C./min. until the sample vial reached −40° C. The chamber temperature was then cooled at 10° C./min. until the chamber reached −100° C. at which point the chamber was then cooled 35° C./min. until the chamber reached −160° C. The chamber temperature was then held at −160° C. for at least 10 minutes, after which the vials were transferred to gas phase liquid nitrogen storage. These cryo-preserved single cells at high concentration were then used as an intermediate/in-process seed material (“ISM”).

Vials of ISM were removed from the liquid nitrogen storage, thawed, and used to inoculate a 3 liter glass, stirred suspension tank bioreactor (DASGIP Information and Process Technology GMBH, Juelich, Germany). The vials were removed from liquid nitrogen storage and quickly transferred to a 37° C. water bath for 120 seconds to thaw. The vials were then moved to a biosafety cabinet (“BSC”) and the thawed contents transferred via 2 mL glass pipette to a 50 mL conical tube. Then 10mL of E8™ medium supplemented with 0.5% w/v FAF-BSA and 10 μM of Rho kinase inhibitor Y-27632, were added to the tube in a drop-wise manner. The cells were centrifuged at 80-200 rcf for 5 min. The supernatant from the tube was aspirated and 10 mL fresh E8™ medium supplemented with 0.5% w/v FAF-BSA and 10 μM Y-27632 were added and the volume containing the cells was pipetted into a media transfer bottle (Cap2V8®, Sanisure, Inc., Moorpark, Calif.) containing 450 mL E8™ media supplemented with 0.5% w/v FAF-BSA and 10 μM Y-27632. The bottle contents were then pumped directly into the bioreactor via a sterile, C-Flex® tubing weld using a peristaltic pump. The bioreactor was prepared with 1000 mL E8TM medium supplemented with 0.5% w/v FAF-BSA and 10 μM Y-27632 pre-warmed to 37° C., stirred at 70 rpm, with a dissolved oxygen set point of 30% (air O₂, and N₂ regulated), and a controlled CO₂ partial pressure of 5%. The reactor was inoculated to give a target concentration of 0.225×10⁶ cells/mL (concentration range: 0.2 to 0.5×10⁶ cells/mL).

Once the reactor was inoculated, the cells formed round aggregated clusters in the stirred reactor. After 24 hours in culture, the medium was partially exchanged as more than 80% of the original volume was removed and 1.5 L of E8™ media supplemented with 0.5% w/v FAF-BSA was added back (fresh medium). This media exchange process was repeated 48 hours after inoculation. After three days in suspension culture as round aggregated clusters, the cells were pumped out of the bioreactor and transferred into three, 0.5 L disposable spinner flasks (Corning; Catalog No. 3153) for differentiation. All of the spinner flasks were maintained in a 37° C. humidified incubator supplemented with 5% CO2, and a constant stir speed of 60 RPM (55-65 RPM). The differentiation protocols are described below as conditions A, B and C.

Throughout the differentiation process, the spinners were moved from dynamic agitation in the incubator to a BSC for media exchanges. The spinners were held without agitation for 6 minutes, allowing the majority of cell clusters to settle to the bottom of the vessel. After 6 minutes, the spinner flask side arm cap was detached and 90% or more of the spent media was removed via aspiration. Once the spent media was removed, 300 mL of fresh media was added back to the spinner flask through the open side arm. The spinner cap was then replaced and returned to dynamic suspension in the incubator under previously described conditions.

Stage 1 (3 Days):

For condition A, a base medium (“Stage 1 Base Medium”) was prepared using MCDB-131 medium containing 1.18 g/L sodium bicarbonate (Life Technologies; Catalog No. 10372-019); supplemented with an additional 2.4 g/L sodium bicarbonate (Sigma Aldrich; Catalog No. S3187), 2% w/v FAF-BSA, previously re-constituted in MCDB-131; 1× concentration of GlutaMAX™ (Life Technologies; Catalog No. 35050-079); 2.5 mM glucose (45% in water; Sigma Aldrich; Catalog No. G8769); and a 1:50,000 dilution of insulin-transferrin-selenium-ethanolamine (“ITS-X”)(Life Technologies; Catalog No. 51500056). Cells were cultured for one day in 300 mL of the Stage 1 Base Medium supplemented with 100 ng/ml Growth/Differentiation Factor 8 (“GDF8”) (Peprotech, Inc., Rocky Hill, N.J.; Catalog No. 120-00); and 2 μM of 14-prop-2-en-1-yl-3,5,7,14,17,23,27-heptaazatetracyclo[19.3.1.1˜2,6˜.1˜8,12˜]heptacosa-1(25),2(27),3,5,8(26),9,11,21,23-nonaen-16-one (“MCX compound”). After 24 hours, a media exchange was completed as described above, and fresh 300 mL of Stage 1 Base Medium supplemented with 100 ng/mL of GDF8, but no MCX compound, were added to the flask. Cells were maintained without further media exchange for 48 hours.

In condition B, cells were cultured as described for condition A except that 3 μM MCX compound was used for the first day.

In condition C, cells were cultured as described for condition A except that 100 ng/mL of activin A was used in place of GDF8 and 30 μM of glycogen synthase kinase 3β inhibitor (6-[[2-[[4-(2,4-dichlorophenyl)-5-(5-methyl-1H-imidazol-2-yl)-2pyrimidinyl]amino]ethyl]amino]-3-pyridinecarbonitirile (“CHIR99021”) (Stemgent Inc, Cambridge Massachusetts, Catalog No. 04004-10) was used in place of the MCX compound.

Stage 2 (3 Days):

For condition A, a base medium (“Stage 2 Base Medium”) was prepared using MCDB-131 medium containing 1.18 g/L sodium bicarbonate and supplemented with an additional 1.2 g/L sodium bicarbonate; 2% w/v FAF-BSA, previously re-constituted in MCDB-131; 1× concentration of GlutaMAX™; 2.5 mM glucose; and a 1:50,000 dilution of ITS-X. After the completion of Stage 1, a media exchange was completed as described above, whereby the spent Stage 1 media was removed and replaced with 300 mL of Stage 2 Base Medium supplemented with 50 ng/mL fibroblast growth factor 7 (“FGF7”) (R&D Systems, Minneapolis, Minn.; Catalog No.251-KG). Forty-eight hours after the media exchange, the spent media was again removed and replaced with 300 mL fresh Stage 2 Base Medium supplemented with 50 ng/mL FGF7.

In condition B, cells were cultured as for condition A.

In condition C, cells were cultured as for conditions A and B, with the further addition of 250 μL of a 1M ascorbic acid (Sigma Aldrich; Catalog No. A4544 reconstituted in water) to 1 L of the Stage 2 Base Medium.

Stage 3 (3 Days for Conditions A and B and 2 days for Condition C):

For condition A, a base medium (“Stage 3-4 Base Medium”) was prepared using MCDB-131 medium containing 1.18 g/L sodium bicarbonate supplemented with an additional 1.2 g/L sodium bicarbonate; 2% w/v FAF-BSA, previously re-constituted in MCDB-131; 1× concentration of GlutaMAX™; 2.5 mM glucose; and a 1:200 dilution of ITS-X. After the completion of Stage 2, a media exchange was completed to replace the spent media with 300 mL of Stage 3-4 Base Medium supplemented with 50 ng/mL FGF-7; 100 nM of the bone morphogenic (“BMP”) receptor inhibitor ((6-(4-(2-(piperidin-1-yl)ethoxy)phenyl)-3-(pyridin-4-yl)pyrazolo[1,5-a]pyrimidine hydrochloride)) (“LDN-193189”, Shanghai ChemPartner Co Ltd., Shanghai, China); 2 μM retinoic acid (“RA”) (Sigma Aldrich; Catalog No. R2625); 0.25 μM N-[(3,5-dimethyl-1-phenyl-1H-prazol-4-yl)methylene]-4-(phenylmethyl)-1-piperazineamine (“SANT-1”) (Sigma Aldrich; Catalog No. S4572); and 400 nM of the PKC activator ((2S, 5S-(E,E)-8-(5-(4-trifluoromethyl)phenyl-2,4-pentadienoylamino)benzolactam (“TPB”) (Shanghai ChemPartner Co Ltd., Shanghai, China). Twenty-four hours post media exchange, the spent media was again replaced with 300 mL fresh Stage 3-4 Base Medium containing the above supplements with the exception of LDN-193189. Cells were cultured in the media for 48 hours.

In condition B, cells were cultured as for condition A.

In condition C, cells were cultured as for conditions A and B with the further addition of 250 μL/L of 1M ascorbic acid solution to the Stage 3-4 Base Medium. Furthermore, 48 hours post initiation of Stage 3, the cells were moved to Stage 4 media as described below.

Stage 4 (3 Days for Conditions A and B and 4 Days for Condition C):

For condition A, after the completion of Stage 3, the spent media was removed and replaced with 300 mL of Stage 3-4 Base Medium supplemented with 0.25 μM SANT-1 and 400 nM of TPB. Forty-eight hours after initiation of Stage 4, 3.2 mL/L of a 45% glucose solution (8 mM glucose bolus) was added to the flask and the cells were cultured in the media for an additional 24 hours.

In condition B, cells were cultured as for condition A.

In condition C, cells were cultured as for conditions A and B, except the Stage 3-4 Base Medium was further supplemented with 0.1 μM RA, 50 ng/mL of FGF7, and 250 μL/L of 1M ascorbic acid solution. Forty-eight hours later, the spent media was exchanged with the same fresh media (with condition C media supplements) and the cells were cultured for 48 more hours.

Stage 5 (7 Days):

For conditions A, B and C, a base medium (“Stage 5+ Base Medium”) was prepared using MCDB-131 medium base containing 1.18 g/L sodium bicarbonate supplemented with an additional 1.75 g/L sodium bicarbonate; 2% w/v FAF-BSA previously re-constituted in MCDB-131; 1× concentration of GlutaMAX™; 20 mM glucose; 1:200 dilution of ITS-X; 250 μL/L of 1M ascorbic acid; 10 mg/L heparin (Sigma Aldrich; Catalog No. H3149-100KU). After the completion of Stage 4, media exchanges were completed and 300 mL of Stage 5+Base Medium supplemented with 1 μM T3 as 3,3′,5-Triiodo-L-thyronine sodium salt (“T3”) (Sigma Aldrich; Catalog No. T6397), 10 μM of 2-(3-(6-methylpyridin-2-yl)-1H-pyrazol-4-yl)-1,5-nathyridine (“ALK5 inhibitor II”) (Enzo Life Sciences, Inc.; Catalog No. ALX-270-445), 100 nM of gamma secretase inhibitor XX (EMD Millipore Corporation, Gibbstown, N.J., Catalog No. 565789); 20 ng/mL of betacellulin (R&D Systems, Catalog No. 261-CE-050); 0.25 μM SANT-1; and 100 nM RA. Forty-eight hours after initiation of Stage 5, the spent media was removed and replaced with 300 mL of the same media and supplements. Forty-eight hours later, the medium was removed and replaced with Stage 5+ Base Medium supplemented with 1 μM T3,10 μM ALK5 inhibitor II, 20 ng/mL of betacellulin, and 100 nM RA. Forty-eight hours later the medium was again exchanged and replaced with Stage 5+ Base Medium supplemented with 1 μM T3, 10 μM ALK5 inhibitor II, 20 ng/mL of betacellulin, and 100 nM RA.

Stage 6 (7 Days):

Twenty-four hours after the last Stage 5 media exchange, media for conditions A, B, and C were exchanged with Stage 5+ Base Medium supplemented with 1 μM T3 and 10 μM of ALK5 inhibitor II. Media exchanges were done at the end of days 2, 4 and 6 of Stage 6 with this supplemented medium.

Throughout the differentiation process, samples were collected from the suspension cultures on a daily basis. Daily cell samples were isolated for mRNA (qRT-PCR) and spent media were collected for metabolic analysis. At the end of chosen stages, protein expression was measured via flow cytometry or fluorescent immune-histochemistry. Spent media was analyzed using a NOVA® BioProfile® FLEX bio-analyzer (Nova Biomedical Corporation, Waltham, Mass.).

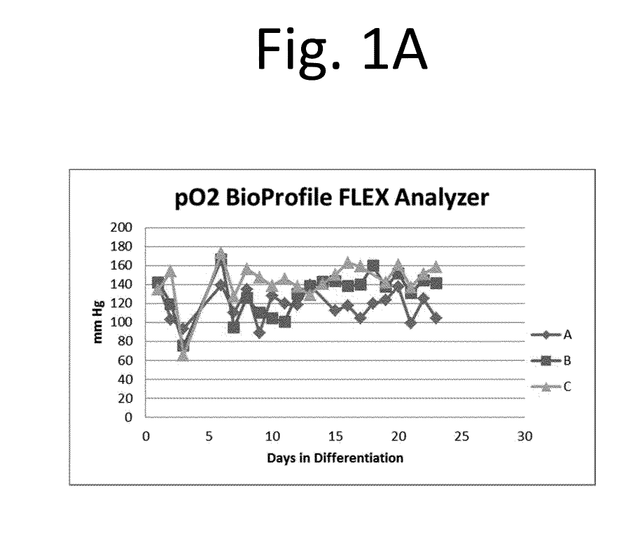

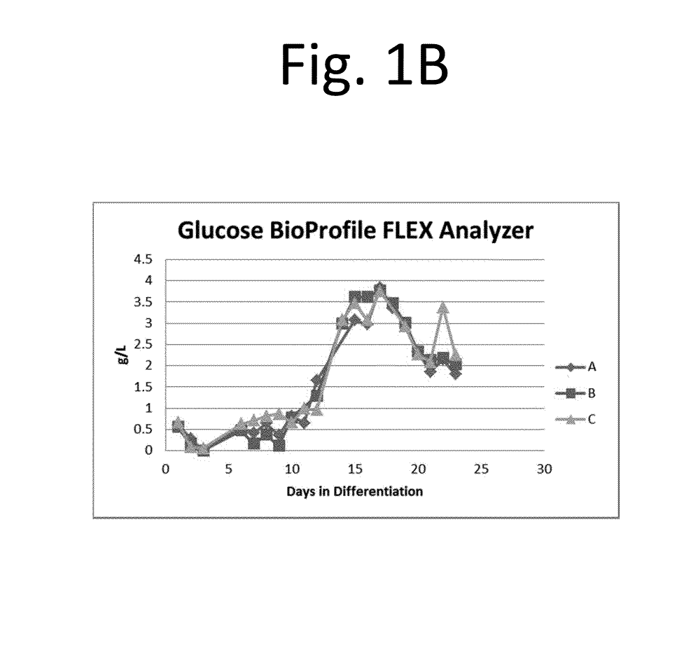

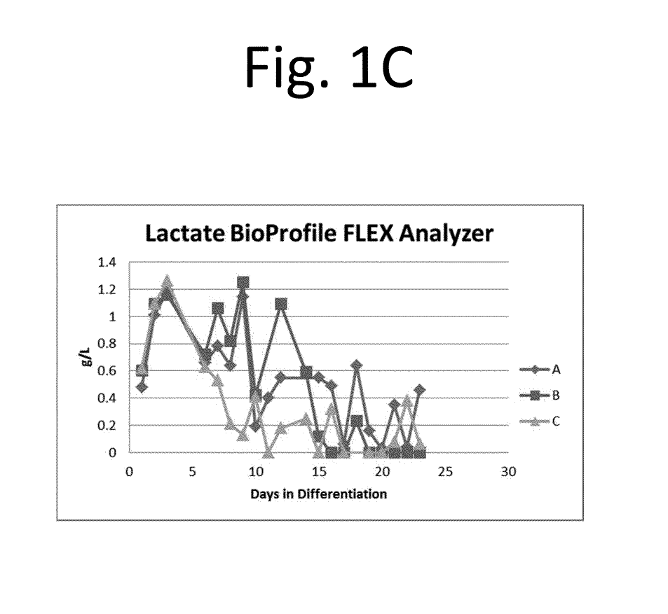

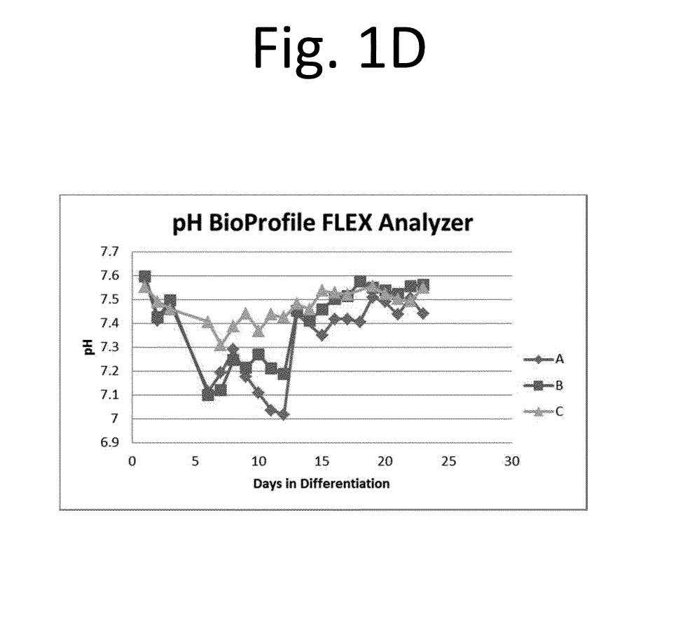

FIG. 1A through D depict data from a NOVA® BioProfile FLEX Analyzer obtained from spent media samples at the end of each day of differentiation (FIG. 1A-pO2/partial oxygen pressure; FIG. 1B—glucose concentration; FIG. 1C—lactate concentration; FIG. 1D—medium pH). These data demonstrate that for the first 3 days of Stage 1 of differentiation cells were most oxygen consumptive when compared to later stages of differentiation. Cells in Stage 1 reduced pO₂ levels from saturated levels of 140+mm Hg to below 100 mm Hg as detected by NOVA® analyzer (FIG. 1A). Furthermore Stage 1 cells consumed nearly all of the glucose in the medium (FIG. 1B) and generated more than 1 gram per liter of lactate in the first three days of the process (FIG. 1C).

As the cells moved into Stages 2 and 3 of differentiation, their oxygen and glucose consumption and lactate production changed as compared to Stage 1. Cells that had been treated with GDF8 and the MCX Compound (condition A or B) in Stage 1 were more oxygen consumptive in Stage 2 (FIG. 1A) than cells treated with activin A and CHIR99021 in Stage 1 (condition C). This observation of increased oxygen consumption correlated with a lower pH in spent medium (FIG. 1D and Table 1), increased lactate production (FIG. 1C), and higher glucose consumption (FIG. 1B) when comparing conditions A or B to condition C.

As the cells progressed to Stage 4 (days 10, 11, and 12 for Conditions A and B; days 9, 10, 11, and 12 for Condition C), the cells treated with conditions A and B retained an increased level of glucose consumption and a lower medium pH as compared to cells treated with Condition C (FIG. 1B and table 1). However, from day 14 (the second day of Stage 5) to day 19 (end of Stage 5) it was observed that glucose levels did not drop below 3 grams per liter in all treatment conditions. Once Stage 6 began, in all three conditions (FIG. 1B, day 20 onward) spent media glucose levels trended below 2.4 grams per liter. This increase in glucose consumption was not accompanied by an increase in total lactate production above 0.5 grams per liter (FIG. 1C) nor acidification of the spent media (FIG. 1D) suggesting the cells were converting to a less glycolytic and more mature metabolism, consistent with a pancreatic-islet, endocrine hormone cell population.

In addition to monitoring the metabolic profile of the spent media through daily sampling, representative samples of cells were obtained throughout the differentiation process and tested for mRNA expression of a panel of genes via Applied Biosystems® OpenArray® (Life Technologies) and calculated as fold difference in expression compared to pluripotent ISM cells after 24 hours in culture from the beginning of the experiment. FIGS. 2A through M depict data for expression of the following genes in cells differentiated through the first day of Stage 5: PDX1 (FIG.2A); NKX6.1 (FIG. 2B); PAX4 (FIG. 2C); PAX6 (FIG. 2D); NEUROG3(NGN3) (FIG. 2E); ABCC8 (FIG. 2F); Chromogranin-A (“CHGA”) (FIG. 2G); G6PC2 (FIG. 2H); IAPP (FIG. 2I); insulin (“INS”) (FIG. 2J); glucagon (“GCG”) (FIG. 2K); PTF1a (FIG. 2L); and NEUROD1 (FIG. 2M).

As shown in FIG. 2A, in all three differentiation conditions, by the end of Stage 2 day 3 (“S2D3”) the cells begin to express PDX1 and adopt a pancreatic fate. As the cells entered Stage 3 the cells began to express genes indicating endocrine pancreas specification (NGN3, NEUROD1, and CHGA; FIGS. 2E, 2M, and 2G) and by the end of Stage 3 and the beginning of Stage 4 they began to express genes required for beta cell formation (PAX4, PAX6, and NKX6.1; FIGS. 2C, 2D, and 2B). By the beginning of Stage 5, the cells began to express markers required for formation and function of islet and beta cells (GCG, INS, IAPP, G6PC2, and ABCC8; FIGS. 2K, 2J, 2I, 2H, and 2F).

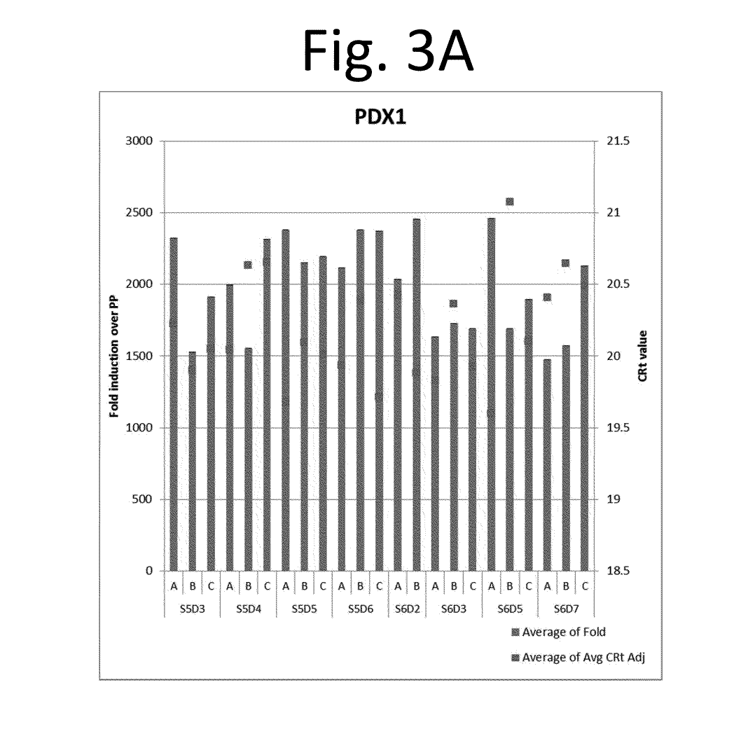

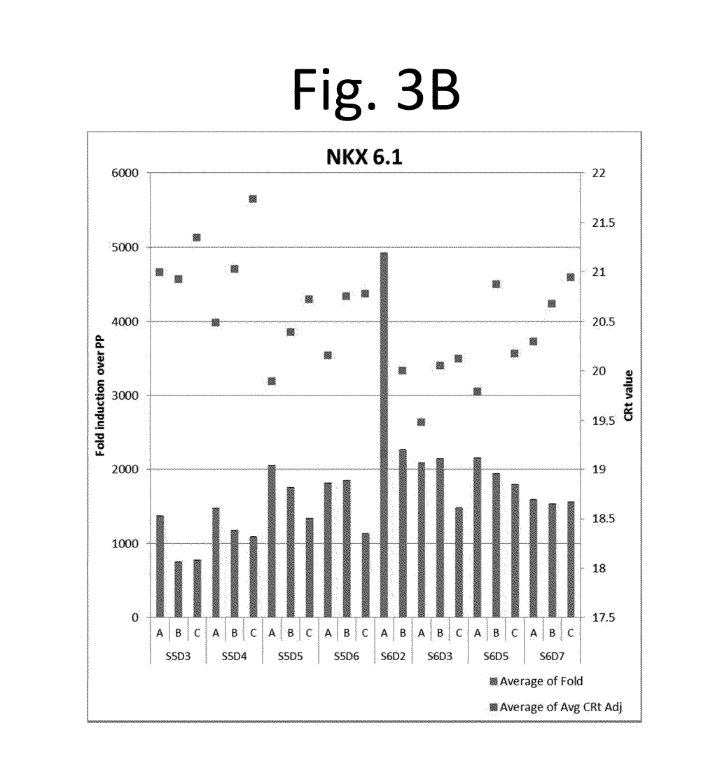

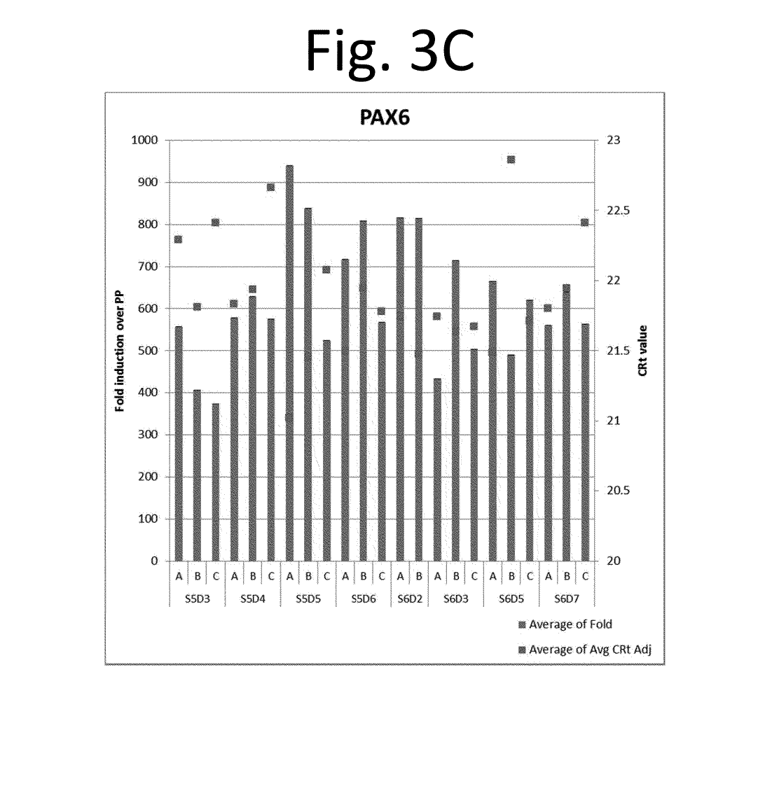

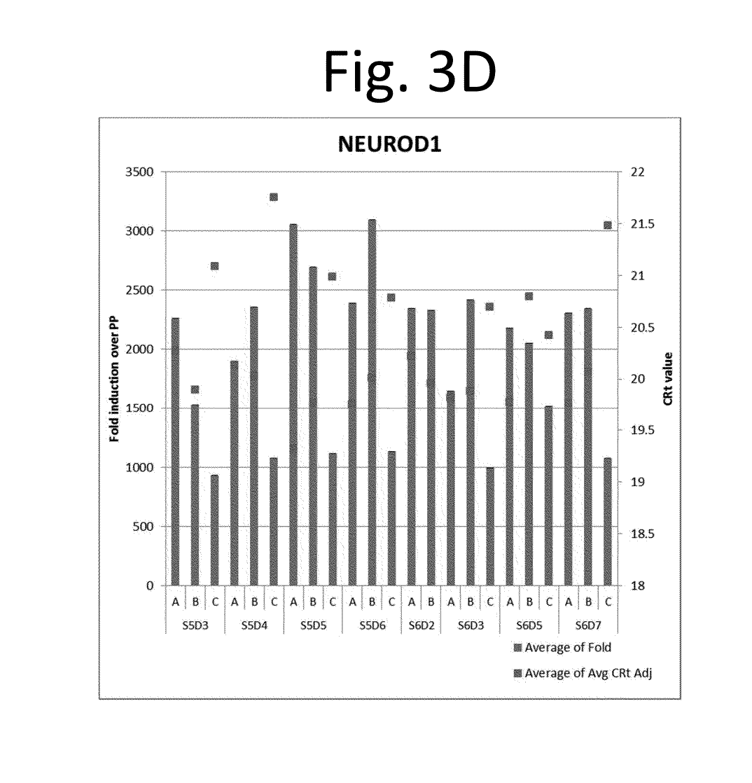

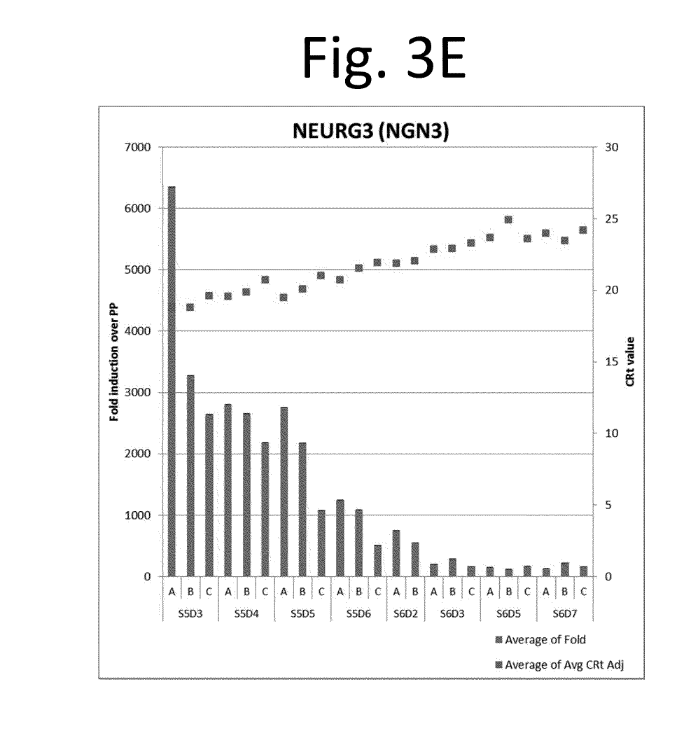

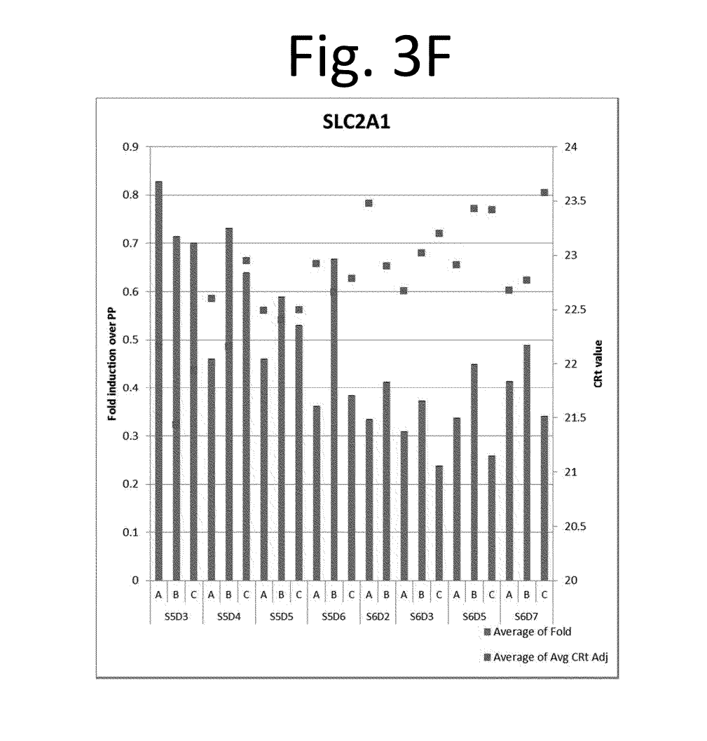

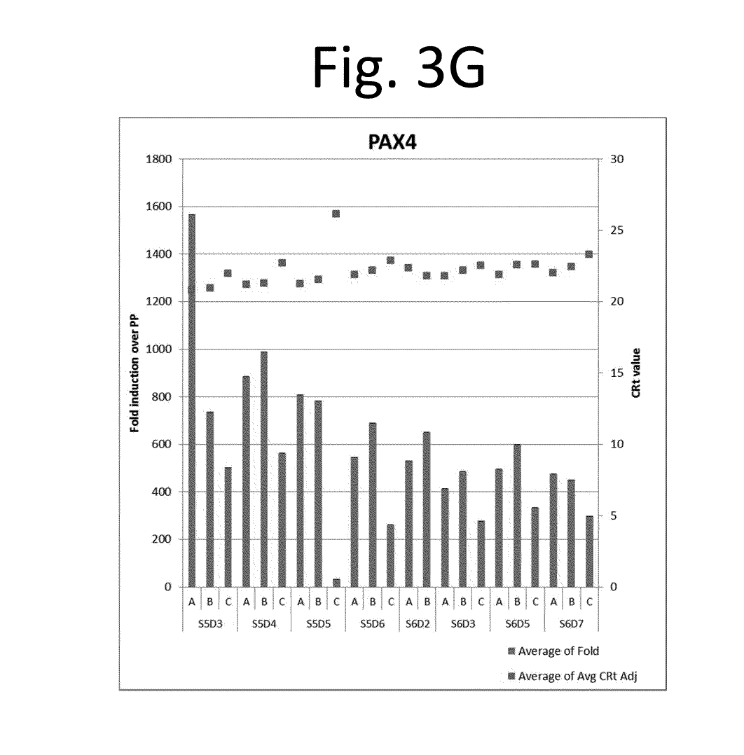

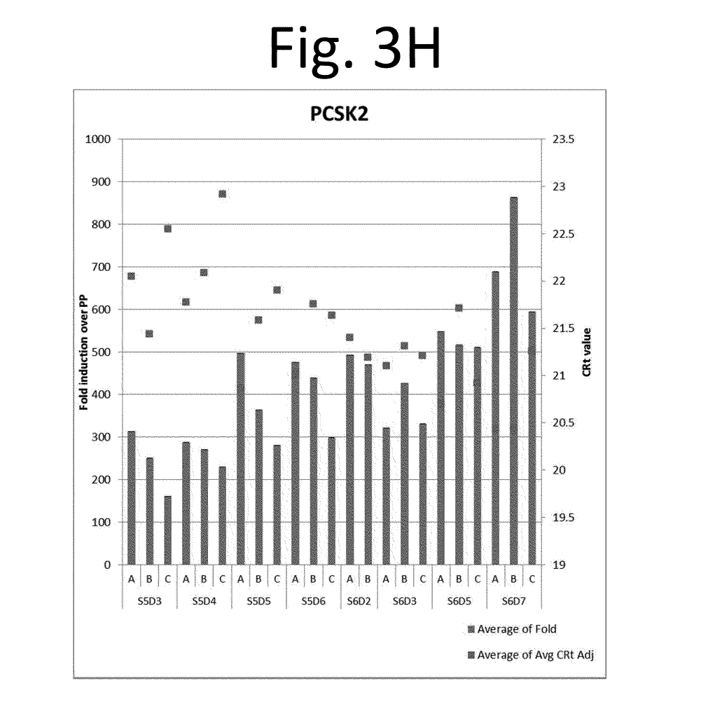

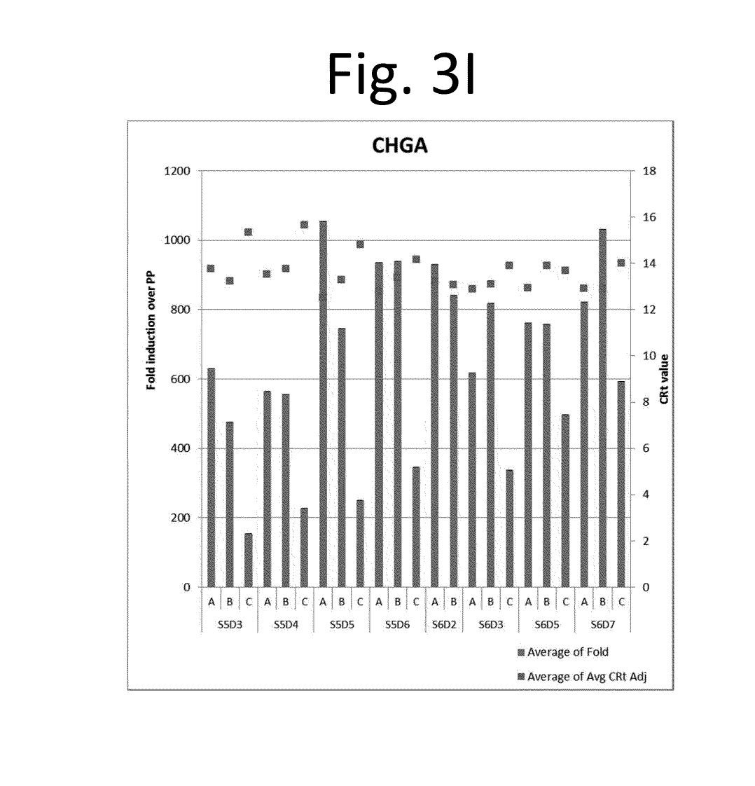

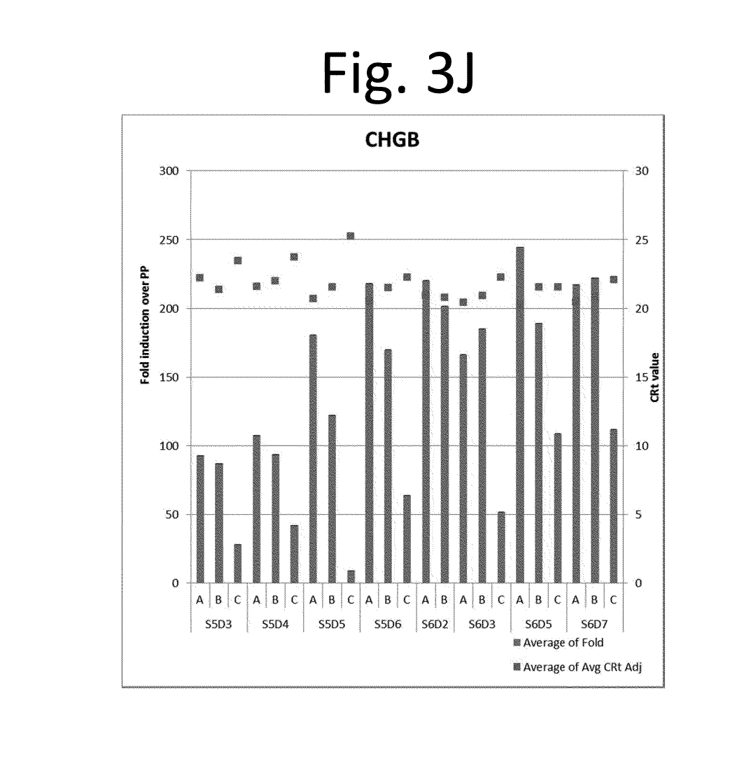

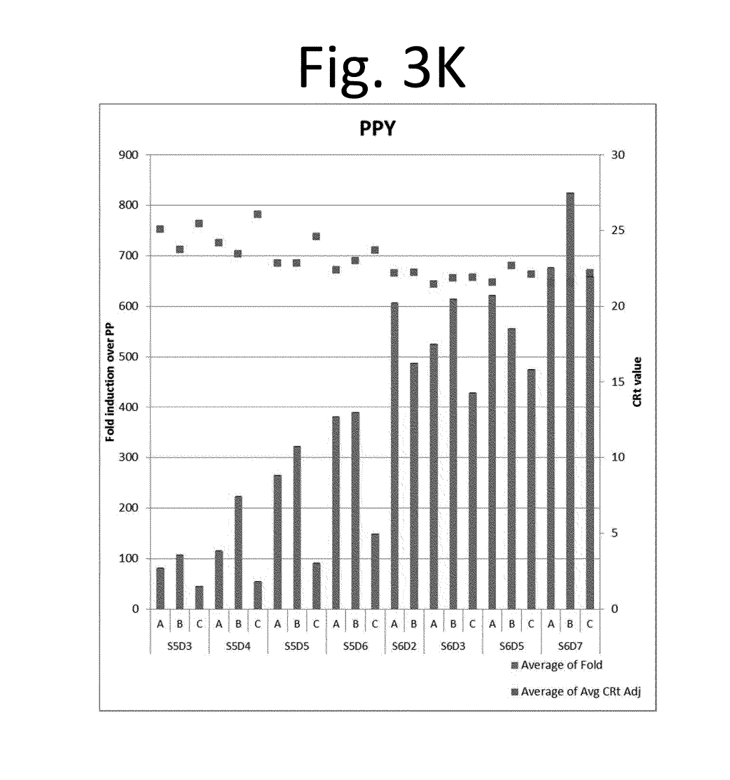

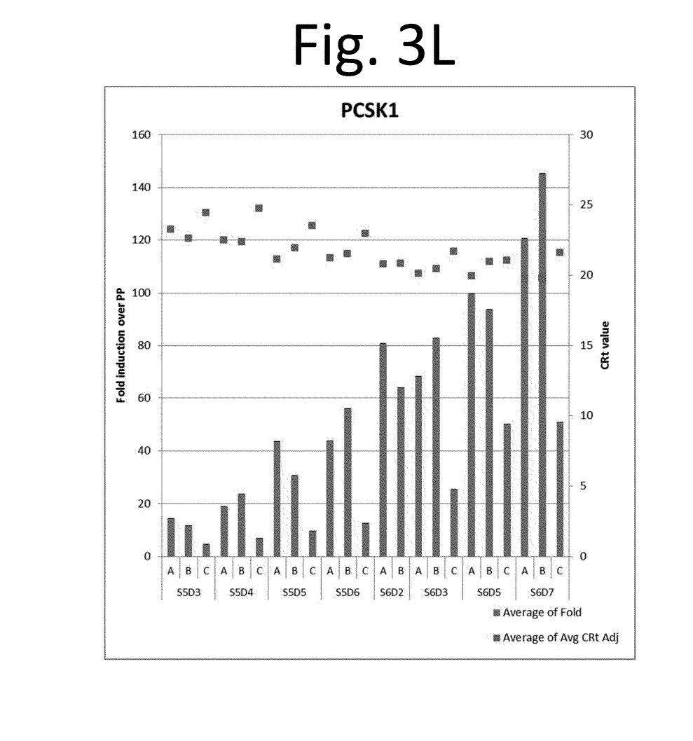

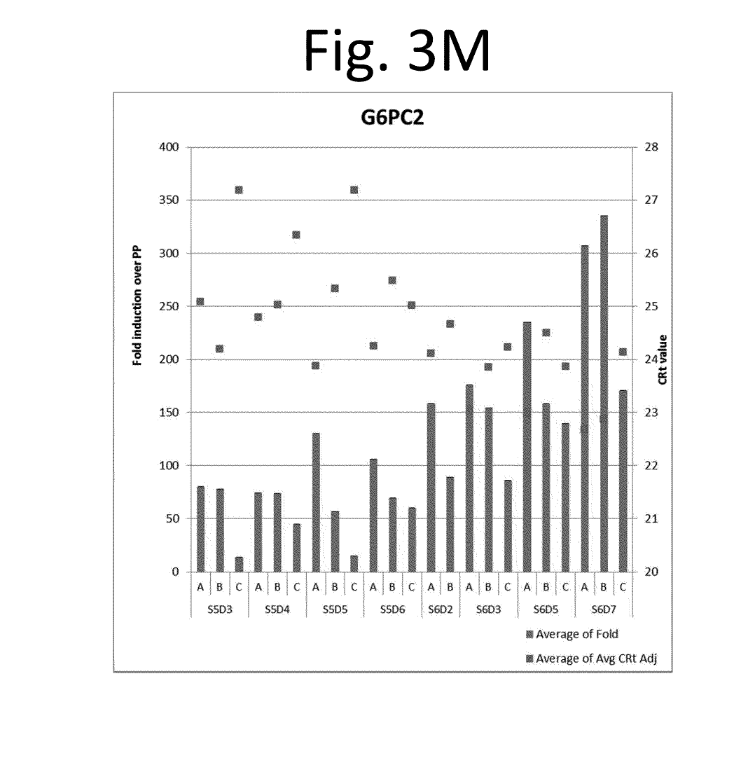

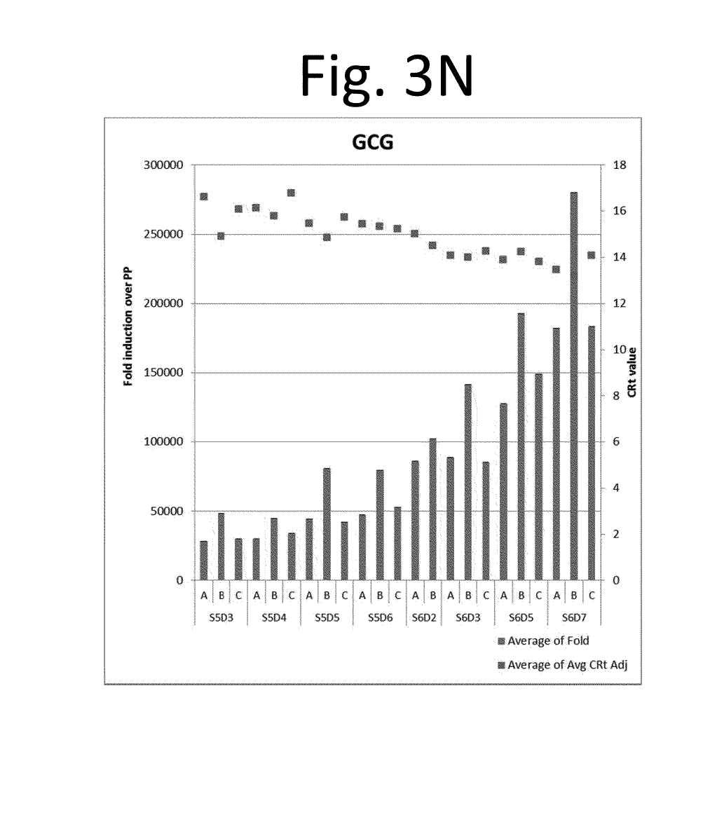

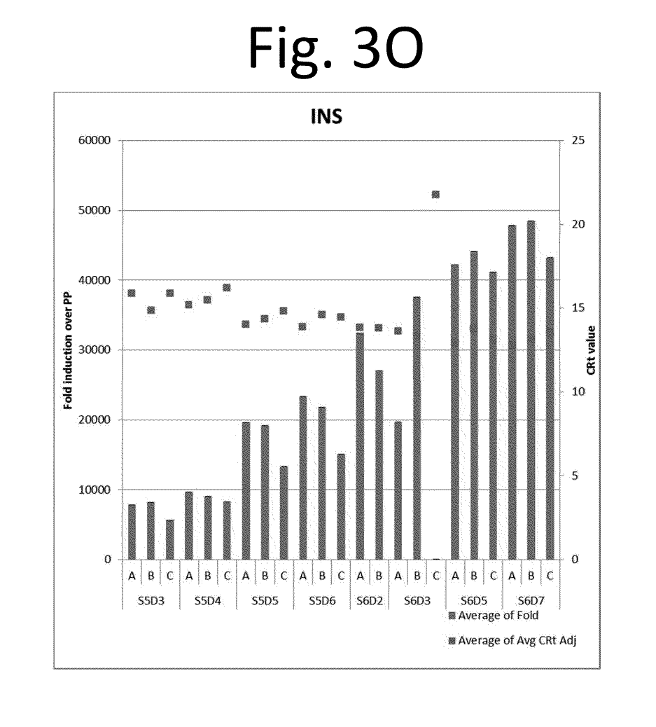

Samples were also collected throughout Stages 5 and 6 and analyzed by OpenArray® real-time PCR analyses for gene expression of PDX1 (FIG.3A); NKX6.1 (FIG. 3B); PAX6 (FIG. 3C); NEUROD1 (FIG. 3D); NEUROG3(NGN3) (FIG. 3E); SLC2A1 (FIG. 3F); PAX4 (FIG. 3G); PCSK2 (FIG. 3H); Chromogranin-A (FIG. 3I); Chromogranin-B (FIG. 3J); PPY (FIG. 3K); PCSK1 (FIG. 3L); G6PC2 (FIG. 3M); glucagon (FIG. 3N); and insulin (FIG. 3O). As shown in FIGS. 3A-3D, it was observed that PDX1, NKX6.1, PAX6, and NEUROD1 expression levels were stable from Stage 5 day 3 (“S5D3”) through the end of Stage 6 day 7 (S6D7). mRNA expression levels for NGN3, SLC2A1, and PAX4 were at the highest levels while the cells were exposed to gamma secretase inhibitor (Stage 5 days 1 through 4) and expression levels declined following removal of gamma secretase inhibitor (FIGS. 3E-3G). The genes PCSK2, CHGA, and CHGB showed an increase in expression at the end of Stage 5 (FIGS. 3M-3O), while the genes PPY, PCSK1, G6PC2, GCG, and INS rose continuously from the beginning of Stage 5 through to the end of Stage 6 (FIGS. 3K, 3L, 3M, 3N, 3O).

For additional characterization of various stages, cells were harvested at the end of Stages 1, 4, 5, and 6 and analyzed by flow cytometry. In brief, cell aggregates were dissociated into single cells using TrypLE™ Express (Life Technologies; Catalog No. 12604) for 3-5 minutes at 37° C. For surface staining, the released single cells were re-suspended in 0.5% human gamma globulin diluted 1:4 in staining buffer at a final concentration of 2 million cells/mL. Added to the cells at a final dilution of 1:20 were directly conjugated primary antibodies followed by incubation at 4° C. for 30 minutes. The stained cells were twice washed in the staining buffer, followed by re-suspension in 300 μL staining buffer and then incubated in 10 μL of 7-AAD for live/dead discrimination before flow cytometric analysis on a BD FACSCanto™ II. For intracellular antibody staining, single cells were first incubating with Violet Fluorescent LIVE/DEAD cell dye (Life Technologies, Catalog No. L34955) at 4° C. for 20-30 minutes followed by a single wash in cold PBS^{-/-}. The washed cells were then fixed in 280 μL of Cytofix/Cytoperm Fixation and Permeabilization Solution (BD Catalog No. 554722) at 4° C. for 30 minutes. The cells were then washed 2 times in 1× Perm/Wash Buffer (BD Catalog No. 51-2091 KZ), before being re-suspended at a final concentration of 2 million cells/mL. Fixed cell suspensions were then blocked using a 20% normal goat serum for 10-15 minutes at room temperature. Cells were incubated at 4° C. for 30 minutes with primary antibodies at empirically pre-determined dilutions followed by two washes in Perm/Wash buffer. Cells were then incubated with the appropriate antibodies at 4° C. for 30 minutes and then washed twice prior to analysis on a BD FACSCanto™ II. The concentration of antibodies used is shown on Table II. The antibodies for pancreas markers were tested for specificity using human islets or undifferentiated H1 cells as a positive control. For secondary antibodies, the following were added and incubated at 4° C. for 30 minutes: anti-mouse Alexa Fluor® 647 at 1:4,000 (Life Technologies, Catalog No. A21235) or goat anti-rabbit PE at 1:100 1:200 or 1:800 (Life Technologies, Catalog No. A10542) followed by a final wash in Perm/Wash buffer and analysis on BD FACSCanto™ II using BD FACSDiva™ Software with at least 30,000 events being acquired.

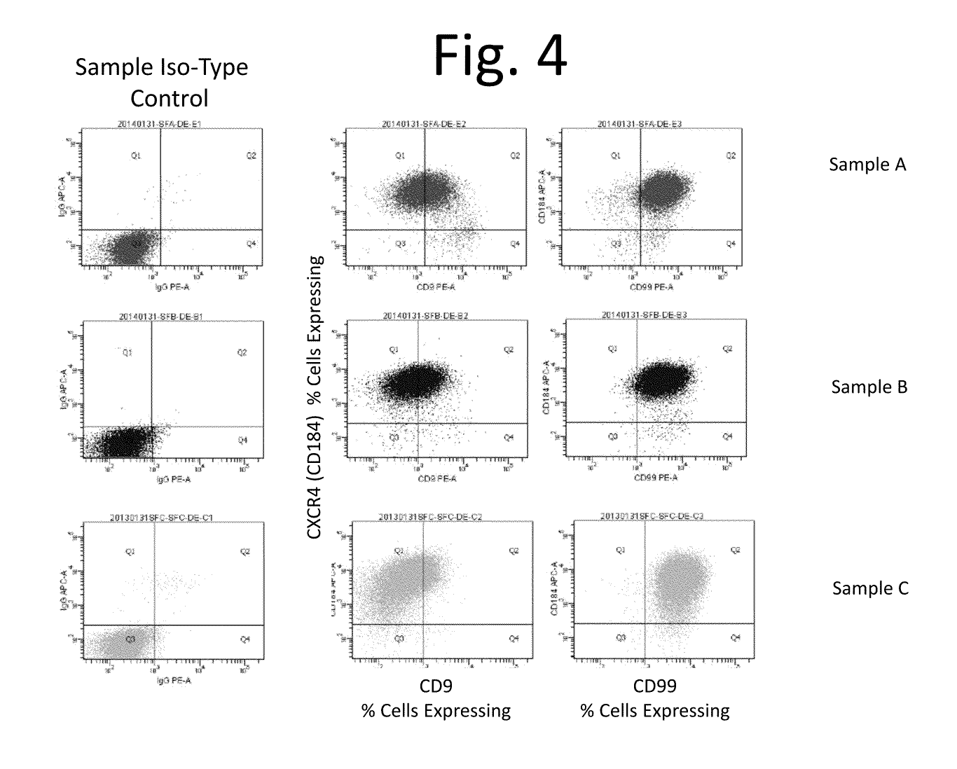

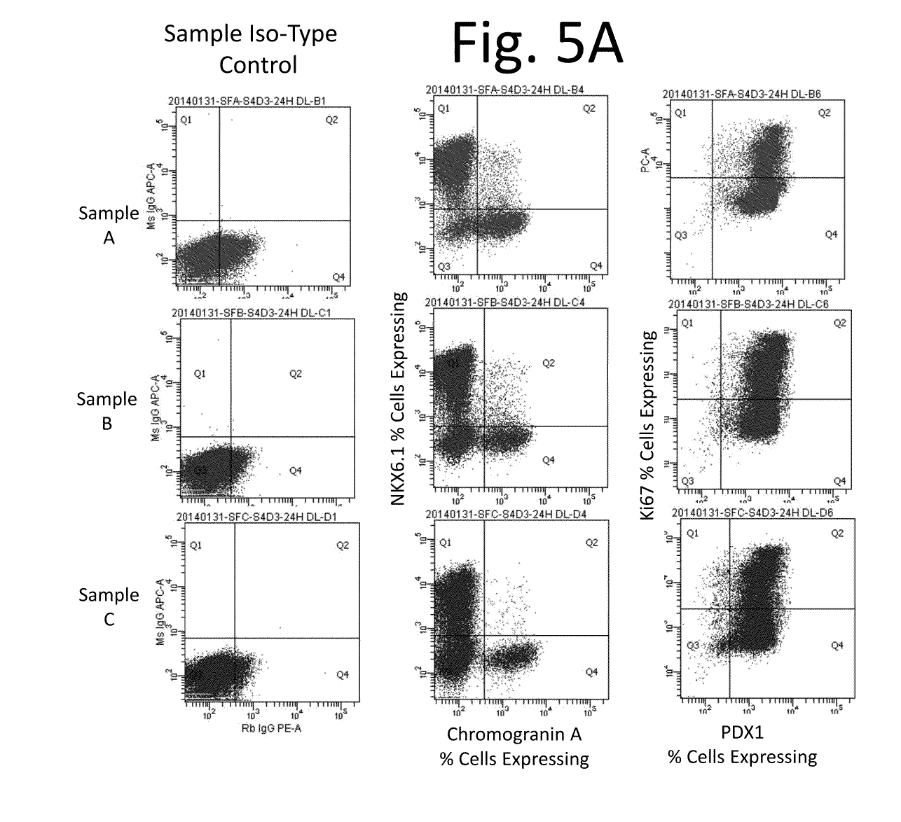

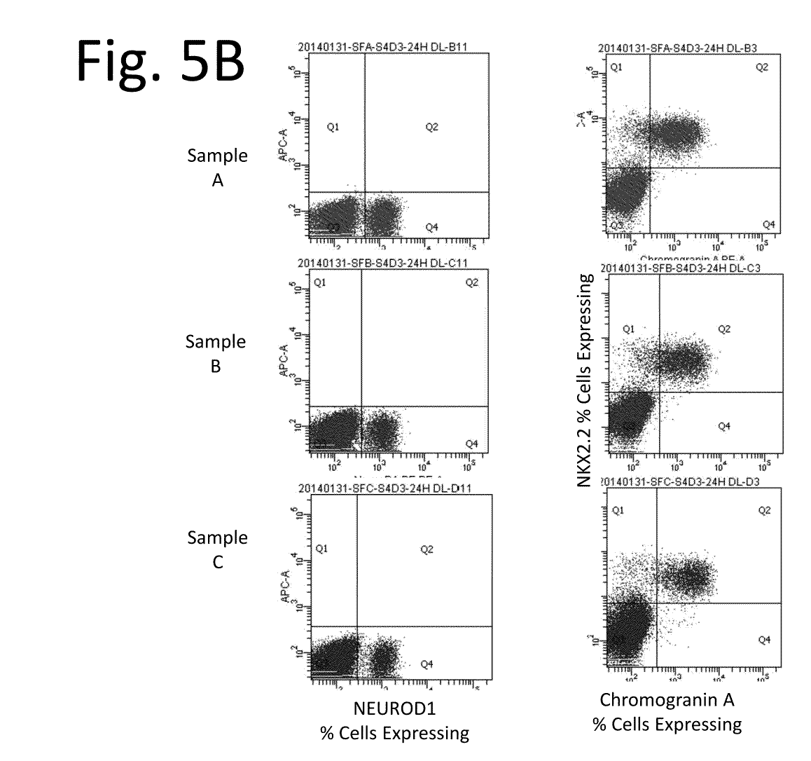

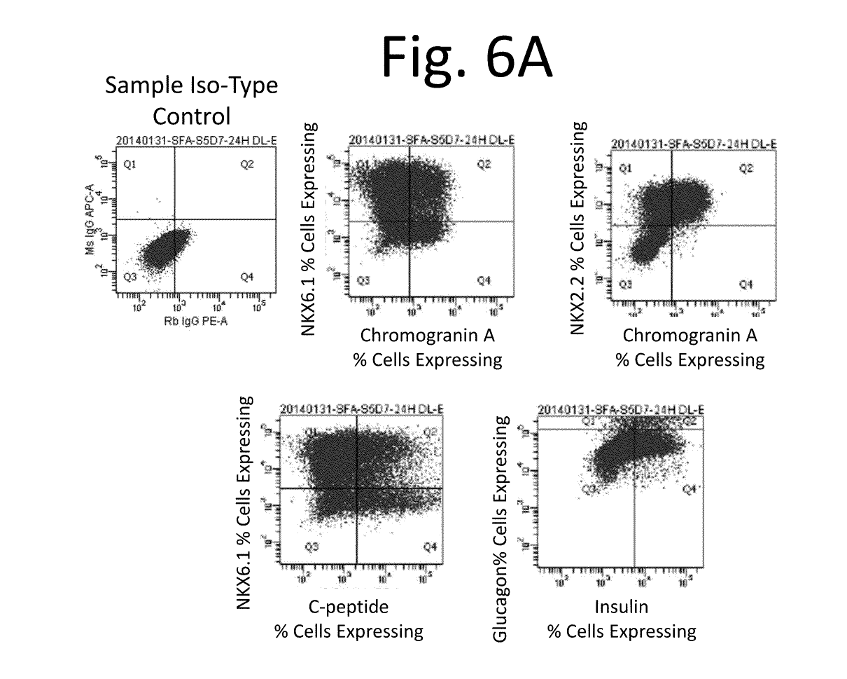

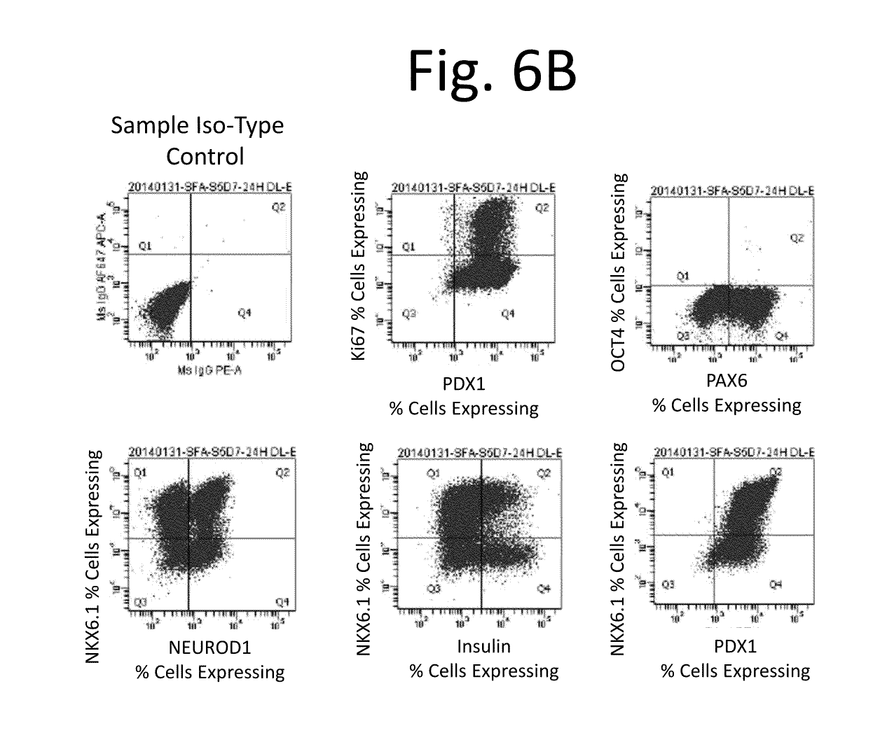

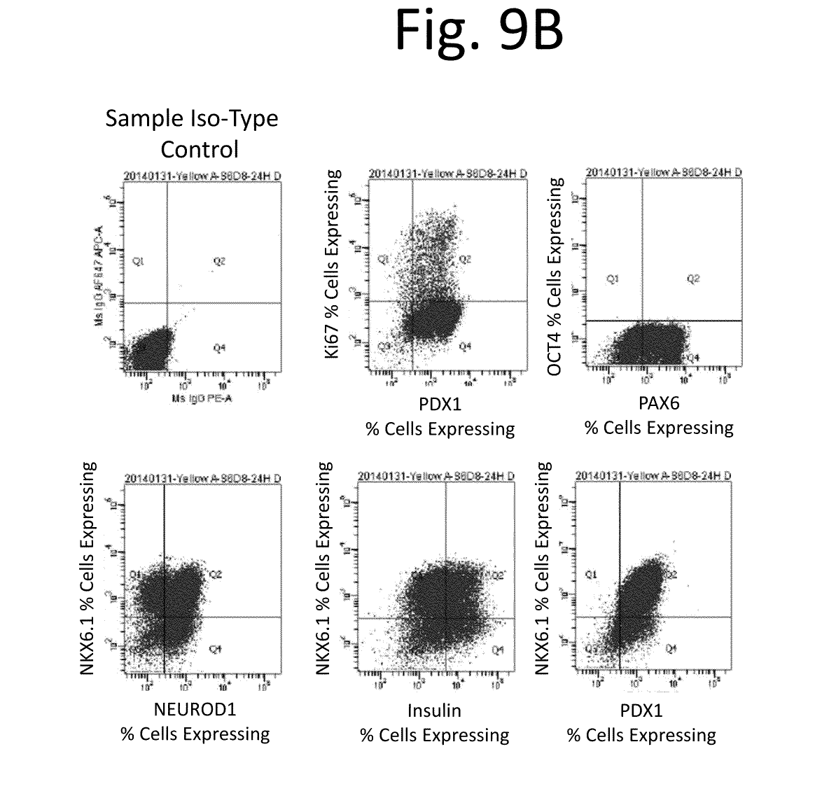

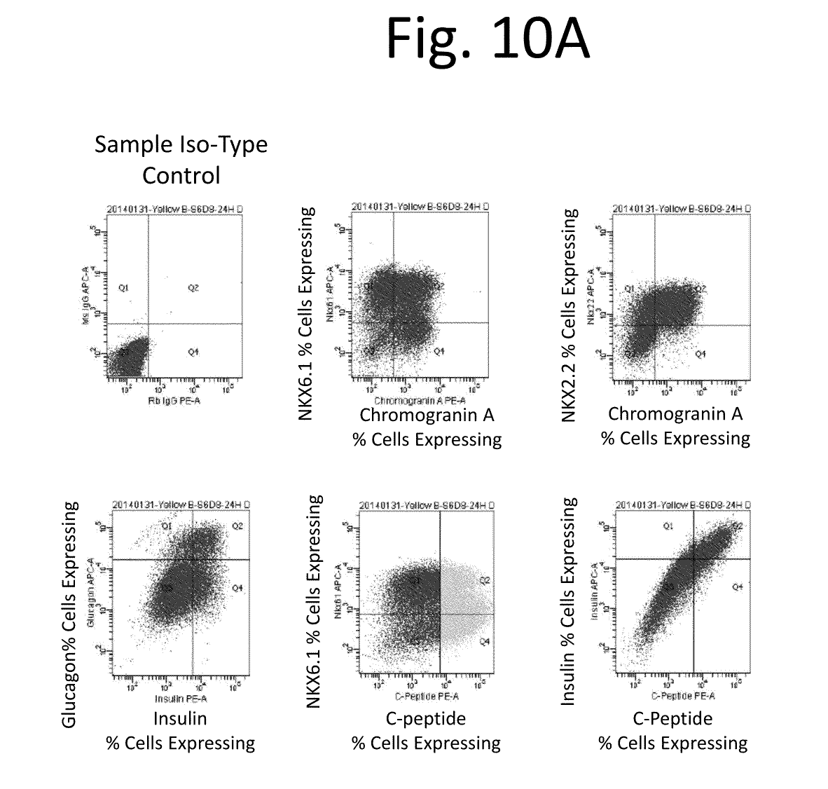

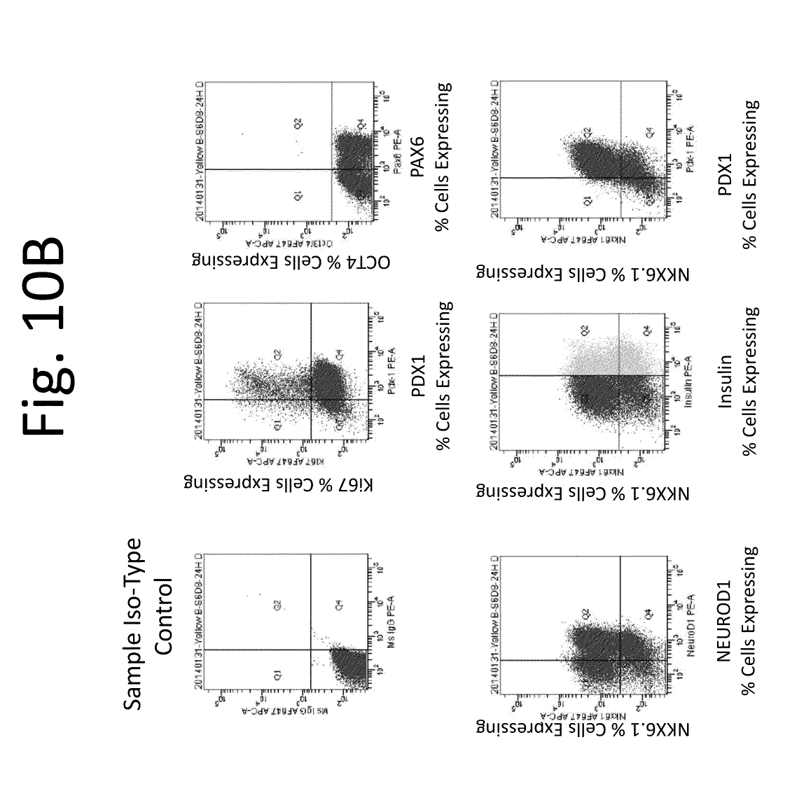

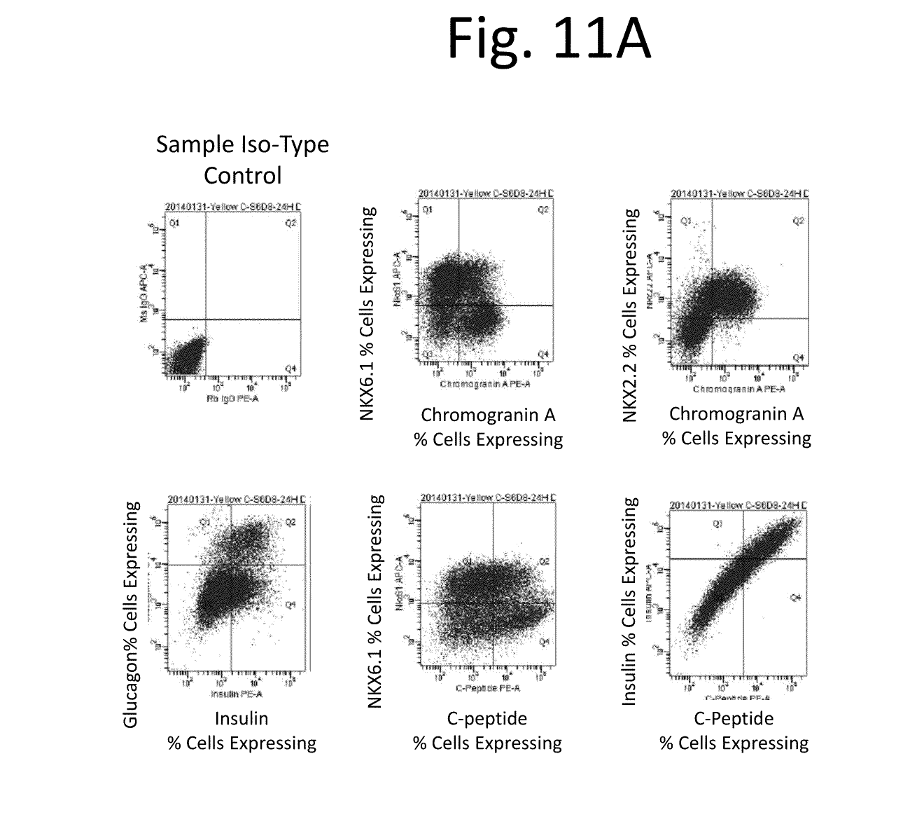

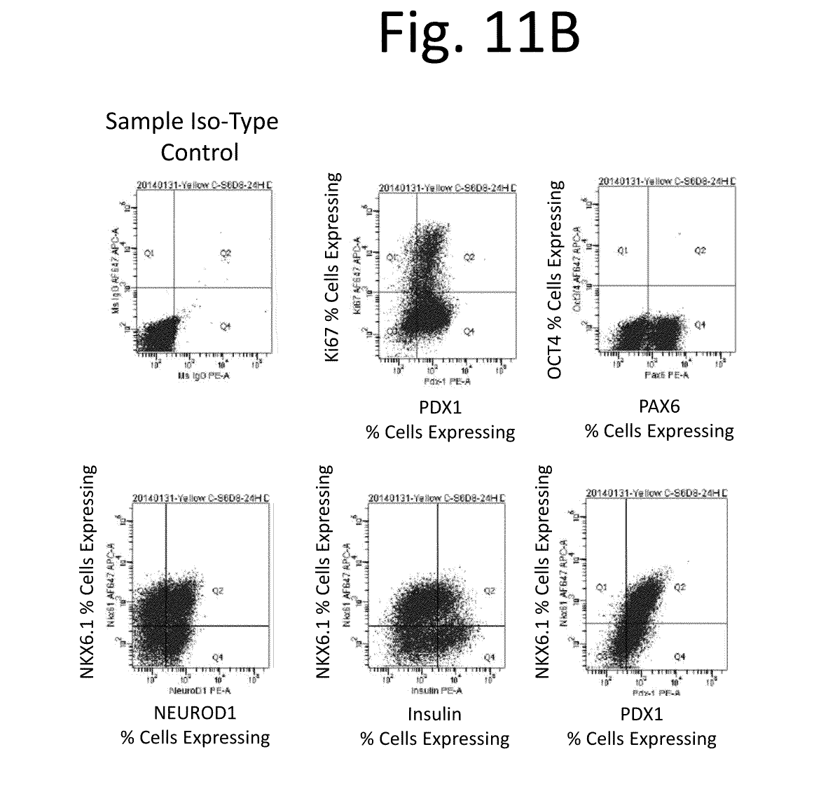

FIG. 4 depicts flow cytometry dot plots for live cells from the end of Stage 1 co-stained for the surface markers CD 184 and CD9; or CD 184 and CD99 (summarized in Table IIIA). FIG. 5 depicts flow cytometry dot plots for fixed and permeabilized cells from the end of Stage 4 co-stained for the following paired intra-cellular markers: NKX6.1 and Chromogranin-A; Ki67 and PDX1; and NKX2.2 and PDX1 (summarized in Table IIIA). FIGS. 6A and B (Condition A), 7A and B (Condition B), and 8A and B (Condition C) show flow cytometry dot plots for fixed and permeabilized cells from the end of Stage 5 co-stained for the following paired intra-cellular markers: NKX6.1 and Chromogranin-A; NKX2.2 and Chromogranin-A; NKX6.1 and C-peptide; Glucagon and Insulin; Ki67 and PDX1; OCT4 and PAX6; NKX6.1 and NEUROD1; NKX6.1 and Insulin; and NKX6.1 and PDX1. FIGS. 9A and B (Condition A), 10A and B (Condition B), and 11A and B (Condition C) depict fixed and permeabilized cells from the end of Stage 6 stained and measured by flow cytometry for the co-stained and paired intra-cellular markers: NKX6.1 and Chromogranin-A; NKX2.2 and Chromogranin-A; Glucagon and Insulin; NKX6.1 and C-peptide; Insulin and C-peptide; Ki67 and PDX1; OCT4 and PAX6; NKX6.1 and NEUROD1; NKX6.1 and Insulin; and NKX6.1 and PDX1.

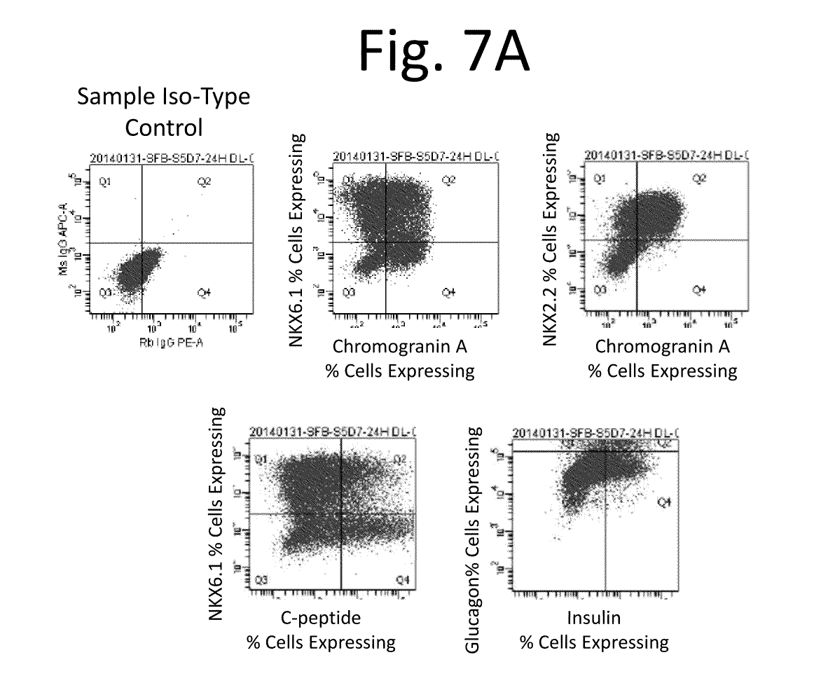

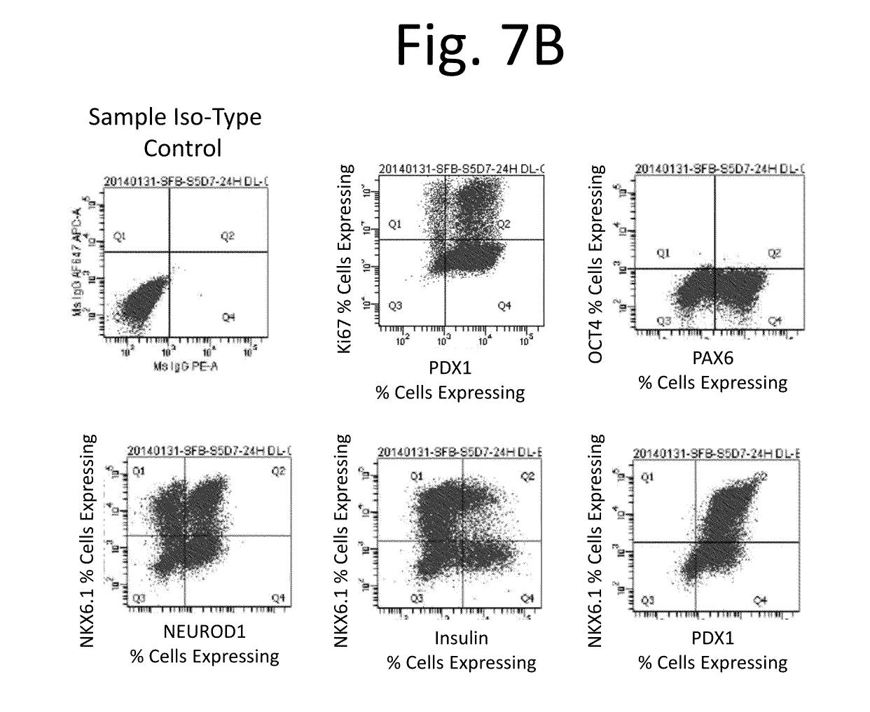

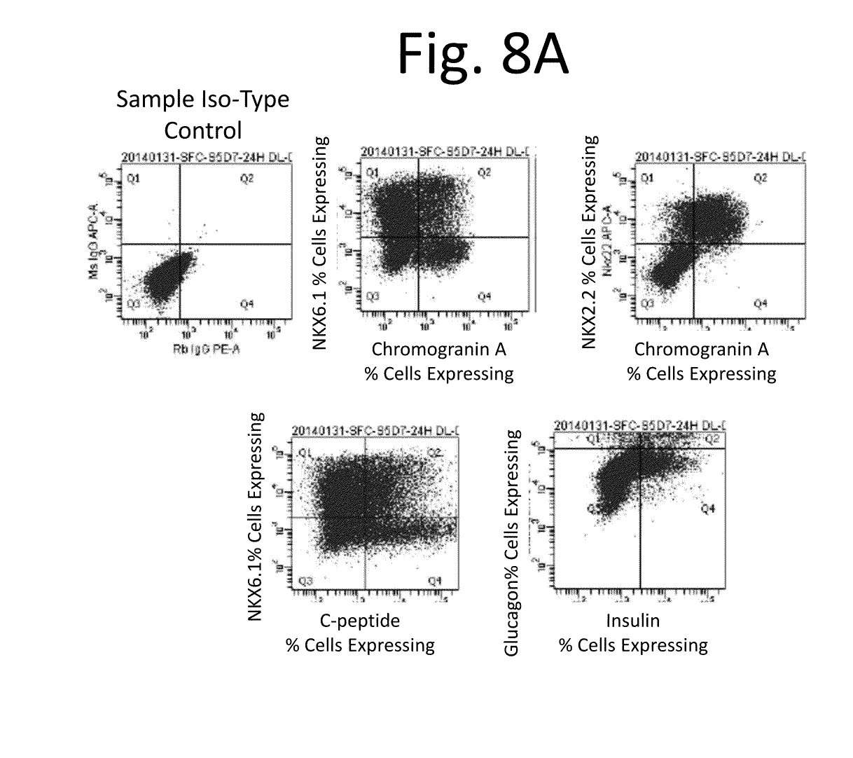

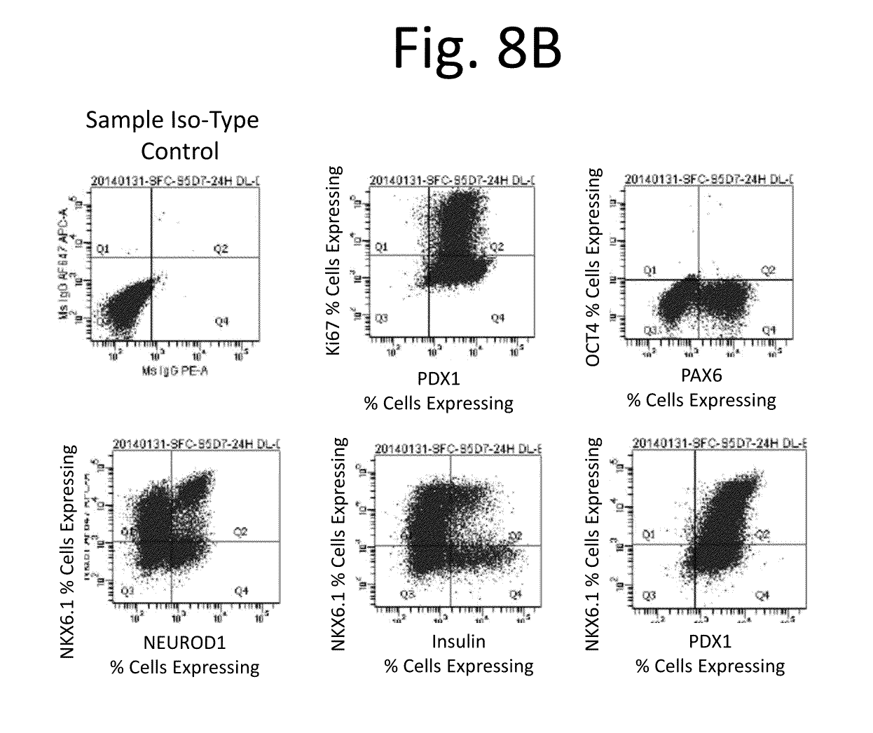

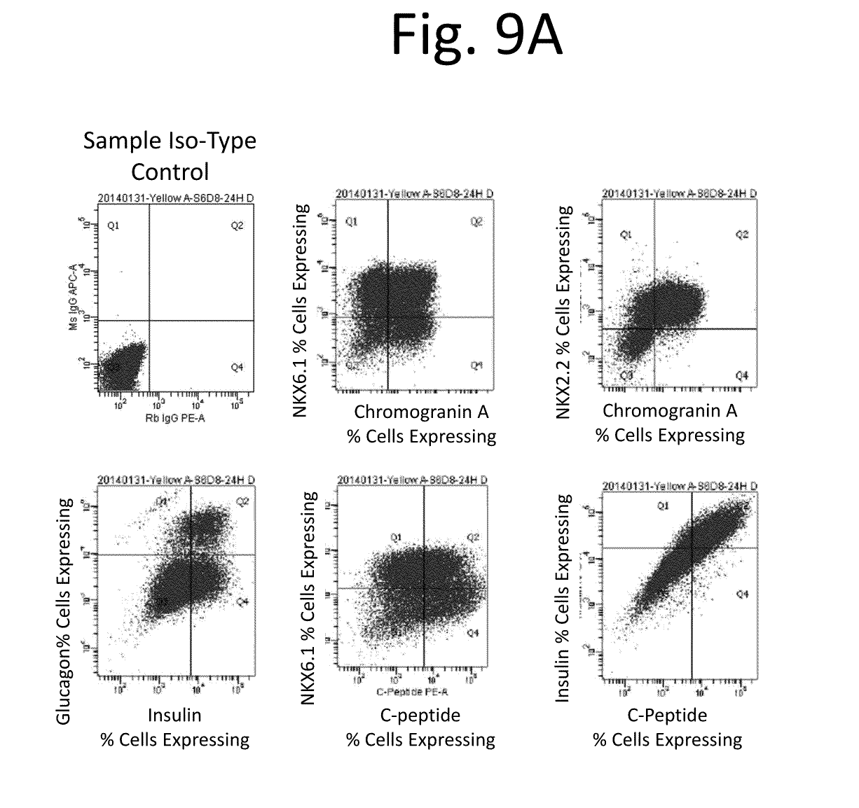

At the end of Stage 5, as shown in FIGS. 6A, 7A, and 8A and summarized in Table IIIB, 17%, 12%, or 10% of cells differentiated with conditions A, B, or C co-expressed insulin and NKX6.1; respectively. At the completion of Stage 6, an increase was observed in the number of NKX6.1 and insulin co-expressing cells (31% condition A; 15% condition B; 14% condition C). Moreover, it was noted that a substantial majority of cells at the end of Stage 6 expressed the beta cell precursor marker NKX6.1, the endocrine precursor marker NKX2.2, and the endocrine precursor marker NEUROD1 (condition A-74% NKX6.1, 82% NKX2.2, 74% NEUROD1; condition B-75% NKX6.1, 76% NKX2.2, 67% NEUROD1; condition C-60% NKX6.1, 64% NKX2.2, 53% NEUROD1).

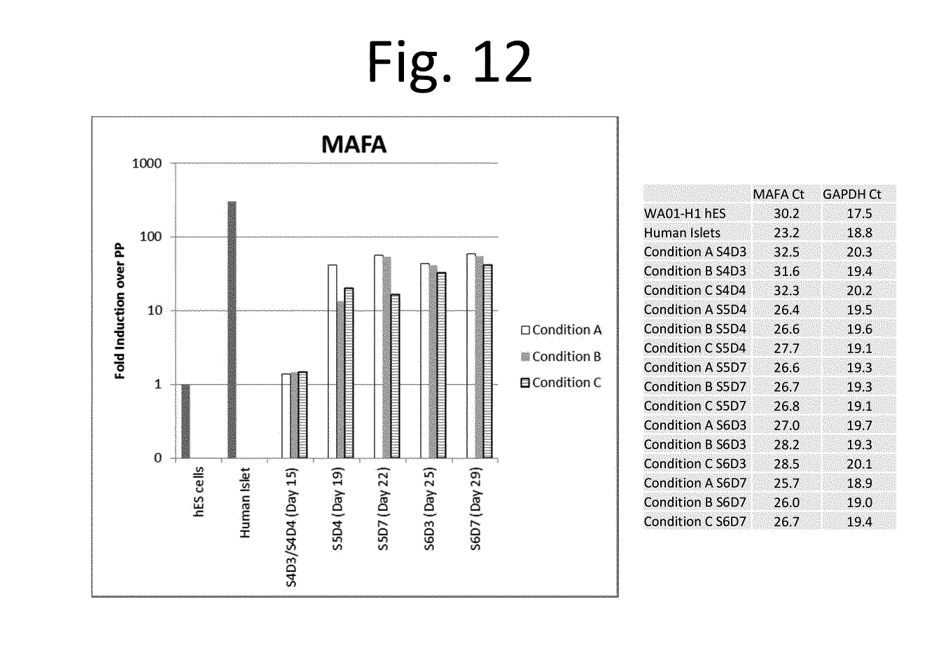

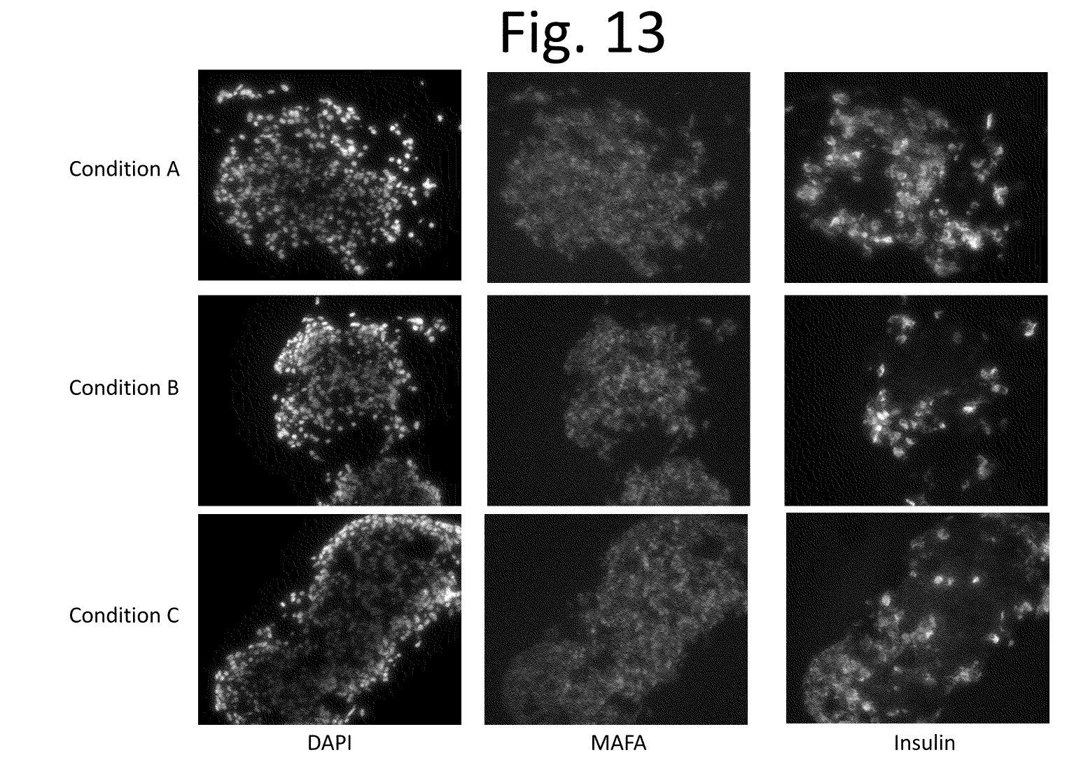

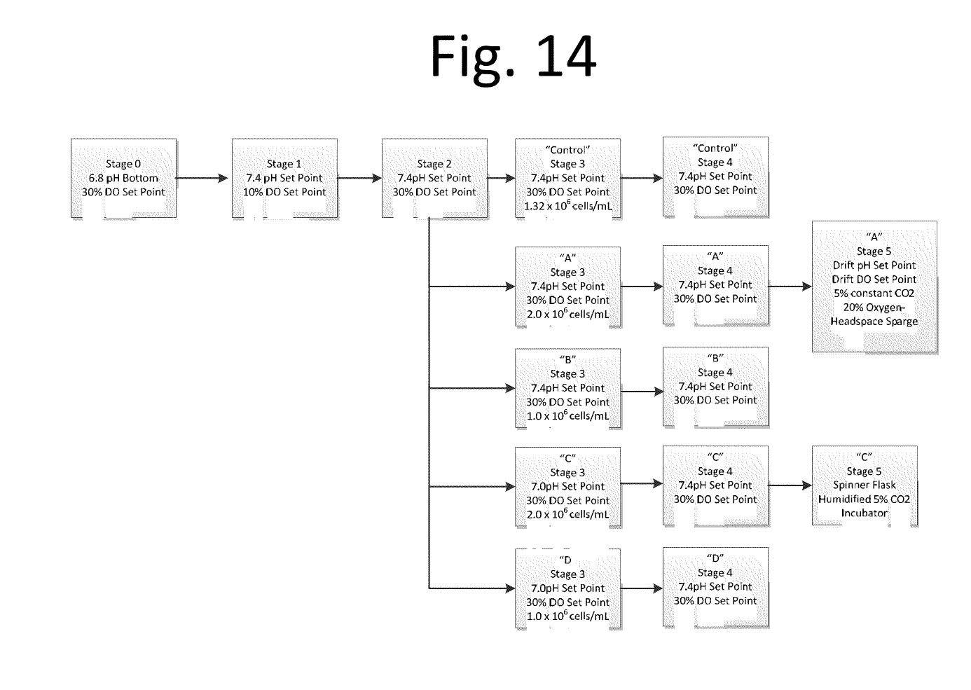

In addition to increased expression of markers required for beta cell maturation and function, it was observed that the percentage of PDX1 positive cells in active cell cycle as measured by co-expression for PDX1 and Ki-67 dropped from Stage 5 to Stage 6 (26% dropping to 9%, condition A; 22% dropping to 10%, condition B; 43% dropping to 19%, condition C). Furthermore, as the expression of Ki-67 measured by flow cytometry dropped over the course of Stages 5 and 6 in all 3 tested conditions, we detected increasing levels of the beta-cell specific transcription factor MAFA by TaqMan® qRT-PCR. MAFA expression at the end of Stage 6 was 40+ fold higher than undifferentiated pluripotent stem cells and reached a level that was approximately 25% of expression observed in human islet tissue (FIG. 12). The protein expression of MAFA was confirmed by immuno-fluorescent cytochemistry, as shown in FIG. 13, depicting micrographs obtained by 20× objective of immuno-fluorescent nuclear MAFA staining, immuno-fluorescent cytoplasmic insulin staining, and a pan-nuclear stain (“DAPI”).

These results, described above, indicate that cells moving from Stage 5 to Stage 6 converted from proliferating pancreatic endocrine progenitors to endocrine cells. These endocrine tissues, and specifically the insulin positive cells, expressed key markers associated with and required for functional beta cells. Conditions A and B, in which cells were cultured at a significantly lower pH than in condition C for Stages 3 and 4, generated more chromogranin positive, C-peptide/NKX6.1 co-positive cells and NEUROD1/NKX6.1 co-positive cells by the end of the six stage differentiation process compared to condition C. Condition C is a method known in the art and disclosed in Cell, 159: 428-439 (2014).

Cells differentiated through Stage 6 by conditions A and C were isolated from the media in a 50 mL conical, then washed 2 times with MCDB-131 medium containing 1.18 g/L sodium bicarbonate supplemented with an additional 1.2 g/L sodium bicarbonate and 0.2% w/v FAF-BSA. The cells were then re-suspended in the wash media and held at room temperature for approximately 5 hours prior to implantation under the kidney capsule of NSG mice (N=7). The animals were monitored for blood glucose and C-peptide levels at 4, 8, 10, and 14 weeks post engraftment. The animals were fasted overnight, given an intra-peritoneal injection of glucose, and blood was drawn via retro-orbital bleed 60 minutes after (“post”) the IP glucose bolus injection (Table III). At the earliest measured time point (4 weeks post-engraftment) the grafts functioned as measured by secretion of detectable levels of C-peptide (Table IV). Furthermore, C-peptide levels rose from week 4 to week 14.

At 10 weeks post-implantation, each animal was bled immediately prior (“pre”) to and immediately after (“post”) the glucose bolus injection. For reference, “post” C-peptide levels that were higher than “pre” levels would indicate glucose stimulated insulin secretion. We noted that 6 of 7 animals treated with a graft differentiated by condition C showed higher “post” levels of C-peptide and 3 of 7 animals treated with a graft differentiated by condition A had higher “post” levels of C-peptide.

TABLE I
Daily pH measurement from spent media; Example
1, Stage 3, day 1 through Stage 5, day 2.

| Stage | Condition | Condition | Stage | Condition |
|---|---|---|---|---|
| and Day | A pH | B pH | and Day | C pH |
| S3D1 | 7.19 | 7.12 | S3D1 | 7.31 |
| S3D2 | 7.29 | 7.25 | S3D2 | 7.39 |
| S3D3 | 7.18 | 7.22 | S4D1 | 7.44 |
| S4D1 | 7.11 | 7.28 | S4D2 | 7.37 |
| S4D2 | 7.04 | 7.21 | S4D3 | 7.48 |
| S4D3 | 7.08 | 7.19 | S4D4 | 7.43 |
| S5D1 | 7.44 | 7.45 | S5D1 | 7.48 |
| S5D2 | 7.35 | 7.41 | S5D2 | 7.46 |

TABLE II
List of Antibodies used for FACS analysis
of cells generated in Example 1

| Antigen | Species | Source/Catalogue Number | Dilution |
|---|---|---|---|
| Glucagon | Mouse | Sigma-Aldrich Co. LLC/G2654 | 1:500 |
| Insulin | Rabbit | Cell Signaling Technology. | 1:10 |
| | | Inc., Danvers. MA/3014B | |
| NKX6.1 | Mouse | Developmental Studies Hybridoma | 1:50 |
| | | Bank. Iowa City, Iowa/F55A12 | |
| NKX2.2 | Mouse | Developmental Studies Hybridoma | 1:100 |
| | | Bank/74.5A5 | |
| PDX1 | Mouse | BD BioSciences, San Jose, | 1:20 |
| | | CA/562161 | |
| Ki67 | Mouse | BD Biosciences/561126 | 1:20 |
| PAX6 | Mouse | BD Biosciences, 561552 | 1:20 |
| Chromogranin A | Rabbit | Dako, Carpinteria, CA/1S502 | 1:10 |
| ISL-1 | Mouse | BD Biosciences/562547 | 1:20 |
| NEUROD1 | Mouse | BD Bioscience/563566 | 1:40 |
| FOXA2 | Mouse | BD Bioscience/561589 | 1:80 |
| OCT3/4 | Mouse | BD Biosciences/560329 | 1:20 |
| C-peptide | Rabbit | Cell Signaling Technology/ | 1:100 |
| | | #4593S | |
| Insulin | Mouse | Abcam/#7760 | 1:800 |

TABLE IIIA

| Name | CD9 | CD184 | SSEA4 | TRA-1-60 | TRA-1-81 |
|---|---|---|---|---|---|
| Pluripotentcy | | | | | |
| SOD3-24H | | | | | |
| BX1 | 82 | 0 | 100 | 90 | 76 |
| | CD9 | CD184 | CD99 | | |
| DE; | | | | | |
| S1D3-24H | | | | | |
| SF A | 52 | 99 | 95 | — | — |
| SF B | 43 | 99 | 98 | — | — |
| SF C | 17 | 99 | 100 | — | — |
| | NKX6.1 | CHGA | NKX2.2 | PDX1 | NEUROD1 |
| Stage 4 | | | | | |
| S4D3-24H | | | | | |
| SF A | 73 | 21 | 25 | 100 | 23 |
| SF B | 69 | 12 | 14 | 99 | 14 |
| Stage 4 | | | | | |
| S4D4-24H | | | | | |
| SF C | 53 | 10 | 13 | 96 | 12 |

TABLE IIIB

| | Stage 5 | Stage 5 | Stage 5 | Stage 6 | Stage 6 | Stage 6 |
|---|---|---|---|---|---|---|
| | S5D7-24 H | S5D7-24 H | S5D7-24 H | S6D8-24 H | S6D8-24 H | S6D8-24 H |
| | SF A | SF B | SF C | SF A | SF B | SF C |
| NKX6.1 | 74 | 69 | 62 | 74 | 75 | 60 |
| CHGA | 39 | 55 | 32 | 61 | 61 | 47 |
| NKX6.1+/ | 25 | 35 | 17 | 44 | 43 | 21 |
| CHGA | | | | | | |
| PDX1 | 99 | 95 | 97 | 94 | 89 | 82 |
| NKX6.1+/ | 68 | 61 | 99 | 74 | 66 | 54 |
| PDX1+ | | | | | | |
| Ki67+/ | 26 | 22 | 43 | 9 | 10 | 19 |
| PDX1+ | | | | | | |
| NEUROD1 | 60 | 63 | 33 | 74 | 67 | 53 |
| NKX6.1+/ | 40 | 38 | 19 | 53 | 45 | 28 |
| NEUROD1+ | | | | | | |
| NKX6.1+/ | 26 | 15 | 22 | 29 | 27 | 21 |
| C-PEP+ | | | | | | |
| NKX6.1+/ | 17 | 12 | 10 | 31 | 15 | 14 |
| INS+ | | | | | | |
| C-PEP+/INS+ | 30 | 27 | 22 | 38 | 25 | 28 |
| NKX2.2 | 65 | 71 | 38 | 82 | 76 | 64 |

TABLE IV

| | Cell Dose | | | | |
|---|---|---|---|---|---|
| | (% INS/ | C-peptide | C-peptide | C-peptide | C-peptide |
| | NKX6.1 | ng/mL | ng/mL | ng/mL | ng/mL |
| Condition | copositive) | (4 wk) | (8 wk) | (10 wk) | (14 wk) |
| A | 5M (32%) | 0.716 | 1.056 | 0.975 | 2.18 |
| C | 5M (14%) | 0.406 | 0.641 | 1.052 | 1.39 |

### Example 2

This example demonstrates formation of insulin expressing cells from a population of cells expressing PDX1 in a stirred-tank closed loop which allowed for direct computer control of medium pH and dissolved oxygen concentration via feedback pH and DO sensors in the reactor. The insulin positive cells generated from this process retained PDX1 expression and co-expressed NKX6.1. The insulin positive cells were generated from cells exposed to four different conditions (A, B, C, and D) in Stages 3 through 5 (Table V). It was observed that, when the cells differentiated according to condition C (pH 7.0 and cell concentration of 2 million/mL at the beginning of Stage 3) were transplanted into the kidney capsule of immune-compromised mice, the graft produced detectable blood levels of human C-peptide within four weeks of engraftment.

Cells of the human embryonic stem cell line H1 (WA01 cells, WiCell Research Institute, Madison, Wisconsin) were grown in E8TM supplemented with 0.5% w/v FAF-BSA in dynamic suspension for ≧4 passages as round aggregated clusters. The clusters were then frozen as single cells and clusters of 2 to 10 cells per the following method. Approximately 600-1000 million cells in aggregated clusters were transferred to a centrifuge tube and washed using 100 mL of 1× DPS −/−. After the wash, the cell aggregates were then enzymatically disaggregated by adding a 30mL solution of 50% StemPro®Accutase® enzyme and 50% DPBS −/− by volume to the loosened cell aggregate pellet. The cell clusters were pipetted up and down 1 to 3 times and then intermittently swirled for approximately 4 minutes at room temperature, then centrifuged for 5 min, at 80 to 200 rcf. The Accutase® supernatant was then aspirated as completely as possible without disturbing the cell pellet. The centrifuge tube was then tapped against a hard surface for approximately 4 minutes, to disaggregate the clusters into single cells and clusters comprised of 2 to10 cells. After 4 minutes, the cells were re-suspended in 100 mL of E8™ media supplemented with 10 μM Y-27632 (Enzo Life Sciences, Inc., Farmingdale, N.Y.; Catalog No. ALX-270-333) and 0.5% w/v FAF-BSA, and centrifuged for 5 to 12 minutes at 80 to 200 rcf. The supernatant was then aspirated and cold (≦4° C.) Cryostor® Cell Preservation Media CS10 was added drop-wise to achieve a final concentration of 100 to 150 million cells per mL. This cell solution was held in an ice bath while being aliquoted to 2 mL cryogenic vials after which the cells were frozen using a controlled rate freezer (CryoMed™ 34 L Controlled-Rate Freezer) as follows. The chamber was cooled to 4° C. and the temperature was held until a sample vial temperature reached 6° C. and then the chamber temperature was lowered 2° C. per minute until the sample reached −7° C. at which point the chamber was cooled 20° C./min. until the chamber reached −45° C. The chamber temperature was then allowed to briefly rise at 10° C./min. until the temperature reached −25° C., and then the chamber was cooled further at 0.8° C./min. until the sample vial reached −40° C. The chamber temperature was then cooled at 10° C./min. until the chamber reached −100° C. at which point the chamber was then cooled 35° C./min. until the chamber reached −160° C. The chamber temperature was then held at −160° C. for at least 10 minutes, after which the vials were transferred to gas phase liquid nitrogen storage. These cryo-preserved single cells at high concentration were then used as an intermediate/in-process seed material ISM.

Vials of ISM were removed from the liquid nitrogen storage, thawed, and used to inoculate a 3 liter glass, stirred suspension tank DASGIP bioreactor. The vials were removed from liquid nitrogen storage and quickly transferred to a 37° C. water bath for 120 seconds to thaw. The vials were then moved to a BSC and the thawed contents transferred via 2 mL glass pipette to a 50 mL conical tube. Then 10 mL of E8™ medium supplemented with 0.5% w/v FAF-BSA and 10 μM of Rho kinase inhibitor Y-27632, were added to the tube in a drop-wise manner. The cells were centrifuged at 80-200 rcf for 5 min. The supernatant from the tube was aspirated and 10 mL fresh E8™ medium supplemented with 0.5% w/v FAF-BSA and 10 μM Y-27632 were added and the volume containing the cells was pipetted into a media transfer bottle (Cap2V8) containing 450 mL E8™ media supplemented with 0.5% w/v FAF-BSA and 10 μM Y-27632. The bottle contents were then pumped directly into the bioreactor via a sterile, C-Flex® tubing weld using a peristaltic pump. The bioreactor was prepared with 1000 mL E8™ medium supplemented with 0.5% w/v FAF-BSA and 10 μM Y-27632 pre-warmed to 37° C., stirred at 70 rpm, with a dissolved oxygen set point of 30% (air O₂, and N₂ regulated), and a controlled CO₂ partial pressure of 5% . The reactor was inoculated to give a target concentration of 0.225×10⁶ cells/mL (concentration range: 0.2 to 0.5×10⁶ cells/mL).

Once the reactor was inoculated, the cells formed round aggregated clusters in the stirred reactor. After 24 hours in culture, the medium was partially exchanged as more than 80% of the original volume was removed and 1.5 L of E8™ media supplemented with 0.5% w/v FAF-BSA was added back (fresh medium). This media exchange process was repeated 48 hours after inoculation. After three days in suspension culture as round aggregated clusters, directed differentiation was initiated. In order to initiate differentiation, spent medium was removed and differentiation media was pumped into the bioreactor and exchanged over the course of the process using medial) exchange and differentiation protocols as described below.

Stage 1 (3 Days):

A base medium was prepared using MCDB-131 medium containing 1.18 g/L sodium bicarbonate; supplemented with an additional 2.4 g/L sodium bicarbonate, 2% w/v FAF-BSA, previously re-constituted in MCDB-131; 1× concentration of GlutaMAX™; 2.5 mM glucose (45% in water); and a 1:50,000 dilution of ITS-X. Cells were cultured for one day in 1.5 L of the base medium supplemented with 100 ng/ml GDF8 and 3 μM MCX compound. After 24 hours, spent medium was removed and fresh 1.5 L of base medium supplemented with 100 ng/mL of GDF8 were added to the reactor. Cells were maintained without further media exchange for 48 hours.

Stage 2 (3 Days):

A base medium was prepared using MCDB-131 medium containing 1.18 g/L sodium bicarbonate and supplemented with an additional 2.4 g/L sodium bicarbonate; 2% w/v FAF-BSA, previously re-constituted in MCDB-131; 1× concentration of GlutaMAX™; 2.5 mM glucose; and a 1:50,000 dilution of ITS-X. After the completion of Stage 1, a media exchange was completed as described above, whereby the spent Stage 1 media was removed and replaced with 1.5 L of Stage 2 base medium supplemented with 50 ng/mL FGF7. Forty-eight hours after the media exchange, the spent media was again removed and replaced with 1.5 L fresh Stage 2 Base Medium supplemented with 50 ng/mL FGF7.

### Stage 3 (3 Days):

At the completion of Stage 2, and just prior to medium exchange, 900 million cells were removed from the 3 liter reactor via sterile weld and peristaltic pump. The medium in the 3 liter reactor was then exchanged as previously described and replaced with the following Stage 3 media: MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of G1utaMAXTM; 2.5 mM glucose; and a 1:200 dilution of ITS-X. The Stage 3 medium was supplemented with 50 ng/mL FGF-7; 100 nM of LDN-193189; 2 μM RA; 0.25 μM SANT-1; and 400 nM of TPB. The removed cells were then spun down in a sterile conical tube, the spent media was removed, and the cells were re-suspended in the Stage 3 medium and supplements. These cells were then transferred via sterile weld and peristaltic pump to four separate 0.2 liter glass stirred suspension tank bioreactors (reactors A, B, C, and D) from DASGIPTM. The cells in the 0.2 liter bioreactors and the 3 liter control bioreactor were exposed to different combinations of cell concentration and media pH as shown in FIG. 14 and the Table V for Stages 3 through 5. Twenty-four hours post media exchange, the spent media was again replaced in each of the control and reactors A through D with 300 mL fresh Stage 3 medium containing the above supplements with the exception of LDN-193189. Cells were cultured in the media for 48 hours.

TABLE V

| | | DO | | | | | |
|---|---|---|---|---|---|---|---|
| | pH Set | Set | pH Set | DO Set | Drift pH | Drift DO | |
| | Point | Point | Point | Point | Set Point | Set Point | Cell |
| | Stage 3 | Stage 3 | Stage 4 | Stage 4 | Stage 5 | Stage 5 | Concentration |
| Control | 7.4 | 30% | 7.4 | 30% | Moved to | Moved to | 1.32 × 10⁶ cells/mL |
| Reactor | | | | | spinner | spinner | |
| | | | | | flask | flask | |
| Reactor A | 7.4 | 30% | 7.4 | 30% | Headspace | Headspace | 2.0 × 10⁶ cells/mL |
| | | | | | sparge | sparge | |
| | | | | | 5%; | 20%; | |
| | | | | | constant | constant | |
| | | | | | CO₂ | O₂ | |
| Reactor B | 7.4 | 30% | 7.4 | 30% | Moved to | Moved to | 1.0 × 10⁶ cells/mL |
| | | | | | spinner | spinner | |
| | | | | | flask | flask | |
| Reactor C | 7.0 | 30% | 7.4 | 30% | Headspace | Headspace | 2.0 × 10⁶ cells/mL |
| | | | | | sparge | sparge | |
| | | | | | 5%; | 20%; | |
| | | | | | constant | constant | |
| | | | | | CO₂ | O₂ | |
| Reactor D | 7.0 | 30% | 7.4 | 30% | Moved to | Moved to | 1.0 × 10⁶ cells/mL |
| | | | | | spinner | spinner | |
| | | | | | flask | flask | |

Stage 4 (3 Days):

After the completion of Stage 3, the spent media was removed and replaced with 150 mL of the following Stage 4 medium: 150 mL MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of GlutaMAX™; 2.5 mM glucose; and a 1:200 dilution of ITS-X. The medium was supplemented with 0.25 μM SANT-1 and 400 nM of TPB. Forty-eight hours after initiation of Stage 4, 3.2 mL/L of a 45% glucose solution (8 mM glucose bolus) was added to each of the bioreactors and the cells were cultured in the media for an additional 24 hours.

Stage 5 (7 Days):

A Stage 5 base medium was prepared for each bioreactor using 150 mL MCDB-131 medium base containing 1.18 g/L sodium bicarbonate supplemented with an additional 1.754 g/L sodium bicarbonate; 2% w/v FAF-BSA previously re-constituted in MCDB-131; 1× concentration of GlutaMAX™; 20 mM glucose; 1:200 dilution of ITS-X; 250 μL/L of 1M ascorbic acid; and 10 mg/L heparin (Sigma Aldrich; Catalog No. H3149-100KU). After the completion of Stage 4, spent media in each bioreactor was exchanged for 150 mL of Stage 5 base medium supplemented with 1μM T3, 10 μM ALK5 inhibitor II, 1 μM of gamma secretase inhibitor XXI (EMD Millipore; Catalog No. 565790); 20 ng/mL of betacellulin; 0.25 μM SANT-1; and 100 nM RA. Forty-eight hours after initiation of Stage 5, the spent media was removed and replaced with 150 mL of the same fresh media and supplements. Forty-eight hours later, the medium was removed and replaced with Stage 5 base medium supplemented with 1μM T3, 10 μM Alk5 inhibitor II, 20 ng/ml betacellulin and 100 nM RA. Forty-eight hours later the medium was again exchanged and replaced with the same fresh medium and supplements. Twenty-four hours later marked the end of Stage 5 and the cells generated were processed for characterization and analysis.

Throughout the differentiation process, in addition to real-time continuous monitoring for pH and dissolved oxygen (“DO”), media samples were collected from the reactors on a daily basis. The spent medium at the end of each day was analyzed by NOVA bio-analyzer. Samples were also analyzed for cell number (Nucleocounter 100), mRNA expression (qRT-PCR), and protein expression (flow cytometry and florescent immune-histochemistry).

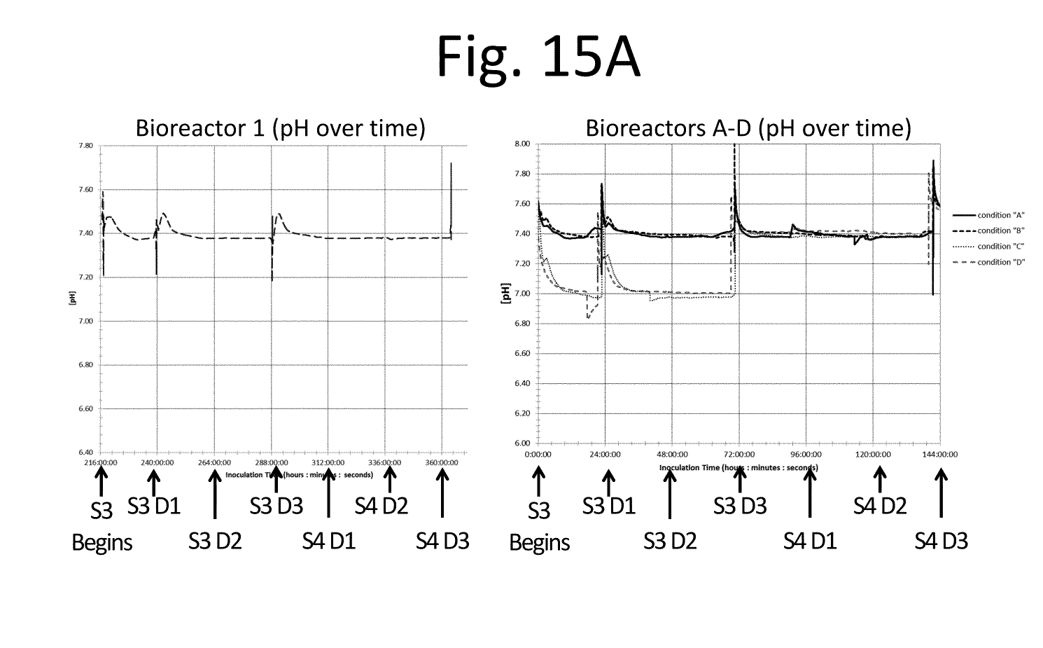

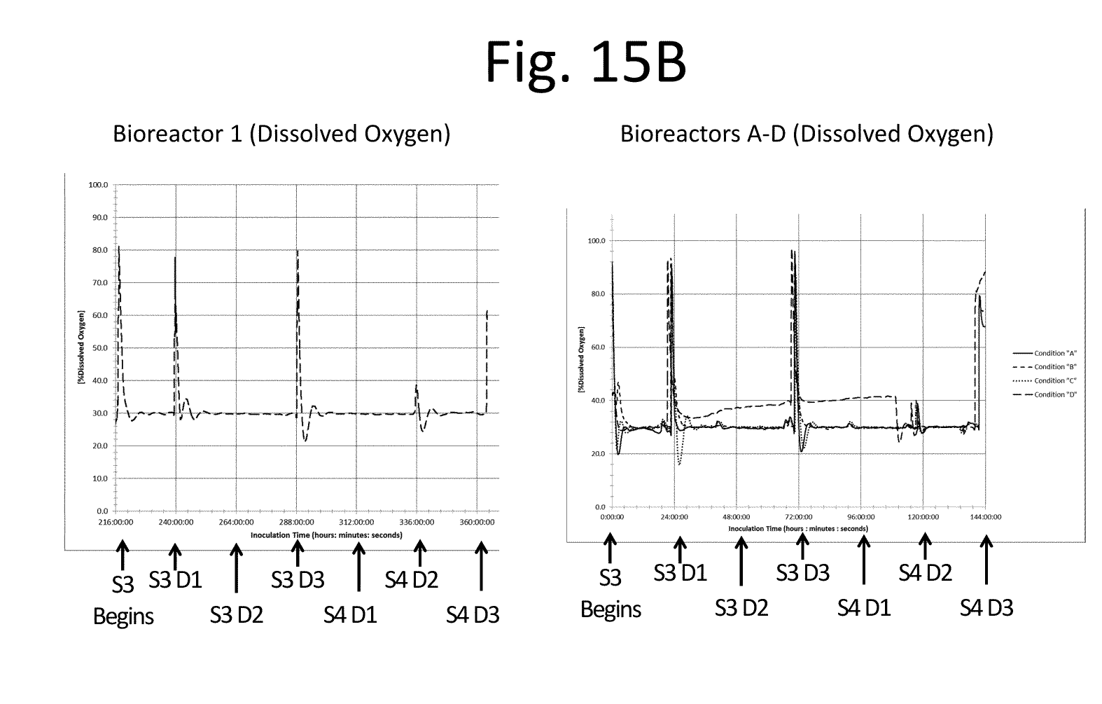

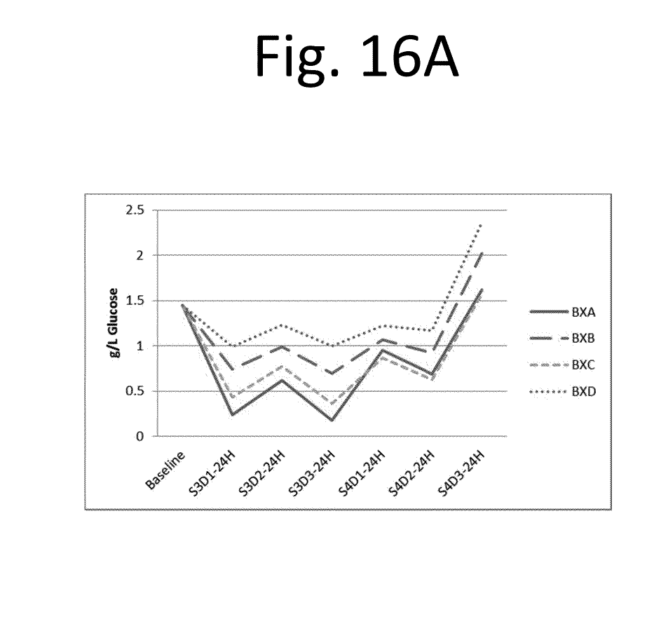

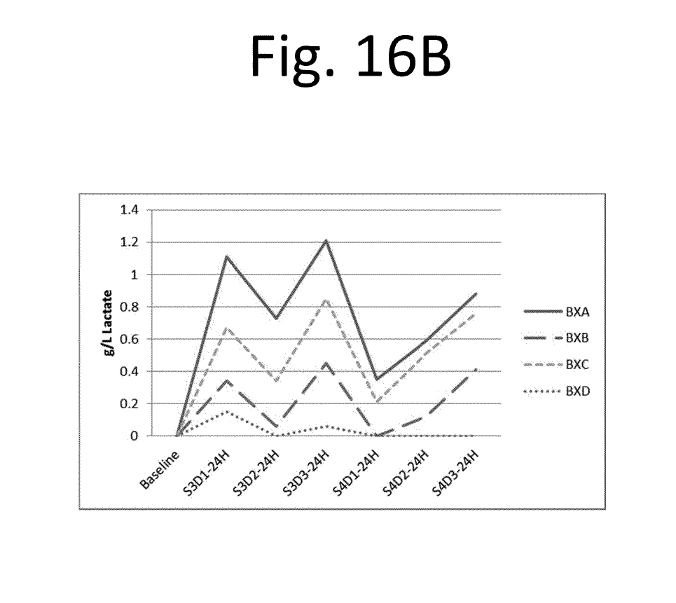

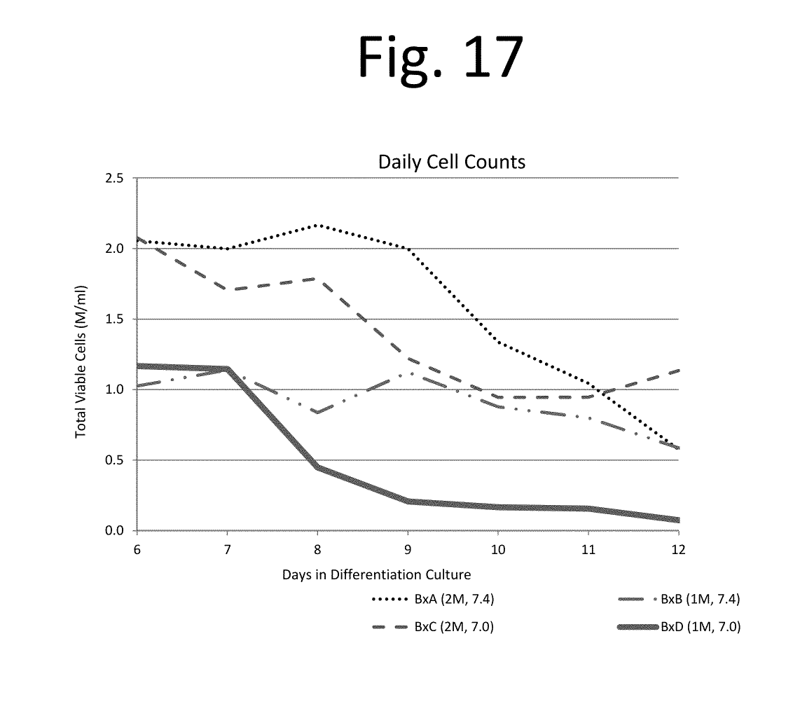

FIGS. 15A and B depict continuous monitoring graphs of pH (FIG. 15A) and dissolved oxygen levels (FIG. 15B) in media for reactors 1, A, B, C, and D over the course of Stages 3 and 4. FIGS. 16A and B depict data from a NOVA® BioProfile FLEX Analyzer obtained from spent media samples at the end of each day of differentiation in Stages 3 and 4 (FIG. 16A—glucose concentration; FIG. 16B—lactate concentration). FIG. 17 depicts cell count trend lines for reactors and conditions A, B, C, and D (also listed as B×A, B×B , B'3C, and BxD). These data demonstrate that in reactors set to pH 7.0, there is cell loss over the course of Stage 3 which correlates with the low pH (Bioreactors C and D) set-point. However, reactor C which was seeded at 2×10⁶ cells per mL recovered cell population by the end of Stage 4, while Reactor D which had a pH of 7.0 but cell seeding of 1.0×10⁶ cells per mL did not. Also, reactors A and B, pH of 7.4 and seeded at 2×10⁶ and 1.0×10⁶cells per mL, respectively, exhibited significant cell loses in Stage 4 although they both had maintained cell concentration through Stage 3 (FIG. 17). These data indicate that use of a pH setpoint of 7.0 in combination with a concentration of equal to or greater than about 1.5×10⁶cells per mL, preferably equal to or greater than about 2.0×10⁶cells per mL, at Stage 3 promotes higher cell concentration throughout subsequent differentiation stages as compared to cells maintained at pH 7.4 in Stage 3.

The effects in cell concentration were mirrored by daily spent medium levels of glucose and lactate. Both reactors C and D had more residual glucose and less lactate at the end of each day than their concentration paired pH 7.4 controls, A and B respectively. These results indicated that reactors C and D had less metabolic activity during Stage 3. However, as reactor C progressed through Stage 4, residual glucose levels were comparable to those in reactor A by the end of the first and second day of Stage 4, although lactate levels remained lower in reactor C. From these data we can infer that the cells in reactor C had begun to adopt a more differentiated, mature, and less glycolytic phenotype than those in reactor A.

At the completion of Stage 3 nearly all of the cells maintained in pH 7.0 at a starting concentration of 1×10⁶ (reactor D) or 2×10⁶ (reactor C) cells/mL were observed to express both the endoderm transcription factor (FOXA2) and the pancreatic specific transcription factor (PDX1), as did cells kept at pH 7.4 in a starting density of 1M (Reactor B) or 2M (Reactor A) indicating that low pH treated cells retain a pancreatic endodermal specification. Furthermore, in all five of the tested conditions the percentage of cells expressing NKX6.1 was similarly low (Range: 5.4-13.6%) at the end of Stage 3. Cells maintained at pH 7.4 (reactors A and B, and the control reactor, “1”) began to express NEUROD1 at the end of Stage 3 while cells kept at pH 7.0 (reactors C and D) showed reduced levels of NEUROD1 expression as measured by flow cytometry (Table Vi). At the initiation of Stage 4, the pH set-point for reactors C and D was returned to 7.4 (FIGS. 14 and 15A). Three days later, at the end of Stage 4, samples from each of the reactors were analyzed by flow cytometry for expression of NKX6.1, NEUROD1, PDX1, FOXA2, CDX2, and Ki67. It was observed that cells maintained at pH 7.0 in Stage 3 (Reactors C and D) had substantially more NKX6.1 positive cells and cells in active cell cycle (Ki67 positive) at the end of Stage 4 as detected by intracellular flow cytometry when compared to cells maintained in reactors set to a pH of 7.4 (Bioreactors 1, A, and B) as summarized in Table VI.

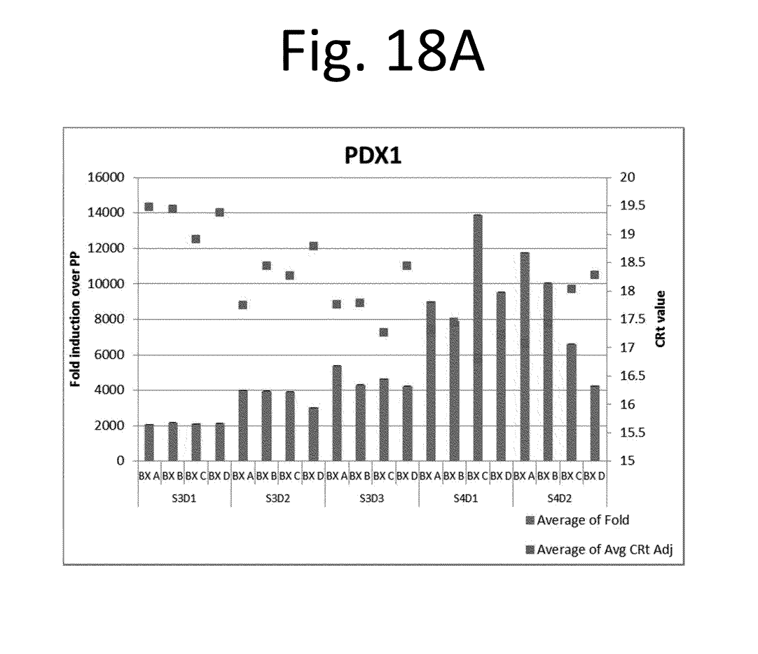

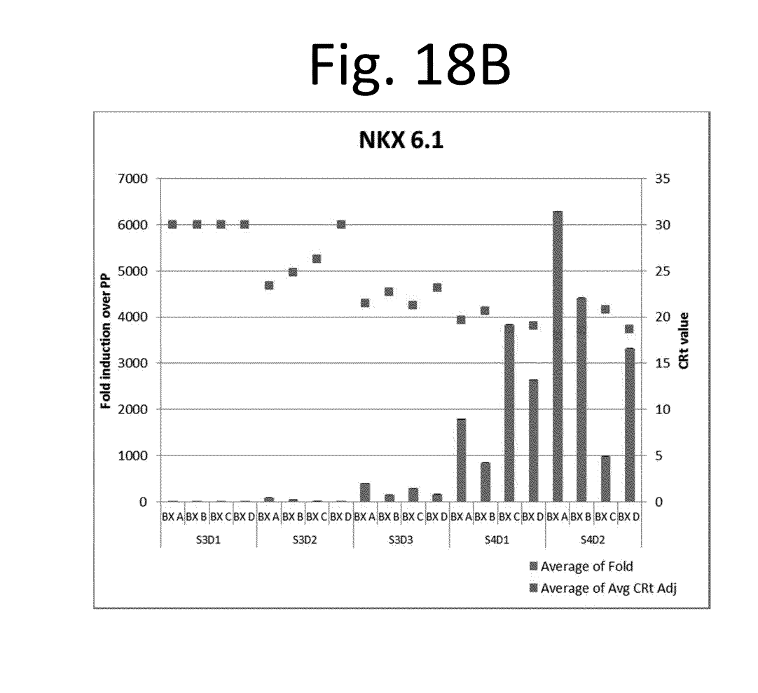

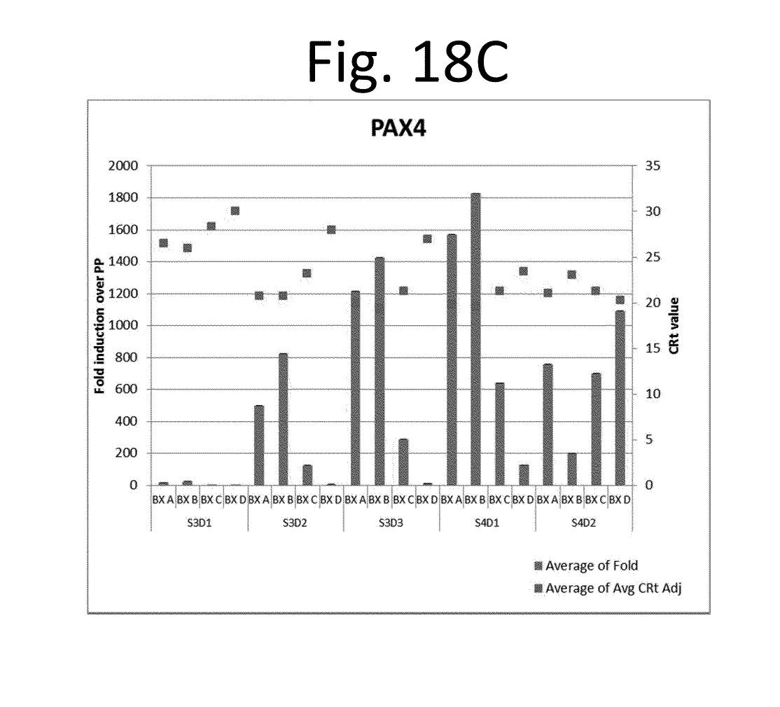

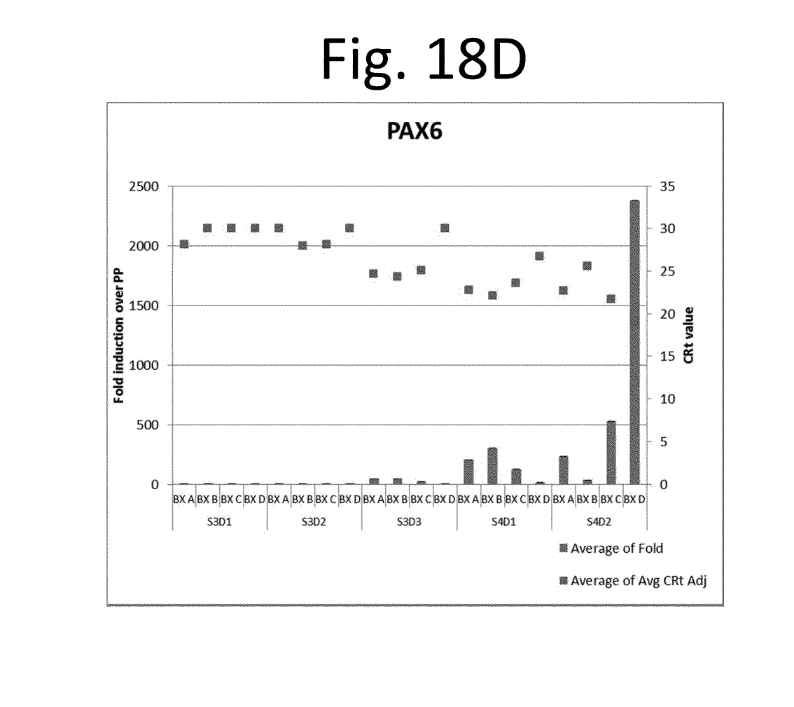

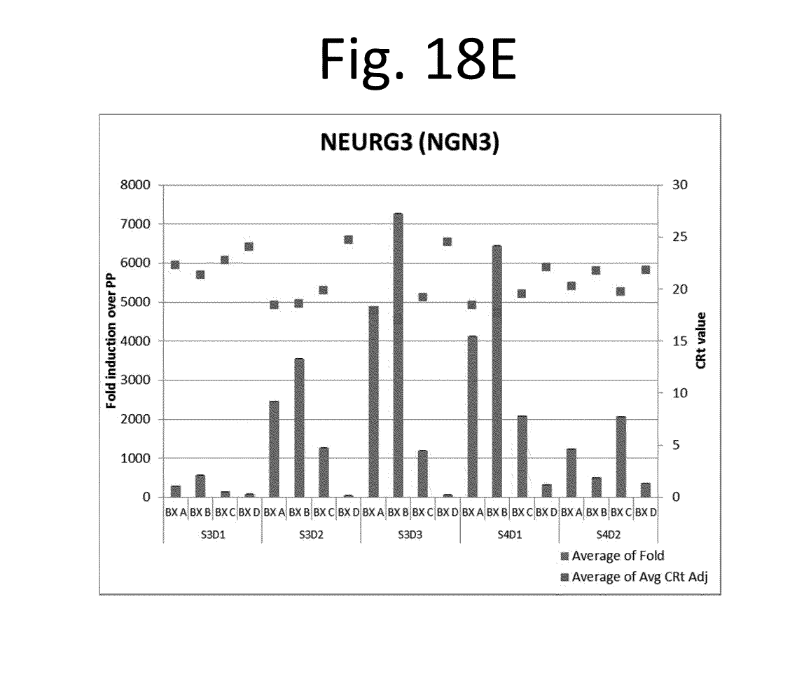

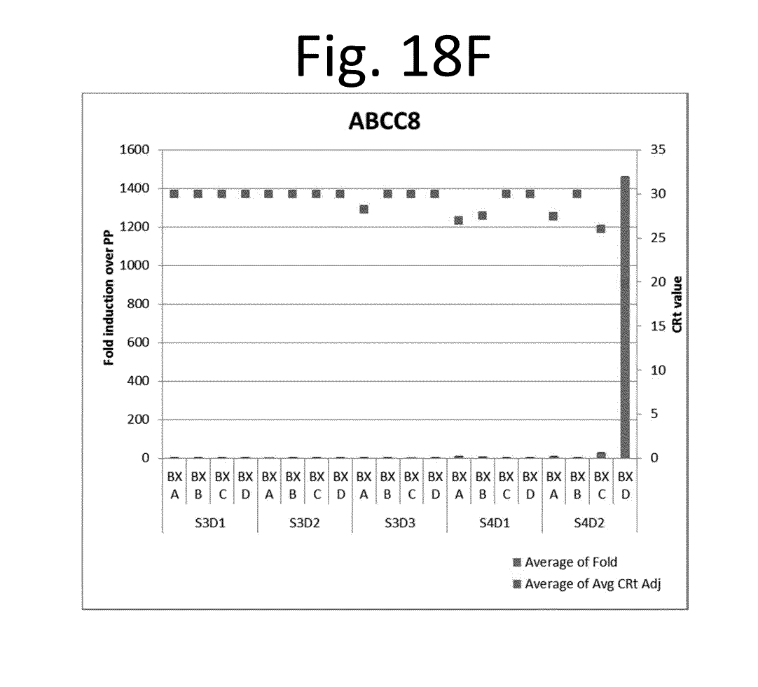

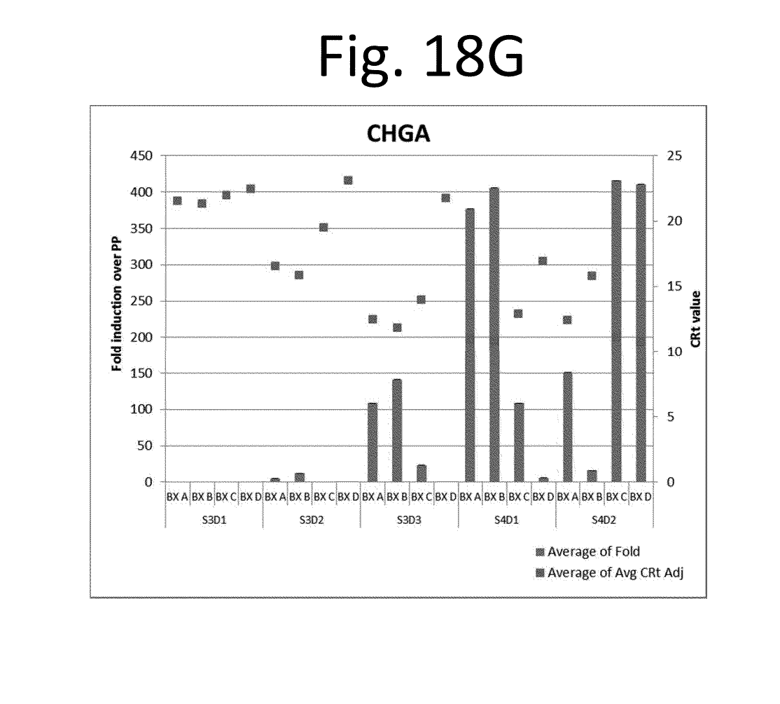

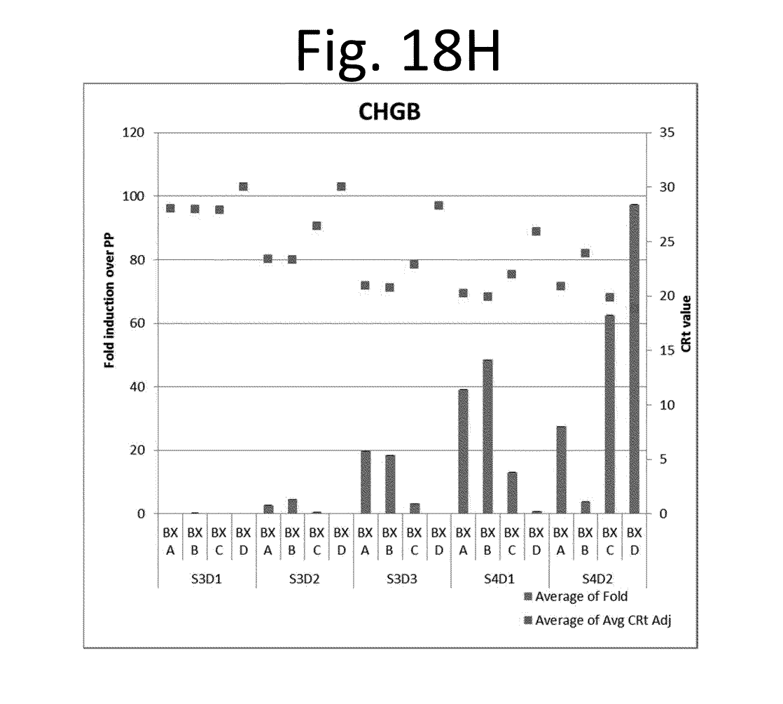

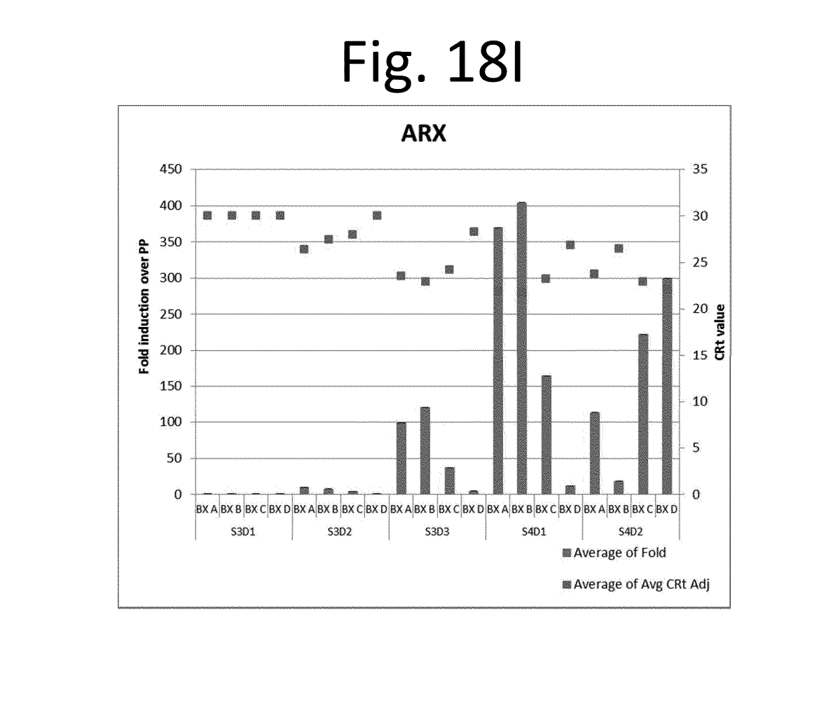

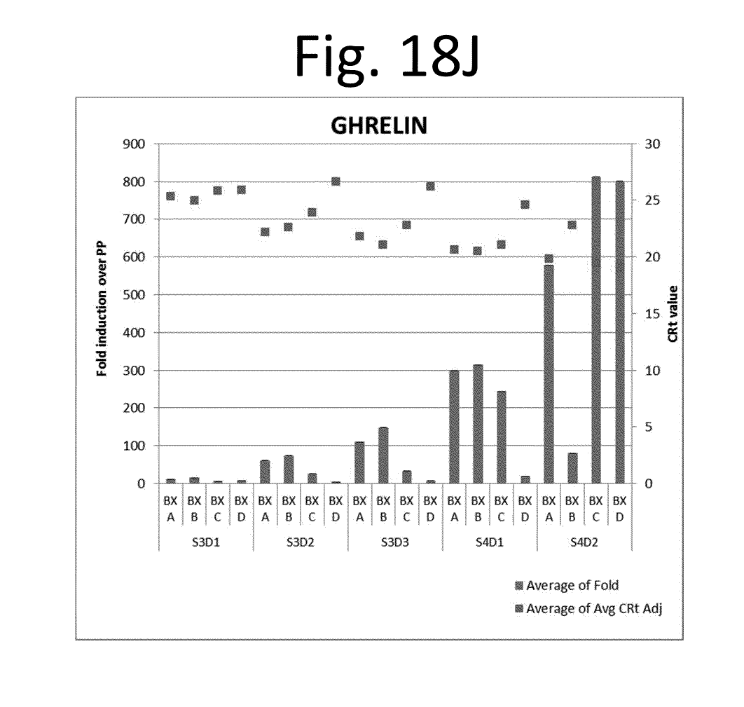

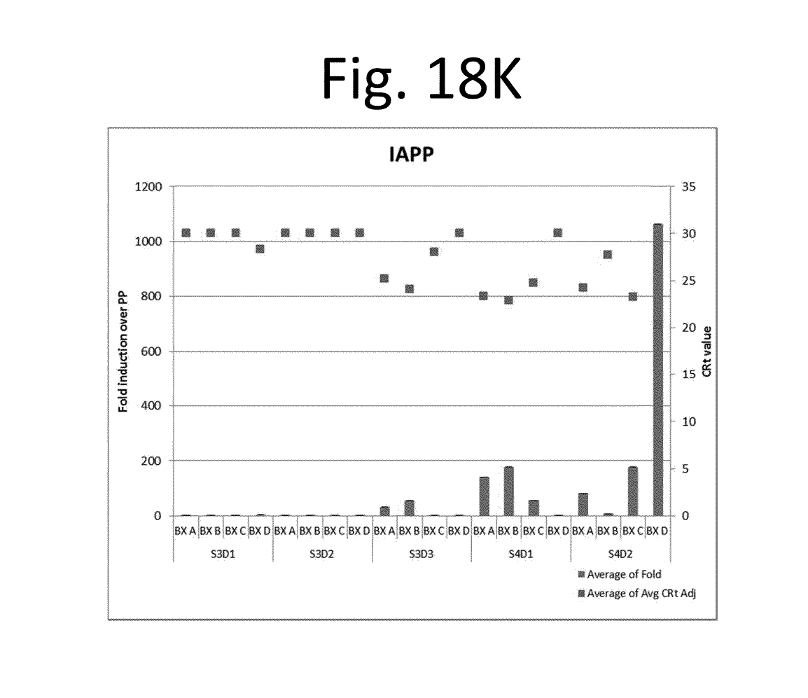

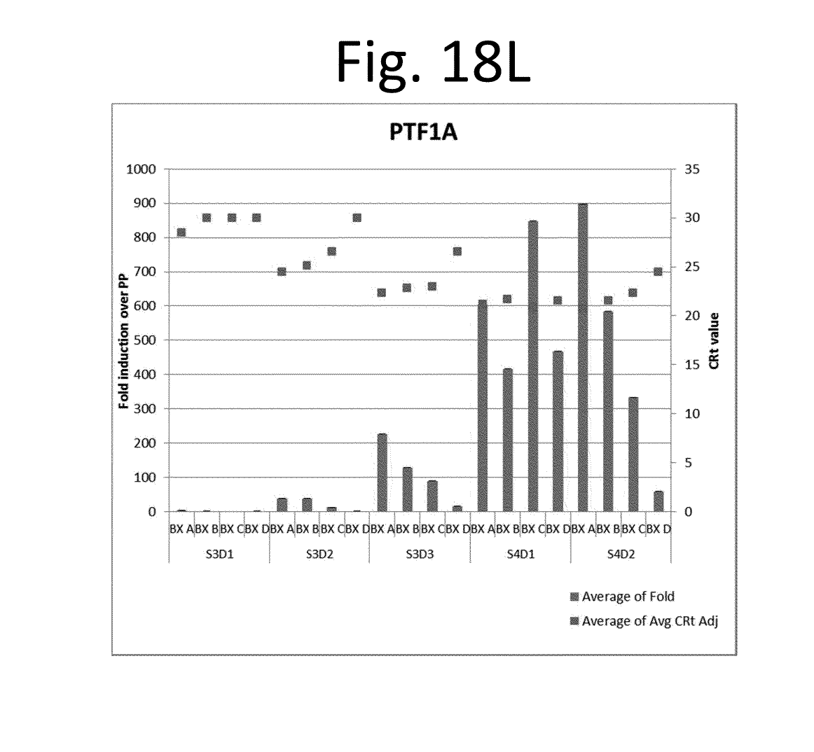

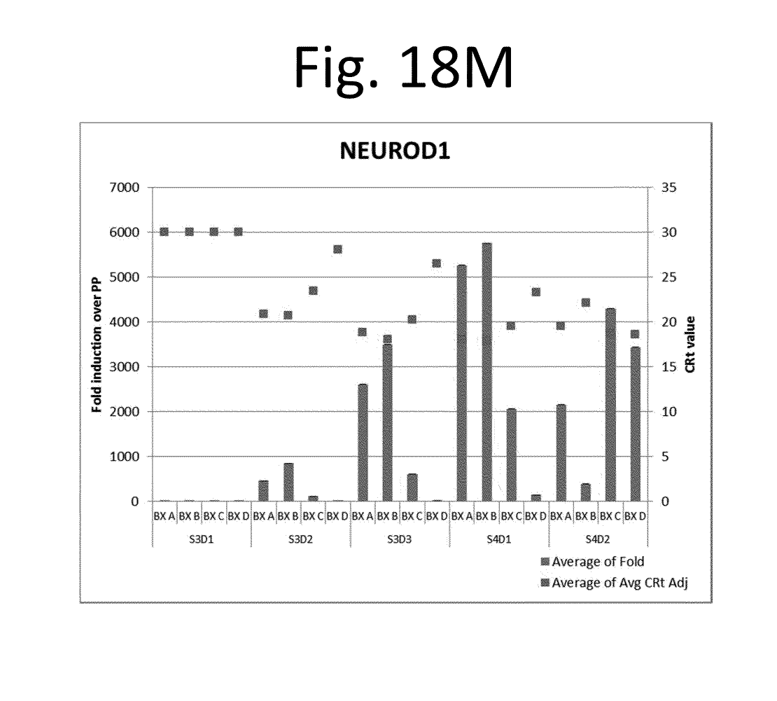

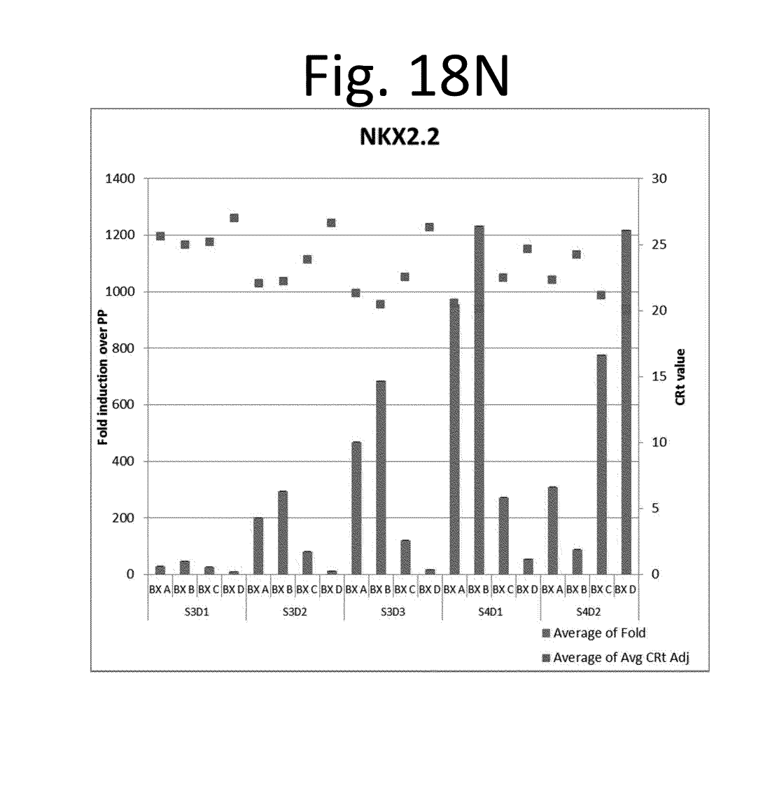

In addition to determining cell protein expression by flow cytometry, samples throughout Stages 3 and 4 of the differentiation process were tested for mRNA expression of a gene panel using OpenArray® qRT-PCR. FIGS. 18A through N depict data from real-time PCR analyses of the following genes in cells of the human embryonic stem cell line H1 differentiated through the second day of Stage 4: PDX1 (FIG.18A); NKX6.1 (FIG. 18B); PAX4 (FIG. 18C); PAX6 (FIG. 18D); NeuroG3(NGN3) (FIG. 18E); ABCC8 (FIG. 18F); chromogranin-A (FIG. 18G); chromogranin-B (FIG. 18H); ARX (FIG. 18I); Ghrelin (FIG. 18J); IAPP (FIG. 18K); PTF1a (FIG. 18L); NEUROD1 (FIG. 18M); and NKX2.2 (FIG. 18N).

As shown in FIG. 18A, under both low (7.0) or standard (7.4) pH differentiation conditions, cells expressed similar levels of PDX1 throughout Stage 3 as the cells adopted a pancreatic fate. As the cells from pH 7.4 reactors progressed through Stage 3 (reactors BX A and BX B), in the relative absence of NKX6.1 expression (FIG. 18B), they began to express multiple genes required for and characteristic of early endocrine pancreatic cell development: PAX4, PAX6, NGN3, NEUROD1, NKX2.2, ARX, Ghrelin, CHGA and CHGB as shown in FIGS. 18C, 18D, 18E, 18M, 18N, 18I, 18J, 18G, and 18H. This pattern of gene expression combined with low NKX6.1 expression, indicated some precocious (non-beta cell) endocrine pancreas specification.

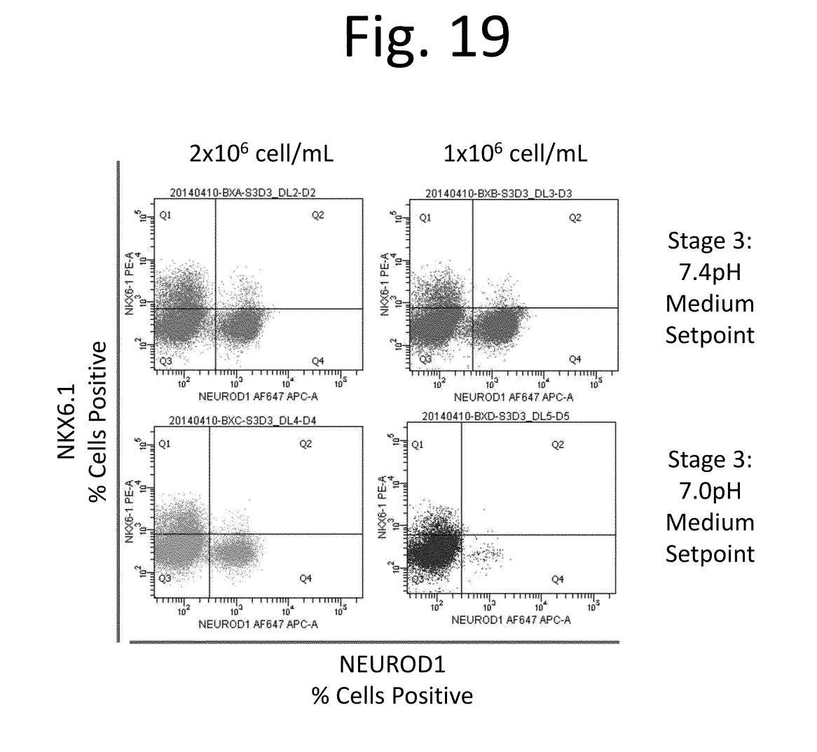

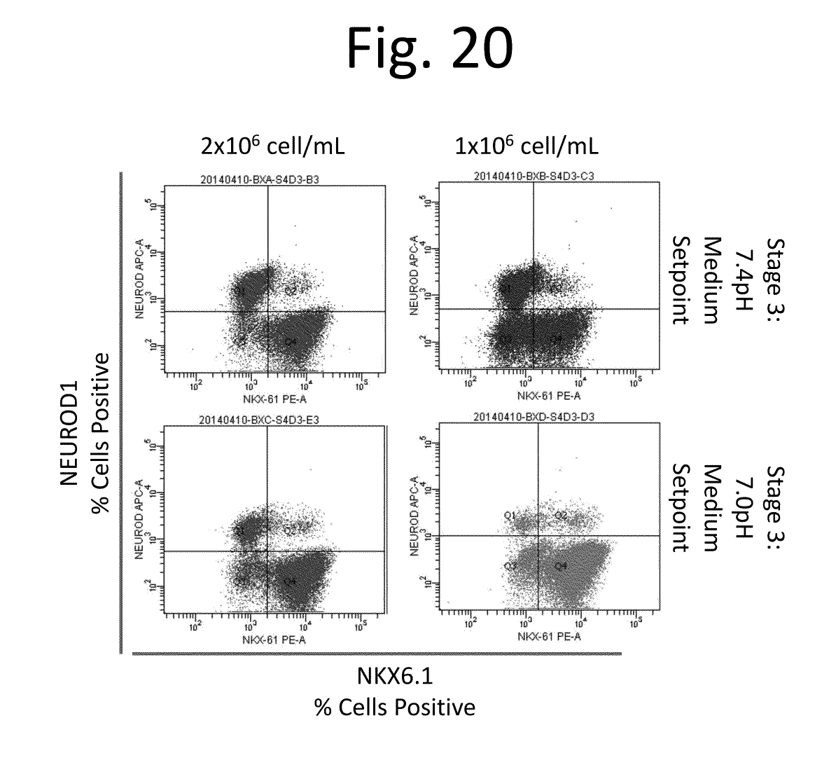

In contrast, cells from reactors C and D (stage 3 pH 7.0) when measured by OpenArray® qRT-PCR, expressed significantly lower levels of transcription factors required for endocrine development (PAX4, PAX6, NGN3, NEUROD1, NKX2.2, and ARX) in Stage 3 when compared to reactor A and B (FIGS. 18C, 18D, 18E, 18M, 18N, and 18I). Furthermore, it was observed that cells from reactors C and D had an increase in NKX6.1 (transcription factor required for beta cell formation) on the first day of Stage 4 that was followed by increased expression of PAX6, NEUROD1, and NKX2.2 on the second day of Stage 4 (FIGS. 18D, 18M, 18N, and 18B). These qRT-PCR data correlated with flow cytometry results that demonstrated, for cells maintained at 7.0 pH in Stage 3, a reduced percentage of cells expressing NEUROD1 and increased numbers of cells expressing NKX6.1 at the end of Stages 3 and 4 (Table VI, FIG. 19, and FIG. 20). These data suggest that low pH (7.0) at Stage 3 inhibits precocious (non-beta cell) endocrine pancreas specification and promotes a transcription factor expression sequence required to form beta cells.

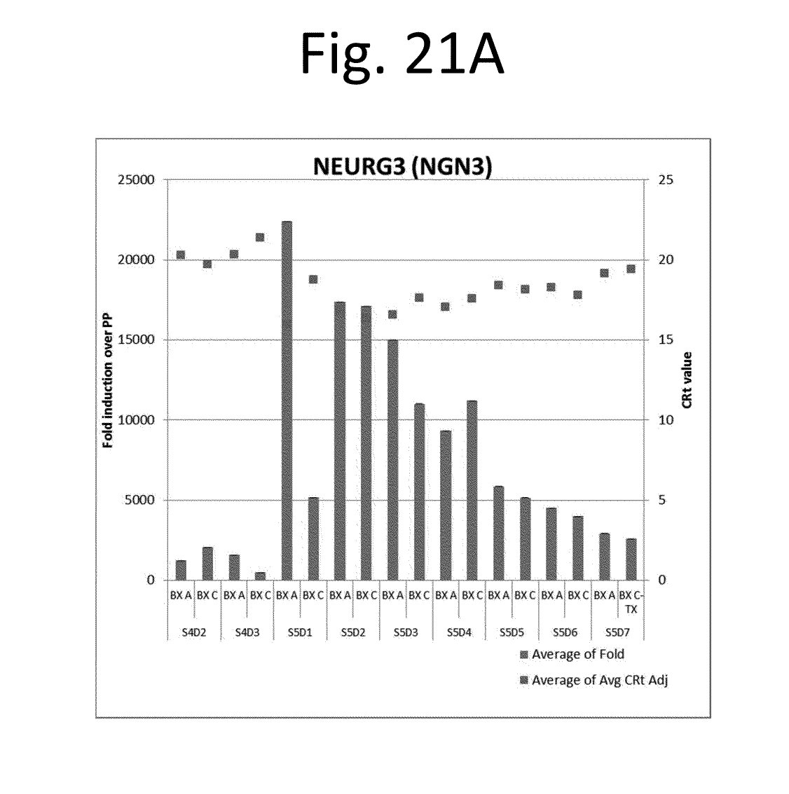

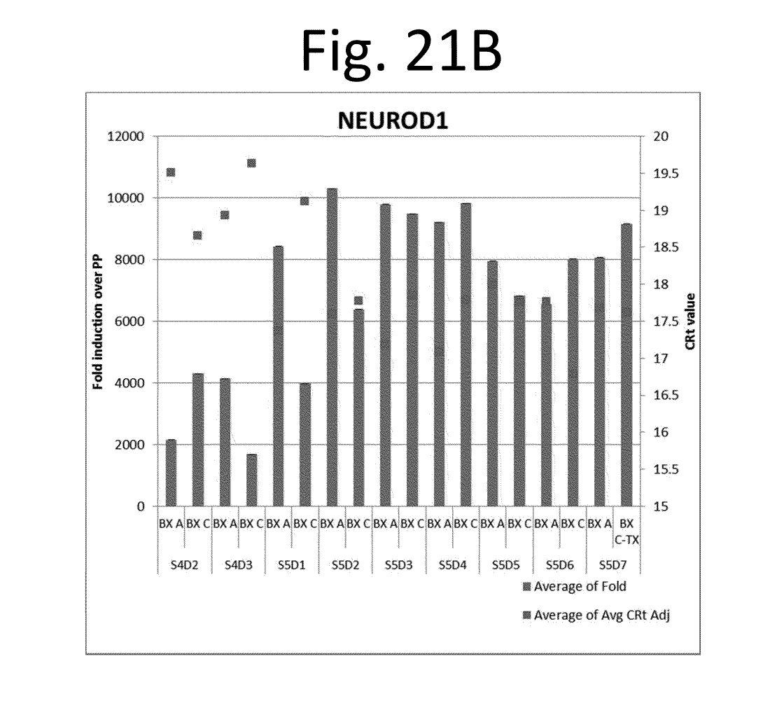

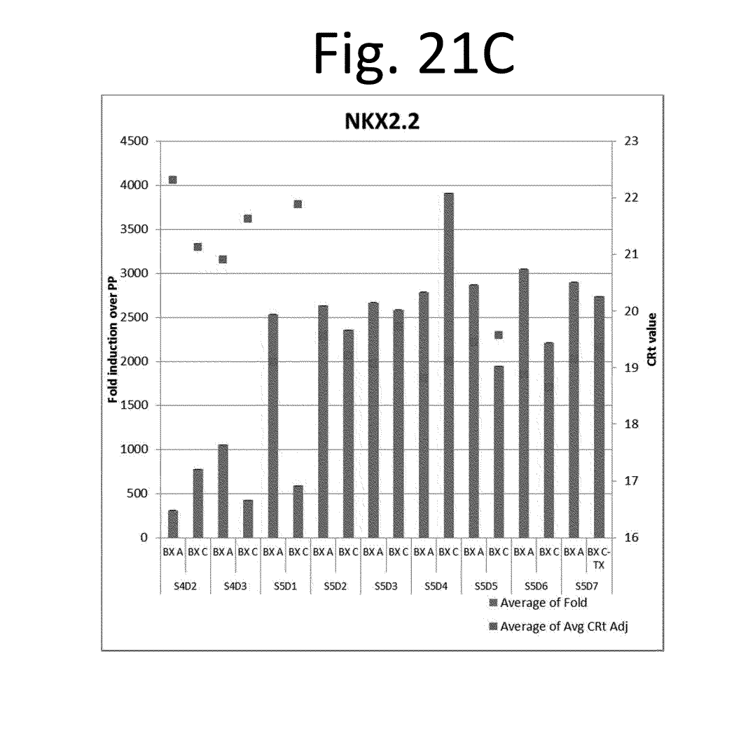

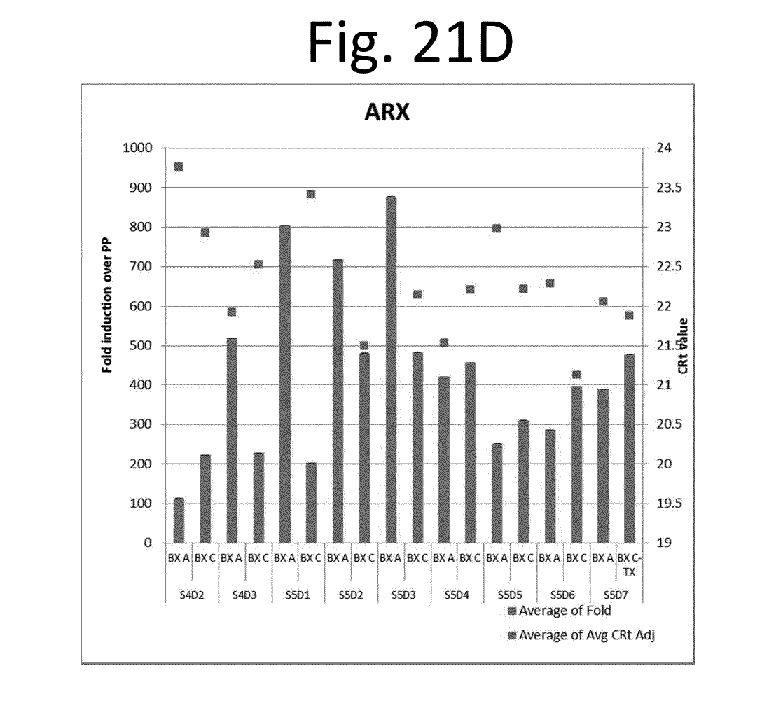

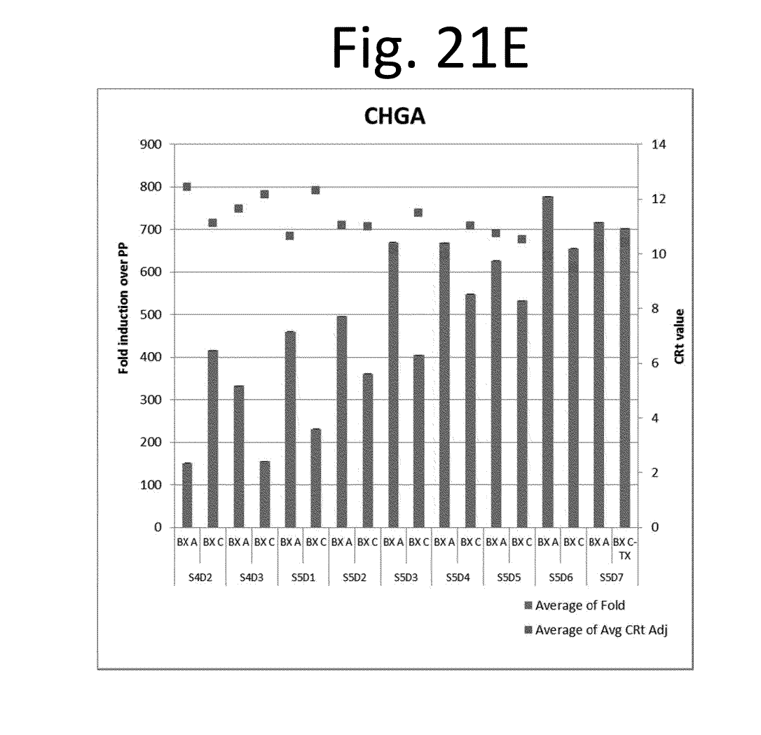

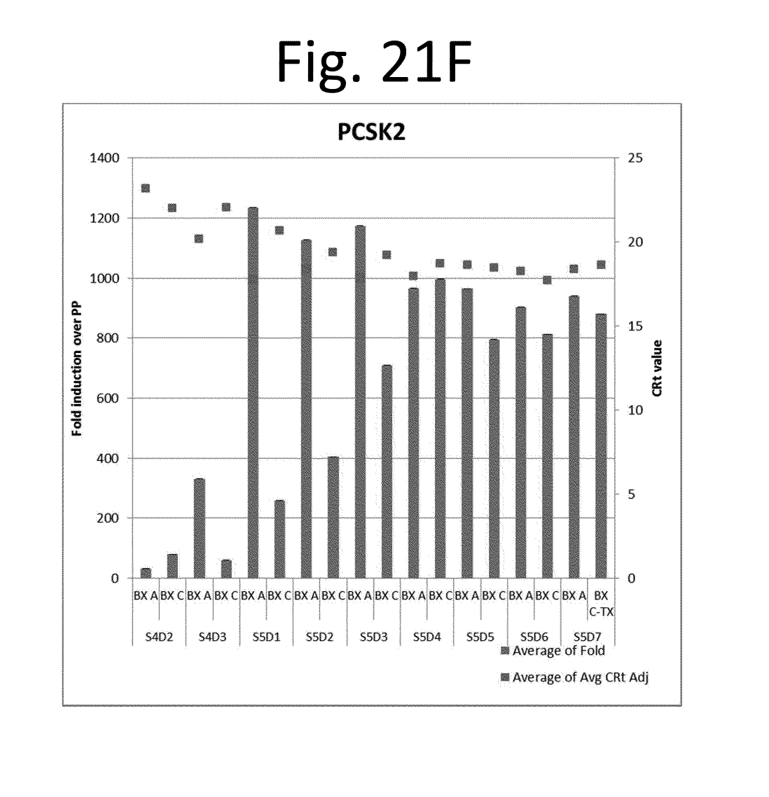

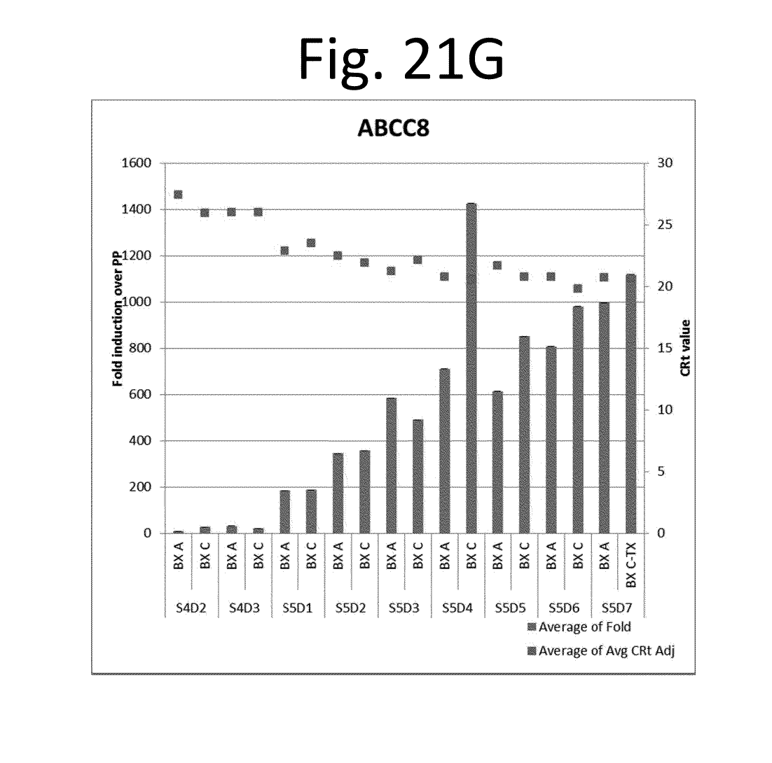

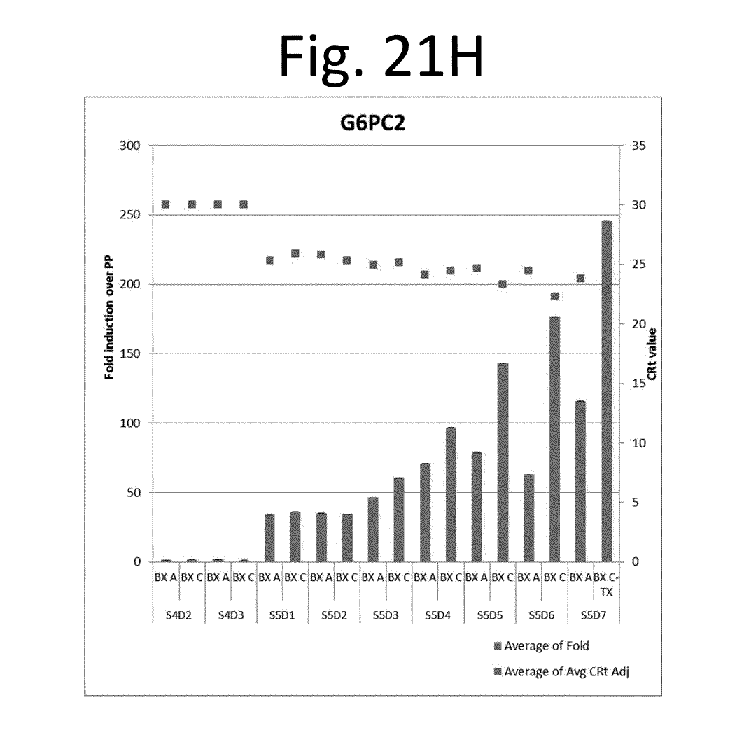

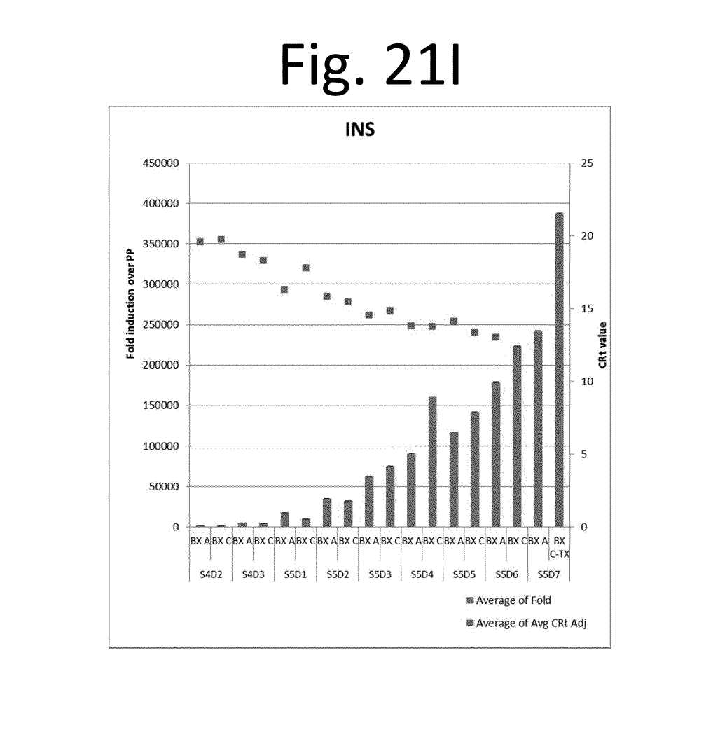

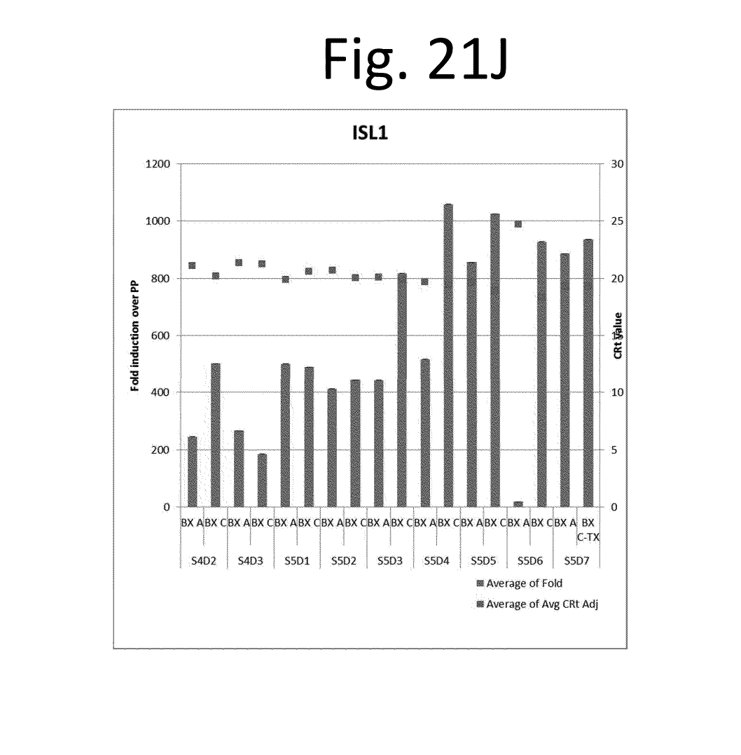

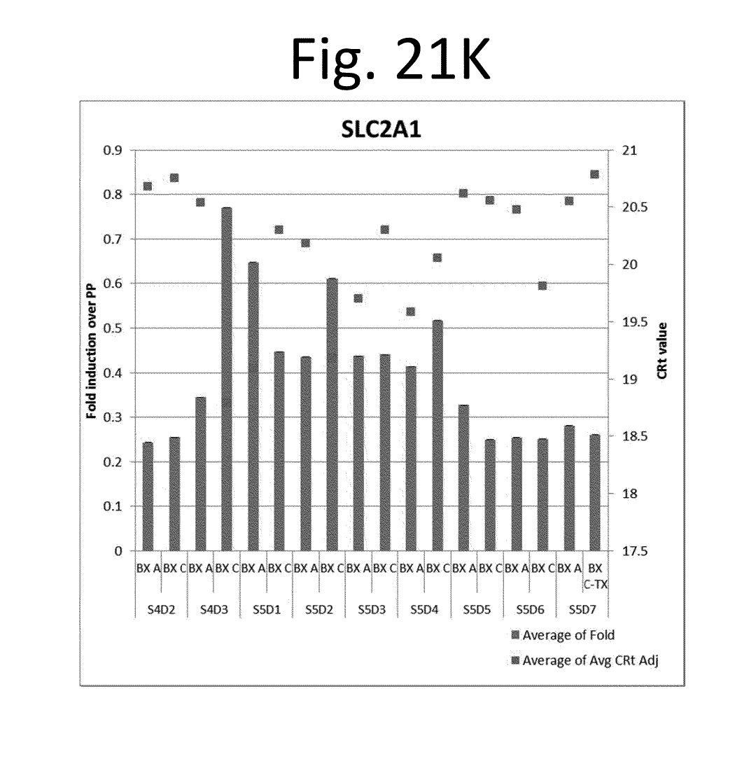

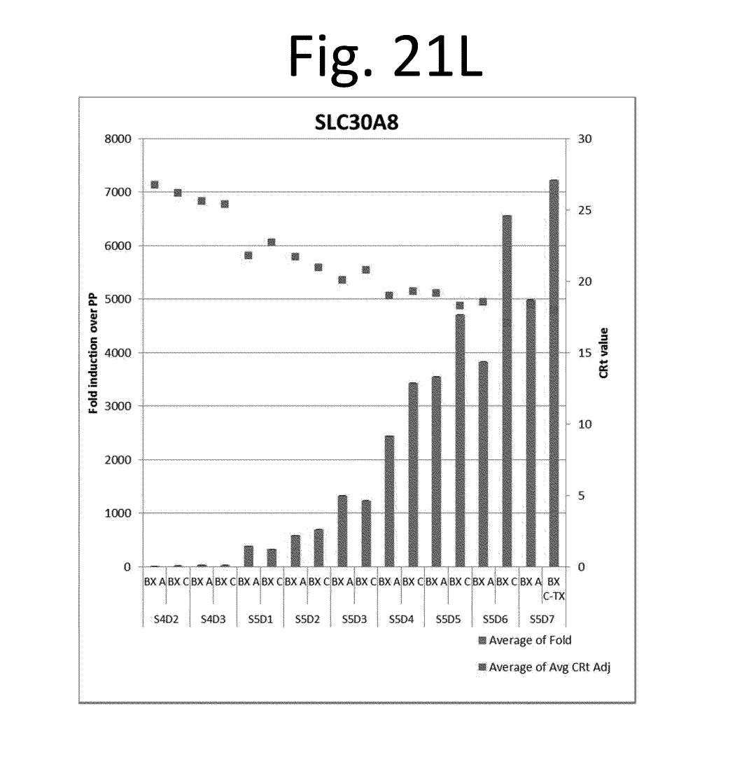

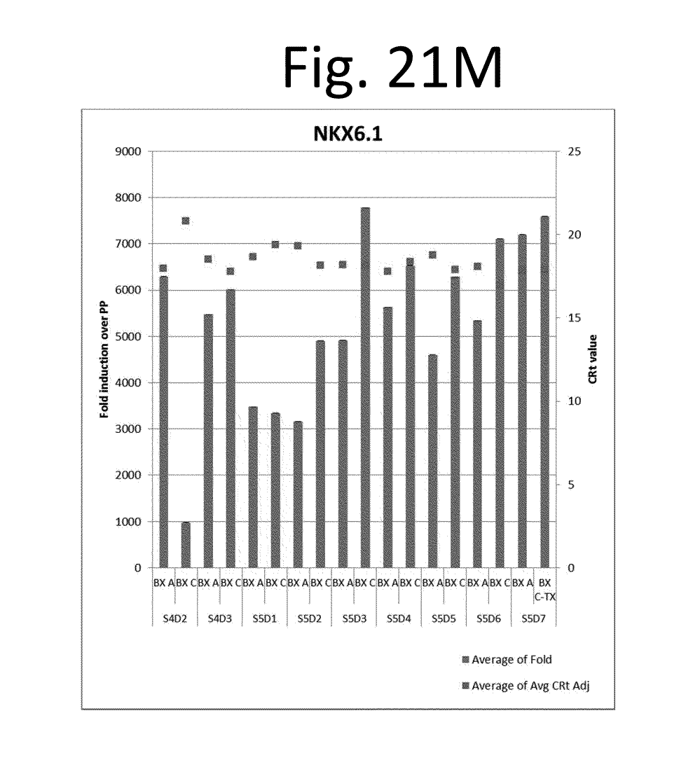

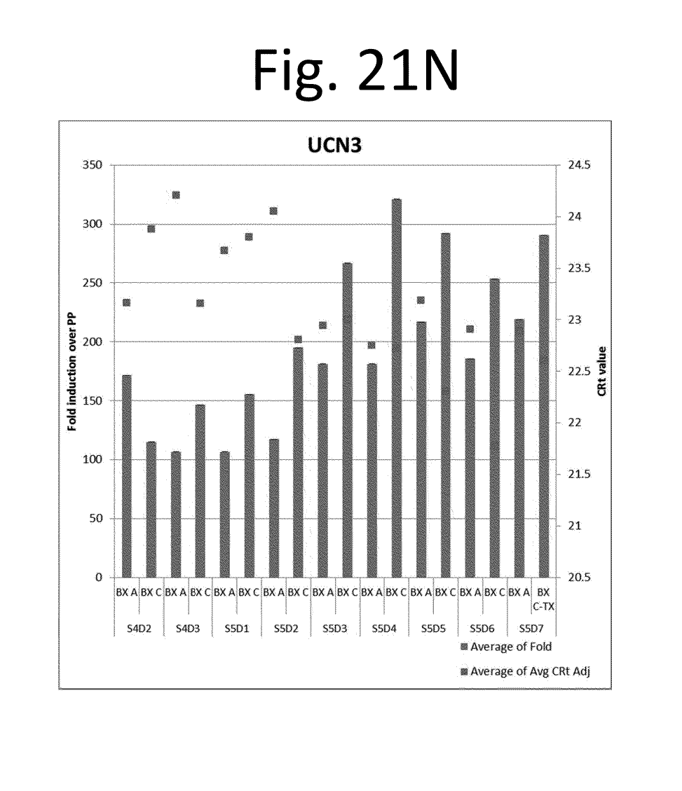

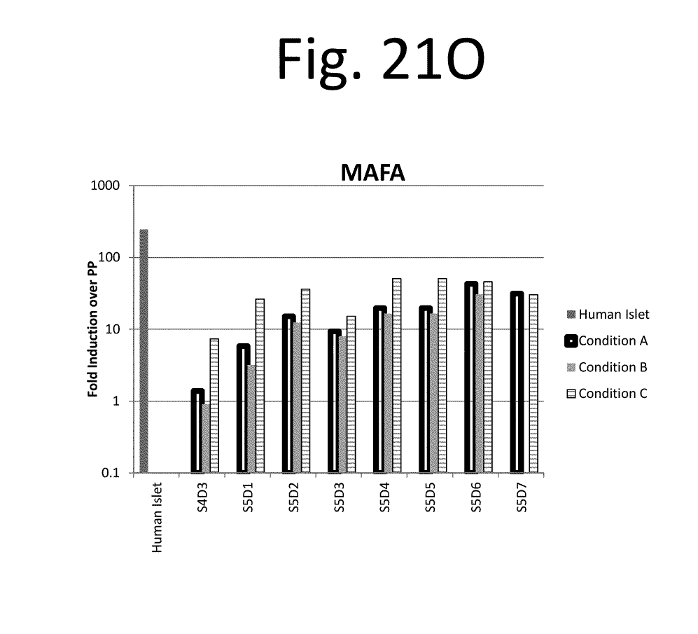

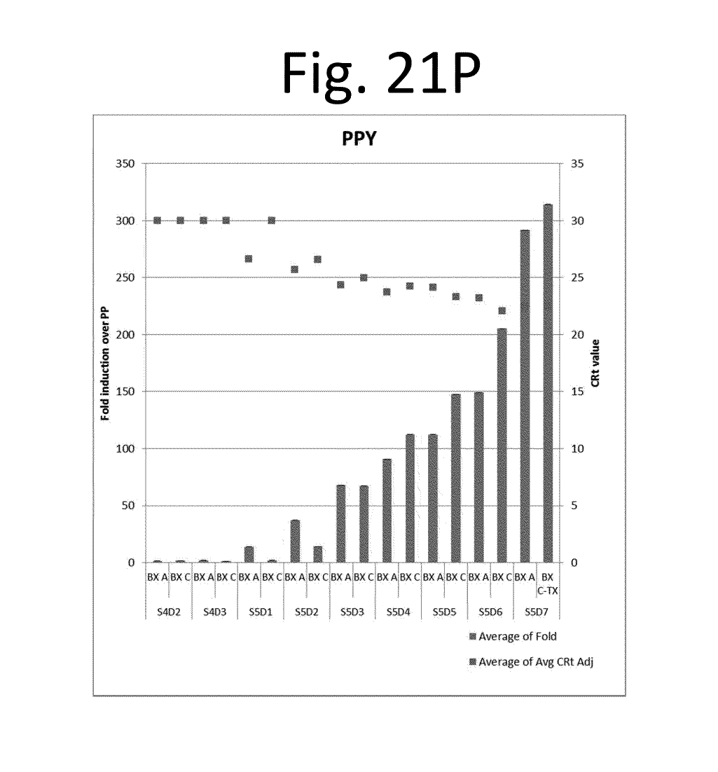

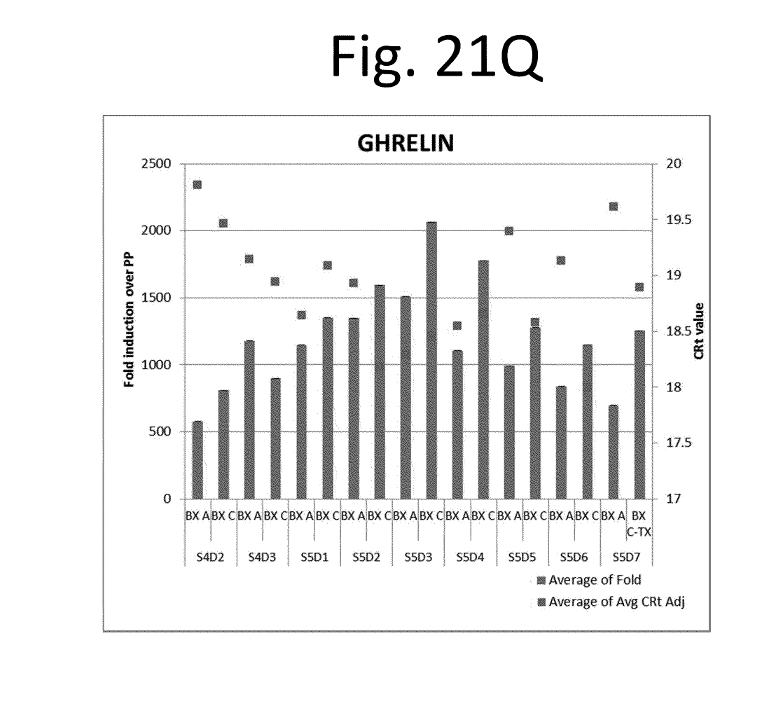

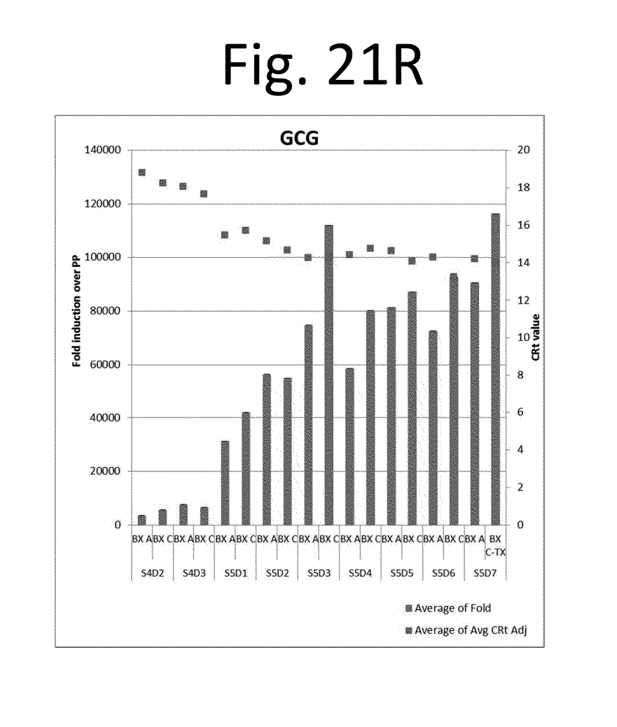

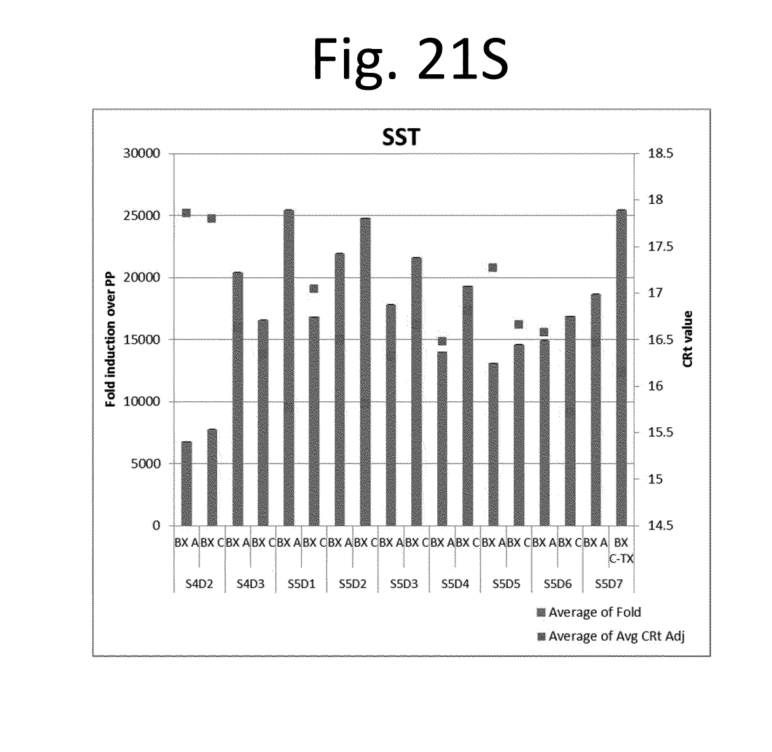

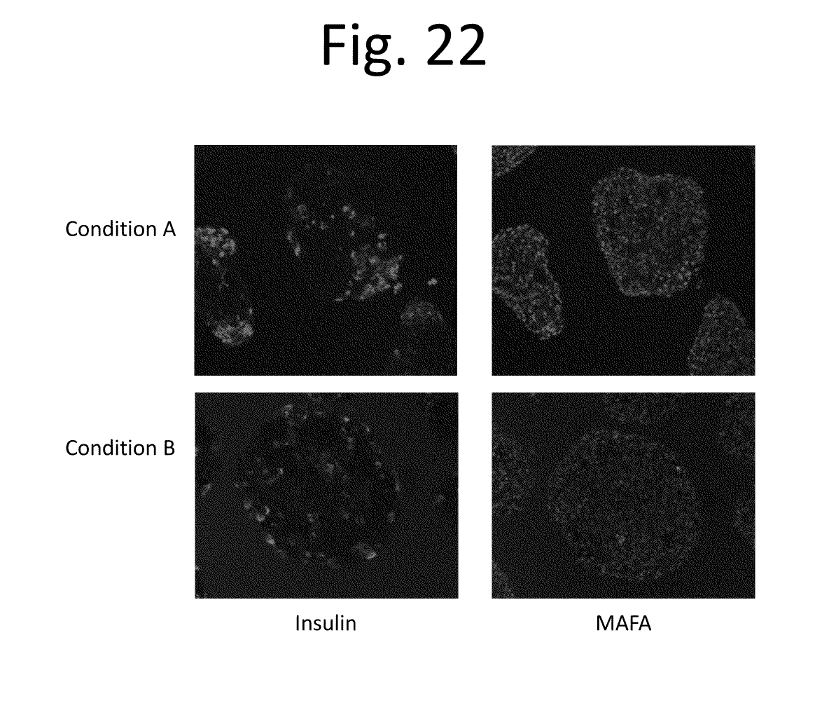

The effect of delayed or reduced expression of genes involved in non-beta cell endocrine pancreas specification, through reduced medium pH at Stage 3, persisted through Stage 5 of differentiation. NGN3 gene expression is required in the developing pancreas for proper endocrine hormone cell development and, in both conditions A (pH 7.4) and C (pH 7.0 at Stage 3), expression of NGN3 was induced in response to treatment of cells with Stage 5 medium containing gamma secretase inhibitor. However, for cells differentiated according to condition C cells, a delay of one day in peak NGN3 expression (FIG. 21A) was noted. Furthermore, multiple genes induced or regulated by NGN3 expression were also delayed in cell differentiated by condition C (pH 7.0 at stage 3). Endocrine specific genes such as NEUROD1 (FIG. 21B), NKX2.2 (FIG. 21C), ARX (FIG. 21D), Chromogranin A/CHGA (FIG. 21E), and PCSK2 (FIG. 21F) all showed a lag in expression similar to NGN3. However, genes associated specifically with beta cells—ABCC8 (FIG. 21G), G6CP2/glucose 6 phosphatase (FIG. 21H), Insulin/INS (FIG. 21I), Isletl/ISL1 (FIG. 21J), Glucose Transporter 1/SLC2A1 (FIG. 21K), Zinc Transporter/SLC30A8 (FIG. 21L), and NKX6.1 (FIG. 21M) appear at the same time and magnitude in cells from conditions A and C. Furthermore, expression of UCN3—a gene associated with proper maturation of functional beta cells—was increased throughout Stage 5 in cells differentiated in reactor C (pH 7.0 at Stage 3) as compared to cells maintained at pH 7.4 (reactor A) as shown in FIG. 21N indicating that exposure to pH 7.0 in Stage 3 promotes later stage maturation to beta-lie cells in this process.

In addition to an increase in UCN3 expression, an increase in expression of the beta-cell specific transcription factor-MAFA by qRT-PCR was also observed. MAFA expression was first detectable in all three conditions tested (A, B, and C) by single primer-probe qRT-PCR assay on Stage 5 day 1 (FIG. 210) following the addition of gamma secretase inhibitor. From Stage 4 day 3 through Stage 5 day 5, the detectable mRNA expression of MAFA was higher in condition C than in conditions A or B. Protein expression of MAFA was confirmed at the end of stage 6 by immuno-florescent cytochemistry. As shown in FIG. 22, micrographs obtained by 20× objective depict immune-florescent staining for nuclear MAFA and cytoplasmic insulin staining.

These gene expression patterns suggests that suppression of early endocrine specification through exposure to low pH at Stage 3, prior to expression of beta cell specific transcription factors, can promote later differentiation to a beta cell like fate by reducing early non-beta cell fate adoption. Flow cytometry results supported this hypothesis, as cells differentiated in reactor C had an increased percentage of insulin positive cells (27.3%, Table VI) when compared to condition A cells (20.3%, Table VI) along with an increase in NKX6.1/insulin co-positive cells (21.3%, condition C versus 15.6%, condition A).

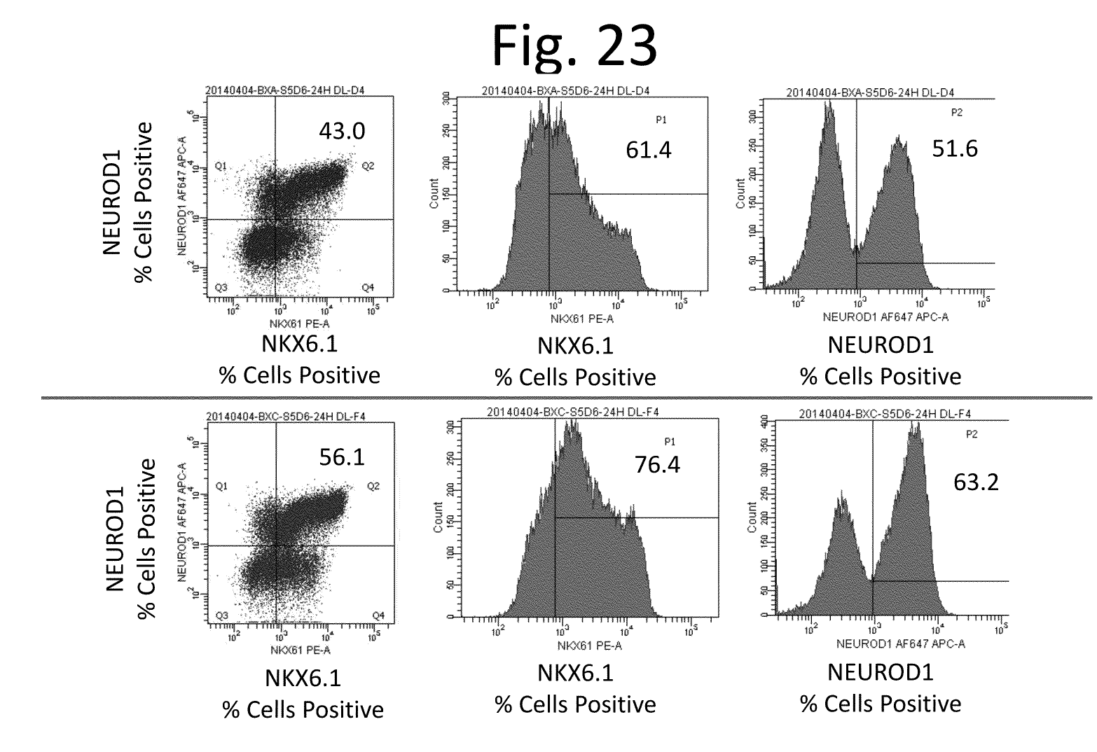

Interestingly, low pH in Stage 3 and later differentiation to a beta-cell like fate did not suppress gene expression characteristic of other pancreatic endocrine fates. Gene expression by qRT-PCR was observed for the endocrine hormones pancreatic polypeptide (“PPY”), ghrelin, glucagon (“GCG”), and somatostatin (“SST”) in samples assayed at the end of Stage 5 (FIGS. 21P-PPY, 21Q-Ghrelin, 21R-GCG, and 21S-SST). This observation was further supported by flow cytometry data showing differentiated cells were positive for a pan-endocrine transcription factor, NEUROD1 (63.1% NEUROD1 positive, and 56.1% of cells NEUROD1/NKX6.1 co-positive for condition C; 51.6% NEUROD1 positive, and 43% NEUROD1/NKX6.1 co-positive for condition A); as shown in Table VII and FIG. 23.

At the end of the seventh day of Stage 5, 5×10⁶ cells differentiated with a set-point of pH 7.0 in Stage 3 (condition C) were isolated from the media in a 50 mL conical, then washed 2 times with MCDB-1313 medium containing a total of 2.4 g/L sodium bicarbonate and 0.2% w/v FAF-BSA. The cells were re-suspended in the wash media and held at room temperature for approximately 5 hours prior to transplantation under the kidney capsule of NSG mice. At the earliest measured time point, 4 weeks post-implant, a mean human C-peptide blood level of 0.3 ng/mL was observed following an overnight fast, intra-peritoneal glucose injection, and retro-orbital blood draw 60 minutes after the IP glucose bolus (N=7 animals).

TABLE VI
Flow Cytometry Results (% of cells positive for marker)

| | | NKX6.1 | NEUROD1 | PDX1 | FOXA2 | CDX2 | Ki67 |
|---|---|---|---|---|---|---|---|
| S3D3 | BX 1 | 8.3 | 30.9 | 99.9 | 99.7 | 0.3 | 43.8 |
| S3D3 | BX A | 13.6 | 36.5 | 99.8 | 99.4 | 5.2 | 41.8 |
| S3D3 | BX B | 6.1 | 37.3 | 99.6 | 99.8 | 1.5 | 46.7 |
| S3D3 | BX C | 11.6 | 15.8 | 99.5 | 99.1 | 8.3 | 51.2 |
| S3D3 | BX D | 5.8 | 0.6 | 99.9 | 99.8 | 5.9 | 78.9 |
| S4D3 | BX 1 | 45 | 44.7 | 98.2 | 98.6 | 5.7 | 39.9 |
| S4D3 | BX A | 60.5 | 35.1 | 99.3 | 99.3 | 4.3 | 45.6 |
| S4D3 | BX B | 39.7 | 37.5 | 98.8 | 99.3 | 4.2 | 47.5 |
| S4D3 | BX C | 80 | 13.6 | 99.7 | 99.8 | 2.7 | 58.9 |
| S4D3 | BX D | 89.8 | 5.3 | 98.3 | 98 | 5.1 | 68 |

TABLE VII
End of Stage 5 Day 6 (S5D6) Flow Cytometry Results
(% of cells positive for marker)

| | | | NEUROD1 | Insulin | |
|---|---|---|---|---|---|
| | | | (NEUROD1+/ | (NKX6.1+/ | |
| Condition | NKX6.1 | PDX1 | NKX6.1+) | Insulin+) | Ki67 |
| BX A | 61.4 | 94.5 | 51.6 | 20.3 | 21.7 |
| (pH 7.4) | | | (43) | (15.6) | |
| BX C | 76.4 | 94.7 | 63.2 | 27.3 | 20.9 |
| (pH 7.0) | | | (56.1) | (21.3) | |

### Example 3

This example demonstrates formation of insulin expressing cells from a population of cells expressing PDX1, in a stirred-tank, aseptically closed bioreactor. The insulin positive cells were generated from cells exposed to one of three conditions during Stage 3. The three conditions: reactor B-pH 7.0 throughout Stage 3 (treatment with retinoic acid); reactor C-pH 7.4 on the first day of Stage 3, then pH 7.0 for days 2 and 3 of Stage 3; or reactor D-pH 7.4 throughout Stage 3. It was observed that longer exposure to pH 7.0 in stage 3 reduced Ki67 and increased expression of NEUROD1, NEUROD1 co-positive with NKX6.1, PAX6, Islet 1, and PDX1/NKX6.1—protein later in the differentiation process.

Cells of the human embryonic stem cell line H1 (WA01 cells, WiCell Research Institute, Madison, Wis.) were grown in Essential 8TM medium supplemented with 0.5% w/v of a fatty acid free bovine serum albumin in dynamic suspension for ≧4 passages as round aggregated clusters. The clusters were then frozen as single cells and clusters of 2 to 10 cells per the following method. Approximately 600-1000 million cells in aggregated clusters were transferred to a centrifuge tube and washed using 100 mL of 1× DPS −/−. After the wash, the cell aggregates were then enzymatically disaggregated by adding a 30 mL solution of 50% StemPro®Accutase® enzyme and 50 DPBS −/− by volume to the loosened cell aggregate pellet. The cell clusters were pipetted up and down 1 to 3 times and then intermittently swirled for approximately 4 minutes at room temperature, then centrifuged for 5 min, at 80 to 200 rcf. The Accutase® supernatant was then aspirated as completely as possible without disturbing the cell pellet. The centrifuge tube was then tapped against a hard surface for approximately 4 minutes, to disaggregate the clusters into single cells and clusters comprised of 2 to10 cells. After 4 minutes, the cells were re-suspended in 100 mL of E8™ media supplemented with 10 Y-27632 and 0.5% w/v FAF-BSA, and centrifuged for 5 to12 minutes at 80 to 200 rcf. The supernatant was then aspirated and cold (≦4° C.) Cryostor® Cell Preservation Media CS10 was added drop-wise to achieve a final concentration of 100 to 150 million cells per mL. This cell solution was held in an ice bath while being aliquoted to 2 mL cryogenic vials (Corning) after which the cells were frozen using a controlled rate CryoMed™ 34 L freezer as follows. The chamber was cooled to 4° C. and the temperature was held until a sample vial temperature reached 6° C. and then the chamber temperature was lowered 2° C. per minute until the sample reached −7° C. at which point the chamber was cooled 20° C./min. until the chamber reached −45° C. The chamber temperature was then allowed to briefly rise at 10° C./min. until the temperature reached −25° C., and then the chamber was cooled further at 0.8° C./min. until the sample vial reached −40° C. The chamber temperature was then cooled at 10° C./min. until the chamber reached −100° C. at which point the chamber was then cooled 35° C./min. until the chamber reached −160° C. The chamber temperature was then held at −160° C. for at least 10 minutes, after which the vials were transferred to gas phase liquid nitrogen storage. These cryo-preserved single cells at high concentration were then used as ISM.

ISM vials were removed from the liquid nitrogen storage, thawed, and used to inoculate a 3 liter glass, stirred suspension tank bioreactor (DASGIP) at a seeding concentration of 0.295 million viable cells per mL. The vials were removed from liquid nitrogen storage and quickly transferred to a 37° C. water bath for 120 seconds to thaw. The vials were then moved to a BSC and the thawed contents transferred via 2 mL glass pipette to a 50 mL conical tube. Then 10 mL of E8™ medium supplemented with 0.5% w/v FAF-BSA and 10 μM of Rho kinase inhibitor Y-27632, were added to the tube in a drop-wise manner. The cells were centrifuged at 80-200 rcf for 5 min. The supernatant from the tube was aspirated and 10 mL fresh E8™ medium supplemented with 0.5% w/v FAF-BSA and 10 μM Y-27632 were added and the volume containing the cells was pipetted into a media transfer bottle (Cap2V8®) containing 450 mL E8™ media supplemented with 0.5% w/v FAF-BSA and 10 μM Y-27632. The bottle contents were then pumped directly into the bioreactor via a sterile, C-Flex® tubing weld using a peristaltic pump. The bioreactor was prepared with 1000mL E8™ medium supplemented with 0.5% w/v FAF-BSA and 10 μM Y-27632 pre-warmed to 37° C., stirred at 70 rpm, with a dissolved oxygen set point of 30% (air O₂, and N₂ regulated), and a controlled CO₂ partial pressure of 5% . The reactor was inoculated to give a target concentration of 0.225×10⁶ cells/mL (concentration range: 0.2 to 0.5×10⁶ cells/mL).

Once the reactor was inoculated, the cells formed round aggregated clusters in the stirred reactor. After 24 hours in culture, the medium was partially exchanged as more than 80% of the original volume was removed and 1.5 L of E8™ media supplemented with 0.5% w/v FAF-BSA was added back (fresh medium). This media exchange process was repeated 48 hours after inoculation. After three days in suspension culture as round aggregated clusters differentiation in the 3 liter reactor was initiated by removing spent E8™ medium and adding differentiation medium. The differentiation protocol is described below.

Stage 1 (3 Days):

The reactor was set to a temperature of 37° C. and stirred continuously at 70 rpm. Gas and pH controls were set to a dissolved oxygen set point of 10% (air, oxygen, and nitrogen regulated) and the pH was set to 7.4 via CO₂ regulation. A base medium was prepared using 1.5 L MCDB-131 medium containing 1.18 g/L sodium bicarbonate; supplemented with an additional 2.4 g/L sodium bicarbonate, 2% w/v FAF-BSA, previously re-constituted in MCDB-131; 1× concentration of GlutaMAX™; 2.5 mM glucose (45% in water); and a 1:50,000 dilution of ITS-X. Cells were cultured for one day in 1.5 L of the base medium supplemented with 100 ng/ml GDF8; and 3 μM of MCX compound. After 24 hours, a media exchange was completed as described above, and fresh 1.5 L of base medium supplemented with 100 ng/mL of GDF8 were added to the reactor. Cells were maintained without further media exchange for 48 hours.

Stage 2 (3 Days):

The reactor was set to a temperature of 37° C. and stirred continuously at 70 rpm. Gas and pH controls were set to a dissolved oxygen set point of 30% (air, oxygen, and nitrogen regulated) and the pH was set to 7.4 via CO₂ regulation. A base medium was prepared using 1.5 L MCDB-131 medium containing 1.18 g/L sodium bicarbonate and supplemented with an additional 2.4 g/L sodium bicarbonate; 2% w/v FAF-BSA, previously re-constituted in MCDB-131; 1× concentration of GlutaMAX™; 2.5 mM glucose; and a 1:50,000 dilution of ITS-X. After the completion of Stage 1, a media exchange was completed as described above, whereby the spent Stage 1 media was removed and replaced with 1.5 L of Stage 2 base medium supplemented with 50 ng/mL FGF7. Forty-eight hours after the media exchange, the spent media was again removed and replaced with 1.5 L fresh Stage 2 base medium supplemented with 50 ng/mL FGF7.

Stage 3 (3 Days):

At the completion of Stage 2, and just prior to medium exchange, all cells were removed from the 3 liter reactor via sterile weld and peristaltic pump. The cells were counted, gravity settled and re-suspended in the following Stage 3 media at a normalized distribution of 2.0 million cells/mL: 1.5 L MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of GlutaMAX™; 2.5 mM glucose; and a 1:200 dilution of ITS-X. The Stage 3 medium was supplemented with 50 ng/mL FGF-7; 100 nM of LDN-193189; 2 μM RA; 0.25 μM SANT-1; and 400 nM of TPB. The cells were seeded at a normalized distribution of the 2.0 million cells/mL cell concentration into three 0.2 liter glass, stirred suspension tank DASGIP™ bioreactors B, C and D (also referred to as B×B, B×C, and B×D) via sterile weld and peristaltic pump. The reactors were set to a temperature of 37° C. and stirred continuously at 55 rpm. Gas and pH controls were set to a dissolved oxygen set point of 30% (air, oxygen, and nitrogen regulated) and the pH for Stage 3 was set to three different media pH variables as listed in Table VIII. Twenty-four hours post media exchange, the spent media was again replaced in each of the reactors B through D with 150 mL fresh Stage 3 medium containing the above supplements with the exception of LDN-193189. Cells were thereafter cultured in the media for 48 hours until the end of Stage 3.

TABLE VIII

| | pH Set Point | pH Set Point | pH Set Point | Cell |
|---|---|---|---|---|
| | Stage 3, | Stage 3, | Stage 3, | Concen- |
| | Day 1 | Day 2 | Day 3 | tration |
| Reactor B | 7.0 | 7.0 | 7.0 | 2.0 × 10⁶ |
| (Bx B) | | | | cells/mL |
| Reactor C | 7.4 | 7.0 | 7.0 | 2.0 × 10⁶ |
| (Bx C) | | | | cells/mL |
| Reactor D | 7.4 | 7.4 | 7.4 | 2.0 × 10⁶ |
| (Bx D) | | | | cells/mL |

Stage 4 (3 Days):

At the completion of Stage 3, the spent media was removed and replaced in each bioreactor with 150 mL of the following Stage 4 medium: 150 mL MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of GlutaMAX™; 2.5 mM glucose; and a 1:200 dilution of ITS-X. The medium was supplemented with 0.25 μM SANT-1 and 400 nM of TPB. The reactor was maintained at 37° C. and stirred continuously at 55 rpm. Gas and pH controls were regulated to a dissolved oxygen set point of 30% (air, oxygen, and nitrogen regulated) and a pH set point of 7.4 via CO₂ regulation. Forty-eight hours after initiation of Stage 4, 3.2 mL/L of a 45% glucose solution (8 mM glucose bolus) was added to the each bioreactor and the cells were cultured in the media for an additional 24 hours.

Stage 5 (7 Days):

A Stage 5 base medium was prepared for each bioreactor using: 150 mL MCDB-131 medium base containing 1.18 g/L sodium bicarbonate supplemented with an additional 1.754 g/L sodium bicarbonate; 2% w/v FAF-BSA previously re-constituted in MCDB-131; 1× concentration of GlutaMAX™; 20 mM glucose; 1:200 dilution of ITS-X; 250 μL/L of 1M ascorbic acid; and 10 mg/L heparin (Sigma Aldrich; Catalog No. H3149-100KU). After the completion of Stage 4, spent media in each bioreactor was replaced with 150 mL of Stage 5 medium supplemented with 1 μM T3, 10 μM ALK5 inhibitor II, 1 μM of gamma secretase inhibitor XXI; 20 ng/mL of betacellulin; 0.25 μM SANT-1; and 100 nM RA. Forty-eight hours after initiation of Stage 5, the spent media was removed and replaced with the same fresh base medium and supplements. Forty-eight hours later, the media was again exchanged and replaced with the same fresh medium and supplements, except the gamma secretase XXI and SANT were excluded. Forty-eight hours later the medium was again exchanged and replaced with the same fresh medium and supplements and the cells were cultured for an additional 24 hours to the end of Stage 5. Throughout Stage 5, a 30% DO and 7.4 pH were maintained.

Throughout the differentiation process, in addition to real-time continuous monitoring for pH and DO, media samples were collected from the reactors on a daily basis. Samples were analyzed for cell number, mRNA expression, and protein expression.

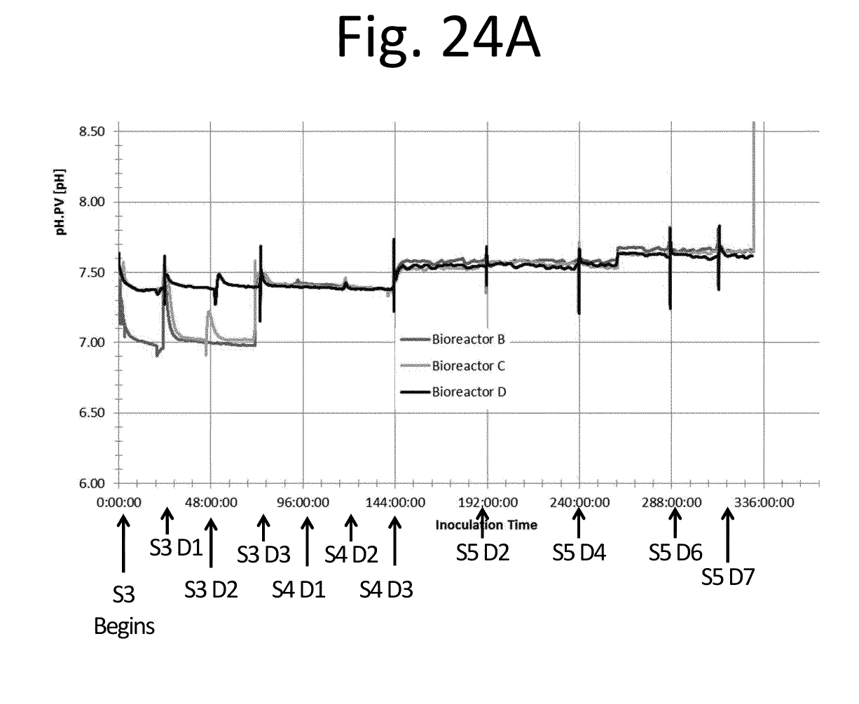

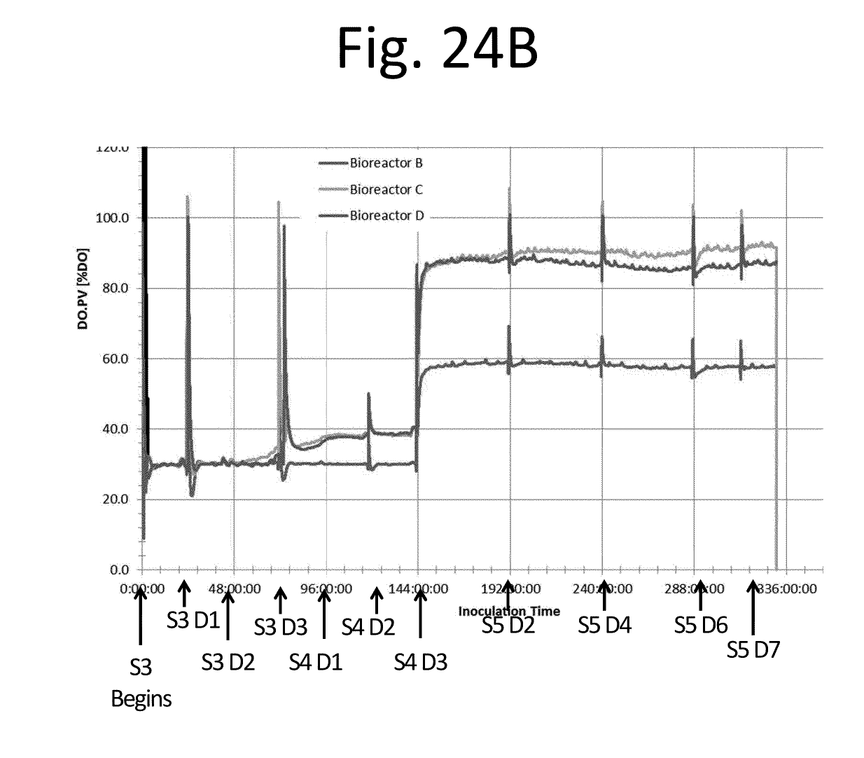

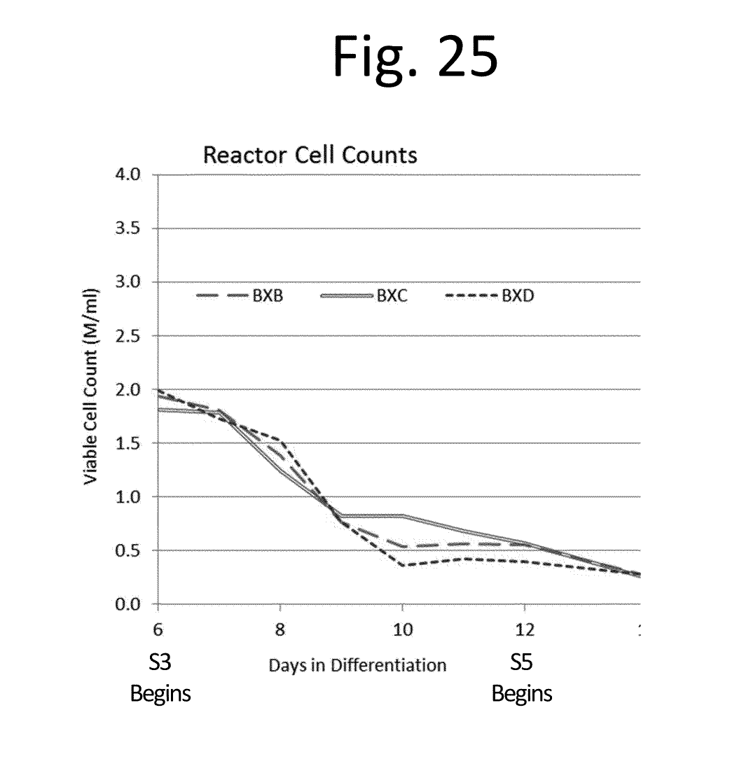

FIGS. 24A and B depict continuous monitoring graphs of pH (FIG. 24A) and dissolved oxygen levels (FIG. 24B) in media for reactors B, C, and D over the course of Stages 3, 4 and 5. These data demonstrate that cells in reactor B, set to pH 7.0 throughout Stage 3, showed increased oxygen consumption in Stages 4 and 5 as measured by lower levels of dissolved oxygen (FIG. 24B) compared to reactors C and D. Furthermore, as cell concentrations in reactors B, C, and D were comparable through Stage 5 (FIG. 25 and Table VIII) the differences in oxygen consumption were not due to significant differences in cell density. This suggests the cells in reactor B treated with pH 7.0 during Stage 3 had begun to adopt a more mature and oxygen consumptive phenotype than cells from reactors C or D (exposed to one or three days of pH 7.4 during stage three, respectively) by the end of Stage 4.

At the completion of Stage 3 and again three days later at the end of Stage 4, samples from each of the reactors were analyzed by flow cytometry for protein expression. Data demonstrating expression of NKX6.1, NEUROD1, PDX1, and CDX2 are shown in Table IX. It was observed by intracellular flow cytometry that cells maintained at pH 7.0 throughout Stage 3 or for the last two days of Stage 3 (reactors B and C, respectively) had proportionally more NKX6.1 positive cells and fewer NEUROD1 positive cells at the end of Stage 4 when compared to cells maintained in reactor D (set to a pH of 7.4 through Stage 3). These data indicate that even partial exposure to pH 7.0 at Stage 3 is sufficient to suppress NEUROD1 expression.

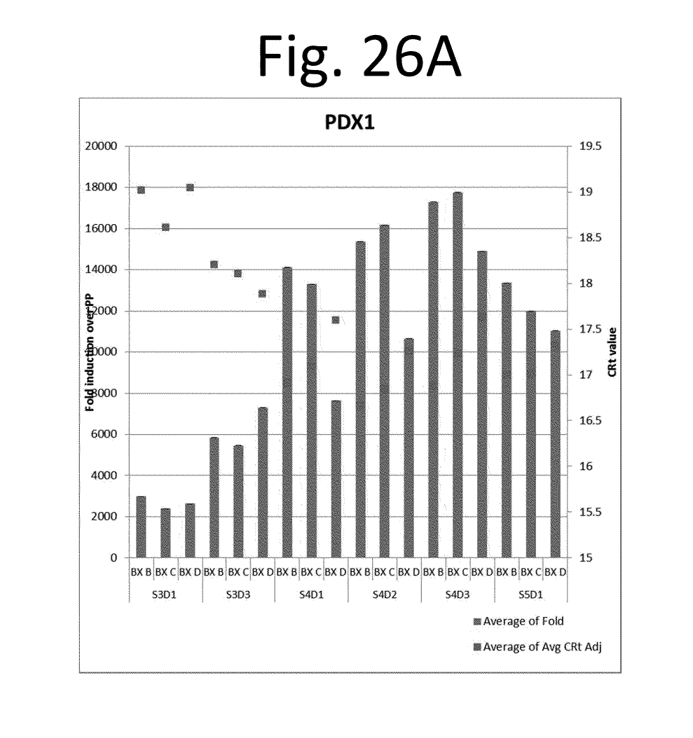

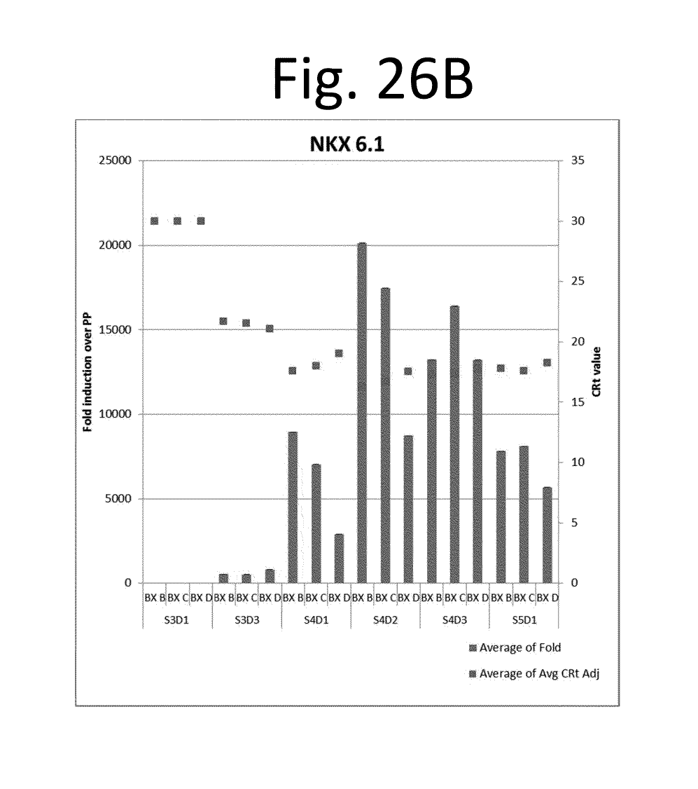

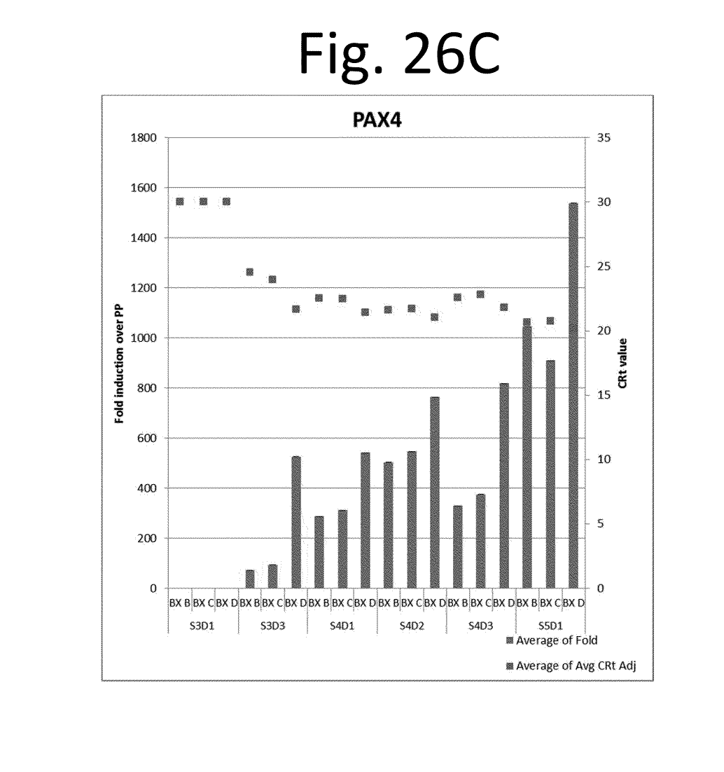

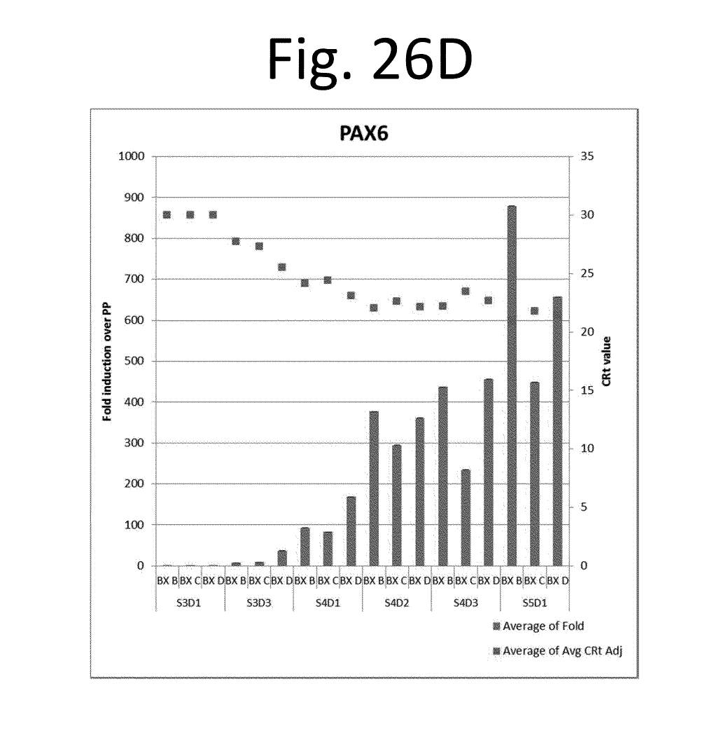

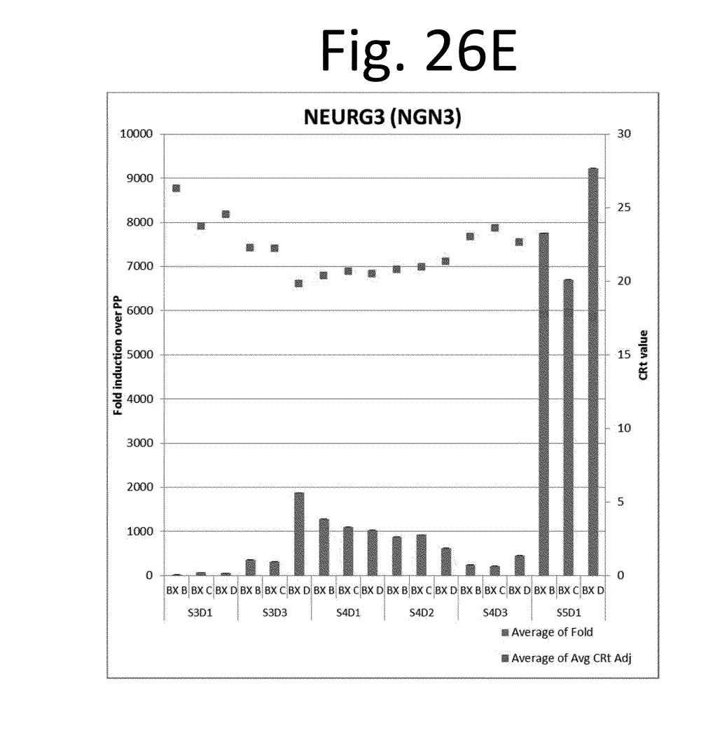

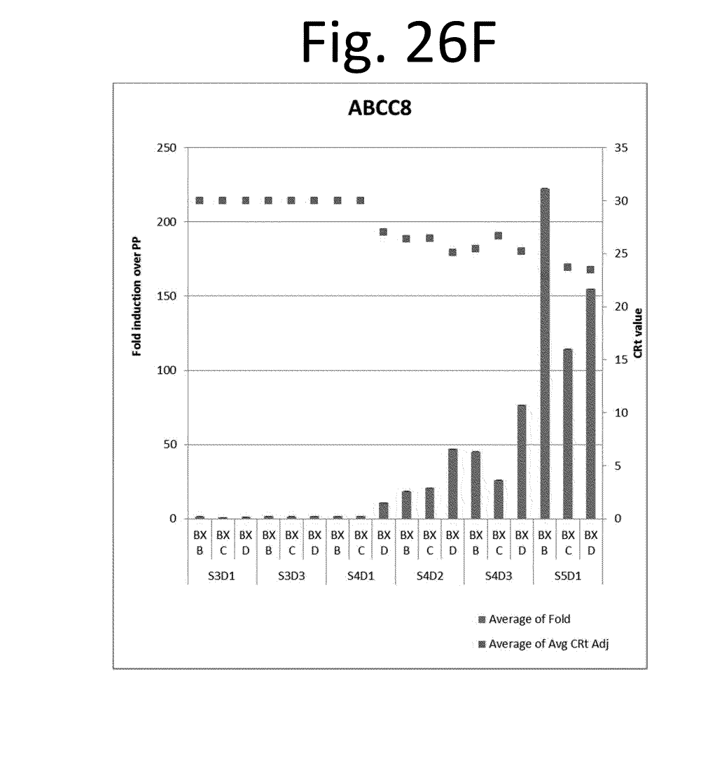

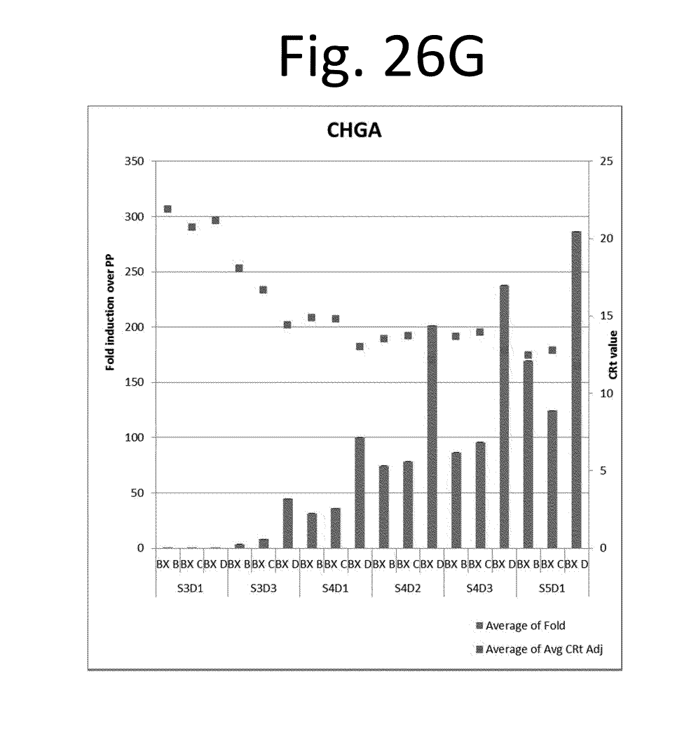

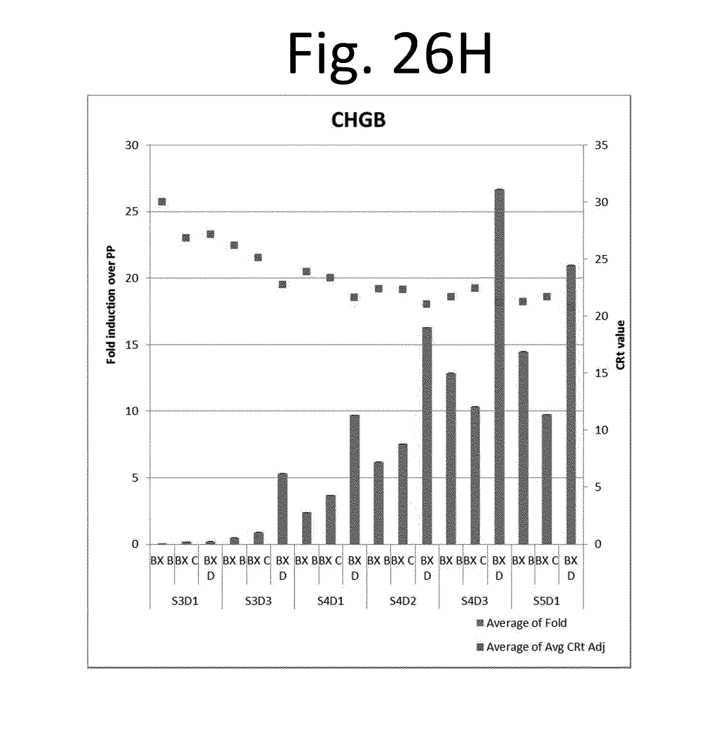

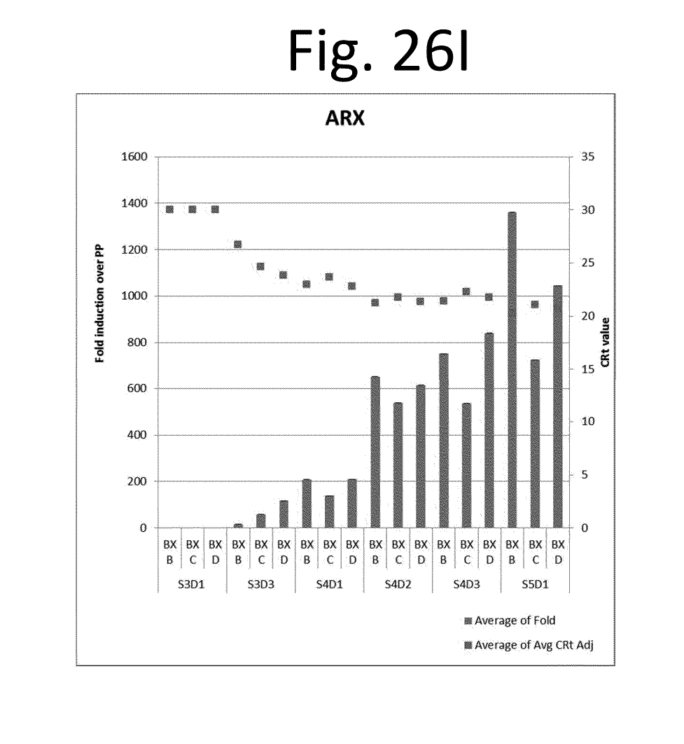

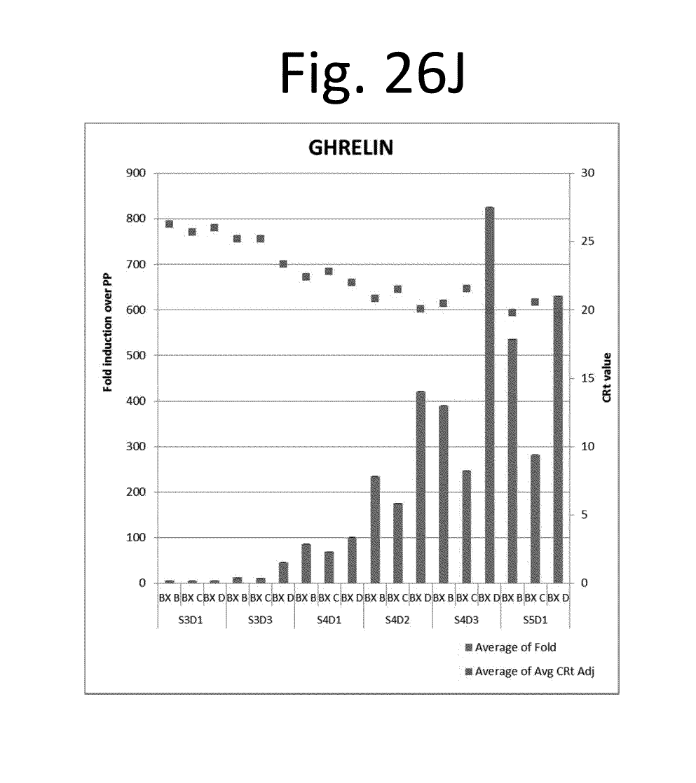

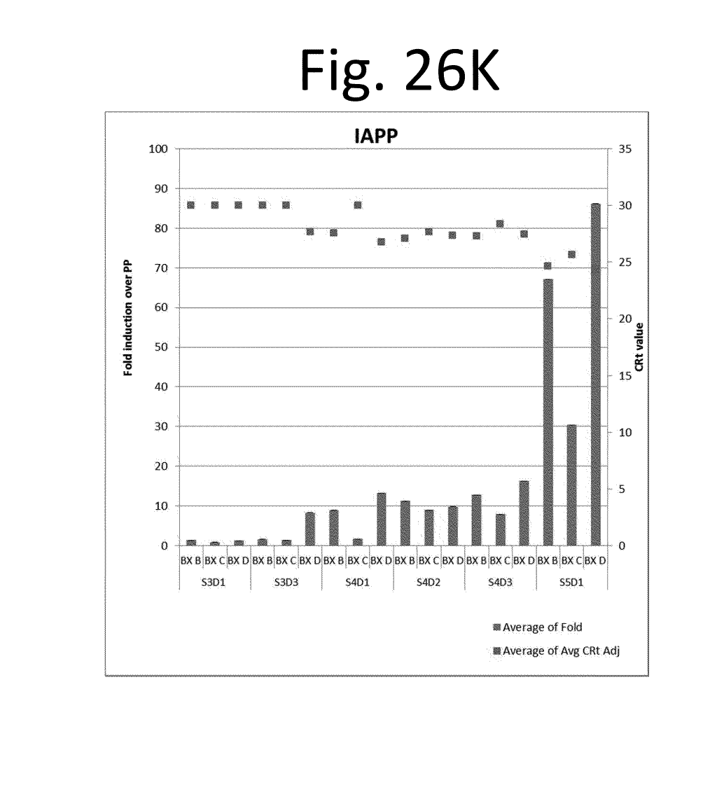

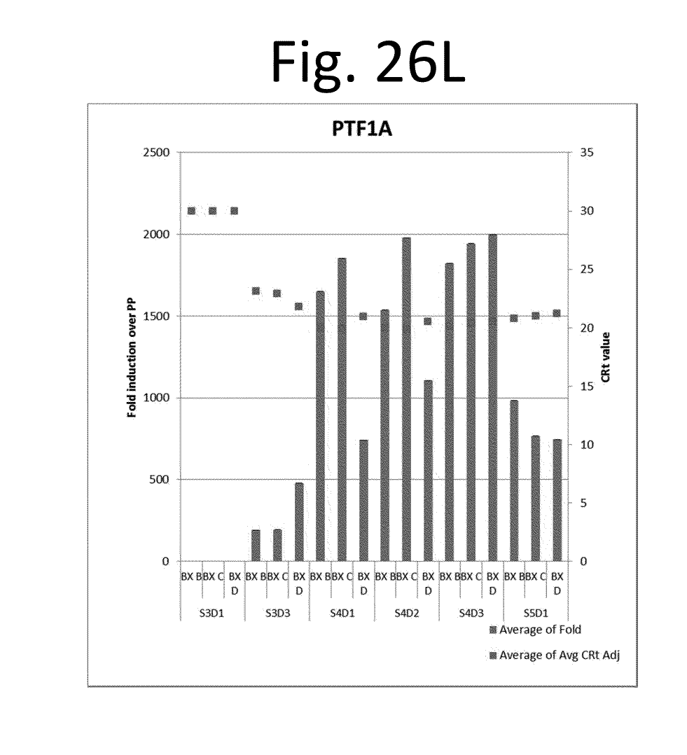

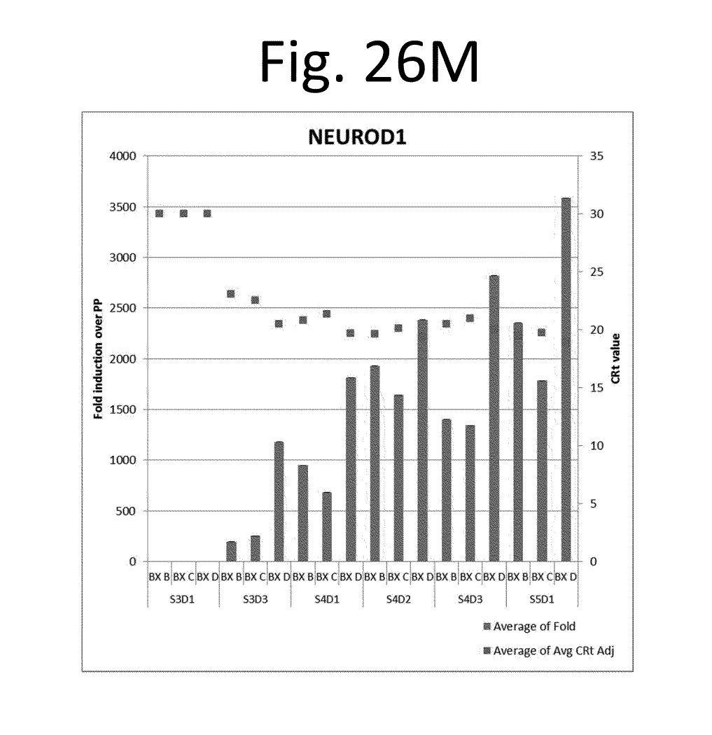

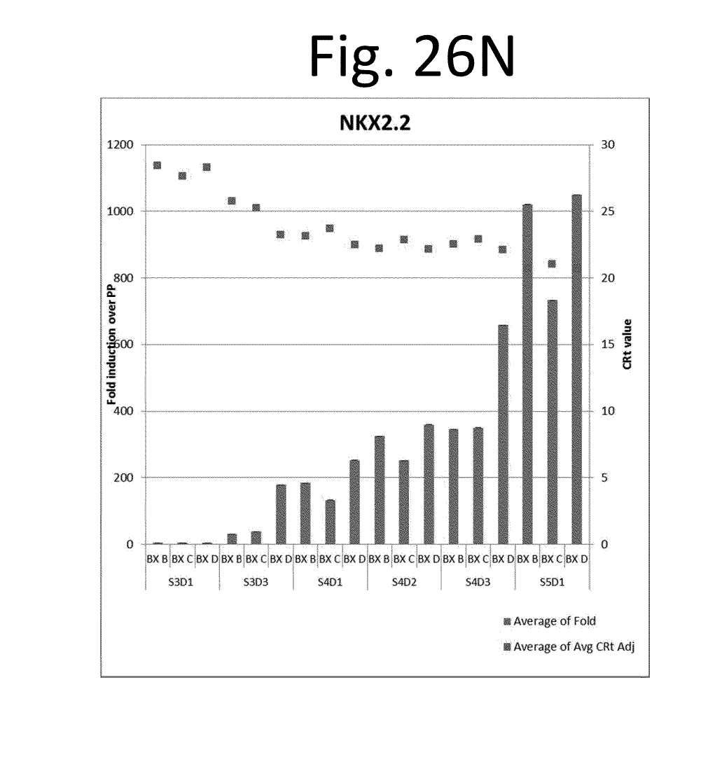

In addition to determining cell protein expression by flow cytometry, we tested samples throughout Stages 3 and 4 of the differentiation process for mRNA expression of a gene panel using OpenArray® qRT-PCR. FIGS. 26A through N depict data from real-time PCR analyses of the following genes in cells of the human embryonic stem cell line H1 differentiated through the first day of Stage 5: PDX1 (FIG. 26A); NKX6.1 (FIG. 26B); PAX4 (FIG. 26C); PAX6 (FIG. 26D); NeuroG3 (NGN3) (FIG. 26E); ABCC8 (FIG. 26F); chromogranin-A (FIG. 26G); chromogranin-B (FIG. 26H); ARX (FIG. 26I); Ghrelin (FIG. 26J); IAPP (FIG. 26K); PTF1a (FIG. 26L); NEUROD1 (FIG. 26M); and NKX2.2 (FIG. 26N) .

As shown in FIG. 26A, under both low Stage 3 pH (7.0) or standard Stage 3 pH (7.4) differentiation conditions, cells expressed similar levels of PDX1 in Stage 3 indicating the cells adopted a pancreatic fate. However, as cells from reactors B and C (pH 7.0 exposed) entered Stage 4, PDX1 expression increased in comparison to cells maintained consistently at pH 7.4 (reactor D). This increase in PDX expression was matched by an induction in NKX6.1 expression (FIG. 26B). Interestingly, cells from reactor D in Stage 3 and 4 began to express multiple genes required for and characteristic of early endocrine pancreatic cell development: PAX4, PAX6, NGN3, NEUROD1, NKX2.2, ARX, Ghrelin, CHGA and CHGB as shown in FIGS. 26C, 26D, 26E, 26M, 26N, 26I, 26J, 26G, and 26H. This pattern of gene expression combined with relatively lower NKX6.1 expression, indicated increased precocious (non-beta cell) endocrine pancreas specification in reactor D as compared to reactors B and C.

In contrast, cells from reactors B and C when measured by qRT-PCR, expressed significantly lower levels of transcription factors characteristic of precocious endocrine development (PAX4, PAX6, NGN3, NEUROD1, NKX2.2, and ARX) in Stage 3 when compared to reactor D (FIGS. 26C, 26D, 26E, 26M, 26N, and 26I). Furthermore, we observed that cells from reactors B and C had an increase in NKX6.1 message (FIG. 26B), the transcription factor required for beta cell formation, on the first day of Stage 4 that was followed by increased mRNA expression of PAX6, NEUROD1, and NKX2.2 on the second day of Stage 4 (FIGS. 26D, 26M, and 26N). These OpenArray® qRT-PCR data correlated with flow cytometry results that demonstrated cells maintained at 7.0 pH for two or three days in Stage 3 were less likely to express NEUROD1 and more likely to express NKX6.1 at the end of Stages 3 and 4 (Table XI). These results indicate exposure to low pH (7.0) for all or even some part of Stage 3 inhibited precocious (non-beta cell) endocrine pancreas specification and promoted a transcription factor expression sequence required to form beta cells.

The effect of delayed or reduced expression of genes involved in non-beta cell endocrine pancreas specification, through reduced medium pH at Stage 3, persisted through the end of Stage 5 of differentiation. Cells differentiated in reactor B (pH 7.0 for all of Stage 3) had an increased percentage of insulin positive cells (25.4%, Table XIV) when compared to reactor D cells (19.5%, Table XIV) along with an increase in NKX6.1/insulin co-positive cells (17.9%, condition B versus 14%, condition D). These results were mirrored by an increase in markers required for proper endocrine islet formation such as PAX6 and Isletl expression (Table XIV) as reactor B produced 53.8% PAX6 and 31% isletl positive cells compared to reactor D-44.9% PAX6 and 24.7% Isletl positive cells. A measure of proliferation, Ki67 expression, was also reduced in cells treated with pH 7.0 at Stage 3, as compared to cells from reactor D (Table XIV), indicating transition from a growing and less differentiated population to a more terminally differentiated tissue.

Interestingly, although low pH in Stage 3 suppressed precocious endocrine differentiation, cells from reactors B and C retained high expression of the pan-pancreatic transcription factor—PDX1—in Stages 4 and 5. Furthermore, although reactor B and C cells had low NEUROD1 expression (a pan-endocrine transcription factor) in Stages 3 and 4 compared to reactor D (Table XI), they showed a higher percentage of NEUROD1 and NEUROD1/NKX6.1 co-positive cells (Table X) by the end of Stage 5. These results indicate that low pH at Stage 3 suppressed precocious early differentiation to an endocrine fate; later promoted increased co-expression of transcription factors required for proper beta cell specification; and increased the overall expression of markers and transcription factors characteristic of islet tissue and beta cells by the end of Stage 5.

TABLE IX
Flow Cytometry Results (% of cells positive for marker)

| | | Viable | | | |
|---|---|---|---|---|---|
| | | Cell | | | |
| | | Concen- | | | |
| | | tration | | | |
| | | (10⁶ cells/ | | | |
| | Condition | mL) | NKX6.1 | PDX1 | NEUROD1 |
| S3D3- | BX B | 0.767 | 14.2 | 99.6 | 1.9 |
| 24 H | BX C | 0.818 | 16.1 | 99.3 | 2.6 |
| (Day 9) | BX D | 0.761 | 21.4 | 99.4 | 10.8 |

| | | Viable | | | | |
|---|---|---|---|---|---|---|
| | | Cell | | | | |
| | | Concen- | | | | |
| | | tration | | | | |
| | Con- | (10⁶ | | | | |
| | dition | cells/mL) | NKX6.1 | PDX1 | NEUROD1 | CDX2 |
| S4D3- | BX B | 0.551 | 82.1 | 98.1 | 15.8 | 0.6 |
| 24 H | BX C | 0.569 | 85.9 | 99.7 | 10.3 | 0.1 |
| (Day | BX D | 0.4 | 71.9 | 98.5 | 22.4 | 4.7 |
| 12) | | | | | | |

TABLE X
Stage 5 Flow Cytometry Results (% of cells positive for marker)

| | | | | | | | (NEUROD1+)/ | |
|---|---|---|---|---|---|---|---|---|
| | | (PDX1+)/ | | | | | NKX6.1+/ | |
| | NKX6.1 | NKX6.1+/PDX1+ | (INS+) NKX6.1+/INS+ | PAX6 | ISLET1 | CDX2 | NEUROD1+ | Ki67 |
| BX B | 57.5 | (87.7) 72.8 | (25.4) 17.9 | 53.8 | 31 | 2.3 | (56.7) 45.7 | 17 |
| BX C | 72.3 | (86.4) 72.7 | (21.9) 16.3 | 50.1 | 25.4 | 2.1 | (50.3) 42.5 | 22.7 |
| BX D | 66.4 | (89.6) 67.4 | (19.5) 14 | 44.9 | 24.7 | 0.8 | (46.1) 35.6 | 30 |

### Example 4

This example demonstrates formation of insulin expressing cells from a population of cells expressing PDX1 in a 3 liter stirred-tank, aseptically closed bioreactor. The insulin positive cells were generated from this process retained PDX1 expression and co-expressed NKX6.1. At the end of Stage 5, the insulin positive cells were transferred to 500 mL spinner flasks stirred at 55 RPM and held in a 5% CO2 humidified 37° C. incubator in either a medium containing high glucose (25.5 mM) or low glucose (5.5 mM) during a Stage 6. The majority of cells using either glucose concentration at Stage 6 were PDX1, NKX6.1 or NEUROD1 positive, and nearly half of all cells in the reactor were NKX6.1/PDX1/insulin co-positive.

Cells of the human embryonic stem cell line H1 (WA01 cells, WiCell Research Institute, Madison, Wis.) were grown in E8™ medium supplemented with 0.5% w/v of FAF-BSA in dynamic suspension for ≧4 passages as round aggregated clusters. The clusters were then frozen as single cells and clusters of 2 to 10 cells per the following method. Approximately 600-1000 million aggregated cells in clusters were transferred to a centrifuge tube and washed using 100mL of 1× DPS −/−. After the wash, the cell aggregates were then enzymatically disaggregated by adding a 30 mL solution of 50% StemPro®Accutase® enzyme and 50% DPBS −/− by volume to the loosened cell aggregate pellet. The cell clusters were pipetted up and down 1 to 3 times and then intermittently swirled for approximately 4 minutes at room temperature, then centrifuged for 5 min, at 80 to 200 ref. The Accutase® supernatant was then aspirated as completely as possible without disturbing the cell pellet. The centrifuge tube was then tapped against a hard surface for approximately 4 minutes, to disaggregate the clusters into single cells and clusters comprised of 2 to10 cells. After 4 minutes, the cells were re-suspended in 100 mL of E8™ medium supplemented with 10 μM Y-27632 and 0.5% w/v FAF-BSA, and centrifuged for 5 to12 minutes at 80 to 200 ref. The supernatant was then aspirated and cold (<4° C.) Cryostor® Cell Preservation Media CS10 was added drop-wise to achieve a final concentration of 100 to 150 million cells per mL. This cell solution was held in an ice bath while being aliquoted to 2 mL cryogenic vials after which the cells were frozen using a controlled rate freezer CryoMed™ 34 L Controlled-Rate Freezer as follows. The chamber was cooled to 4° C. and the temperature was held until a sample vial temperature reached 6° C. and then the chamber temperature was lowered 2° C. per minute until the sample reached −7° C. at which point the chamber was cooled 20° C./min. until the chamber reached −45° C. The chamber temperature was then allowed to briefly rise at 10° C./min. until the temperature reached −25° C., and then the chamber was cooled further at 0.8° C./min. until the sample vial reached −40° C. The chamber temperature was then cooled at 10° C./min. until the chamber reached −100° C. at which point the chamber was then cooled 35° C./min. until the chamber reached −160° C. The chamber temperature was then held at −160° C. for at least 10 minutes, after which the vials were transferred to gas phase liquid nitrogen storage. These cryo-preserved single cells at high density were then used as an ISM.

Vials of ISM were removed from the liquid nitrogen storage, thawed, and used to inoculate a 3 liter glass, stirred suspension tank bioreactor (DASGIP) at a seeding concentration of 0.295 million viable cells per mL. The vials were removed from liquid nitrogen storage and quickly transferred to a 37° C. water bath for 120 seconds to thaw. The vials were then moved to a BSC and the thawed contents transferred via 2 mL glass pipette to a 50 mL conical tube. Then 10 mL of E8™ medium supplemented with 0.5% w/v FAF-BSA and 10 μM of Rho kinase inhibitor Y-27632, were added to the tube in a drop-wise manner. The cells were centrifuged at 80-200 rcf for 5 min. The supernatant from the tube was aspirated and 10 mL fresh E8™ medium supplemented with 0.5% w/v FAF-BSA and 10 μM Y-27632 were added and the volume containing the cells was pipetted into a Cap2V8® media transfer bottle containing 450 mL E8™ media supplemented with 0.5% w/v FAF-BSA and 10 μM Y-27632. The bottle contents were then pumped directly into the bioreactor via a sterile, C-Flex® tubing weld using a peristaltic pump. The bioreactor was prepared with 1000 mL E8™ medium supplemented with 0.5% w/v FAF-BSA and 10 μM Y-27632 pre-warmed to 37° C., stirred at 70 rpm, with a dissolved oxygen set point of 30% (air O₂, and N₂ regulated), and a controlled CO₂ partial pressure of 5% . The reactor was inoculated to give a target concentration of 0.225×10⁶ cells/mL (concentration range: 0.2 to 0.5×10⁶ cells/mL).

Once the reactor was inoculated, the cells formed round aggregated clusters in the stirred reactor. After 24 hours in culture, the medium was partially exchanged as more than 80% of the original volume was removed and 1.5 L of E8™ media supplemented with 0.5% w/v FAF-BSA were added back (fresh medium). This media exchange process was repeated 48 hours after inoculation. After three days in suspension culture as round aggregated clusters, the impeller and heat jacket were stopped for 5-20 minutes to allow the clusters to settle, the medium was removed and replaced by peristaltic pump through a dip tube connected to C-Flex® tubing using a Terumo™ tube welder to maintain a closed system. The impeller and heat jacket were re-energized once sufficient medium was added to submerge the impeller. The differentiation protocol is described below.

Stage 1 (3 Days):

A Stage 1 base medium was prepared using 900 mL MCDB-131 medium containing 1.18 g/L sodium bicarbonate and supplemented with an additional 3.6 g/L sodium bicarbonate; 100 mL 2% w/v FAF-BSA, previously re-constituted in MCDB-131; 10 mL of 1× concentration of GlutaMAX™; 1 mL of a 2.5 mM glucose (45% in water); and a 1:50,000 dilution of ITS-X. Cells were cultured for one day in the base medium supplemented with 100 ng/ml GDF8 and 3 μM of MCX compound. After 24 hours, a media exchange was completed as described above, and fresh base medium supplemented with 100 ng/mL of GDF8 was added to the flask. Cells were maintained without further media exchange for 48 hours. The dissolved oxygen content was maintained at 10% and pH at 7.4 throughout Stage 1

Stage 2 (3 Days):

After the completion of Stage 1, a media exchange was completed as described above, whereby the spent Stage 1 medium was removed and replaced with the base medium of Stage 1, but supplemented with 50 ng/mL FGF7. Forty-eight hours after the media exchange, the spent media was again removed and replaced with fresh base medium supplemented with 50 ng/mL FGF7. The DO was maintained at 30% DO and pH at 7.4% throughout Stage 2.

Stage 3 (3 Days):

After the completion of Stage 2, a media exchange was completed as described above, whereby the spent Stage 2 medium was removed and replaced with the following base medium: 900 mL MCDB-131 medium containing 1.18 g/L sodium bicarbonate and supplemented with an additional 3.6 g/L sodium bicarbonate; 100 mL 2% w/v FAF-BSA, previously re-constituted in MCDB-131; 10 mL of 1× concentration of GlutaMAX™; 1 mL of a 2.5 mM glucose (45% in water); and a 1:200 dilution of ITS-X. The Stage 3 base medium was supplemented with 50 ng/mL FGF-7; 100 nM of LDN-193189; 2 μM RA; 0.25 μM SANT-1; and 400 nM of TPB. Twenty-four hours post media exchange, the spent media was again replaced fresh medium containing the above supplements with the exception of LDN-193189. Cells were cultured in the media for 48 hours. Throughout Stage 3, a 30% DO and pH of 7.0 were maintained.

Stage 4 (3 Days):

After the completion of Stage 3, a media exchange was completed as described above, whereby the spent Stage 3 medium was removed and replaced with the same base medium as used in Stage 3, but supplemented with 0.25 μM SANT-1 and 400 nM of TPB. Forty-eight hours after initiation of Stage 4, 3.2mL/L of a 45% glucose solution (8 mM glucose bolus) was added to the each bioreactor and the cells were cultured in the media for an additional 24 hours. Throughout Stage 4, a 30% DO and pH of 7.4 were maintained.

Stage 5 (7 Days):

After the completion of Stage 4, a media exchange was completed as described above, whereby the spent Stage 4 medium was removed and replaced with the following Stage 5 base medium: 900 mL MCDB-131 medium base containing 1.18 g/L sodium bicarbonate supplemented with an additional 1.754 g/L sodium bicarbonate; 100 mL 2% w/v FAF-BSA previously re-constituted in MCDB-131; 1× concentration of GlutaMAX™; 8 mL/L of a 45% glucose solution; 1:200 dilution of ITS-X; 250 μL/L of 1M ascorbic acid; and 1 mL, 10 mg/L heparin solution. The Stage 5 base medium was supplemented with 1 μM T3, 10 μM ALK5 inhibitor II, 1 μM of gamma secretase inhibitor XXI; 20 ng/mL of betacellulin; 0.25 μM SANT-1; and 100 nM RA. Forty-eight hours after initiation of Stage 5, the spent media was removed and replaced with the same fresh base medium and supplements. Forty-eight hours later, the media was again exchanged and replaced with the same fresh medium and supplements. Forty-eight hours later the medium was again exchanged and replaced with the same fresh medium and supplements, except the gamma secretase inhibitor XXI and SANT were excluded. Forty-eight hours later, the spent media was removed and replaced with the same fresh medium and supplements. The cells were cultured for an additional 24 hours to the end of Stage 5. Throughout Stage 5, a 30% DO and pH 7.4 were maintained.

Stage 6 (7 Days):

At the end of Stage 5, (day 19 of differentiation), cells were removed from the 3 liter reactor via sterile weld and peristaltic pump. The cells were then counted, gravity settled, and resuspended in Stage 6 medium (detailed below) at a normalized distribution of 0.5 million cells/mL and added to two, 0.5 liter disposable spinner flasks (Corning) stirred at 55 RPM and maintained for 7 days under drift conditions in a 5% CO₂ humidified 37° C. incubator in either a medium containing high glucose (25.5 mM) or low glucose (5.5 mM). One flask contained the following medium and supplements: 300 mL MCDB-131 medium base containing 1.18 g/L sodium bicarbonate supplemented with an additional 1.754 g/L sodium bicarbonate; 100 mL 2% w/v FAF-BSA previously re-constituted in MCDB-131; 1× concentration of GlutaMAX™; 8 mL/L of a 45% glucose solution (25.5 mM final glucose concentration); 1:200 dilution of ITS-X; 250 μL/L of 1M ascorbic acid; and 1 mL, 10 mg/L heparin; and 10 μM ALK5 inhibitor II. The second flask contained the following medium and supplements: 300 mL MCDB-131 medium base containing 1.18 g/L sodium bicarbonate and the basal glucose concentration of 5.5 mM supplemented with an additional 1.754 g/L sodium bicarbonate; 100 mL 2% w/v FAF-BSA previously re-constituted in MCDB-131; 1× concentration of GlutaMAX™; 1:200 dilution of ITS-X; 250 μL/L of 1M ascorbic acid; and 1 mL, 10 mg/L heparin; and 10 μM ALK5 inhibitor II. Forty-eight hours, ninety-six hours and one hundred twenty hours after initiation of Stage 5, the spent media was removed and replaced with the same fresh base medium and supplements. Stage 6 was ended 144 hours (Day 26 differentiation) after initiation.

Throughout the differentiation process, samples were collected from the reactors and analyzed for total cell number as shown in Table X and mRNA expression (OpenArray® qRT-PCR) as shown in FIG. 27. At the end of Stages 3, 4, 5, and 6 samples were assayed for protein expression using flow cytometry (Table XII).

At the completion of Stage 3, it was observed that nearly all cells expressed both the endoderm transcription factor (FOXA2) and the pancreatic specific transcription factor (PDX1). A minority of cells were detected that expressed NKX6.1 (˜20%) and almost no NEUROD1 expressing cells by flow cytometry (Table XII). At the end of Stage 4, samples were again analyzed by flow cytometry for expression of NKX6.1, NEUROD1, PDX1, FOXA2, CDX2, and Ki67 (Table XII). Interestingly, from the end of Stage 3 to the end of Stage 4, the NKX6.1 expressing population increased to over 91% of cells and these cells retained endodermal and pancreatic specification (>99% PDX1 and FOXA2 expressing cells). However, only a limited population of cells (<8%) expressed markers characteristic of endocrine hormone cells (Islet1, CHGA, NEUROD1, and NKX2.2). At the completion of Stage 5, the percentage of cells positive for markers characteristic of endocrine hormone cells increased substantially-rising from less than 10% at the end of stage 4 to 76% of cells positive for NEUROD1 and 57% positive for insulin. Furthermore, the total population of cells remained predominantly NKX6.1 (81%) and PDX1 (>97%) expressing. The level of proliferation as measured by percent of cells positive for Ki67 was about 18% and CDX2, a marker for endodermal gut cells, was very low at <3.0%. These data indicate that an islet-like , and specifically a beta cell-like population, was forming in the reactor.

At the completion of Stage 5, cells were removed from the 3 liter stirred tank reactor and split into 500 mL spinner flasks maintained in a 5% CO2, 37° C., humidified incubator. The spinner flasks were treated under similar conditions with the exception of the basal media glucose concentration. The two glucose conditions tested were: low glucose-5.5 mM starting basal glucose concentration (“LG”), or a high glucose-25.5 mM starting basal glucose concentration (“HG”) (Table XIV). Cells treated in Stage 6 for seven days in either condition showed a substantial increase in markers characteristic of endocrine hormone cells, and especially pancreatic beta islet cells. At the end of day seven of Stage 6, almost half of the cells were positive for PAX6, while 60% were co-positive for NEUROD1 & NKX6.1, or Insulin & NKX6.1 (Table XIII) Additionally, cells generated in this system retained high levels of PDX1 (>81%) and demonstrated a reduced level of proliferation as measured by the percent of cells positive for Ki67 (about 12%, per Table XIV).

These results were supported by OpenArray®qRT-PCR data showing that as the cells enter Stage 5 there is a dramatic and transitory induction of NGN3 (FIG. 27A). This is followed by a sustained induction in NEUROD1 expression (FIG. 27B) and other genes associated with islet formation and endocrine hormone cells such as Chromogranin A (CHGA), Chromogranin B (CHGB), Glucagon (GCG), Islet Associated Polypeptide (IAPP), Isletl (ISL1), MAFB, PAX6, and Somatostatin (SST) as shown in FIGS. 27C through J, respectively. In addition to the induction of islet specific genes, beta cell specific genes were also induced in Stage 5 and sustained through Stage 6, as observed for insulin (INS; FIG. 27K), glucose 6 phosphatase 2 (G6PC2; FIG. 27L), PCSK1 and 2 (FIGS. 27M and N), zinc transporter (SLC30A8; FIG. 27O) as were transcription factors required for beta cell formation and function such as NKX6.1, NKX2.2, MNX1/HB9, and UCN3 (FIGS. 27P-S, respectively). The expression of genes such as CDX2 and ZIC 1, indicating formation of alternative fates, was near or below the limits of detection by qRT-PCR (data not shown).

TABLE XI
Total Cell Counts at specified day of differentiation

| Days in Differentiation | Total cells (×10⁶/mL) |
|---|---|
| −3 | 0.23 |
| 0-pre Adjust | 0.75 |
| 0-0.5 × 106/mL Adjust | 0.50 |
| 4 | 1.49 |
| 6-pre Adjust | 1.61 |
| 6-2M/mL Adjust | 2.15 |
| 7 | 2.21 |
| 11 | 1.47 |
| 12 | 1.24 |
| 13 | 0.68 |
| 19 | 0.57 |

TABLE XII
Flow Cytometry Results (% of cells positive for marker) at end of Stage 3 (S3D3-
24 H) and Stage 4 (S4D3-24 H)

| | Viable Cell | | | | | | | |
|---|---|---|---|---|---|---|---|---|
| | Concentration | | | | | | | |
| | (10⁶ cells/mL) | NKX6.1 | CHGA | NKX2.2 | PDX1 | FOXA2 | NEUROD1 | ISLET1 |
| S3D3- | 1.14 | 21 | 1.6 | N/A | 99.6 | 99.9 | 4 | N/A |
| 24 H | | | | | | | | |
| S4D3- | 1.24 | 91.7 | 5.2 | 7.2 | 99.8 | 99.4 | 7.2 | 2.8 |
| 24 H | | | | | | | | |

TABLE XIII
Flow Cytometry Results (% of cells positive for marker) at end of Stage 5
(S5D7-24 H)

| Viable Cell | | | (NEUROD1+) | (INS+) | | | |
|---|---|---|---|---|---|---|---|
| Concentration | | | NKX6.1+/ | NKX6.1+/ | | | |
| (10⁶ cells/mL) | NKX6.1 | PDX1 | NEUROD1+ | INS+ | ISLET1 | Ki67 | CDX2 |
| 0.42 | 81.1 | 97.6 | (76.3) 60.5 | (56) 45.7 | 36.8 | 18.5 | 2.6 |

TABLE XIV
Flow Cytometry Results (% of cells positive for marker) at end of Stage 6
(S6D7-24 H) (note: LG = 5.5 mM glucose; HG = 25.5 mM glucose)

| | | | (NEUROD1+) | (C-pep+) | | (C-pep+) | (CHGA+)/ | | |
|---|---|---|---|---|---|---|---|---|---|
| | | | NKX6.1+/ | NKX6.1+/ | (INS+) | C-pep+/ | NKX6.1+/ | | |
| | NKX6.1 | PDX1 | NEUROD1+ | C-pep+ | NKX6.1+/INS+ | INS+ | CHGA+ | Ki67 | PAX6 |
| LG | 87 | 81.2 | (71.1) 61.1 | (43.4) | (72.6) 64.8 | (41) 37.7 | (56.7) 48 | 12 | 51.3 |
| | | | | 38.4 | | | | | |
| HG | 83.8 | 86.7 | (69.1) 60.5 | (46.9) | (71.5) 61.9 | (47.4) 42.1 | (66.1) 53.8 | 11.6 | 46.7 |
| | | | | 38.6 | | | | | |

### Example 5

This example demonstrates formation of insulin expressing cells from a population of cells expressing the transcription factor, PDX1, in a stirred-tank, aseptically closed bioreactor. The insulin positive cells generated from this process retained PDX1 expression and co-expressed NKX6.1. When this population of cells was transplanted into the kidney capsule of immune-compromised mice the graft produced detectable blood levels of human C-peptide within four weeks of engraftment.

Cells of the human embryonic stem cell line H1 (WA01 cells, WiCell Research Institute, Madison, Wis.) were grown in E8TM medium supplemented with 0.5% w/v FAF-BSA in dynamic suspension for ≧4 passages as round aggregated clusters. The clusters were then frozen as single cells and clusters of 2 to 10 cells per the following method. Approximately 600-1000 million aggregated cells in clusters were transferred to a centrifuge tube and washed using 100mL of 1× DPS −/−. After the wash, the cell aggregates were then enzymatically disaggregated by adding a 30 mL solution of 50% StemPro®Accutase® enzyme and 50% DPBS −/− by volume to the loosened cell aggregate pellet. The cell clusters were pipetted up and down 1 to 3 times and then intermittently swirled for approximately 4 minutes at room temperature, then centrifuged for 5 min, at 80 to 200 ref. The Accutase® supernatant was then aspirated as completely as possible without disturbing the cell pellet. The centrifuge tube was then tapped against a hard surface for approximately 4 minutes, to disaggregate the clusters into single cells and clusters comprised of 2 to10 cells. After 4 minutes, the cells were re-suspended in 100 mL of E8™ media supplemented with 10 μM Y-27632 (Enzo Life Sciences) and 0.5% w/v FAF-BSA, and centrifuged for 5 to12 minutes at 80 to 200 rcf. The supernatant was then aspirated and cold (≦4° C.) Cryostor® Cell Preservation Media CS10 was added drop-wise to achieve a final concentration of 100 to 150 million cells per mL. This cell solution was held in an ice bath while being aliquoted to 2 mL cryogenic vials (Corning) after which the cells were frozen using a controlled rate CryoMed™ 34L freezer as follows. The chamber was cooled to 4° C. and the temperature was held until a sample vial temperature reached 6° C. and then the chamber temperature was lowered 2° C. per minute until the sample reached −7° C. at which point the chamber was cooled 20° C./min. until the chamber reached −45° C. The chamber temperature was then allowed to briefly rise at 10° C./min. until the temperature reached −25° C., and then the chamber was cooled further at 0.8° C./min. until the sample vial reached −40° C. The chamber temperature was then cooled at 10° C./min. until the chamber reached −100° C. at which point the chamber was then cooled 35° C./min. until the chamber reached −160° C. The chamber temperature was then held at −160° C. for at least 10 minutes, after which the vials were transferred to gas phase liquid nitrogen storage. These cryo-preserved single cells at high density were then used as an ISM.

ISM vials were removed from the liquid nitrogen storage, thawed, and used to inoculate a 3 liter glass, stirred suspension tank bioreactor (DASGIP) at a seeding concentration of 0.295 million viable cells per mL. The vials were removed from liquid nitrogen storage and quickly transferred to a 37° C. water bath for 120 seconds to thaw. The vials were then moved to a BSC and the thawed contents transferred via 2 mL glass pipette to a 50 mL conical tube. Then 10 mL of E8™ medium supplemented with 0.5% w/v FAF-BSA and 10 μM of Rho kinase inhibitor Y-27632, were added to the tube in a drop-wise manner. The cells were centrifuged at 80-200 rcf for 5 min. The supernatant from the tube was aspirated and 10 mL fresh E8™ medium supplemented with 0.5% w/v FAF-BSA and 10 μM Y-27632 were added and the volume containing the cells was pipetted into a media transfer bottle (Cap2V8®, Sanisure, Inc) containing 450 mL E8™ media supplemented with 0.5% w/v FAF-BSA and 10 μM Y-27632. The bottle contents were then pumped directly into the bioreactor via a sterile, C-Flex® tubing weld using a peristaltic pump. The bioreactor was prepared with 1000 mL E8™ medium supplemented with 0.5% w/v FAF-BSA and 10 μM Y-27632 pre-warmed to 37° C., stirred at 70 rpm, with a dissolved oxygen set point of 30% (air O₂, and N₂ regulated), and a controlled CO₂ partial pressure of 5% . The reactor was inoculated to give a target concentration of 0.225×10⁶ cells/mL (concentration range: 0.2 to 0.5×10⁶ cells/mL).

Once the reactor was inoculated, the cells formed round aggregated clusters in the stirred reactor. After 24 hours in culture, the medium was partially exchanged as more than 80% of the original volume was removed and 1.5 L of E8™ medium supplemented with 0.5% w/v FAF-BSA was added back (fresh medium). This media exchange process was repeated 48 hours after inoculation. After three days in suspension culture as round aggregated clusters, differentiation in the 3 liter reactor was initiated by removing the spent E8™ medium and adding differentiation medium. The differentiation protocol is described below.

Stage 1 (3 Days):

The reactor was set to a temperature of 37° C. and stirred continuously at 70 rpm. Gas and pH controls were set to a dissolved oxygen set point of 10% (air, O2, and N2 regulated), and the pH was set to 7.4 via CO2 regulation. A Stage 1 base medium was prepared using 1.5 L MCDB-131 medium containing 1.18 g/L sodium bicarbonate; supplemented with an additional 2.4 g/L sodium bicarbonate, 2% w/v FAF-BSA, previously re-constituted in MCDB-131; 1× concentration of GlutaMAX™; 2.5 mM glucose (45% in water); and a 1:50,000 dilution of ITS-X. Cells were cultured for one day in 1.5 L of the base medium supplemented with 100 ng/ml GDF8; and 3 μM of MCX compound. After 24 hours, a media exchange was completed as described above, and fresh 1.5 L of base medium supplemented with 100 ng/mL of GDF8 were added to the reactor. Cells were maintained without further media exchange for 48 hours.

Stage 2 (3 Days):

The reactor was set to a temperature of 37° C. and stirred continuously at 70 rpm. Gas and pH controls were set to a dissolved oxygen set point of 30% (air O2, and N2regulated), and the pH was set to 7.4 via CO2 regulation. After the completion of Stage 1, a media exchange was completed as described above, whereby the spent Stage 1 media was removed and replaced with the 1.5 L of the same medium, but supplemented with 50 ng/mL FGF7. Forty-eight hours after the media exchange, the spent media was again removed and replaced with 300 mL fresh Stage 2 base medium supplemented with 50 ng/mL FGF7.

Stage 3 (3 Days):

At the completion of Stage 2, and just prior to medium exchange, the cells were counted, gravity settled and re-suspended in the following Stage 3 base medium at a normalized distribution of 2.0 million cells/mL in 1.5 liters: 1.5 L MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of GlutaMAX™; 2.5 mM glucose; and a 1:200 dilution of ITS-X. The Stage 3 base medium was supplemented with 50 ng/mL FGF-7; 100 nM of LDN-193189; 2 μM RA; 0.25 μM SANT-1; and 400 nM of TPB. The reactor was set to a temperature of 37° C. and stirred continuously at 70 rpm. Gas and pH controls were set to a dissolved oxygen set point of 30% (air O2, and N2 regulated), and 7.0 pH via CO2 regulation. Twenty-four hours post media exchange, the spent media was again replaced with 1.5 L fresh Stage 3 medium containing the above supplements with the exception of LDN-193189. Cells were thereafter cultured in the media for 48 hours, until the end of Stage 3.

Stage 4 (3 Days):

At the completion of Stage 3, the spent media was removed and replaced in each bioreactor with 1.5 L of Stage 4 base medium composed of: 1.5 L MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of GlutaMAX™; 2.5 mM glucose; and a 1:200 dilution of ITS-X. The Stage 4 base medium was supplemented with 0.25 μM SANT-1 and 400 nM of TPB. The reactor was maintained at 37° C. and stirred at 70 rpm. Gas and pH were regulated to a dissolved oxygen set point of 30% (air, O2, and N2 regulated) and a pH set point of 7.4 via CO2 regulation. Forty-eight hours after initiation of Stage 4, 3.2 mL/L of a 45% glucose solution (8 mM glucose bolus) was added to the bioreactor and the cells were cultured in the media for an additional 24 hours.

Stages 5 and 6:

At the conclusion of the third day of Stage 4, round aggregated clusters were pumped out of the bioreactor and transferred to two separate 0.5 liter Corning disposable spinner flasks stirred at 55 RPM and maintained in a 37° C. humidified incubator supplemented with 5% CO2. Thereafter the cells in each vessel were maintained in a 300 mL working volume of Stage 5 base medium composed of: 300 mL of MCDB-131 medium containing 1.18 g/L sodium bicarbonate supplemented with an additional 1.75 g/L sodium bicarbonate; 2% w/v FAF-BSA previously re-constituted in MCDB-131; 1× concentration of GlutaMAX™; 20 mM glucose; 1:200 dilution of ITS-X; 250 μL/L of 1M ascorbic acid; 10 mg/L heparin; 1 μM T3 as 3,3′,5-Triiodo-L-thyronine sodium salt and 10 μM of ALK5 inhibitor II.

The Stage 5 base medium used was supplemented according to two different conditions, A or B as follows:
a. For condition A, Stage 5 was initiated by applying Stage 5+ base medium supplemented with 100 nM LDN, 100 nM SANT, and 10 μM Zinc Sulfate. This medium was exchanged 24 and 48 hours after beginning stage. 72 hours after beginning Stage 5, Stage 6 was initiated by removing the spent medium and treating the cells with Stage 5 base medium supplemented with 100 nM XX gamma secretase inhibitor, 100 nM LDN, and 10 μM Zinc Sulfate. This medium was thereafter replaced every 24 hours for eleven days, except at the beginning of days 8, 9, and 11.
b. For condition B, Stage 5 was initiated by applying Stage 5 base medium supplemented with 100 nM of gamma secretase inhibitor, XX; 20 ng/mL of betacellulin; 0.25 μM SANT-1; and 100 nM RA. Forty-eight hours after initiation of Stage 5, the spent media was removed and replaced with 300 mL of the same media and supplements. Forty-eight hours later, the medium was removed and replaced with Stage 5 base medium supplemented with 20 ng/mL of betacellulin, and 100 nM RA. Forty-eight hours later the medium was again exchanged and replaced with the same medium.

Throughout the differentiation process cell samples were collected from the suspension cultures for analysis. Samples were analyzed for mRNA expression (OpenArray® qRT-PCR) and protein expression (flow cytometry and florescent immune-histochemistry).

Six days after the end of Stage 4 (Condition A—Stage 6, Day 3; Condition B-Stage 5, Day 6) it was observed that cells from both treatments expressed a panel of proteins, detectable by flow cytometry, consistent with the formation of endocrine pancreas and beta cells (Table XV). Both treatments generated a high percentage of PDX1 (>91%) expressing cells and cells began to co-express insulin and NKX6.1 (not shown). Interestingly, it was observed that cells treated according to condition A had reduced levels of proliferation-15.5% of cells in A and 27.3% in B expressed Ki67 (Table XV). Furthermore, cells treated with condition A had more NKX6.1 expressing, NEUROD1 expressing, and NKX6.1/NEUROD1 co-expressing cells than condition B (Table XV), indicating that treatment with condition A generated a larger population of cells expressing genes characteristic of endocrine pancreas and capable of forming beta cells.

These flow cytometry data were supported by OpenArray qRT-PCR data that showed that, as cells entered Stage 5, there was an induction of NGN3 (FIG. 28 A) under both conditions correlating with sustained induction of NEUROD1 expression (FIG. 28B). In Condition A, after the initial induction of NGN3 in Stage 5 there was a second induction of NGN3 at the beginning of Stage 6 that corresponded to treatment with a gamma secretase inhibitor, XX. This double peak of NGN3 expression for condition A occurred in conjunction with sustained expression of NKX6.1 (FIG. 28C) and correlated with expression of multiple genes associated with islet formation and endocrine hormone cells such as Chromogranin A (CHGA), Chromogranin B (CHGB), Glucagon (GCG), Islet Associated Polypeptide (IAPP), MAFB, PAX6, and Somatostatin (SST) (FIGS. 28D through J, respectively). Furthermore, genes required for beta cell function were also induced in stage 5 and sustained through stage 6, as observed for insulin (INS; FIG. 28K), glucose 6 phosphatase 2 (G6PC2; FIG. 28L), PCSK1 (FIG. 28M), and zinc transporter (SLC30A8; FIG. 28N) as were MNX1/HB9, and UCN3—transcription factors required for beta cell formation, maturation, and function (FIGS. 28O and P, respectively).

At the end of the eleventh day of stage six, 5×10⁷ differentiated cells from condition A were isolated from the media in a 50 mL conical, then washed 2 times with MCDB-1313 medium containing 1.18 g/L sodium bicarbonate and 0.2% w/v FAF-BSA. The cells were re-suspended in the was media and held at room temperature for approximately 5 hours prior to transplantation under the kidney capsule of NSG mice. Each animal received a dose of 5×10⁶ cells. Prior to implantation, these cells expressed proteins consistent with endocrine pancreas and beta cells (Table XVI) and at the earliest measured time point, 4 weeks post-implant, and throughout the 18 week course of the study, human C-peptide was detected in response to intra-peritoneal glucose injection following an overnight fast and retro-orbital blood draw 60 minutes after the IP glucose bolus (N=7 animals, FIG. 29).

TABLE XV
Flow Cytometry Results Six days after the end of stage 4
(Condition A- Stage 6, Day 3; Condition B-Stage 5, Day 6)

| | | | NEUROD1 | |
|---|---|---|---|---|
| | | | (NEUORD1+/ | |
| | NKX6.1 | PDX1 | NKX6.1+) | Ki67 |
| Condition A | 67.5 | 91.9 | 67.9 (45.5) | 15.5 |
| Condition B | 49.4 | 92.1 | 44.0 (31.5) | 27.3 |

TABLE XVI
Flow Cytometry Results Condition A- Stage 6, Day 11

| | | INS | | | |
|---|---|---|---|---|---|
| | | (NKX6.1+/ | | | |
| | NKX6.1 | INS+) | NKX2.2 | PDX1 | Ki67 |
| Condition A | 90.4 | 28.9 | 90.3 | 96.2 | 5.3 |
| | | (25.8) | | | |

### Example 6

This example demonstrates formation of insulin expressing cells from a population of cells expressing PDX1 in a stirred-tank, aseptically closed bioreactor. The insulin positive cells generated from this process retained PDX1 expression and co-expressed NKX6.1. When this population of cells was transplanted into the kidney capsule of immune-compromised mice, the graft produced detectable blood levels of human C-peptide within two weeks of engraftment.

Cells of the human embryonic stem cell line H1 (WA01 cells, WiCell Research Institute, Madison, Wis.) were grown in E8™ medium supplemented with 0.5% w/v FAF-BSA in dynamic suspension for ≧4 passages as round aggregated clusters. The clusters were then frozen as single cells and clusters of 2 to 10 cells per the following method. Approximately 600-1000 million aggregated cells in clusters were transferred to a centrifuge tube and washed using 100 mL of 1× DPS −/−. After the wash, the cell aggregates were then enzymatically disaggregated by adding a 30 mL solution of 50% StemPro®Accutase® enzyme and 50% DPBS −/− by volume to the loosened cell aggregate pellet. The cell clusters were pipetted up and down 1 to 3 times and then intermittently swirled for approximately 4 minutes at room temperature, then centrifuged for 5 min, at 80 to 200 ref. The Accutase® supernatant was then aspirated as completely as possible without disturbing the cell pellet. The centrifuge tube was then tapped against a hard surface for approximately 4 minutes, to disaggregate the clusters into single cells and clusters comprised of 2 to10 cells. After 4 minutes, the cells were re-suspended in 100mL of E8™ media supplemented with 10 μM Y-27632 (Enzo Life Sciences) and 0.5% w/v FAF-BSA, and centrifuged for 5 to12 minutes at 80 to 200 rcf. The supernatant was then aspirated and cold (<4° C.) Cryostor® Cell Preservation Media CS10 was added drop-wise to achieve a final concentration of 100 to 150 million cells per mL. This cell solution was held in an ice bath while being aliquoted to 2 mL cryogenic vials (Corning) after which the cells were frozen using a controlled rate CryoMed™ 34 L freezer as follows. The chamber was cooled to 4° C. and the temperature was held until a sample vial temperature reached 6° C. and then the chamber temperature was lowered 2° C. per minute until the sample reached −7° C. at which point the chamber was cooled 20° C./min. until the chamber reached −45° C. The chamber temperature was then allowed to briefly rise at 10° C./min. until the temperature reached −25° C., and then the chamber was cooled further at 0.8° C./min. until the sample vial reached −40° C. The chamber temperature was then cooled at 10° C./min. until the chamber reached −100° C. at which point the chamber was then cooled 35° C./min. until the chamber reached −160° C. The chamber temperature was then held at −160° C. for at least 10 minutes, after which the vials were transferred to gas phase liquid nitrogen storage. These cryo-preserved single cells at high density were then used as an ISM.

ISM vials were removed from the liquid nitrogen storage, thawed, and used to inoculate a 3 liter glass, stirred suspension tank bioreactor (DASGIP) at a seeding concentration of 0.295 million viable cells per mL. The vials were removed from liquid nitrogen storage and quickly transferred to a 37° C. water bath for 120 seconds to thaw. The vials were then moved to a BSC and the thawed contents transferred via 2 mL glass pipette to a 50 mL conical tube. Then 10mL of E8™ medium supplemented with 0.5% w/v FAF-BSA and 10 μM of Rho kinase inhibitor Y-27632, were added to the tube in a drop-wise manner. The cells were centrifuged at 80-200 rcf for 5 min. The supernatant from the tube was aspirated and 10mL fresh E8TM medium supplemented with 0.5% w/v FAF-BSA and 10 μM Y-27632 were added and the volume containing the cells was pipetted into a media transfer bottle (Cap2V8®, Sanisure, Inc) containing 450 mL E8™ media supplemented with 0.5% w/v FAF-BSA and 10 μM Y-27632. The bottle contents were then pumped directly into the bioreactor via a sterile, C-Flex® tubing weld using a peristaltic pump. The bioreactor was prepared with 1000 mL E8™ medium supplemented with 0.5% w/v FAF-BSA and 10 μM Y-27632 pre-warmed to 37° C., stirred at 70 rpm, with a dissolved oxygen set point of 30% (air O₂, and N₂ regulated), and a controlled CO₂ partial pressure of 5% . The reactor was inoculated to give a target concentration of 0.225×10⁶ cells/mL (concentration range: 0.2 to 0.5×10⁶ cells/mL).

Once the reactor was inoculated, the cells formed round aggregated clusters in the stirred reactor. After 24 hours in culture, the medium was partially exchanged as more than 80% of the original volume was removed and 1.5 L of E8™ medium supplemented with 0.5% w/v FAF-BSA was added back (fresh medium). This media exchange process was repeated 48 hours after inoculation. After three days in suspension culture as round aggregated clusters, differentiation in the 3 liter reactor was initiated by removing the spent E8™ medium and adding differentiation medium. The differentiation protocol is described below.

Stage 1 (3 Days):

The reactor was set to a temperature of 37° C. and stirred continuously at 70 rpm. Gas and pH controls were set to a dissolved oxygen set point of 10% (air, O2, and N2 regulated), and the pH was set to 7.4 via CO2 regulation. A Stage 1 base medium was prepared using 1.5 L MCDB-131 medium containing 1.18 g/L sodium bicarbonate; supplemented with an additional 2.4 g/L sodium bicarbonate, 2% w/v FAF-BSA, previously re-constituted in MCDB-131; 1× concentration of GlutaMAX™; 2.5 mM glucose (45% in water); and a 1:50,000 dilution of ITS-X. Cells were cultured for one day in 1.5 L of the base medium supplemented with 100 ng/ml GDF8 and 3 μM of MCX compound. After 24 hours, a media exchange was completed as described above, and fresh 1.5 L of base medium supplemented with 100 ng/mL of GDF8 were added to the reactor. Cells were maintained without further media exchange for 48 hours.

Stage 2 (3 Days):

The reactor was set to a temperature of 37° C. and stirred continuously at 70 rpm. Gas and pH controls were set to a dissolved oxygen set point of 30% (air O2, and N2 regulated), and the pH was set to 7.4 via CO2 regulation. After the completion of Stage 1, a media exchange was completed as described above, whereby the spent Stage 1 media was removed and replaced with the 1.5 L of the same medium used as the Stage 1 base medium, but supplemented with 50 ng/mL FGF7. Forty-eight hours after the media exchange, the spent media was again removed and replaced with 300 mL fresh base medium supplemented with 50 ng/mL FGF7.

Stage 3 (3 Days):

At the completion of Stage 2, and just prior to medium exchange, the cells were counted, gravity settled and re-suspended in the following Stage 3 base medium at a normalized concentration of 2.0 million cells/mL in 1.5 liters: 1.5 L MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of GlutaMAX™; 2.5 mM glucose; and a 1:200 dilution of ITS-X. The Stage 3 base medium was supplemented with 50 ng/mL FGF-7; 100 nM of LDN-193189; 2 μM RA; 0.25 μM SANT-1; and 400 nM of TPB. The reactor was set to a temperature of 37° C. and stirred continuously at 70 rpm. Gas and pH controls were set to a dissolved oxygen set point of 30% (air, O2, and N2 regulated), and 7.0 pH via CO2 regulation. Twenty-four hours post media exchange, the spent media was again replaced with 1.5 L fresh Stage 3 base medium containing the above supplements with the exception of LDN-193189. Cells were thereafter cultured in the media for 48 hours, until the end of Stage 3.

At the conclusion of Stage, 150 mL of cells (1.05×10⁶ viable cells/mL) were removed from the parent 3 liter reactor and aseptically transferred to a 0.2 L reactor. The remaining 1.35 L reactor volume was further differentiated according to Stage 4 described below and this process and the cells are hereinafter referred to as the “Standard process” and the “Standard cells.” The cells transferred to the 0.2 L reactor, however, instead were not differentiated in accordance with Stage 4 below, but rather were further differentiated in accordance with Stage 5 as described below and this process and the cells are hereinafter referred to as the “Skip 4 process” and the Skip 4 cells.” For the Skip 4 process, aggregated cell clusters were removed after Stage 3 using a sterile weld and peristaltic pump to a 0.2 L bioreactor (labeled as “Skip 4 ”) to begin Stage 5 medium exposure at 1.05×10⁶ cells/mL.

Stage 4 (3 Days):

At the completion of Stage 3, the spent media was removed and replaced in each bioreactor with 1.5 L of Stage 4 base medium composed of: 1.5 L MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of GlutaMAX™; 2.5 mM glucose; and a 1:200 dilution of ITS-X. The Stage 4 base medium was supplemented with 0.25 μM SANT-1 and 400 nM of TPB. The reactor was maintained at 37° C. and stirred at 70 rpm. Gas and pH were regulated to a dissolved oxygen set point of 30% (air, O2, and N2 regulated) and a pH set point of 7.4 via CO2 regulation. Forty-eight hours after initiation of Stage 4, 3.2 mL/L of a 45% glucose solution (8 mM glucose bolus) was added to the bioreactor and the cells were cultured in the media for an additional 24 hours.

Aggregated cell clusters (150 mL, 0.9×10⁶ viable cells/mL) were removed at the conclusion of the third day of Stage 4 for the Standard process using a sterile weld and peristaltic pump and transferred to a 0.2 L bioreactor (labeled as “Standard”) to begin Stage 5 medium exposure. Additionally, some Stage 4, day 3 cells (45×10⁶ cells/mL) were isolated from the media in a 50 mL conical, then washed 2 times with MCDB-1313 medium containing 1.18 g/L sodium bicarbonate and 0.2% w/v FAF-BSA. The cells were re-suspended in the wash media and held at room temperature for approximately 5 hours and then at 5×10⁶ cells per animal were transplanted under the kidney capsule of NSG mice for assay of in vivo function using human C-peptide detection in response to intra-peritoneal glucose injection following an overnight fast and retro-orbital blood draw 60 minutes after the IP glucose bolus (N=7 animals).

Stages 5 (7 Days):

Following inoculation of cells into the Standard and Skip 4 0.2 L bioreactors, the spent media was removed and replaced with 150 mL of Stage 5+ Base Medium, comprised of MCDB-131 medium base containing 1.18 g/L sodium bicarbonate supplemented with an additional 1.75 g/L sodium bicarbonate; 2% w/v FAF-BSA previously re-constituted in MCDB-131; 1× concentration of GlutaMAX™; 20 mM glucose; 1:200 dilution of ITS-X; 250 μL/L of 1M ascorbic acid; 10 mg/L heparin (Sigma Aldrich; Catalog No. H3149-100KU). This Stage 5 base medium was supplemented with 1 μM T3, 10 μM of 2-(3-(6-methylpyridin-2-yl)-1H-pyrazol-4-yl)-1,5-nathyridine (“ALK5 inhibitor II”), 1μM of gamma secretase inhibitor XXI; 20 ng/mL of betacellulin (R&D Systems, Catalog No. 261-CE-050); 0.25 μM SANT-1; and 100 nM RA. Forty-eight hours after initiation of Stage 5, the spent media was removed and replaced with 150 mL of the same media and supplements. Forty-eight hours later, the medium was removed and replaced with Stage 5+ Base Medium supplemented with 1 μM T3,10 μM ALK5 inhibitor II, 20 ng/mL of betacellulin, and 100 nM RA. Forty-eight hours later the medium was again exchanged and replaced with Stage 5+ Base Medium supplemented with 1 μM T3, 10 μM ALK5 inhibitor II, 20 ng/mL of betacellulin, and 100 nM RA, and cultured for 24 hours to end Stage 5. At the conclusion of the 7 days of Stage 5, cells from each of the Standard and Skip 4 processes were transplanted into the kidney capsule of NSG mice to assay for in vivo function by the method described above.

Throughout the differentiation process cell samples were collected from the suspension cultures for analysis. Samples were analyzed for mRNA expression OpenArray® qRT-PCR and protein expression by flow cytometry. It was observed that moving differentiation directly from Stage 3 medium to Stage 5 medium, the Skip 4 process, resulted in an increased expression of genes associated with islet cells, endocrine hormone expressing cells, and beta cells as compared to cells differentiated in accordance with the Standard process. Using the Skip 4 process, genes associated with alternative gut fates showed lower expression (ALB and CDX2; FIGS. 30B and D), while genes required for endocrine hormone cell formation and function had more expression than found in the Standard process (ABCC8, ARX, CHGA, CHGB, G6PC2, GCG, IAPP, MAFB, NEUROD1, NKX2.2, PAX4, PAX6, PPY and SST as shown in FIGS. 30A, C, E, F, G, H, J, M, O, Q, S, T, X, and AA). Furthermore, genes required for beta cell formation (NKX6.1 and PDX1; FIGS. 30R and W) were expressed at similar levels by the 6^{th} day of Stage 5 for both the Skip 4 and Standard processes cells. Genes required for beta cell function and maintenance (IAPP, INS, ISL1, HB9, PCSK1, PCSK2, SLC30A8, and UNC3; FIGS. 30J, K, L, M, U, V, Z, and BB) or beta cell proliferation (WNT4A, FIG. 30CC) were expressed at similar or higher levels in Skip 4 cells treated with Stage 5 medium.

These data correlated with data that showed higher levels of NGN3 induction (required for endocrine specification) at an earlier time-point in the Skip 4 cells and for a longer period, while PTF1A expression (required for exocrine pancreas) peaked at only 1/20^{th} of the level generated by the Standard process. These results indicate that cells in the Skip 4 reactor were robustly specified to an endocrine pancreas fate in the absence of even a brief induction of PTF1A, suggesting that PTF1A is not required to form beta cells in vitro. This observation is significant as it differs from results seen in the art in which PTF1A was expressed at Stage 4 prior to further differentiation, or the postulated model of development described in U.S. Patent Publication No. 2014/0271566 A1 in which Stage 4 cells are characterized by a PDX1/NKX6.1/PTF1A signature at Stage 4 and then further developed into a beta-like cell in vitro.

The PTF1A expression (FIG. 30Y) cell population present at Stage 4, day 3 had a nearly homogeneous PDX1/NKX6.1 co-expressing population and very few NEUROD1 positive cells (96.2% KX6.1, 99.6% PDX1 and 2.4% NEUROD1 by flow cytometry). The cells were inserted into the kidney capsule of NSG mice (5 million cells/animal; N=7) and over a 16 week period, no human c-peptide in blood sample (data not shown) was detected. This result was unexpected since it has previously been demonstrated in the art that an enriched NXK6.1/PDX1/PTF1A expressing cell population derived in a four stage differentiation process could reverse diabetes within 3 months of engraftment.

When Stage 4 , day 3 (PTF1A expressing) cell were further differentiated through Stage 5 according to the Standard process, the grafts secreted detectable blood levels of human c-peptide by 2 weeks (FIG. 31) and reached >0.5 ng/mL of human c-peptide by 12 weeks after transplant similar to the cells of the Skip 4 process (low/no PTF1A). These data indicate that PTF1A expression is neither necessary nor sufficient to ensure further maturation to a functional beta cell. Rather, the rise of PTF1A expression likely indicates the appearance of an alternative cell population that can be avoided by skipping the Standard Stage 4 and transitioning cells from a medium containing ≧0.5 μM retinoic acid, FGF7, and PKC agonist (TPPB) directly to a medium containing a gamma secretase inhibitor, thyroid hormone (T3), and with or without an ALK5 inhibitor.

These results demonstrate that regulation of pH at Stage 3 to <7.2 can suppress NGN3 expression by at least 80% (see FIG. 26E: B×B and B×C vs. B×D) and promote a PDX1/NKX6.1 co-positive, PTF1A negative cell that may be further directly differentiated into an islet-like cell population containing PDX1/NKX6.1/insulin positive beta-like cells, without passing through a PTF1A positive Stage 4 cell population.

### Example 7

This example demonstrates formation of insulin expressing cells via a five stage differentiation process in a stirred-tank, aseptically closed bioreactor using a low medium pH (<7.2), FGF7, retinoic acid, and a PKC antagonist (TPPB). It was found that use of low pH at Stage 3 eliminated the need to use any sonic hedgehog inhibitor (such as SANT01 or cyclopamine) or TGF-beta/BMP signaling inhibitors or activators at Stage 3 and yielded a population of PDX1 (94%) and NKX6.1 (87%) expressing cells at the end of Stage 4. The Stage 5 reactor population generated from these cells had a high percentage of NEUROD1/NKX6.1 co-positive cells, and insulin positive cells with PDX1 and NKX6.1 co-expression, and this trio (NEUROD1, PDX1, NKX6,1) must be co-expressed with insulin for proper pancreatic beta cell function. Concordantly, when this Stage 5 population of cells was cryo-preserved, thawed and transplanted into the kidney capsule of immune-compromised mice, the graft produced detectable blood levels of human C-peptide within two weeks of engraftment and, on average, >1 ng/mL of C-peptide by four weeks engraftment.

Cells of the human embryonic stem cell line H1 (WA01 cells, WiCell Research Institute, Madison, Wis.) were grown in E8TM medium supplemented with 0.5% w/v FAF-BSA in dynamic suspension for ≧4 passages as round aggregated clusters. The clusters were then frozen as single cells and clusters of 2 to 10 cells per the following method. Approximately 600-1000 million aggregated cells in clusters were transferred to a centrifuge tube and washed using 100mL of 1× DPS −/−. After the wash, the cell aggregates were then enzymatically disaggregated by adding a 30 mL solution of 50% StemPro®Accutase® enzyme and 50% DPBS −/− by volume to the loosened cell aggregate pellet. The cell clusters were pipetted up and down 1 to 3 times and then intermittently swirled for approximately 4 minutes at room temperature, then centrifuged for 5 min, at 80 to 200 ref. The Accutase® supernatant was then aspirated as completely as possible without disturbing the cell pellet. The centrifuge tube was then tapped against a hard surface for approximately 4 minutes, to disaggregate the clusters into single cells and clusters comprised of 2 to10 cells. After 4 minutes, the cells were re-suspended in 100 mL of E8™ media supplemented with 10 μM Y-27632 (Enzo Life Sciences) and 0.5% w/v FAF-BSA, and centrifuged for 5 to12 minutes at 80 to 200rcf. The supernatant was then aspirated and cold (≦4° C.) Cryostor® Cell Preservation Media CS10 was added drop-wise to achieve a final concentration of 100 to 150 million cells per mL. This cell solution was held in an ice bath while being aliquoted to 2 mL cryogenic vials (Corning) after which the cells were frozen using a controlled rate CryoMed™ 34L freezer as follows. The chamber was cooled to 4° C. and the temperature was held until a sample vial temperature reached 6° C. and then the chamber temperature was lowered 2° C. per minute until the sample reached −7° C. at which point the chamber was cooled 20° C./min. until the chamber reached −45° C. The chamber temperature was then allowed to briefly rise at 10° C./min. until the temperature reached −25° C., and then the chamber was cooled further at 0.8° C./min. until the sample vial reached −40° C. The chamber temperature was then cooled at 10° C./min. until the chamber reached −100° C. at which point the chamber was then cooled 35° C./min. until the chamber reached −160° C. The chamber temperature was then held at −160° C. for at least 10 minutes, after which the vials were transferred to gas phase liquid nitrogen storage. These cryo-preserved single cells at high density were then used as an ISM.

ISM vials were removed from the liquid nitrogen storage, thawed, and used to inoculate a 3 liter glass, stirred suspension tank bioreactor (DASGIP) at a seeding density of 0.295 million viable cells per mL. The vials were removed from liquid nitrogen storage and quickly transferred to a 37° C. water bath for 120 seconds to thaw. The vials were then moved to a BSC and the thawed contents transferred via 2 mL glass pipette to a 50 mL conical tube. Then 10 mL of E8™ medium supplemented with 0.5% w/v FAF-BSA and 10 μM of Rho kinase inhibitor Y-27632, were added to the tube in a drop-wise manner. The cells were centrifuged at 80-200 rcf for 5 min. The supernatant from the tube was aspirated and 10 mL fresh E8™ medium supplemented with 0.5% w/v FAF-BSA and 10 μM Y-27632 were added and the volume containing the cells was pipetted into a media transfer bottle (Cap2V8®, Sanisure, Inc) containing 450 mL E8™ media supplemented with 0.5% w/v FAF-BSA and 10 μM Y-27632. The bottle contents were then pumped directly into the bioreactor via a sterile, C-Flex® tubing weld using a peristaltic pump. The bioreactor was prepared with 1000 mL E8™ medium supplemented with 0.5% w/v FAF-BSA and 10 μM Y-27632 pre-warmed to 37° C., stirred at 70 rpm, with a dissolved oxygen set point of 30% (air O₂, and N₂ regulated), and a controlled CO₂ partial pressure of 5% . The reactor was inoculated to give a target concentration of 0.225×10⁶ cells/mL (concentration range: 0.2 to 0.5×10⁶ cells/mL).

Once the reactor was inoculated, the cells formed round aggregated clusters in the stirred reactor. After 24 hours in culture, the medium was partially exchanged as more than 80% of the original volume was removed and 1.5 L of E8™ medium supplemented with 0.5% w/v FAF-BSA was added back (fresh medium). This media exchange process was repeated 48 hours after inoculation. After three days in suspension culture as round aggregated clusters, differentiation in the 3 liter reactor was initiated by removing the spent E8™ medium and adding differentiation medium. The differentiation protocol is described below.

Stage 1 (3 Days):

The reactor was set to a temperature of 37° C. and stirred continuously at 70 rpm. Gas and pH controls were set to a dissolved oxygen set point of 10% (air, O2, and N2 regulated), and the pH was set to 7.4 via CO2 regulation. A Stage 1 base medium was prepared using 1.5 L MCDB-131 medium containing 1.18 g/L sodium bicarbonate; supplemented with an additional 2.4 g/L sodium bicarbonate, 2% w/v FAF-BSA, previously re-constituted in MCDB-131; 1× concentration of G1utaMAXTM; 2.5 mM glucose (45% in water); and a 1:50,000 dilution of ITS-X. Cells were cultured for one day in 1.5 L of the Stage 1 base medium supplemented with 100 ng/ml GDF8 and 2 μM of MCX compound. After 24 hours, a media exchange was completed as described above, and fresh 1.5 L of base medium supplemented with 100 ng/mL of GDF8 were added to the reactor. Cells were maintained without further media exchange for 48 hours.

Stage 2 (3 Days):

The reactor was set to a temperature of 37° C. and stirred continuously at 70 rpm. Gas and pH controls were set to a dissolved oxygen set point of 30% (air O2, and N2 regulated), and the pH was set to 7.4 via CO2 regulation. After the completion of Stage 1, a media exchange was completed as described above, whereby the spent Stage 1 media was removed and replaced with the 1.5 L of the same medium used as the Stage 1 base medium, but supplemented with 50 ng/mL FGF7. Forty-eight hours after the media exchange, the spent media was again removed and replaced with 1.5 L fresh base medium supplemented with 50 ng/mL FGF7.

Stage 3 (3 Days):

At the completion of Stage 2, and just prior to medium exchange, the cells were counted, gravity settled and re-suspended in the following Stage 3 base medium at a normalized concentration of 2.0 million cells/mL in 1.5 liters: 1.5 L MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of GlutaMAX™; 2.5 mM glucose; and a 1:200 dilution of ITS-X. The Stage 3 base medium was supplemented with 50 ng/mL FGF-7; 1 μM RA; and 400 nM of TPB. The reactor was set to a temperature of 37° C. and stirred continuously at 70 rpm. Gas and pH controls were set to a dissolved oxygen set point of 30% (air O2, and N2 regulated), and 7.0 pH via CO2 regulation. Twenty-four hours post media exchange, the spent media was again replaced with 1.5 L fresh Stage 3 base medium containing the above supplements. Cells were thereafter cultured in the media for 48 hours, until the end of Stage 3.

Stage 4 (3 Days):

At the completion of Stage 3, the spent media was removed and replaced in each bioreactor with 1.5 L of Stage 4 base medium composed of: 1.5 L MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of GlutaMAX™; 2.5 mM glucose; and a 1:200 dilution of ITS-X. The Stage 4 base medium was supplemented with 0.25 μM SANT-1 and 400 nM of TPB. The reactor was maintained at 37° C. and stirred at 70 rpm. Gas and pH were regulated to a dissolved oxygen set point of 30% (air, O2, and N2 regulated) and a pH set point of 7.4 via CO2 regulation. Forty-eight hours after initiation of Stage 4, 3.2 mL/L of a 45% glucose solution (8mM glucose bolus) was added to the bioreactor and the cells were cultured in the media for an additional 24 hours.

Stages 5 (8 Days):

At the conclusion of the third day of Stage 4, the spent media was removed and replaced 1.5 L of Stage 5 base medium composed of: 1.5 L of MCDB-131 medium containing 1.18 g/L sodium bicarbonate supplemented with an additional 1.75 g/L sodium bicarbonate; 2% w/v FAF-BSA previously re-constituted in MCDB-131; 1× concentration of GlutaMAX™; 20 mM glucose; 1:200 dilution of ITS-X; 250 μL/L of 1M ascorbic acid; 10 mg/L heparin. For the first feeding, the Stage 5 base medium was supplemented with 1 μM T3 as 3,3′,5-Triiodo-L-thyronine sodium salt, 10 μM of ALK5 inhibitor II, 1 μM of the gamma secretase inhibitor, XXI; 20 ng/mL of betacellulin; 0.25 μM SANT-1; and 100 nM RA. 48 hours after beginning Stage 5, the spent media was removed and replaced with 1.5 L of the same fresh media and supplements. Forty-eight hours later, the medium was removed and replaced with Stage 5 base medium supplemented with 1 μM T3,10 μM ALK5 inhibitor II, 20 ng/mL of betacellulin, and 100 nM RA. Forty-eight hours later the medium was again exchanged and replaced with Stage 5 base medium supplemented with 1 μM T3, 10 μM ALK5 inhibitor II, 20 ng/mL of betacellulin, and 100 nM RA, and cultured for 48 hours to end Stage 5.

At the conclusion of the eighth day of Stage 5 (48 hours after the last feeding) aggregated cell clusters were removed from the reactor via sterile weld and peristaltic pump and centrifuged into a pellet. In order to cryopreserve the cells, they were transferred to cryopreservation media comprised of 57.5% MCDB131 with 2.43 g/L sodium bicarbonate, 30% Xeno-free KSR, 10% DMSO, and 2.5% HEPES (final concentration 25 mM). Once the cell clusters were suspended in cryopreservation media at ambient temperature the cryo-vials were moved to the controlled rate freezer (CRF) within 15 minutes. The chamber temperature was then reduced to 4° C. for 45min, and further reduced by 2.00° C./min to −7.0° C. (sample). The sample was then quickly cooled, reducing the temperature of the chamber at a rate of 25.0° C. /min to −45.0° C. A compensation increase was then provided by increasing the chamber temp 10.0° C./min to −25.0° C. (chamber). The sample was then cooled at 0.2° C./min until the temperature reached −40.0° C. The chamber was then cooled to −160° C. at a rate of 35.0° C./min and held at that temperature for 15 minutes. The samples were moved to a gas phase liquid nitrogen storage container at the termination of the CRF run.

After the cells had been stored in gas phase liquid nitrogen the cells were thawed by removal from storage and transferred to a 37° C. water bath. The vial was gently swirled in the water bath for less than 2 minutes until a small ice crystal remained in the vial. The vial contents were then transferred to a 50 ml conical and diluted drop-wise over two minutes using MCDB131 media with 2.43 g/L sodium bicarbonate and 2% BSA to a final volume of 20 ml total. The total cell number was then determined by Nucleocounter®. The cells were then isolated from the media in a 50 ml conical, the supernatant removed and cells re-suspended in fresh MCDB131 media with 2.43 g/L sodium bicarbonate and 2% BSA and transferred to a 125 mL Corning® spinner flask filled to a volume of 75mL with a cell concentration of 1 million cells per mL. The cells were maintained overnight in a humidified, 5% CO2 incubator stirred at 55 RPM, and the next day the cells were analyzed by flow cytometry. The cells were greater than 50% NKX6.1/NEUROD1 co-positive (FIG. 32), greater than 80% NKX6.1/NEUROD1 co-positive (FIG. 33) and at least 35% NKX6.1/insulin co-positive after thaw (FIG. 34) in three replicates. Furthermore, when these cells were transplanted under the kidney capsule of NSG mice (5 million cells per dose; N=7), all animals had detectable levels of C-peptide and they secreted, by mean average, >1 ng/mL of C-peptide within 4 weeks of implantation. At 6 weeks post implant, 5 of 7 grafted animals showed glucose responsive insulin (human C-peptide) secretion greater than unstimulated levels (FIG. 35), and by 12 weeks all 7 animals showed glucose responsive insulin (human C-peptide) secretion (FIG. 36).

These data indicate that NKX6.1/insulin co-expressing cells can be generated using pH and dissolved oxygen modulation at Stage 3 to eliminate the need for proteins or small molecules to block TGF-beta/BMP or sonic hedgehog signaling while also maximizing the yield of NKX6.1/PDX1 positive cells at Stage 4 which may be further differentiated to NEUORD1/NKX6.1/PDX1/insulin co-expression via a fifth stage in a stirred tank reactor. The cells may be cryopreserved, thawed, and implanted and will function in vivo as measured by glucose induced insulin secretion (>1 ng/mL C-peptide) within 4 weeks of implantation and demonstrate glucose responsiveness by 12 weeks after implantation.

### Example 8

This example demonstrates formation of insulin expressing cells in a stirred suspension culture using 3 L disposable spinner flasks. Media and gases were exchanged through removable, vented side arm caps. The insulin positive cells were formed in a step-wise process in which cells first expressed PDX1 and then also co-expressed NKX6.1. These co-expressing cells then gained expression of insulin and later MAFA, in combination with PDX1 and NKX6.1 while in suspension culture.

Cells of the human embryonic stem cell line H1 (WA01 cells, WiCell Research Institute, Madison, Wis.) were grown in adherent culture conditions in mTeSR1™ medium using Matrigel™ as an attachment matrix for 4 passages, continuously expanded into larger vessels. The cells were seeded into multiple 5 layer cell stacks (“C55”) on the 4^{th} passage. 72 hours after passage, the cells confluency in each CS5 reached 70-80%. The spent media was removed and the cells were washed with PBS. 300 mL of Versene™ pre-warmed to 37° C. were then added to the cells and the cells were then incubated at 37° C. (5% CO₂) for 8.5 minutes. After the incubation time, EDTA was carefully removed from the flask leaving approximately 50 mL of residual Versene™ in the flask. The cell layers were then allowed to continue incubating for 3 minutes with residual Versene™ while undergoing intermittent tapping of the vessel to dislodge cell clusters. After 3 minutes of this residual incubation, 250 mL mTeSR1™ containing 10 μM Y-27632 (Enzo Life Sciences) were added to the flask to quench the cell dissociation process and collect the lifted cell clusters. The wash media was then transferred to a round bottle and the CS5 was washed with an additional 150 mL mTeSR1™ containing 150 μM Y-27632 and pooled with the first wash. 200 million cells were then transferred to a non-coated, but tissue culture treated CS1 and additional media was supplemented to obtain a final volume of 200 mL with a cell density of 1 million cells per mL.

The CS1 containing the lifted cells were incubated at 37° C. for 2 hours. Using closed-loop C-flex tubing with pump tubing attached between 2 CELI stack ports, the cell suspension was triturated for 5 minutes at 75 rpm by peristaltic pump to homogenize the aggregates. The pump tubing assembly was then replaced with 0.2 μM vented caps and returned to a 37° C. incubator for overnight incubation of between 12 and 22 hours. After incubation, the cells formed rounded, spherical aggregated clusters of pluripotent cells.

Three CS 1 vessels, 600 mL of containing the newly formed clusters were each then transferred to a 3 L disposable spinner flask with an additional 1200 mL of fresh, pre-warmed mTeSR1™ containing 10 μM Y-27632 with a resulting cell density of approximately 0.3 million cells per mL. The spinner flasks were then incubated at 37° C. and an agitation rate of 40 rpm. After 24 hours of incubation, the cells were removed from the agitation and the clusters were allowed to settle to the bottom of the flask for 8 minutes, after which 1.5 L of spent media was aspirated from the top avoiding the clusters sitting on the bottom of the vessel. 1.5 mL of fresh mTeSR1™ media was added to the cells and they were placed back in the incubator at 40 rpm for an additional 24 hours of growth. At the end of 72 hours, the pluripotent clusters were transitioned to differentiation media. The differentiation protocol is described below.

Stage 1 (3 Days):

Each of 4 spinner flasks was transferred from dynamic suspension to the incubator in a BSC without agitation. A complete media exchange, as described below, was performed to ensure that only residual, spent media carried over to the new media. In order to perform a complete media exchange, the clusters were allowed to settle to the bottom of the flask for 8 minutes. The spent media was then removed using a vacuum aspiration starting from the top of the liquid until only 300 mL remained. The remaining cell volume was transferred to 150 mL conical tubes and centrifuged at 800 rpm for 3 minutes. Using a vacuumed aspiration system, remaining spent media was removed without disruption of the cell cluster pellets. The pellets were then re-suspended in 1.8 L of basal media containing 1.5 L MCDB-131 medium containing 1.18 g/L sodium bicarbonate; supplemented with an additional 2.4 g/L sodium bicarbonate, 2% w/v FAF-BSA, previously re-constituted in MCDB-131; 1X concentration of GlutaMAX™; 2.5 mM glucose (45% in water); and a 1:50,000 dilution of ITS-X. Cells were cultured for one day in 1.8 L of the Stage 1 base medium supplemented with 1.8 ml GDF8 and 540 μL of MCX compound. Cell counts were taken to confirm a starting density of 0.5 million cells per mL at the start of differentiation. The flasks were then placed back in the incubator on spinner plates at 2 speeds per condition as shown in Table XVII below. The spinner flasks were incubated overnight.

TABLE XVII
Conditions Used Throughout Differentiation

| | Lifting Agent During | Agitation Rate During |
|---|---|---|
| Condition | Cluster Formation | Differentiation |
| A | EDTA | 27 rpm |
| B | EDTA | 33 rpm |
| C | Accutase | 27 rpm |
| D | Accutase | 33 rpm |

After approximately 24 hours, a media exchange was completed to remove approximately 90% off the spent media and replace with fresh 1.8 L of base medium supplemented with 1.8 mL of GDF8. To perform the media exchange, clusters were allowed to settle to the flask bottom for 8 minutes and the spent media was removed using vacuum aspiration until only 300 mL remained. The remaining cells were transferred into a 250 mL circular bottle and the clusters allowed to settle for 6 minutes after which media was removed using a pipette to ensure only 180 mL of media containing cells was left to ensure no more than 10% of the residual spent media was transferred over to the next feed. The remaining cells and media were then returned to a spinner flask with 1.8 L of fresh media and allowed to incubate for 48 hours.

Stage 2 (3 Days):

A complete media exchange, as described above, was performed to remove all Stage 1 spent media and transfer the cells into 1.8 L of the same medium used as the Stage 1 base medium, but supplemented with 1.8 mL FGF7. The flasks were then returned to the incubator and allowed to stay in dynamic agitation for 48 hours without media exchange, after which the spent media was again removed leaving 180 mL of spent media and adding 1.8 L fresh base medium supplemented with 1.8 mL FGF7. The cells were then incubated for 24 hours.

Stage 3 (3 Days):

At the completion of Stage 2, a complete media exchange was performed to remove all Stage 2 media and transfer cells to 1.5 L medium: 1.5 L MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of G1utaMAXTM; 2.5 mM glucose; and a 1:200 dilution of ITS-X. The Stage 3 base medium was supplemented with 1.5 mL FGF-7; 75 μL RA; and 120 uL TPB. The media was prepared under “dark conditions.” The total volume of the flask was reduced from 1.8-2.0 L to 1.5-1.65 L to target a cell density of approximately 1.5-2 million cells per mL. The flasks were incubated for 24 hours, after which a media exchange was performed leaving behind 150 mL of spent media and adding h 1.5 L fresh Stage 3 base medium containing the above supplements. Cells were thereafter cultured in the media for 48 hours, until the end of Stage 3.

Stage 4 (3 Days):

At the completion of Stage 3, a complete media exchange was performed and transfer the cells into 1.5 L of Stage 4 base medium composed of: 1.5 L MCDB-131 medium containing 1.18 g/L sodium bicarbonate supplemented with an additional 2.4 g/L sodium bicarbonate; 2% w/v FAF-BSA, previously re-constituted in MCDB-131; 1× concentration of GlutaMAX™; 2.5 mM glucose; and a 1:200 dilution of ITS-X. The Stage 4 base medium was supplemented with 150 μL SANT-1 and 120 μL of TPB. The flasks were then returned to the incubator and allowed to stay in dynamic agitation for 48 hours without media exchange. At the end of 48 hours, 5.28 mL of a 45% D-glucose solution was added to the spinner and the flasks were returned to incubation for an additional 24 hours.

Stages 5 (3 Days):

At the conclusion of the third day of Stage 4, the spent media was removed and replaced 1.5 L of Stage 5 base medium composed of: 1.5 L of MCDB-131 medium containing 1.18 g/L sodium bicarbonate supplemented with an additional 1.75 g/L sodium bicarbonate; 2% w/v FAF-BSA previously re-constituted in MCDB-131; 1× concentration of GlutaMAX™; 20 mM glucose; 1:200 dilution of ITS-X; 250 L/L of 1M ascorbic acid; 10 mg/L heparin. The Stage 5 base medium was supplemented with 1 82 M T3 as 3,3′,5-Triiodo-L-thyronine sodium salt, 10 μM of ALK5 inhibitor II, 1 μM of the gamma secretase inhibitor, XXI; 20 ng/mL of betacellulin; 0.25 μM SANT-1; and 100 nM RA. 48 hours after beginning Stage 5, the spent media was removed and replaced with 1.5 L of the same fresh media and supplements. 48 hours later, the medium was removed and replaced with Stage 5 base medium supplemented with 1 μM T3,10 μM ALK5 inhibitor II, 20 ng/mL of betacellulin, and 100 nM RA and differentiation was continued for 48 hours until the conclusion of Stage 5.

At the conclusion of Stage 5 aggregated cell clusters were allowed to settle to the bottom of the flask for 8 minutes and the media was removed using vacuum aspiration until about 300 mL liquid remained. The remaining cell volume was transferred to 150 mL conical tubes and centrifuged at 800 rpm for 3 minutes followed by removal of the remaining spent media. The cell pellet was re-suspended in wash media, basal MCDB1313. The cells were again spun down at 800 rpm for 5 minutes. In order to cryopreserve the cells, they were transferred to cryopreservation media comprised of 57.5% MCDB131 with 2.43g/L sodium bicarbonate, 20% Xeno-free KSR, 10% DMSO, and 2.5% HEPES (final concentration 25 mM). Once the cell clusters were suspended in cryopreservation media at ambient temperature the cryo-vials were moved to the controlled rate freezer (CRF) within 15 minutes. The chamber temperature was then reduced to 4° C. for 45 min, and further reduced by 2.00° C./min to −7.0° C. (sample). The sample was then quickly cooled, reducing the temperature of the chamber at a rate of 25.0° C. /min to −45.0° C. A compensation increase was then provided by increasing the chamber temp 10.0° C./min to −25.0° C. (chamber). The sample was then cooled at 0.2° C. /min until the temperature reached −40.0° C. The chamber was then cooled to −160° C. at a rate of 35.0° C. /min and held at that temperature for 15 minutes. The samples were moved to a gas phase liquid nitrogen storage container at the termination of the CRF run.

After the cells had been stored in gas phase liquid nitrogen three vials of the cells were thawed by removal from storage and transferred to a 37° C. water bath. The vial was gently swirled in the water bath for less than 2 minutes until a small ice crystal remained in the vial. The vial contents were then transferred to a spinner flask and 10 mL of thaw media was added in a drop-wise fashion while continuously mixing the spinner by hand using MCDB131 media supplemented to attain a final concentration of 1.6 g/L sodium bicarbonate, 8 mM glucose, lx ITS-X, and 2% BSA. After all three vials were thawed, additional thaw media was added to reach a target volume of approximately 80 mL. The spinner flask was then incubated in a humidified incubator with 5% CO₂ overnight (16-24 hours) and under gentle agitation of 38-40 rpm. The next day, the cells were washed as follows. The spinners were allowed to settle in the hood for 6 minutes and approximately 75 mL of spent media was aspirated while the remaining cell suspension was transferred to a 50 mL conical tube using a 10 mL glass pipette and subsequently centrifuged at 600 rpm for 3 minutes. The supernatant was aspirated and cell pellet re-suspended in 10 mL wash media after which the cells were re-centrifuged at 600 rpm for 3 minutes. After aspiration and re-suspension of the cell pellet in 10 mL of wash media, the pellet was transferred back to the spinner flask into which 60 mL of wash media was added. The flask was then placed on a spin plate in a BSC and samples were collected from a homogeneous well mixed spinner to obtain cell recovery as well as collect cells for analysis and transportation.

FIG. 37A and 37B depict the pH profile off the culture media within the spinner flasks. The pH of the media is regulated by the CO₂ in the incubator (setpoint, 5%) and the metabolic activity, specifically lactate production of the cells depicted in FIG. 38. It is shown that the cultures with the lowest pH environments, specifically condition A, also had the highest lactate concentrations. As seen in FIGS. 37A and B, the pH of all spinners during Stage 2 ranged between about 6.8 and 7.2 and about 7.0 and 7.2 throughout Stage 3. After the completion of Stage 3, it was observed that nearly all cells expressed both endoderm transcription factor FOXA2 and the pancreatic specific transcription factor PDX1. At least 50% were also detected to express NKX6.1 with a small population being NEUOD1 positive. Another 48 hours after Stage 3, completion of Stage 4, day 2, the NKX6.1 population increased to about 65% of population, which were originally lifted with Accutase (conditions C and D) and approximately 70-75% of the population of cells originally lifted with EDTA as shown on Table XVIII.

TABLE XVIII

| | | | | | | NKX6.1/NEUROD1 |
|---|---|---|---|---|---|---|
| | | FOXA2 | PDX1 | NKX6.1 | NEUROD1 | Copositive |
| Stage 3 | Condition A | 99.8 | 99.6 | 66.2 | 2.8 | 1.1 |
| | (EDTA 27) | | | | | |
| Stage 3 | Condition B | 99.7 | 98.4 | 61.7 | 4.1 | 1.3 |
| | (EDTA 33) | | | | | |
| Stage 3 | Condition C | 99.1 | 98.1 | 49.8 | 2.5 | 0.5 |
| | (Accutase | | | | | |
| | 27) | | | | | |
| Stage 3 | Condition D | 99.1 | 97.7 | 55.6 | 4.9 | 1.2 |
| | (Accutase | | | | | |
| | 33) | | | | | |
| Stage 4 | Condition A | 99.5 | 99.3 | 73.5 | 23.8 | 5.5 |
| | (EDTA 27) | | | | | |
| Stage 4 | Condition B | 99.3 | 97.8 | 73.7 | 24 | 6.5 |
| | (EDTA 33) | | | | | |
| Stage 4 | Condition C | 98.5 | 94.5 | 68.9 | 19.9 | 4.1 |
| | (Accutase | | | | | |
| | 27) | | | | | |
| Stage 4 | Condition D | 98.0 | 91.5 | 65.9 | 20.1 | 4.9 |
| | (Accutase | | | | | |
| | 33) | | | | | |

Upon completion of the 6 days of Stage 5, the cells were again analyzed by flow cytometry prior to being cryopreserved.

TABLE XIX
Stage 5 Protein Expression

| | | | | | (Ins) | (C-Peptide) | |
|---|---|---|---|---|---|---|---|
| | | | | | NKX6.1/C- | NKX6.1/C- | |
| | | | | NKX6.1/NEUROD1 | Peptide | Peptide | |
| | | PDX1 | NKX6.1 | Copositive | Copositive | Copositive | PAX6 |
| Stage 5 | Condition | 96.0 | 80.8 | (82.6); 70.4 | (34.5); 26.6 | (35.9); 26.4 | 68.7 |
| | A (EDTA | | | | | | |
| | 27) | | | | | | |
| Stage 5 | Condition | 93.6 | 82.1 | (74.9); 66.1 | (39.3); 30.7 | (39.6); 30.6 | 62.3 |
| | B (EDTA | | | | | | |
| | 33) | | | | | | |
| Stage 5 | Condition | 94.4 | 84.3 | (74.0); 65.3 | (33.0); 27.1 | (32.9); 26.2 | 60.0 |
| | C (Accutase | | | | | | |
| | 27) | | | | | | |
| Stage 5 | Condition D | 92.9 | 79.1 | (75.4); 63.5 | N/A | (34.3); 26.9 | 60.9 |
| | (Accutase | | | | | | |
| | 33) | | | | | | |

Thawed cells were evaluated by flow cytometry for comparison to the fresh (pre-cryo-preserved) analysis as shown in Table XX. Cell recovery was assessed by comparing the final cell population to the original population upon thaw, t=0. Cell, viability was qualitatively assessed through LIVE/DEAD fluorescence imaging, as shown in FIG. 39 and compared to that at t=0.

TABLE XX

| | | | | | | (C-PEPTIDE) | |
|---|---|---|---|---|---|---|---|
| | | | | (NEUROD1) | (Chromogranin) | NKX6.1/C- | |
| | | | | NKX6.1/NEUROD1 | NKX6.1/CHG | PEP. | |
| | | PDX1 | NKX6.1 | Copositive | Copositive | Copositive | PAX6 |
| Condition | 24HAT¹* | 87.3 | 72.0 | (77.4) 59.8 | (74.0) 54.0 | (32.6) 22.1 | 26.8 |
| A (EDTA | | | | | | | |
| 27) | | | | | | | |
| Condition | 24HAT² | 83.5 | 77.4 | (82.6) 63.1 | N/A | (29.6) 19.9 | 41.8 |
| A (EDTA | | | | | | | |
| 27) | | | | | | | |
| Condition | 24HAT³ | 82.1 | 73.4 | (74.0) 58.9 | N/A | (30.7) 17.4 | 48 |
| A (EDTA | | | | | | | |
| 27) | | | | | | | |
| Condition B | 24HAT¹ | 89.4 | 77.4 | (75.9) 62.8 | (72.4) 56.6 | (33.5) 25.6 | 33.3 |
| (EDTA 33) | | | | | | | |
| Condition B | 24HAT² | 79.8 | 80 | (80.8) 66.1 | N/A | (27.6) 20.2 | 32.3 |
| (EDTA 33) | | | | | | | |
| Condition C | 24HAT¹ | 82.2 | 68.7 | (68.1) 53.3 | (64.8) 46.6 | (24.2) 17.9 | 17.6 |
| (Accu. 27) | | | | | | | |
| Condition C | 24HAT² | 84.0 | 82.2 | (70.0) 62.0 | N/A | (25.6) 20.9 | 24.3 |
| (Accu. 27) | | | | | | | |
| Condition D | 24HAT¹ | 89.6 | 71.4 | (72.8) 58.6 | (70.1) 52.8 | (40.8) 32.1 | 28.5 |
| (Accu. 33) | | | | | | | |
| Condition D | 24HAT² | 70.3 | 72.3 | (72.4) 53.2 | N/A | (24.7) 17.2 | 42.1 |
| (Accu. 33) | | | | | | | |

*“24HAT” means 24 hours after thaw and the superscripts refer to run numbers.

While the invention has been described and illustrated herein by reference to various specific materials, procedures and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

What is claimed is:

1. A method for differentiation human pluripotent cells, comprising the steps of:
differentiating foregut endoderm cells to pancreatic endoderm cells by culturing the foregut endoderm cells in a dynamic suspension culture at a pH of about 7.2 to about 7.0 for at least about 24 hours.

2. The method of claim 1, further comprising culturing the foregut endoderm cells in culture having a cell concentration of equal to or greater than about 1.5 million cells/mL.

3. The method of claim 1, further comprising culturing the foregut endoderm cells in culture having a cell concentration of greater than or equal to about 2.0 million cells/mL.

4. The method of claiml, wherein the pancreatic endoderm cells are substantially negative for the expression of PTF1A and NGN3.

5. The method of claim 4, further comprising enriching the pancreatic endoderm cells that are substantially negative for the expression of PTF1A and NGN3 to a population of pancreatic endoderm cells having greater than or equal to about 96% cells that are positive for co-expression of PDX1 and NKX6.1 and that are positive for expression of PTF1A.

6. The method of claim 4, further comprising differentiating the pancreatic endoderm cells that are substantially negative for the expression of PTF1A and NGN3 to pancreatic endocrine in the absence of a differentiation stage in which cells positive for PTF1A expression are produced.

7. A method for differentiation human pluripotent cells, comprising the steps of:
differentiating foregut endoderm cells to pancreatic endoderm cells by culturing the foregut endoderm cells in a dynamic suspension culture at a pH of about 7.2 to about 7.0 for at least about 24 hours, a cell concentration of equal to or greater than about 1.5 million cells/mL, and a retinoid concentration of about 0.5 to about 1.0 wherein the culturing is carried out in the absence of components to one or more of inhibit, block, activate or agonize TGF-beta signaling and BMP signaling and a sonic hedgehog signaling pathway inhibitor.
